(19) United States
(12) Patent Application Publication
(10) Pub. No.: US 2025/0115572 A1
(43) Pub. Date: Apr. 10, 2025

(54) SPIROCYCLIC PIPERIDINYL DERIVATIVES AS COMPLEMENT FACTOR B INHIBITORS AND USES THEREOF

(71) Applicant: Novartis AG, Basel (CH)

(72) Inventors: Christopher M. ADAMS, Arlington, MA (US); Lara C. CZABANIUK, Arlington, MA (US); Stefanie FLOHR, Lörrach (DE); François GESSIER, Altkirch (FR); Michael Christoph HEDIGER, Rheinfelden (CH); Konstanze HURTH, Lörrach (DE); Nancy LABBE-GIGUERE, Arlington, MA (US); Fabio LIMA, Saint Louis (FR); Tajesh PATEL, Westford, MA (US); Oliver SIMIC, Basel (CH); Martin SENDZIK, Belmont, MA (US); Angela VITREY, Rosenau (FR); Anna VULPETTI, Basel (CH)

(21) Appl. No.: 18/729,949

(22) PCT Filed: Jan. 20, 2023
(86) PCT No.: PCT/IB2023/050504

§ 371 Date: Jul. 18, 2024

Related U.S. Application Data
(60) Provisional application No. 63/302,246, filed on Jan. 24, 2022.

(51) Int. Cl. C07D 401/06; A61K 31/438; A61K 31/444; C07D 401/14; C07D 405/14

(52) U.S. Cl. CPC C07D 401/06; A61K 31/438; A61K 31/444; C07D 401/14; C07D 405/14

(57) ABSTRACT

Provided herein are compounds of formula (I) and pharmaceutical compositions thereof useful for treating diseases or disorders mediated by the complement factor B. (I).

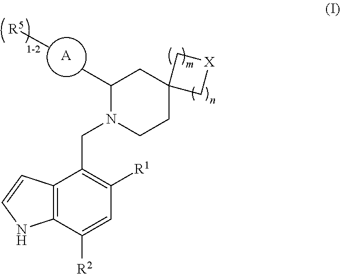

### FIELD OF THE DISCLOSURE

The disclosure relates to the inhibition of the complement alternative pathway and particularly to inhibition of Factor B, in patients suffering from conditions and diseases associated with complement alternative pathway activation such as age-related macular degeneration, diabetic retinopathy and related ophthalmic diseases.

### BACKGROUND OF THE DISCLOSURE

The complement system is a crucial component of the innate immunity system and comprises a group of proteins that are normally present in an inactive state. These proteins are organized in three activation pathways: the classical, the lectin, and the alternative pathways (V. M. Holers, In Clinical Immunology: Principles and Practice, ed. R. R. Rich, Mosby Press; 1996, 363-391). Molecules from microorganisms, antibodies or cellular components can activate these pathways resulting in the formation of protease complexes known as the C3-convertase and the C5-convertase. The classical pathway is a calcium/magnesium-dependent cascade, which is normally activated by the formation of antigen-antibody complexes. It can also be activated in an antibody-independent manner by the binding of C-reactive protein complexed to ligand and by many pathogens including gram-negative bacteria. The alternative pathway is a magnesium-dependent cascade which is activated by deposition and activation of C3 on certain susceptible surfaces (e.g., cell wall polysaccharides of yeast and bacteria, and certain biopolymer materials).

Factor B may be a suitable target for the inhibition of this amplification of the complement pathways because its plasma concentration in humans is typically about 200 μg/mL (or about 2 μM), and it has been shown to be a critical enzyme for activation of the alternative complement pathway (P. H. Lesavre and H. J. Müller-Eberhard. J. Exp. Med., 1978; 148: 1498-1510; J. E. Volanakis et al., New Eng. J. Med., 1985; 312:395-401).

Macular degeneration is a clinical term that is used to describe a family of diseases that are characterized by a progressive loss of central vision associated with abnormalities of Bruch's membrane, the choroid, the neural retina and/or the retinal pigment epithelium. In the center of the retina is the macula lutea, which is about % to % cm in diameter. The macula provides detailed vision, particularly in the center (the fovea), because the cones are higher in density and because of the high ratio of ganglion cells to photoreceptor cells. Blood vessels, ganglion cells, inner nuclear layer and cells, and the plexiform layers are all displaced to the side (rather than resting above the photoreceptor cells), thereby allowing light a more direct path to the cones. Under the retina is the choroid, a part of the uveal tract, and the retinal pigmented epithelium (RPE), which is between the neural retina and the choroid. The choroidal blood vessels provide nutrition to the retina and its visual cells.

Age-related macular degeneration (AMD), the most prevalent form of macular degeneration, is associated with progressive loss of visual acuity in the central portion of the visual field, changes in color vision, and abnormal dark adaptation and sensitivity. Two principal clinical manifestations of AMD have been described as the dry, or atrophic, form and the neovascular, or exudative, form. The dry form is associated with atrophic cell death of the central retina or macula, which is required for fine vision used for activities such as reading, driving or recognizing faces. About 10-20% of these AMD patients progress to the second form of AMD, known as neovascular AMD (also referred to as wet AMD).

Neovascular AMD is characterized by the abnormal growth of blood vessels under the macula and vascular leakage, resulting in displacement of the retina, hemorrhage and scarring. This results in a deterioration of sight over a period of weeks to years. Neovascular AMD cases originate from Intermediate or advanced dry AMD. The neovascular form accounts for 85% of legal blindness due to AMD. In neovascular AMD, as the abnormal blood vessels leak fluid and blood, scar tissue is formed that destroys the central retina.

The new blood vessels in neovascular AMD are usually derived from the choroid and are referred to as choroidal neovascularizaton (CNV). The pathogenesis of new choroidal vessels is poorly understood, but such factors as inflammation, ischemia, and local production of angiogenic factors are thought to be important. A published study suggests that CNV is caused by complement activation in a mouse laser model (Bora P. S., J. Immunol. 2005; 174; 491-497).

Human genetic evidence implicates the involvement of the complement system, particularly the alternative pathway, in the pathogenesis of Age-related Macular Degeneration (AMD). Significant associations have been found between AMD and polymorphisms in complement factor H (CFH) (Edwards A O, et al. Complement factor H polymorphism and age-related macular degeneration. Science. 2005 Apr. 15; 308(5720):421-4; Hageman G S, et al A common haplotype in the complement regulatory gene factor H (HF1/CFH) predisposes individuals to age-related macular degeneration. Proc Natl Acad Sci USA. 2005 May 17; 102(20):7227-32; Haines J L, et al. Complement factor H variant increases the risk of age-related macular degeneration. Science. 2005 Apr. 15; 308(5720):419-21; Klein R J, et al Complement factor H polymorphism in age-related macular degeneration. Science. 2005 Apr. 15; 308(5720):385-9; Lau L I, et al. Association of the Y402H polymorphism in complement factor H gene and neovascular age-related macular degeneration in Chinese patients. Invest Ophthalmol Vis Sci. 2006 August; 47(8):3242-6; Simonelli F, et al. Polymorphism p.402Y>H in the complement factor H protein is a risk factor for age related macular degeneration in an Italian population. Br J Ophthalmol. 2006 September; 90(9):1142-5; and Zareparsi S, et al Strong association of the Y402H variant in complement factor H at 1q32 with susceptibility to age-related macular degeneration. Am J Hum Genet. 2005 July; 77(1):149-53.), complement factor B (CFB) and complement C2 (Gold B, et al. Variation in factor B (BF) and complement component 2 (C2) genes is associated with age-related macular degeneration. Nat Genet. 2006 April; 38(4):458-62 and Jakobsdottir J, et al. C2 and CFB genes image-related maculopathy and joint action with CFH and LOC387715 genes. PLoS One. 2008 May 21; 3(5):e2199), and most recently in complement C3 (Despriet D D, et al Complement component C3 and risk of age-related macular degeneration. Ophthalmology. 2009 March; 116(3):474-480.e2; Maller J B, et al Variation in complement factor 3 is associated with risk of age-related macular degeneration. Nat Genet. 2007 October; 39(10):1200-1 and Park K H, et al Complement component 3 (C3) haplotypes and risk of advanced age-related macular degeneration. Invest Ophthalmol Vis Sci. 2009 July; 50(7):3386-93. Epub 2009 Feb. 21.). Taken together, the genetic variations in the alternative pathway components CFH, CFB, and C3 can predict clinical outcome in nearly 80% of cases.

Currently there is no proven medical therapy for dry AMD and many patients with neovascular AMD become legally blind despite current therapy with anti-VEGF agents such as Lucentis. Thus, it would be desirable to provide therapeutic agents for the treatment or prevention of complement mediated diseases and particularly for the treatment of AMD.

### SUMMARY OF THE DISCLOSURE

The present disclosure provides compounds that modulate, and/or inhibit, activation of the alternative complement pathway. In certain embodiments, the present disclosure provides compounds that modulate, and/or inhibit, Factor B activity and/or Factor B mediated complement pathway activation. Such Factor B modulators are preferably high affinity Factor B inhibitors that inhibit the catalytic activity of complement Factor B, such as primate Factor B and particularly human Factor B.

The compounds of the present disclosure inhibit or suppress the amplification of the complement system caused by C3 activation irrespective of the initial mechanism of activation (including for example activation of the classical, lectin or alternative pathways).

The disclosure also relates to compounds effective as Factor B modulators, pharmaceutically acceptable salts thereof, compositions thereof, and their use in therapies for the conditions and purposes detailed herein.

The disclosure provides, in a first aspect, a compound of formula (I) or a pharmaceutically acceptable salt thereof,

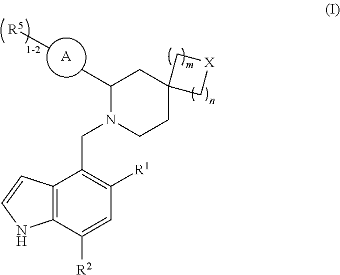

wherein:
X is O or CR^{X1}R^{X2};
R¹ is selected from H, C₁-C₆alkoxyl, C₃-C₅cycloalkoxyl, C₁-C₆alkyl, and C₃-C₅cycloalkyl, wherein the C₁-C₆alkoxyl, C₁-C₆alkyl, and C₃-C₅cycloalkyl are unsubstituted or substituted with 1 or 2 halogen substituents;
R² is C₁-C₃alkyl or C₃cycloalkyl wherein the C₁-C₃alkyl or C₃cycloalkyl are unsubstituted or substituted with 1 or 2 halogen substituents;
R^{X1} is selected from hydrogen, fluoro, C₁-C₆alkyl, and C₃-C₅cycloalkyl;
R^{X2} is selected from hydroxyl, fluoro, C₁-C₆alkyl, and C₃-C₅cycloalkyl;
with the proviso that R^{X1} is not fluoro when R^{X2} is hydroxyl;
or wherein R^{X1} and R^{X2}, in combination with the carbon atom to which they are attached, form a spirocyclic carbocycle having 3-5 ring atoms;
A is a phenyl ring or a 5 or 6 membered heteroaryl ring having 1-4 heteroatoms independently selected from N, O, and S;
each R⁵ is independently selected from H, —CO₂R^{5b}, C₁-C₆alkyl, CH₂CO₂R^{5b}, C₁-C₆hydroxyalkyl, C₃-C₅cycloalkyl, 5- to 6-membered heteroaryl having 1-4 heteroatoms independently selected from N, O, and S, and 4- to 6-membered heterocyclyl having 1-2 heteroatoms independently selected from N, O, and S, wherein the C₁-C₆hydroxyalkyl, C₁-C₆alkyl, 5- to 6-membered heteroaryl and 4- to 6-membered heterocyclyl are unsubstituted or substituted with 1 or 2 R^{5a};
each R^{5a} is independently selected from fluoro, hydroxyl, and C₁-C₆alkyl, wherein the C₁-C₆alkyl is unsubstituted or substituted with 1, 2, or 3 fluoro;
wherein when R⁵ is a 4- to 6-membered heterocyclyl, two R^{5a} are not fluoro and hydroxyl substituted on the same position; and
wherein R^{5b} is selected from H or C₁-C₅ alkyl;
m is 0 or 1;
n is 0, 1, or 2;
wherein both m and n are not 0; and
with the proviso that
when X is 0, then m is 1 and n is 1 or 2.

In a second aspect, there is provided a pharmaceutical composition comprising a therapeutically effective amount of a compound of Formula (I), or a pharmaceutically acceptable salt thereof, and a pharmaceutically acceptable carrier or excipient.

In a third aspect, there is provided a method of treating or preventing a disease or disorder in a subject in need thereof, the method comprising administering to the subject a therapeutically effective amount of a compound of Formula (I), or a pharmaceutically acceptable salt thereof.

In a further aspect, there is provided a method of modulating the complement alternative pathway activity in a subject, the method comprising administering to the subject a therapeutically effective amount of a compound of Formula (I), or a pharmaceutically acceptable salt thereof.

In a further aspect, there is provided a method of treating a disease or disorder mediated by complement activation, in particular mediated by activation of the complement alternative pathway, in a subject in need thereof, the method comprising administering to the subject a therapeutically effective amount of a compound of Formula (I), or a pharmaceutically acceptable salt thereof.

In a further aspect, there is provided a method of treating a disease or disorder that is affected by the modulation of complement alternative pathway comprising administering to the subject a therapeutically effective amount of a compound of Formula (I), or a pharmaceutically acceptable salt thereof.

In a further aspect, there is provided a method of treating a disease or disorder associated with dysregulation of the complement alternative pathway comprising administering to the subject a therapeutically effective amount of a compound of Formula (I), or a pharmaceutically acceptable salt thereof.

In a further aspect, there is provided a method of inhibiting the expression or activity of complement factor B, the method comprising administering to the subject a compound of Formula (I), or a pharmaceutically acceptable salt thereof.

In a further aspect, there is provided a method of treating age-related macular degeneration comprising administering to a subject in need thereof an effective amount of a composition comprising a compound of Formula (I), or a pharmaceutically acceptable salt thereof.

In a further aspect, there is provided a compound of Formula (I), or a pharmaceutically acceptable salt thereof, for use as a medicament.

In a further aspect, there is provided a compound of Formula (I), or a pharmaceutically acceptable salt thereof, for use in inhibiting the expression or activity of complement factor B, in a subject in need thereof.

In a further aspect, there is provided a compound of Formula (I), or a pharmaceutically acceptable salt thereof, for use in treating a disease or disorder associated with dysregulation of the complement alternative pathway.

In a further aspect, there is provided the use of a compound of Formula (I), or a pharmaceutically acceptable salt thereof, in the manufacture of a medicament for the treatment of a disease or disorder mediated by complement activation or activation of the complement alternative pathway.

In a further aspect, there is provided the use of a compound of Formula (I), or a pharmaceutically acceptable salt thereof, for the treatment of a disease or disorder mediated by complement activation or activation of the complement alternative pathway.

In a further aspect, there is provided the use of a compound of Formula (I), or a pharmaceutically acceptable salt thereof, for the treatment of a disease or disorder that is affected by the modulation of complement alternative pathway.

### DETAILED DESCRIPTION OF THE DISCLOSURE

As noted above, the present disclosure provides compounds that modulate Factor B activation and/or Factor B-mediated signal transduction of the complement system. Such compounds may be used in vitro or in vivo to modulate Factor B activity in a variety of contexts. The compounds disclosed herein are effective as Factor B modulators and/or inhibitors. Without wishing to be bound by any theory, it is believed that the disclosed compounds may treat disorders associated with Factor B, including the treatment of age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

### Definitions

Unless specified otherwise, the terms “compounds of the present disclosure,” “compounds of the disclosure,” or “compound of the disclosure” refer to compounds of formulae (I), (I-A), (I-B), exemplified compounds, salts thereof, particularly pharmaceutically acceptable salts thereof, hydrates, solvates, as well as all stereoisomers (including diastereoisomers and enantiomers), rotamers, tautomers, and isotopically labeled compounds (including deuterium substitutions), as well as inherently formed moieties.

In the groups, radicals, or moieties defined below, the number of carbon atoms is often specified preceding the group, for example, C₁-C₁₀alkyl means an alkyl group or radical having 1 to 10 carbon atoms.

Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “and/or” means either “and” or “or” unless indicated otherwise.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. The terms C₁-C₃alkyl and C₁-C₄alkyl are to be construed accordingly. Examples of C₁-C₆alkyl include, but are not limited to, methyl, ethyl, n-propyl, 1-methylethyl (iso-propyl), n-butyl, 1-methylpropyl (sec-butyl), 2-methylpropyl (iso-butyl), 1,1-dimethylethyl (tert-butyl), n-pentyl and n-hexyl.

As used herein, the term “C₁-C₆alkoxyl” refers to a radical of the formula —OR, where Rₐ is a C₁-C₆alkyl radical as generally defined above. Examples of C₁-C₆alkoxyl include, but are not limited to, methoxy, ethoxy, propoxy, iso-propoxy, butoxy, iso-butoxy, tert-butoxy, sec-butoxy, pentoxy, and hexoxy.

The term “halogen” or “halo” means fluorine, chlorine, bromine or iodine.

As used herein, the term “cycloalkyl” means a monocyclic or polycyclic saturated or partially unsaturated carbon ring containing 3-18 carbon atoms wherein there are no delocalized pi electrons (aromaticity) shared among the ring carbon. The term “C₃-C₅cycloalkyl” is to be construed accordingly. The term polycyclic encompasses bridged (e.g., norbornane), fused (e.g., decalin) and spirocyclic cycloalkyl. Preferably, cycloalkyl, e.g., C₃-C₅cycloalkyl, is a monocyclic hydrocarbon group of 3 to 5 carbon atoms.

Examples of cycloalkyl groups include, without limitations, cyclopropenyl, cyclopropyl cyclobutyl, cyclobutenyl, cyclopentyl, bicyclo[1.1.1]pentanyl and derivatives thereof.

Examples of C₃-C₅cycloalkyl include, but are not limited to, cyclopropyl, cyclobutyl, and cyclopentyl.

“Heterocyclyl” means a saturated or partially saturated monocyclic or polycyclic ring containing carbon and at least one heteroatom selected from oxygen, nitrogen, and sulfur (O, N, and S) and wherein there are no delocalized pi electrons (aromaticity) shared among the ring carbon or heteroatoms. The term “4- to 6-membered heterocyclyl” is to be construed accordingly. The heterocyclyl ring structure may be substituted by one or more substituents. The substituents can themselves be optionally substituted. The heterocyclyl may be bonded via a carbon atom or heteroatom. The term polycyclic encompasses bridged, fused and spirocyclic heterocyclyl.

Examples of heterocyclyl rings include, but are not limited to, oxetanyl, azetidinyl, tetrahydrofuranyl, tetrahydropyranyl, pyrrolidinyl, oxazolinyl, isoxazolinyl, oxazolidinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, dihydroisoxazolinyl, pyrrolinyl, pyrazolinyl, oxazepinyl, dithiolanyl, homotropanyl, dihydropyranyl (e.g., 3,6-dihydro-2H-pyranyl), oxaspiroheptanyl (e.g., 2-oxaspiro[3.3]heptan-6-yl) and the like.

Examples of 4- to 6-membered heterocyclyl include, without limitations, azetidinyl, pyrrolidinyl, tetrahydrofuranyl, tetrahydrothienyl, piperidinyl, piperazinyl, dihydroisoxazolinyl, tetrahydropyranyl, morpholinyl, dihydropyranyl (e.g., 3,6-dihydro-2H-pyranyl) and oxaspiroheptanyl (e.g., 2-oxaspiro[3.3]heptan-6-yl).

As used herein, the term “heteroaryl” is intended to include monocyclic heterocyclic aromatic rings containing one or more heteroatoms selected from oxygen, nitrogen, and sulfur (O, N, and S). Representative examples are pyrrolyl, furanyl, thienyl, oxazolyl, thiazolyl, imidazolyl, pyrazolyl, isothiazolyl, isooxazolyl, triazolyl, (e.g., 1,2,4-triazolyl), oxadiazolyl, (e.g., 1,2,3-oxadiazolyl, 1,2,4-oxadiazolyl, 1,2,5-oxadiazolyl, 1,3,4-oxadiazolyl), thiadiazolyl (e.g., 1,2,3-thiadiazolyl, 1,2,4-thiadiazolyl, 1,2,5-thiadiazolyl, 1,3,4-thiadiazolyl), tetrazolyl, pyranyl, pyridinyl, pyridazinyl, pyrimidinyl, pyrazinyl, 1,2,3-triazinyl, 1,2,4-triazinyl, 1,3,5-triazinyl, thiadiazinyl, azepinyl, azecinyl, and the like.

Heteroaryl is also intended to include bicyclic heterocyclic aromatic rings containing one or more heteroatoms selected from oxygen, nitrogen, and sulfur (O, N, and S). Representative examples are indolyl, isoindolyl, benzofuranyl, benzothiophenyl, indazolyl, benzopyranyl, benzimidazolyl, benzothiazolyl, benzisothiazolyl, benzoxazolyl, benzisoxazolyl, benzoxazinyl, benzotriazolyl, naphthyridinyl, phthalazinyl, pteridinyl, purinyl, quinazolinyl, cinnolinyl, quinolinyl, isoquinolinyl, quinoxalinyl, oxazolopyridinyl, isooxazolopyridinyl, pyrrolopyridinyl, furopyridinyl, thienopyridinyl, imidazopyridinyl, imidazopyrimidinyl, pyrazolopyridinyl, pyrazolopyrimidinyl, pyrazolotriazinyl, thiazolopyridinyl, thiazolopyrimidinyl, imdazothiazolyl, triazolopyridinyl, triazolopyrimidinyl, and the like.

Heteroaryl is also intended to include polycyclic heterocyclic aromatic rings containing one or more heteroatoms selected from oxygen, nitrogen, and sulfur (O, N, and S). Representative examples are carbazolyl, phenoxazinyl, phenazinyl, acridinyl, phenothiazinyl, carbolinyl, phenanthrolinyl, and the like.

The heteroaryl ring structure may be substituted by one or more substituents. The substituents can themselves be optionally substituted. The heteroaryl ring may be bonded via a carbon atom or heteroatom.

The term “5 or 6 membered heteroaryl” is to be construed accordingly.

Examples of 5 or 6 membered heteroaryl include, but are not limited to, furan, indolyl, pyridinyl, pyrimidinyl, pyridinonyl, pyridazinyl, triazolyl, (e.g., 1,2,4-triazolyl), pyrazolyl, thiazolyl, oxazolyl, isooxazolyl, pyrrolyl, oxadiazolyl, (e.g., 1,2,3-oxadiazolyl, 1,2,4-oxadiazolyl, 1,2,5-oxadiazolyl, 1,3,4-oxadiazolyl), imidazolyl, thiophenyl, thiadiazolyl (e.g., 1,2,3-thiadiazolyl, 1,2,4-thiadiazolyl, 1,2,5-thiadiazolyl, 1,3,4-thiadiazolyl), pyrazinyl, isooxazolopyridinyl, dihydropyridooxazinyl and tetrazolyl. The term “5 or 6 membered heteroaryl ring having 1-2 heteroatoms independently selected from N, O, and S” is to be construed accordingly.

As used herein “modulator”, means, for example, a compound of the disclosure, that modulates, decreases, or reduces the levels of a specific protein (e.g., complement factor B). The amount of a specific protein (e.g., complement factor B) modulated can be measured by comparing the amount of the specific protein (e.g., complement factor B) remaining after treatment with a compound of the disclosure as compared to the initial amount or level of the specific protein (e.g., complement factor B) present as measured prior to treatment with a compound of the disclosure.

As used herein “inhibitor”, means, for example, a compound of the disclosure, that inhibits or reduces the activity of at least one component of the complement pathway, e.g., the compound inhibits binding of one component to another component of the pathway. For example, the complement pathway is the alternative complement pathway. For example, the component of the complement pathway is complement factor B.

As used herein, the term “inhibit”, “inhibition” or “inhibiting” refers to the reduction or suppression of a given condition, symptom, or disorder, or disease, or a significant decrease in the baseline activity of a biological activity or process.

The term “effective amount” of the compounds described herein, refers to that amount of a therapeutic compound necessary or sufficient to perform its intended function within a mammal. An effective amount of the therapeutic compound can vary according to factors such as the amount of the causative agent already present in the mammal, the age, sex, and weight of the mammal, and the ability of the therapeutic compounds of the present disclosure to treat the conditions wherein complement factor B plays a role.

Thus, the term “a therapeutically effective amount” of a compound of the disclosure refers to an amount of the compound of the disclosure that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound of the disclosure that, when administered to a subject, is effective to (1) at least partially alleviate, prevent and/or ameliorate a condition, or a disorder or a disease (i) mediated by complement factor B, or (ii) associated with complement factor B activity, or (iii) characterized by activity (normal or abnormal) of complement factor B: (2) reduce or inhibit the activity of complement factor B; or (3) reduce or inhibit the expression of complement factor B. In another embodiment, the term “a therapeutically effective amount” refers to the amount of the compound of the disclosure that, when administered to a cell, or a tissue, or a non-cellular biological material, or a medium, is effective to at least partially reducing or inhibiting the activity of complement factor B; or at least partially reducing or inhibiting the expression of complement factor B.

As used herein, the term “treat”, “treating” or “treatment” in connection to a disease or disorder refers in some embodiments, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those, which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder or a symptom thereof.

As used herein, the term “subject” or “patient” refers to human and non-human mammals, including but, not limited to, primates, rabbits, pigs, horses, dogs, cats, sheep, and cows. In particular embodiments, a subject or patient is a human. In some embodiments, the term “patient” or “subject” refers to a human being who is diseased with the condition (i.e., disease or disorder) described herein and who would benefit from the treatment. As used herein, a subject is “in need of” a treatment if such subject (patient) would benefit biologically, medically or in quality of life from such treatment. In particular embodiments, the subject is an adult human at least about 18 years of age. In particular embodiments, the subject is an adult human from about 18 to about 75 years of age. In some embodiments, the subject is a human child up to about 18 years of age.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “about” refers to a range of values +/−10% of a specified value.

As used herein, the term “a,” “an,” “the” and similar terms used in the context of the disclosure (especially in the context of the claims) are to be construed to cover both the singular and plural unless otherwise indicated herein or clearly contradicted by the context.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

Various enumerated embodiments of the disclosure are described herein. It will be recognized that features specified in each embodiment may be combined with other specified features to provide further embodiments of the disclosure.

### ENUMERATED EMBODIMENTS

Embodiment 1. A compound of formula (I) or a pharmaceutically acceptable salt thereof,

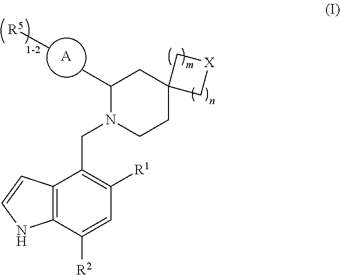

wherein:
X is O or CR^{X1}R^{X2};
R¹ is selected from H, C₁-C₆alkoxyl, C₃-C₅cycloalkoxyl, C₁-C₆alkyl, and C₃-C₅cycloalkyl, wherein the C₁-C₆alkoxyl, C₁-C₆alkyl, and C₃-C₅cycloalkyl are unsubstituted or substituted with 1 or 2 halogen substituents;
R² is C₁-C₃alkyl or C₃cycloalkyl wherein the C₁-C₃alkyl or C₃cycloalkyl are unsubstituted or substituted with 1 or 2 halogen substituents;
R^{X1} is selected from hydrogen, fluoro, C₁-C₆alkyl, and C₃-C₅cycloalkyl;
R^{X2} is selected from hydroxyl, fluoro, C₁-C₆alkyl, and C₃-C₅cycloalkyl;
with the proviso that R^{X1} is not fluoro when R^{X2} is hydroxyl;
or wherein R^{X1} and R^{X2}, in combination with the carbon atom to which they are attached, form a spirocyclic carbocycle having 3-5 ring atoms;
A is a phenyl ring or a 5 or 6 membered heteroaryl ring having 1-4 heteroatoms independently selected from N, O, and S;
each R⁵ is independently selected from H, —CO₂R^{5b}, C₁-C₆alkyl, CH₂CO₂R^{5b}, C₁-C₆hydroxyalkyl, C₃-C₅cycloalkyl, 5- to 6-membered heteroaryl having 1-4 heteroatoms independently selected from N, O, and S, and 4- to 6-membered heterocyclyl having 1-2 heteroatoms independently selected from N, O, and S, wherein the C₁-C₆hydroxyalkyl, C₁-C₆alkyl, 5- to 6-membered heteroaryl and 4- to 6-membered heterocyclyl are unsubstituted or substituted with 1 or 2 R^{5a};
each R^{5a} is independently selected from fluoro, hydroxyl and C₁-C₆alkyl, wherein the C₁-C₆alkyl is unsubstituted or substituted with 1, 2, or 3 fluoro;
wherein when R⁵ is a 4- to 6-membered heterocyclyl, two R^{5a} are not fluoro and hydroxyl substituted on the same position; and
R^{5b} is selected from H or C₁-C₅ alkyl;
m is 0 or 1;
n is 0, 1, or 2;
wherein both m and n are not 0; and
with the proviso that
when X is O, then m is 1 and n is 1 or 2.
Embodiment 2. The compound of formula (I) according to Embodiment 1, or a pharmaceutically acceptable salt thereof, of formula (I-A) or (I-B)

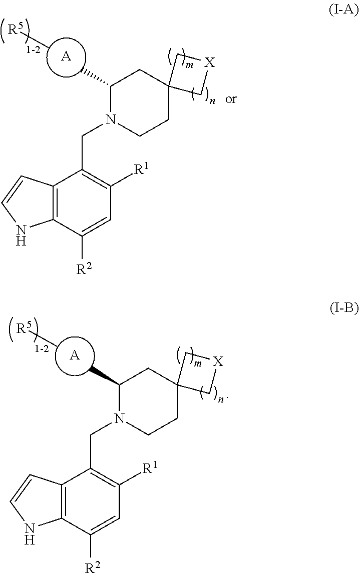

Embodiment 3. The compound I-A according to Embodiment 2, or a pharmaceutically acceptable salt thereof.
Embodiment 4. The compound according to any one of the preceding Embodiments, or a pharmaceutically acceptable salt thereof, wherein R² is selected from methyl, ethyl, and cyclopropyl.
Embodiment 5. The compound of according to Embodiment 4, or a pharmaceutically acceptable salt thereof, wherein R² is methyl.
Embodiment 6. The compound according to any one of the preceding Embodiments, or a pharmaceutically acceptable salt thereof, wherein A is a phenyl ring.
Embodiment 7. The compound of Embodiment 6, or a pharmaceutically acceptable salt thereof, wherein R⁵ is substituted on the para position of the phenyl ring.
Embodiment 8. The compound according to any of Embodiments 1 to 5, or a pharmaceutically acceptable salt thereof, wherein A is selected from furanyl, thiophenyl, pyrrolyl, oxazolyl, isoxazolyl, thiazolyl, pyrazolyl, imiadazolyl, pyridyl, triazolyl, tetrazolyl, oxadiazolyl, isoxadiazolyl, pyrimidinyl, pyrazinyl, and pyridazinyl.
Embodiment 9. The compound according to Embodiment 8, or a pharmaceutically acceptable salt thereof, wherein A is selected from:

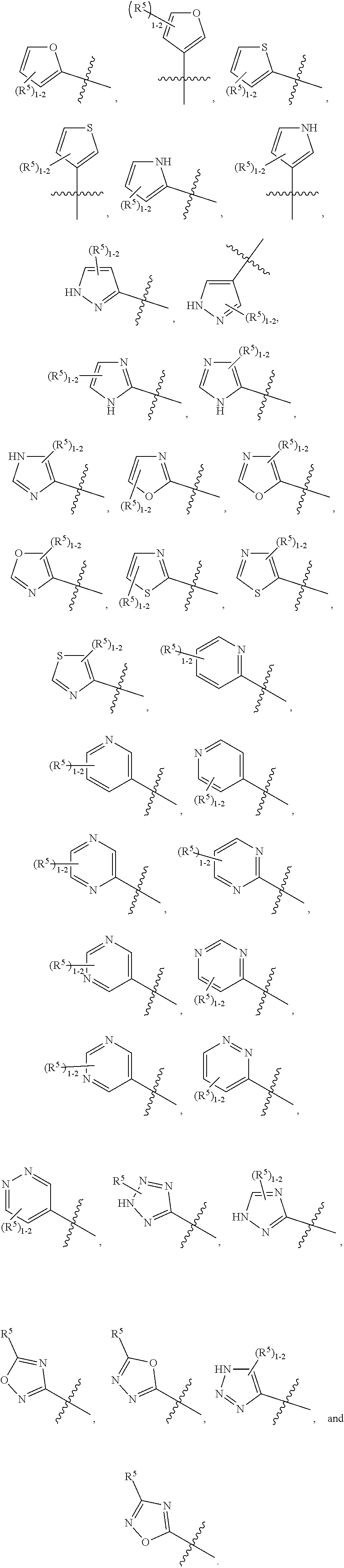

Embodiment 10. The compound according to any one of the preceding Embodiments, or a pharmaceutically acceptable salt thereof, wherein A is substituted with 1 R⁵, and R⁵ is selected from —CO₂H, C₁-C₆hydroxyalkyl, 5- to 6-membered heteroaryl having 1-4 heteroatoms independently selected from N, O, and S, and 4- to 6-membered heterocyclyl having 1 O heteroatom, wherein the 4- to 6-membered heterocyclyl is unsubstituted or substituted with 0-1 R^{5a}.
Embodiment 11. The compound according to any one of the preceding Embodiments, or a pharmaceutically acceptable salt thereof, wherein R⁵ is selected from CO₂R^{5b}, C₁-C₆hydroxyalkyl, 5-membered heteroaryl having 2 N heteroatoms, and 4- to 6-membered heterocyclyl having 1 O heteroatom, wherein 4- to 6-membered heterocyclyl is unsubstituted or substituted with 1 R^{5a}, wherein R^{5a} is hydroxyl and R^{5b} is H.
Embodiment 12. The compound according to any one of the preceding Embodiments, or a pharmaceutically acceptable salt thereof, wherein R⁵ is selected from —CO₂H, tetrazole, and oxetane substituted with hydroxyl.
Embodiment 13. The compound according to any one of the preceding Embodiments, or a pharmaceutically acceptable salt thereof, wherein R⁵ is —CO₂H.
Embodiment 14. The compound according to any one of the preceding Embodiments, or a pharmaceutically acceptable salt thereof, wherein R⁵ is tetrazole.
Embodiment 15. The compound according to any one of the preceding Embodiments, or a pharmaceutically acceptable salt thereof, wherein R¹ is selected from C₁-C₄alkoxyl, C₁-C₄alkyl, and C₃-C₆cycloalkyl, wherein the C₃-C₆cycloalkyl is unsubstituted or substituted with 1 or 2 fluoro substituents.
Embodiment 16. The compound according to any one of the preceding Embodiments, or a pharmaceutically acceptable salt thereof, wherein R¹ is selected from methoxyl, methyl, and cyclopropyl, wherein the cyclopropyl is unsubstituted or substituted with 1 or 2 fluoro substituents.
Embodiment 17. The compound according to any one of the preceding Embodiments, or a pharmaceutically acceptable salt thereof, wherein R¹ is selected from methoxyl, and cyclopropyl, wherein the cyclopropyl is unsubstituted or substituted with 1 or 2 fluoro substituents.
Embodiment 18. The compound according to any one of the preceding Embodiments, or a pharmaceutically acceptable salt thereof, wherein R^{X1} and R^{X2} are both fluoro, fluoro and C₁-C₆alkyl, hydrogen and fluoro, C₁-C₆alkyl and hydroxyl, or hydrogen and hydroxyl.
Embodiment 19. The compound according to Embodiment 18, or a pharmaceutically acceptable salt thereof, wherein R^{X1} and R^{X2} are both fluoro, fluoro and C₁-C₆alkyl, or fluoro and H.
Embodiment 20. The compound according to any one of the preceding Embodiments, or a pharmaceutically acceptable salt thereof, wherein n is 1 or 2.
Embodiment 21. The compound according to any one of the preceding Embodiments, or a pharmaceutically acceptable salt thereof, wherein X is O.
Embodiment 22. The compound according to any one of Embodiments 1 to 20, or a pharmaceutically acceptable salt thereof, wherein X is CR^{X1}R^{X2}.
Embodiment 23. The compound according to Embodiment 22, wherein R^{X1} is fluoro, and R^{X2} is selected from fluoro, and C₁-C₆alkyl, e.g., methyl.
Embodiment 24. The compound according to any one of the preceding Embodiments, or a pharmaceutically acceptable salt thereof, wherein R^{5a} is hydroxyl.
Embodiment 25. The compound of formula (I) according to Embodiment 1, or a pharmaceutically acceptable salt thereof, selected from:

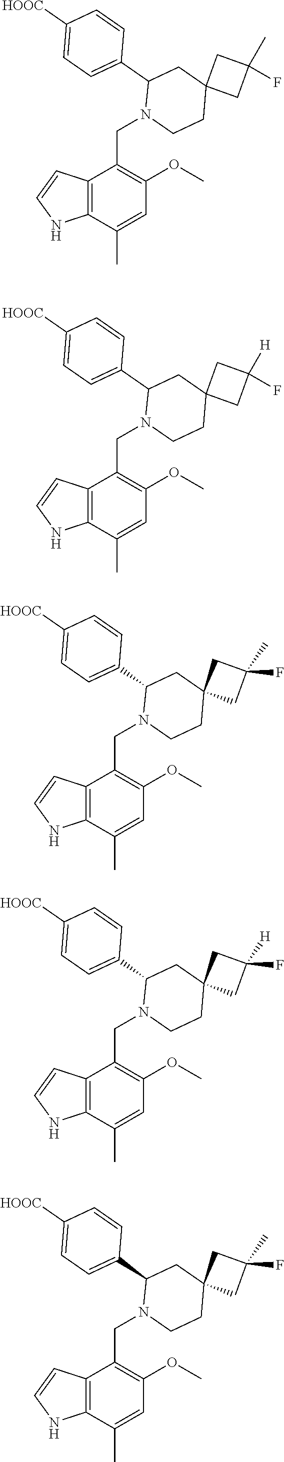

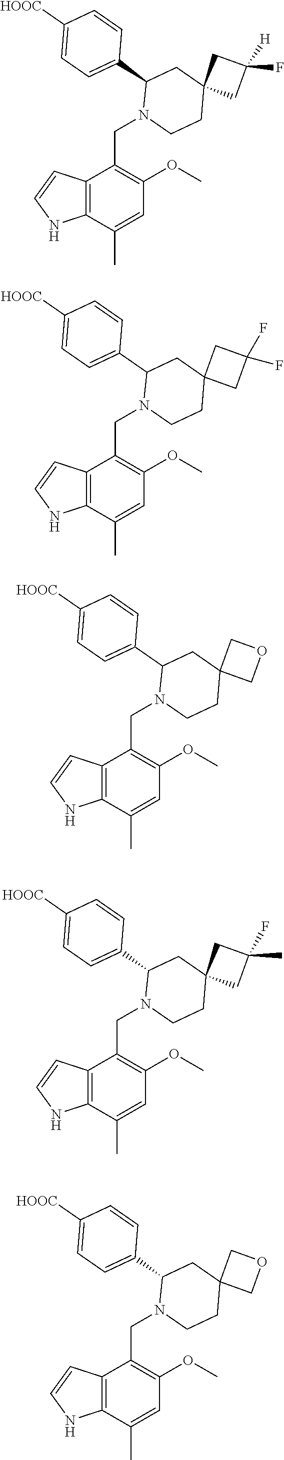

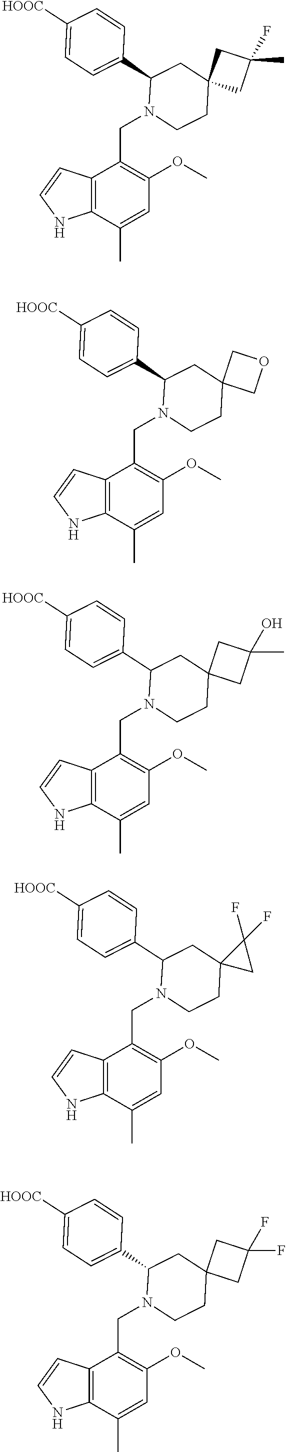

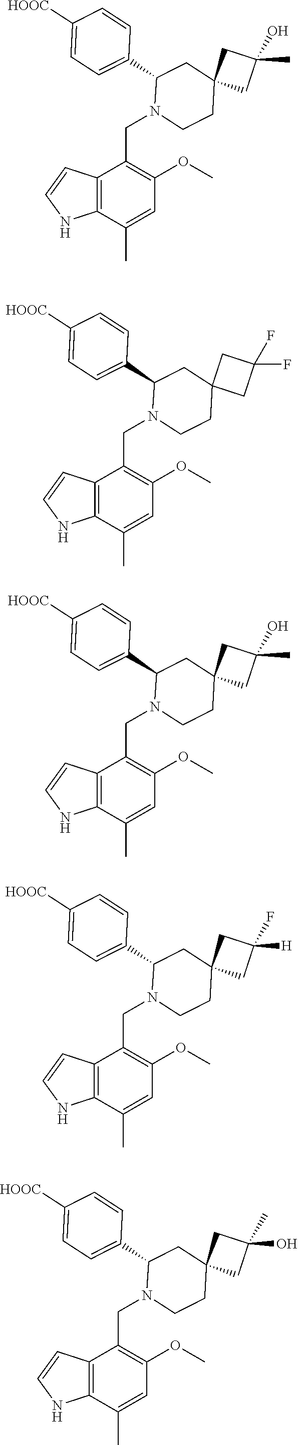

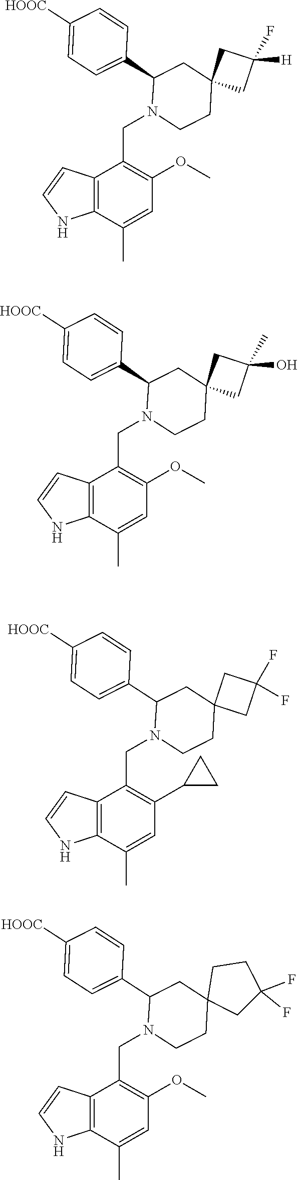 [SMILES: COc1cc(C)c2[nH]ccc2c1CN1CC[C@]2(C[C@@H]1c1ccc(C(=O)O)cc1)C[C@@H](F)C2 ; COc1cc(C)c2[nH]ccc2c1CN1CC[C@]2(C[C@@H]1c1ccc(C(=O)O)cc1)C[C@](C)(O)C2 ; Cc1cc(C2CC2)c(CN2CCC3(CC2c2ccc(C(=O)O)cc2)CC(F)(F)C3)c2cc[nH]c12 ; COc1cc(C)c2[nH]ccc2c1CN1CCC2(CCC(F)(F)C2)CC1c1ccc(C(=O)O)cc1]

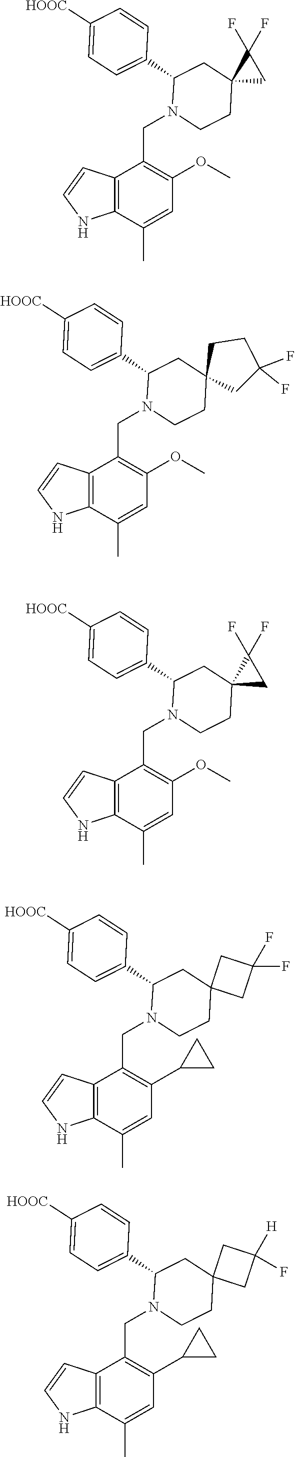

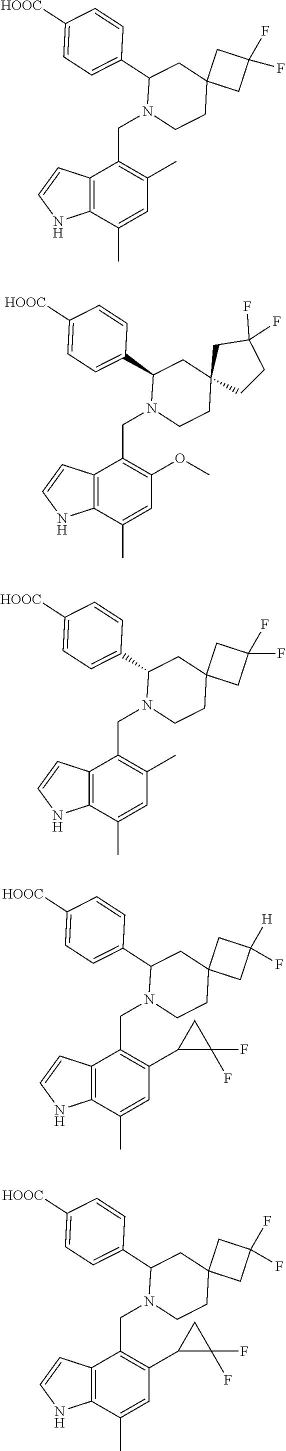 [SMILES: Cc1cc(C)c2[nH]ccc2c1CN1CCC2(CC1c1ccc(C(=O)O)cc1)CC(F)(F)C2 ; COc1cc(C)c2[nH]ccc2c1CN1CC[C@@]2(CCC(F)(F)C2)C[C@@H]1c1ccc(C(=O)O)cc1 ; Cc1cc(C)c2[nH]ccc2c1CN1CCC2(C[C@H]1c1ccc(C(=O)O)cc1)CC(F)(F)C2 ; Cc1cc(C2CC2(F)F)c(CN2CCC3(CC(F)C3)CC2c2ccc(C(=O)O)cc2)c2cc[nH]c12 ; Cc1cc(C2CC2(F)F)c(CN2CCC3(CC2c2ccc(C(=O)O)cc2)CC(F)(F)C3)c2cc[nH]c12]

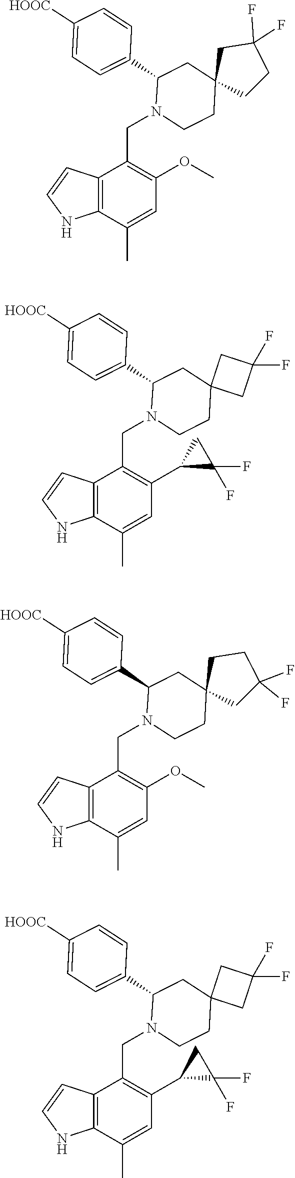

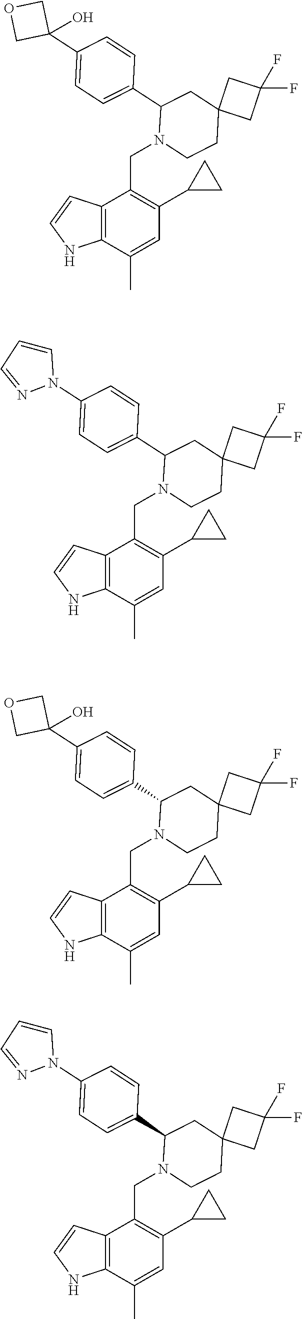 [SMILES: Cc1cc(C2CC2)c(CN2CCC3(CC2c2ccc(C4(O)COC4)cc2)CC(F)(F)C3)c2cc[nH]c12 ; Cc1cc(C2CC2)c(CN2CCC3(CC2c2ccc(-n4cccn4)cc2)CC(F)(F)C3)c2cc[nH]c12 ; Cc1cc(C2CC2)c(CN2CCC3(C[C@H]2c2ccc(C4(O)COC4)cc2)CC(F)(F)C3)c2cc[nH]c12 ; Cc1cc(C2CC2)c(CN2CCC3(C[C@@H]2c2ccc(-n4cccn4)cc2)CC(F)(F)C3)c2cc[nH]c12]

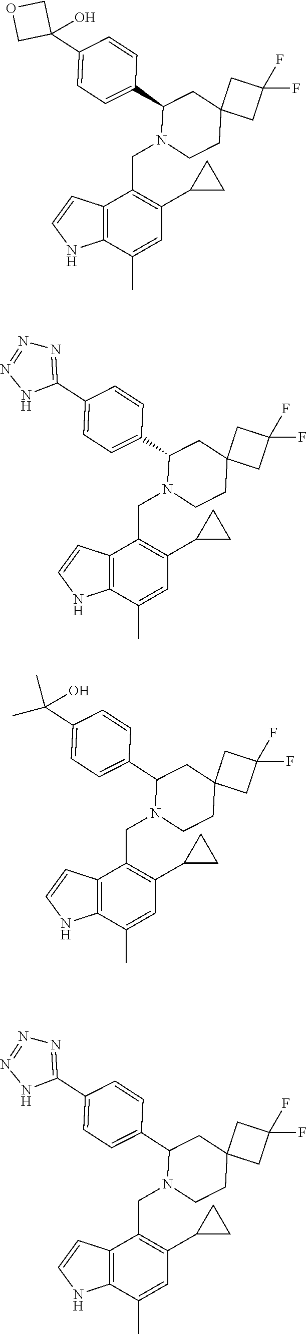 [SMILES: Cc1cc(C2CC2)c(CN2CCC3(C[C@@H]2c2ccc(C4(O)COC4)cc2)CC(F)(F)C3)c2cc[nH]c12 ; Cc1cc(C2CC2)c(CN2CCC3(C[C@H]2c2ccc(-c4nnn[nH]4)cc2)CC(F)(F)C3)c2cc[nH]c12 ; Cc1cc(C2CC2)c(CN2CCC3(CC2c2ccc(C(C)(C)O)cc2)CC(F)(F)C3)c2cc[nH]c12 ; Cc1cc(C2CC2)c(CN2CCC3(CC2c2ccc(-c4nnn[nH]4)cc2)CC(F)(F)C3)c2cc[nH]c12]

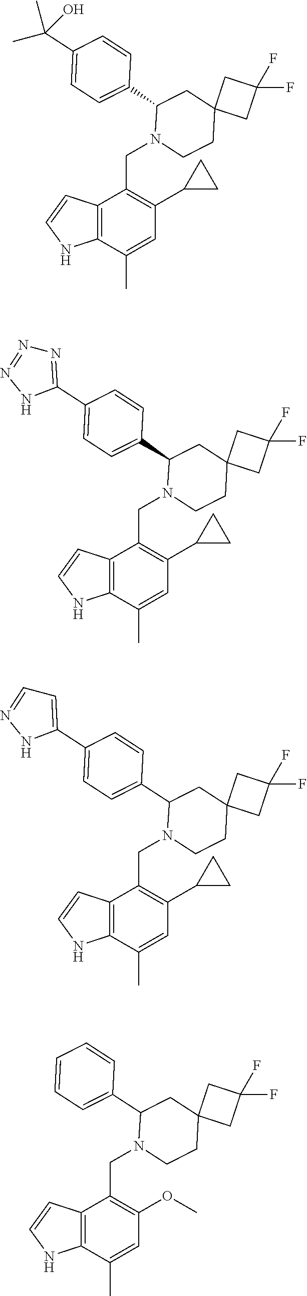 [SMILES: Cc1cc(C2CC2)c(CN2CCC3(C[C@H]2c2ccc(C(C)(C)O)cc2)CC(F)(F)C3)c2cc[nH]c12 ; Cc1cc(C2CC2)c(CN2CCC3(C[C@@H]2c2ccc(-c4nnn[nH]4)cc2)CC(F)(F)C3)c2cc[nH]c12 ; Cc1cc(C2CC2)c(CN2CCC3(CC2c2ccc(-c4ccn[nH]4)cc2)CC(F)(F)C3)c2cc[nH]c12 ; COc1cc(C)c2[nH]ccc2c1CN1CCC2(CC1c1ccccc1)CC(F)(F)C2]

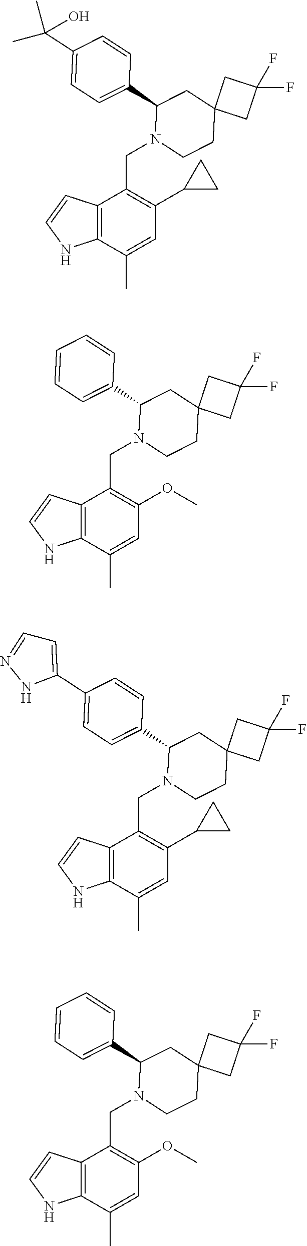 [SMILES: Cc1cc(C2CC2)c(CN2CCC3(C[C@@H]2c2ccc(C(C)(C)O)cc2)CC(F)(F)C3)c2cc[nH]c12 ; COc1cc(C)c2[nH]ccc2c1CN1CCC2(C[C@H]1c1ccccc1)CC(F)(F)C2 ; Cc1cc(C2CC2)c(CN2CCC3(C[C@H]2c2ccc(-c4ccn[nH]4)cc2)CC(F)(F)C3)c2cc[nH]c12 ; COc1cc(C)c2[nH]ccc2c1CN1CCC2(C[C@@H]1c1ccccc1)CC(F)(F)C2]

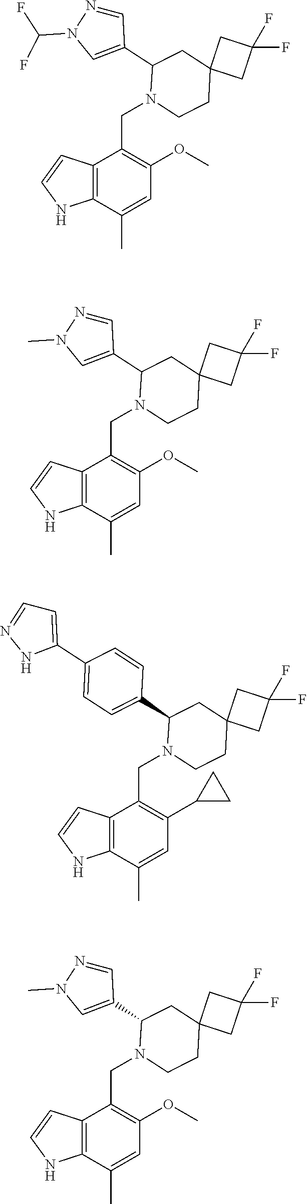 [SMILES: COc1cc(C)c2[nH]ccc2c1CN1CCC2(CC1c1cnn(C(F)F)c1)CC(F)(F)C2 ; COc1cc(C)c2[nH]ccc2c1CN1CCC2(CC1c1cnn(C)c1)CC(F)(F)C2 ; Cc1cc(C2CC2)c(CN2CCC3(C[C@@H]2c2ccc(-c4ccn[nH]4)cc2)CC(F)(F)C3)c2cc[nH]c12 ; COc1cc(C)c2[nH]ccc2c1CN1CCC2(C[C@H]1c1cnn(C)c1)CC(F)(F)C2]

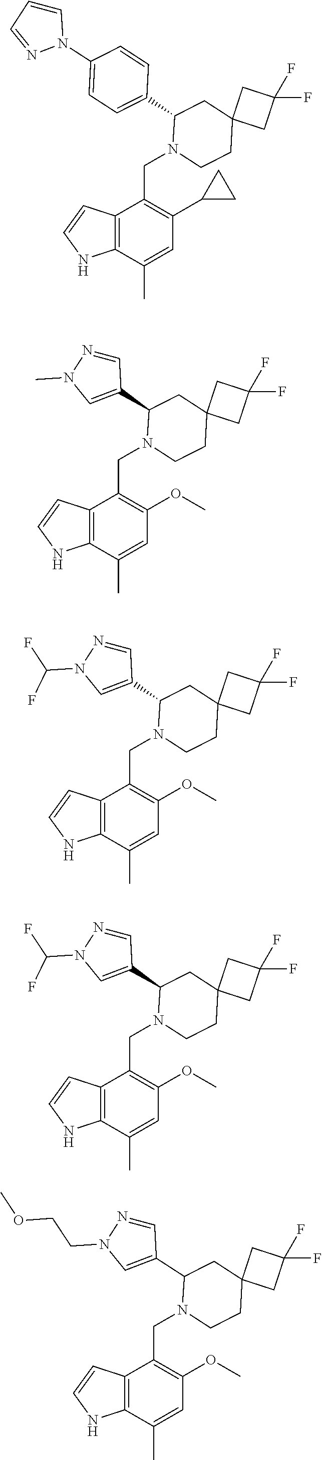

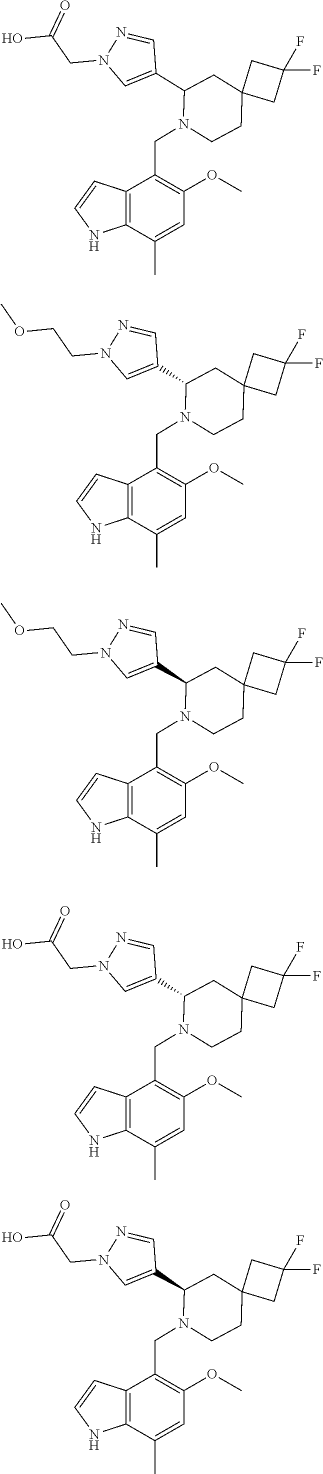 [SMILES: COc1cc(C)c2[nH]ccc2c1CN1CCC2(CC1c1cnn(CC(=O)O)c1)CC(F)(F)C2 ; COCCn1cc([C@@H]2CC3(CCN2Cc2c(OC)cc(C)c4[nH]ccc24)CC(F)(F)C3)cn1 ; COCCn1cc([C@H]2CC3(CCN2Cc2c(OC)cc(C)c4[nH]ccc24)CC(F)(F)C3)cn1 ; COc1cc(C)c2[nH]ccc2c1CN1CCC2(C[C@H]1c1cnn(CC(=O)O)c1)CC(F)(F)C2 ; COc1cc(C)c2[nH]ccc2c1CN1CCC2(C[C@@H]1c1cnn(CC(=O)O)c1)CC(F)(F)C2]

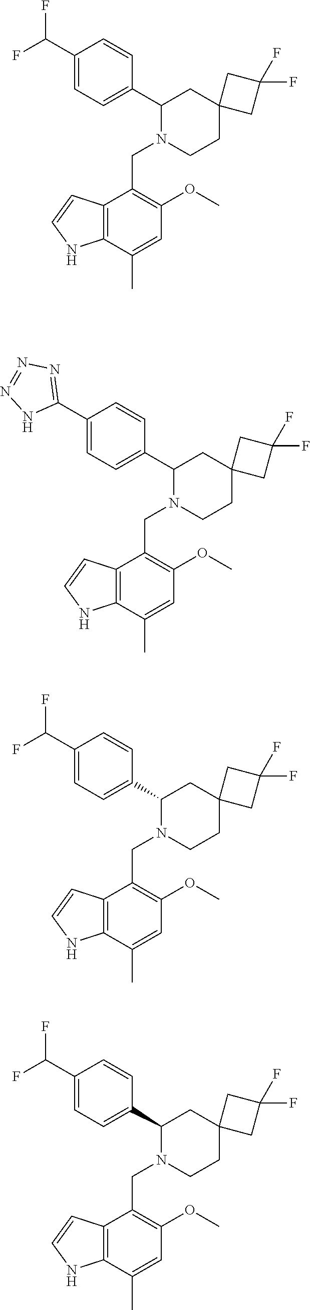 [SMILES: COc1cc(C)c2[nH]ccc2c1CN1CCC2(CC1c1ccc(C(F)F)cc1)CC(F)(F)C2 ; COc1cc(C)c2[nH]ccc2c1CN1CCC2(CC1c1ccc(-c3nnn[nH]3)cc1)CC(F)(F)C2 ; COc1cc(C)c2[nH]ccc2c1CN1CCC2(C[C@H]1c1ccc(C(F)F)cc1)CC(F)(F)C2 ; COc1cc(C)c2[nH]ccc2c1CN1CCC2(C[C@@H]1c1ccc(C(F)F)cc1)CC(F)(F)C2]

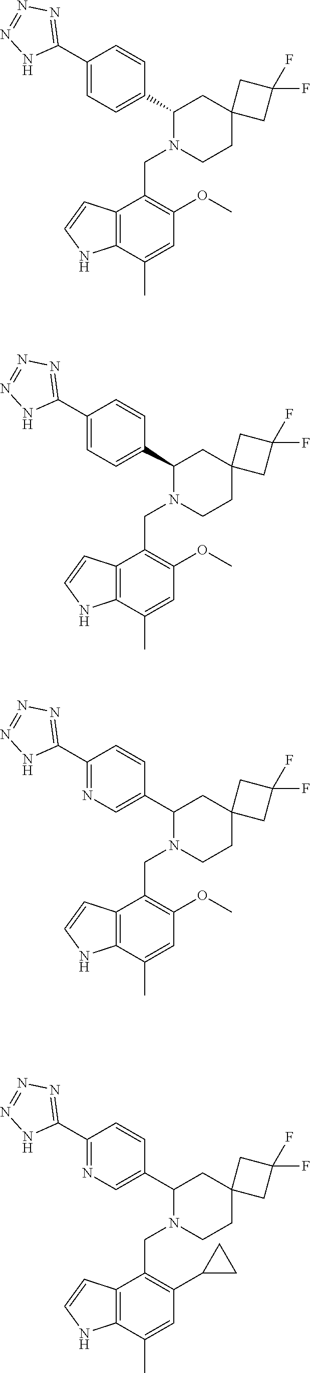 [SMILES: COc1cc(C)c2[nH]ccc2c1CN1CCC2(C[C@H]1c1ccc(-c3nnn[nH]3)cc1)CC(F)(F)C2 ; COc1cc(C)c2[nH]ccc2c1CN1CCC2(C[C@@H]1c1ccc(-c3nnn[nH]3)cc1)CC(F)(F)C2 ; COc1cc(C)c2[nH]ccc2c1CN1CCC2(CC1c1ccc(-c3nnn[nH]3)nc1)CC(F)(F)C2 ; Cc1cc(C2CC2)c(CN2CCC3(CC2c2ccc(-c4nnn[nH]4)nc2)CC(F)(F)C3)c2cc[nH]c12]

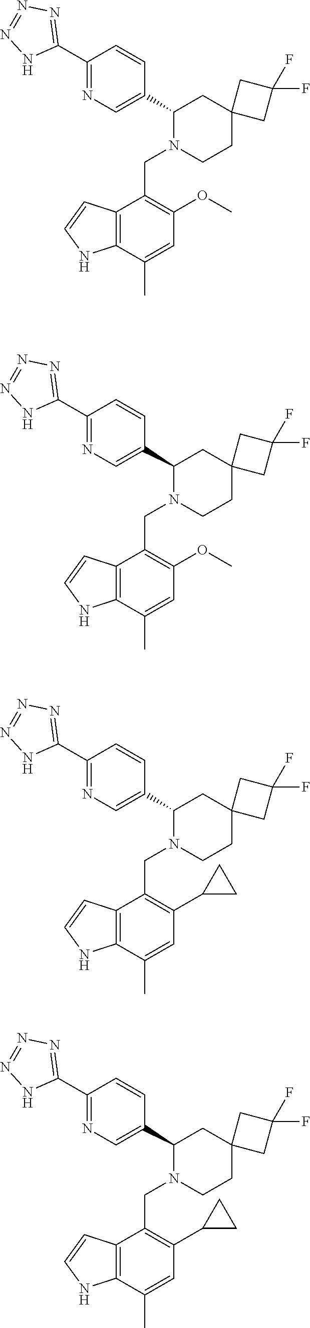 [SMILES: COc1cc(C)c2[nH]ccc2c1CN1CCC2(C[C@H]1c1ccc(-c3nnn[nH]3)nc1)CC(F)(F)C2 ; COc1cc(C)c2[nH]ccc2c1CN1CCC2(C[C@@H]1c1ccc(-c3nnn[nH]3)nc1)CC(F)(F)C2 ; Cc1cc(C2CC2)c(CN2CCC3(C[C@H]2c2ccc(-c4nnn[nH]4)nc2)CC(F)(F)C3)c2cc[nH]c12 ; Cc1cc(C2CC2)c(CN2CCC3(C[C@@H]2c2ccc(-c4nnn[nH]4)nc2)CC(F)(F)C3)c2cc[nH]c12]

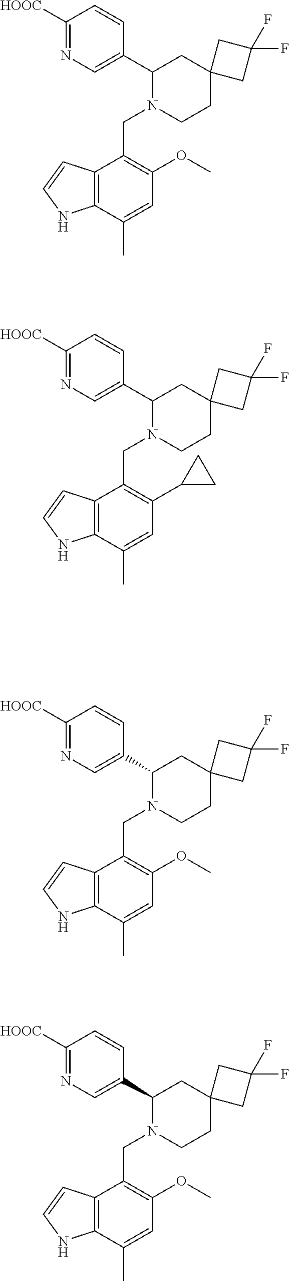 [SMILES: COc1cc(C)c2[nH]ccc2c1CN1CCC2(CC1c1ccc(C(=O)O)nc1)CC(F)(F)C2 ; Cc1cc(C2CC2)c(CN2CCC3(CC2c2ccc(C(=O)O)nc2)CC(F)(F)C3)c2cc[nH]c12 ; COc1cc(C)c2[nH]ccc2c1CN1CCC2(C[C@H]1c1ccc(C(=O)O)nc1)CC(F)(F)C2 ; COc1cc(C)c2[nH]ccc2c1CN1CCC2(C[C@@H]1c1ccc(C(=O)O)nc1)CC(F)(F)C2]

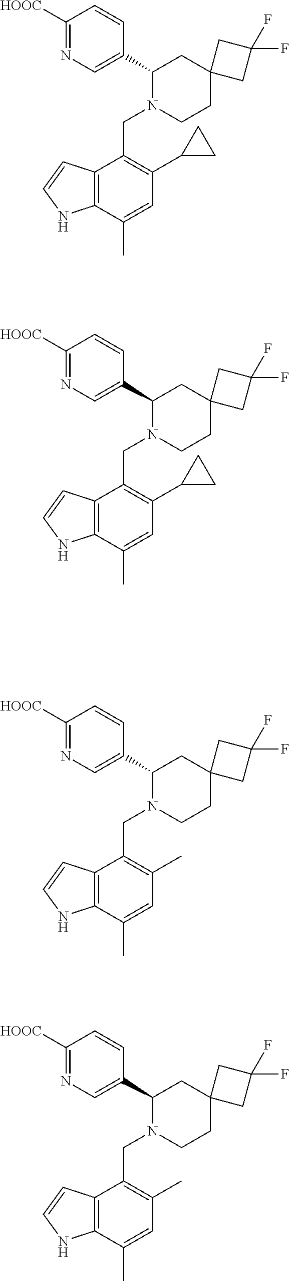 [SMILES: Cc1cc(C2CC2)c(CN2CCC3(C[C@H]2c2ccc(C(=O)O)nc2)CC(F)(F)C3)c2cc[nH]c12 ; Cc1cc(C2CC2)c(CN2CCC3(C[C@@H]2c2ccc(C(=O)O)nc2)CC(F)(F)C3)c2cc[nH]c12 ; Cc1cc(C)c2[nH]ccc2c1CN1CCC2(C[C@H]1c1ccc(C(=O)O)nc1)CC(F)(F)C2 ; Cc1cc(C)c2[nH]ccc2c1CN1CCC2(C[C@@H]1c1ccc(C(=O)O)nc1)CC(F)(F)C2]

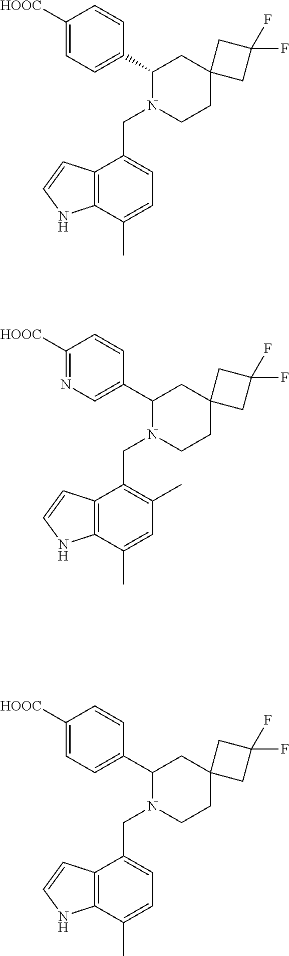 [SMILES: Cc1ccc(CN2CCC3(C[C@H]2c2ccc(C(=O)O)cc2)CC(F)(F)C3)c2cc[nH]c12 ; Cc1cc(C)c2[nH]ccc2c1CN1CCC2(CC1c1ccc(C(=O)O)nc1)CC(F)(F)C2 ; Cc1ccc(CN2CCC3(CC2c2ccc(C(=O)O)cc2)CC(F)(F)C3)c2cc[nH]c12]

Embodiment 26. The compound according to any one of the preceding Embodiments, or a pharmaceutically acceptable salt thereof, wherein the compound is present in at least 90% enantiomeric excess, at least 95% enantiomeric excess, or at least 99% enantiomeric excess.
Embodiment 27. The compound according to any one of the preceding Embodiments, or a pharmaceutically acceptable salt thereof, wherein the compound is present in at least 90% diastereomeric excess, at least 95% diastereomeric excess, or at least 99% diastereomeric excess.
Embodiment 28. A pharmaceutical composition comprising a therapeutically effective amount of a compound according to any one of the preceding Embodiments, or a pharmaceutically acceptable salt thereof, and a pharmaceutically acceptable carrier or excipient.
Embodiment 29. A method of treating or preventing a disease or disorder in a subject in need thereof, the method comprising administering to the subject a therapeutically effective amount of a compound according to any one of Embodiments 1 to 27, or a pharmaceutically acceptable salt thereof.
Embodiment 30. A method of modulating the complement alternative pathway activity in a subject, the method comprising administering to the subject a therapeutically effective amount of a compound according to any one of Embodiments 1 to 27, or a pharmaceutically acceptable salt thereof.
Embodiment 31. A method of treating a disease or disorder mediated by complement activation, in particular mediated by activation of the complement alternative pathway, in a subject in need thereof, the method comprising administering to the subject a therapeutically effective amount of a compound according to any one of Embodiments 1 to 27, or a pharmaceutically acceptable salt thereof.
Embodiment 32. A method of treating a disease or disorder that is affected by the modulation of complement alternative pathway comprising administering to the subject a therapeutically effective amount of a compound according to any one of Embodiments 1 to 27, or a pharmaceutically acceptable salt thereof.
Embodiment 33. A method of treating a disease or disorder associated with dysregulation of the complement alternative pathway comprising administering to the subject a therapeutically effective amount of a compound according to any one of Embodiments 1 to 27, or a pharmaceutically acceptable salt thereof.
Embodiment 34. A method of inhibiting the expression or activity of complement factor B, the method comprising administering to the subject a compound according to any one of Embodiments 1 to 27, or a pharmaceutically acceptable salt thereof.
Embodiment 35. The method according to any one of Embodiments 29 and 31 to 33, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.
Embodiment 36. A method of treating age-related macular degeneration comprising administering to a subject in need thereof an effective amount of a composition comprising a compound according to any one of Embodiments 1 to 27, or a pharmaceutically acceptable salt thereof.
Embodiment 37. A compound according to any one of Embodiments 1 to 27, or a pharmaceutically acceptable salt thereof, for use as a medicament.
Embodiment 38. A compound according to any one of Embodiments 1 to 27, or a pharmaceutically acceptable salt thereof, for use in inhibiting the expression or activity of complement factor B, in a subject in need thereof.
Embodiment 39. A compound according to any one of Embodiments 1 to 27, or a pharmaceutically acceptable salt thereof, for use in treating a disease or disorder associated with dysregulation of the complement alternative pathway.
Embodiment 40. The compound for use according to Embodiment 39, or a pharmaceutically acceptable salt thereof, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.
Embodiment 41. Use of a compound according to any one of Embodiments 1 to 27, or a pharmaceutically acceptable salt thereof, in the manufacture of a medicament for the treatment of a disease or disorder mediated by complement activation or activation of the complement alternative pathway.
Embodiment 42. Use of a compound according to any one of Embodiments 1 to 27, or a pharmaceutically acceptable salt thereof, in the manufacture of a medicament for the treatment of a disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.
Embodiment 43. Use of a compound according to any one of Embodiments 1 to 27, or a pharmaceutically acceptable salt thereof, for the treatment of a disease or disorder mediated by complement activation or activation of the complement alternative pathway.
Embodiment 44. Use of a compound according to any one of Embodiments 1 to 27, or a pharmaceutically acceptable salt thereof, for the treatment of a disease or disorder that is affected by the modulation of complement alternative pathway.
Embodiment 45. Use of a compound according to any one of Embodiments 1 to 27, or a pharmaceutically acceptable salt thereof, for the treatment of a disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.
Embodiment 46. Use of a compound according to any one of Embodiments 1 to 27, or a pharmaceutically acceptable salt thereof, for the treatment of age-related macular degeneration.
Embodiment 47. A pharmaceutical combination comprising a compound according to any one of Embodiment s 1 to 27, or a pharmaceutically acceptable salt thereof, and one or more additional therapeutic agent(s).

Depending on the choice of the starting materials and procedures, the compounds can be present in the form of one of the possible isomers or as mixtures thereof, for example as pure optical isomers, or as isomer mixtures, such as racemates and diastereomeric mixtures, depending on the number of asymmetric centres. The disclosure is meant to include all such possible isomers, including racemic mixtures, enantiomerically enriched mixtures, diastereomeric mixtures and optically pure forms. Optically active (R)- and (S)-isomers may be prepared using chiral synthons or chiral reagents, or resolved using conventional techniques. If the compound contains a disubstituted or trisubstituted cycloalkyl, the cycloalkyl substituent(s) may have a cis- or trans-configuration. The disclosure includes cis and trans configurations of substituted cycloalkyl groups, e.g., cyclobutyl group, as well as mixtures thereof. All tautomeric forms are also intended to be included. In particular, where a heteroaryl ring containing N as a ring atom is 2-pyridone, for example, tautomers where the carbonyl is depicted as a hydroxy (e.g., 2-hydroxypyridine) are included.

Separation of cis and trans isomers can be achieved according to methods known to a person of skill in the art, such as chromatographic methods, with tools such as HPLC (High Performance Liquid Chromatography), Thin Layer Chromatography, SFC (Supercritical Fluid Chromatography), GC (Gas Chromatography), or recrystallization techniques.

### Pharmaceutically Acceptable Salts

As used herein, the terms “salt” or “salts” refers to an acid addition or base addition salt of a compound of the disclosure. “Salts” include in particular “pharmaceutically acceptable salts”. The term “pharmaceutically acceptable salts” refers to salts that retain the biological effectiveness and properties of the compounds of this disclosure and, which typically are not biologically or otherwise undesirable. The compounds of the disclosure may be capable of forming acid and/or base salts by virtue of the presence of amino and/or carboxyl groups or groups similar thereto.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, formic acid, trifluoroacetic acid, and the like. In an embodiment, the compounds of Formula (I) are in HCl or formic acid salt form.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the disclosure provides compounds in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the disclosure provides compounds in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

### Isotopically Labelled Compounds

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, sulfur, fluorine, chlorine and iodine, such as ²H, ³H, ¹¹C, ¹³C, ¹⁴C, ¹⁸O, ¹⁵N, ¹⁸F, ¹⁷O, ¹⁸O, ³⁵S ³⁶Cl, ¹²³I, ¹²⁴I, ¹²⁵I respectively. The disclosure includes various isotopically labeled compounds as defined herein, for example those into which radioactive isotopes, such as ³H and ¹⁴C or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F compound may be particularly desirable for PET or SPECT studies. Isotopically-labeled compounds of formula (I), or sub-formulae thereof, can generally be prepared by conventional techniques known to those skilled in the art or by processes analogous to those described in the accompanying Examples and General Schemes using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I), or any of the sub-formulae thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the disclosure include those wherein the solvent of crystallization may be isotopically substituted, e.g., D₂O, d₆-acetone, d₆-DMSO.

Compounds of the disclosure, i.e., compounds of formulae (I), (I-A), (I-B), that contain groups capable of acting as donors and/or acceptors for hydrogen bonds may be capable of forming co-crystals with suitable co-crystal formers. These co-crystals may be prepared from compounds of formula (I), or sub-formulae thereof, by known co-crystal forming procedures. Such procedures include grinding, heating, co-subliming, co-melting, or contacting in solution compounds of (I), (I-A), (I-B), with the co-crystal former under crystallization conditions and isolating co-crystals thereby formed. Suitable co-crystal formers include those described in WO 2004/078163.

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

Any asymmetric center (e.g., carbon or the like) of the compound(s) of the disclosure can be present in racemic or enantiomerically enriched, for example the (R)-, (S)- or (R,S)-configuration. In certain embodiments, for example, as a mixture of enantiomers, each asymmetric center is present in at least 10% enantiomeric excess, at least 20% enantiomeric excess, at least 30% enantiomeric excess, at least 40% enantiomeric excess, at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess. In certain embodiments, for example, in enantiomerically enriched form, each asymmetric center is present in at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess. Thus, compounds of the disclosure can be present in a racemic mixture or in enantiomerically enriched form or in an enantiopure form or as a mixture of diastereoisomers.

In an embodiment, there is provided a compound of formula (I), or a pharmaceutically acceptable salt thereof, present in at least 90% enantiomeric excess, at least 95% enantiomeric excess, or at least 99% enantiomeric excess.

In an embodiment, there is provided a compound of formula (I), or a pharmaceutically acceptable salt thereof, present in at least 90% diastereomeric excess, at least 95% diastereomeric excess, or at least 99% diastereomeric excess.

In one embodiment, the compound of formula (I) is a compound of formulae (I-A):

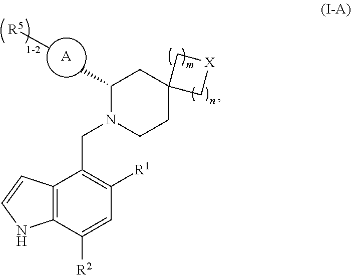

or a pharmaceutically acceptable salt thereof, wherein R¹, R², R⁵, A, X, n and m are defined according to Embodiment 1. In particular, R¹, R², R⁵, A, X, n and m may be defined according to any of Embodiments 1 to 25.

In another embodiment, the compound of formula (I) is a compound of formulae (I-B):

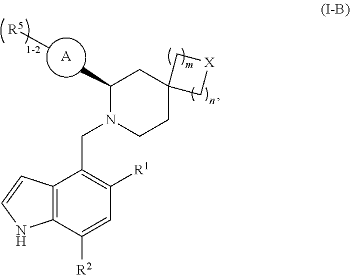

or a pharmaceutically acceptable salt thereof, wherein R¹, R², R⁵, A, X, n and m are defined according to Embodiment 1. In particular, R¹, R², R⁵, A, X, n and m may be defined according to any of Embodiments 1 to 25.

In the formulae of the present application the term “ [symbol] ” on a C-sp³ indicates the absolute stereochemistry, either (R) or (S). In the formulae of the present application the term “ [symbol] ” on a C-sp³ indicates the absolute stereochemistry, either (R) or (S). In the formulae of the present application the term “ [symbol] ” on a C-sp³ represents a covalent bond wherein the stereochemistry of the bond is not defined. This means that the term “ [symbol] ” on a C-sp³ comprises an (S) configuration or an (R) configuration of the respective chiral centre. Furthermore, mixtures may also be present. Therefore, mixtures of stereoisomers, e.g., mixtures of enantiomers, such as racemates, and/or mixtures of diastereoisomers are encompassed by the present disclosure.

For the avoidance of doubt, where compound structures are drawn with undefined stereochemistry with respect to any R group, as represented by a bond ( [symbol] ), this means the asymmetric center has either a (R)- or (S)-configuration, or exists as a mixture thereof and stated as such.

For the avoidance of doubt, in any of formulae of the present application when the R⁵ group is shown with attachment to ring A, this means that the R⁵ group(s) can be bonded via a carbon atom or heteroatom, e.g., nitrogen.

For the avoidance of doubt, the compounds structures shown herein, when appropriate, may exist in their zwitterionic form.

Accordingly, as used herein a compound of the disclosure can be in the form of one of the possible stereoisomers, rotamers, atropisomers, tautomers or mixtures thereof, for example, as substantially pure geometric (cis or trans) stereoisomers, diastereomers, optical isomers, racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the disclosure or of intermediates can be resolved into the optical isomers (enantiomers) by known methods, e.g., by separation of the diastereomeric salts thereof, obtained with an optically active acid or base, and liberating the optically active acidic or basic compound. In particular, a basic moiety may thus be employed to resolve the compounds of the disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the disclosure or racemic intermediates can also be resolved by chiral chromatography, e.g., high pressure liquid chromatography (HPLC) using a chiral adsorbent.

Furthermore, the compounds of the disclosure, including their salts, can also be obtained in the form of their hydrates, or include other solvents used for their crystallization. The compounds of the disclosure may inherently or by design form solvates with pharmaceutically acceptable solvents (including water); therefore, it is intended that the disclosure embrace both solvated and unsolvated forms. The term “solvate” refers to a molecular complex of a compound of the disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The presence of solvates can be identified by a person of skill in the art with tools such as NMR.

The compounds of the disclosure, including salts, hydrates and solvates thereof, may inherently or by design form polymorphs.

### Methods of Making

The compounds of the disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art.

Generally, the compounds of formula (I) can be prepared according to the Schemes provided infra.

Compounds provided herein can be prepared according to the following Examples. In the following Schemes, R¹, R², R⁵, A, X, n and m are defined according to enumerated Embodiment 1. In an embodiment, R¹, R², R⁵, A, X, n and m are defined according to any one of enumerated Embodiments 1 to 25. PG refers to protecting groups. Suitable protecting groups are known to one skilled in the art and the same or different protecting groups may be used in any Scheme. Additional definitions are provided as applicable in the General Schemes below.

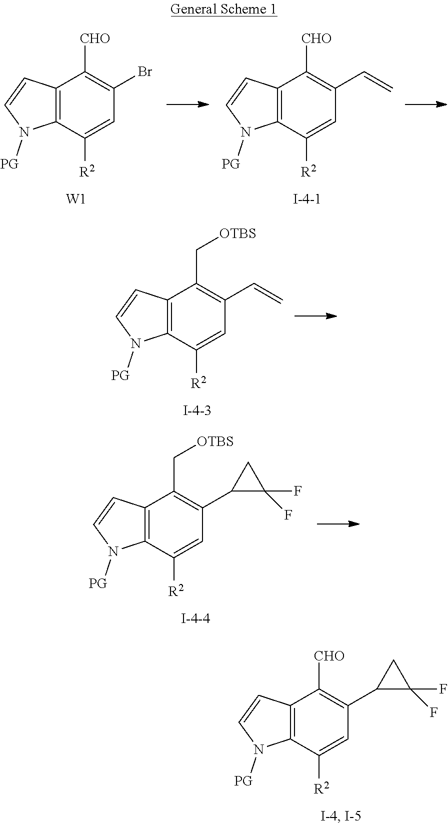

Intermediates I-4 and I-5 can be prepared as outlined in General Scheme 1. Indole W1 can be transformed to I-4-1 utilizing a Suzuki coupling with a vinyl boronate. I-4-1 can be further transformed into I-4-3 after aldehyde reduction with sodium borohydride and introduction of a TBS protecting group via electrophilic substitution with TBSCI and base. The alkene of I-4-3 can be reacted with Me₃SiCF₃ and potassium iodide at elevated temperature to access I-4-4. Intermediates I-4 and I-5 can be accessed via alcohol deprotection employing TBAF, followed by oxidation using MnO₂.

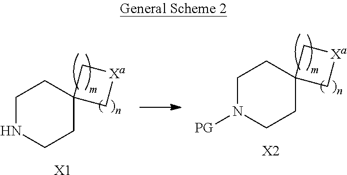

Protected amines X2 can be prepared as outlined by General Scheme 2. Commercially available secondary amines can be protected via reaction with an appropriate electrophile (e.g., Boc₂O). X^{a} is selected from X, C═O, or a ketal (e.g., X^{a} is C(OMe)₂. The ketal can be furnished by reacting the ketone (X^{a} is C═O) with methanol under acidic conditions.

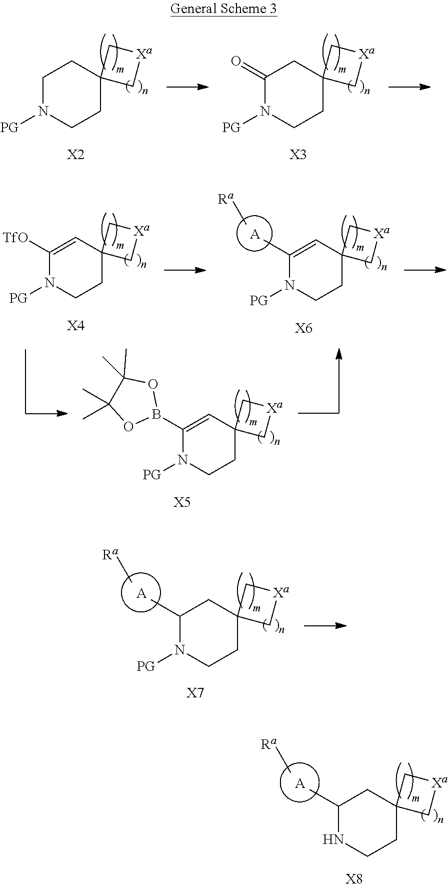

Amines such as X8 can be accessed as described by General Scheme 3. In General Scheme 3, R^{a} has the same definition as R⁵, with the exception that when R⁵ is tetrazole, R^{a} is —CN. X^{a} is as defined in General Scheme 2. X2 is oxidized with ruthenium trichloride and sodium periodiate to furnish the lactam X3. Vinyl triflate X4 can be accessed by treatment with N-phenyl-bis(trifluoromethanesulfonimide) under basic conditions. X4 can be transformed to X6 via palladium-catalyzed Suzuki coupling with an appropriate boronate. Alternatively, Miyura borylation of X4 can furnish X5, which can further react with an appropriate aryl halide to furnish X6. Olefin reduction of X6 is achieved via hydrogenation. Deprotection of X7 can furnish X8.

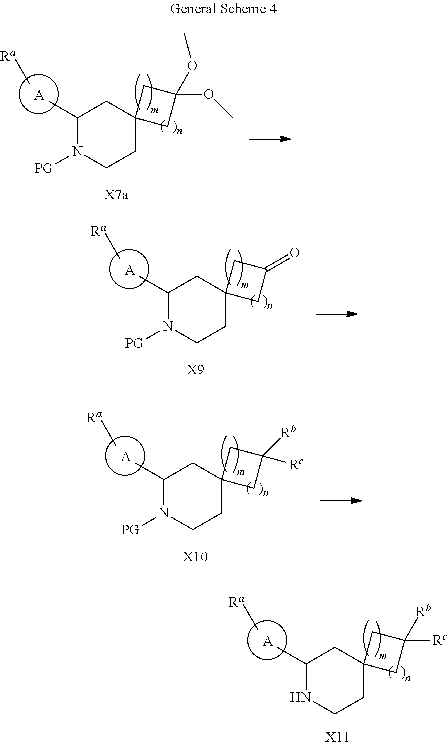

Functionalization of amine X7a, where X^{a} is C(OMe)₂, is described in General Scheme 4. R^{a} is as defined in General Scheme 3. Ketal cleavage is achieved via hydrolysis under acidic conditions. The ketone can be further transformed to X10, wherein the alcohol is formed by reduction with sodium borohydride (R^{b}═H, R^{c}═OH). Alternatively, nucleophilic addition of an organometallic reagent such as methyl magnesium bromide can yield a tertiary alcohol (R^{b}═CH₃, R^{c}═OH). Transformation of R^{c}═OH to R^{c}═F can be achieved using electrophilic fluorination or transformation of the alcohol to a leaving group followed by nucleophilic fluorination. Deprotection of X10 can furnish X11 as described above.

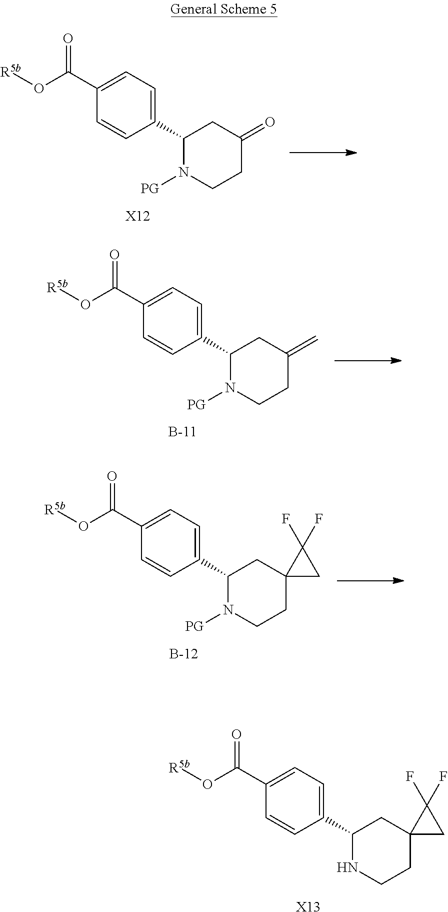

Synthesis of amine X13, is described in General Scheme 5. Ketone X12 is olefinated using the Wittig reagent. Cyclopropanation of B-11 is performed as described above. Lastly, deprotection of B-12 is performed.

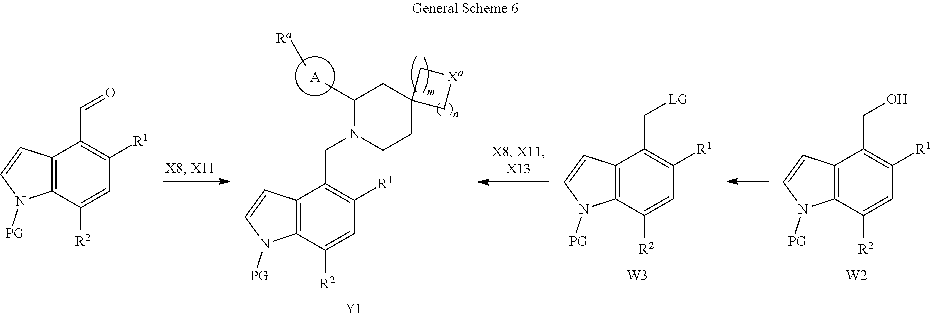

Indole aldehydes can be coupled with the cyclic amines described above employing reductive alkylation conditions, e.g. treatment with sodium triacetoxyborohydride in DCE, to provide Y1, as described in General Scheme 6. R^{a} is as defined in General Scheme 3. Alternatively the hydroxy group in the hydroxymethyl indoles W2 can be converted to a leaving group (LG, e.g., chloride) W3 by treatment with cyanuric chloride. W3 can be reacted with a cyclic amine in the presence of a base such as DIPEA in a solvent such as DMF at temperatures ranging from 0° C. to 50° C. to afford Y1.

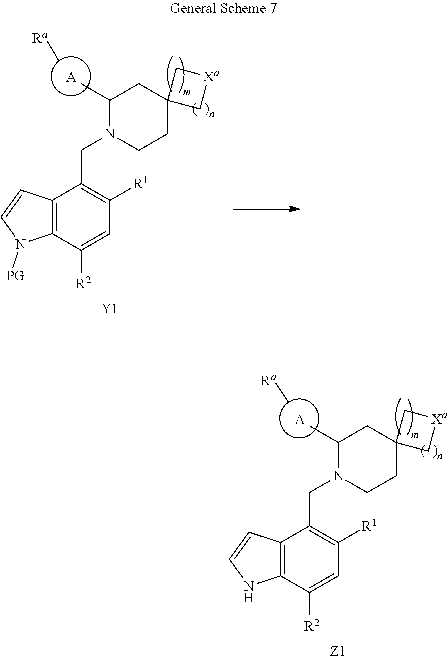

Compounds such as Z1 ca n be prepared according to General Scheme 7. R^{a} is as defined in General Scheme 3. R^{a} is as defined in General Scheme 3. Deprotection of PG (PG=Boc) in compound Y1 to Z1 can be accomplished by a treatment with a source of hydroxide such as KOH or LiOH in a suitable solvent system such as a mixture of THF/MeOH/water at temperatures ranging from RT to 50° C. Deprotection of PG from Y1 when R^{a}═COOMe can also result in concomitant reaction of the —COOMe ester to provide Z1 wherein R^{e}═—COOH. In addition, treatment of Y1, when PG=Boc, with an appropriate base as potassium carbonate in a solvent such as methanol at temperature up to 50° C. can provide Z1.

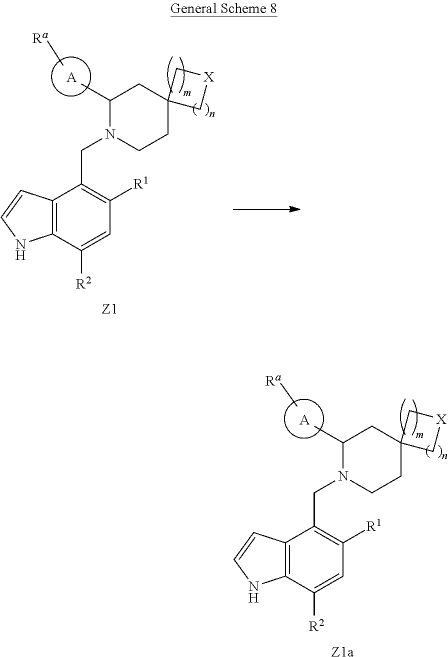

Compounds such as Z1a wherein R⁵=tetrazole, can be prepared according to General Scheme 8. Z1 when R^{a}═CN can be transformed to Z1a wherein R⁵ is a tetrazole ring by a treatment with azide containing reagents such as sodium azide in the presence of catalysts such as triethylamine hydrochloride in a suitable solvent.

In a further aspect, the disclosure provides a process for the preparation of a compound of formula (I), in free form or in pharmaceutically acceptable salt form, comprising the step as described above.

### Pharmaceutical Compositions

In another aspect, the disclosure provides a pharmaceutical composition comprising one or more compounds of described herein or a pharmaceutically acceptable salt thereof, and one or more pharmaceutically acceptable carriers.

In a further embodiment, the composition comprises at least two pharmaceutically acceptable carriers, such as those described herein. For purposes of the disclosure, unless designated otherwise, solvates and hydrates are generally considered compositions.

The compounds of Formula (I), and subformulae thereof, described herein may be administered alone or as an active ingredient of a pharmaceutical composition. Accordingly, provided herein are pharmaceutical compositions comprising a compounds of Formula (I), or subformulae thereof, or a pharmaceutically acceptable salt thereof, and one or more pharmaceutically acceptable carriers.

Methods of preparing various pharmaceutical compositions are known to those of skill in the art and may be described in, for example, in the Handbook of Pharmaceutical Excipients, American Pharmaceutical Association (current edition); Pharmaceutical Dosage Forms Tablets (Lieberman, Lachman and Schwartz, editors) current edition, published by Marcel Dekker, Inc., as well as Remington's Pharmaceutical Sciences (Arthur Osol, editor), 1553-1593 (current edition).

The pharmaceutical composition can be formulated for particular routes of administration such as oral administration, topical administration, parenteral administration, and rectal administration, etc. In addition, the pharmaceutical compositions of the disclosure can be made up in a solid form (including without limitation capsules, tablets, pills, granules, powders or suppositories), or in a liquid form (including without limitation solutions, gels, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.

In an embodiment, the pharmaceutical compositions are capsules comprising the active ingredient only.

Tablets may be either film coated or enteric coated according to methods known in the art.

The mode of administration and pharmaceutical composition are closely related to the therapeutic amounts of the compounds or compositions which are desirable and efficacious for the given treatment application. Pharmaceutical compositions provided herein can be formulated for ophthalmic, ocular, topical, and transdermal administration. In particular embodiments, the pharmaceutical compositions provided herein are suitable for ocular administration. To prepare pharmaceutical compositions, the active ingredient may be mixed with one or more pharmaceutically acceptable carrier(s) according to conventional pharmaceutical compounding techniques. The carrier(s) may take a wide variety of forms depending on the form of preparation desired for administration.

Suitable compositions for oral administration include an effective amount of a compound of the disclosure in the form of tablets, lozenges, aqueous or oily suspensions, dispersible powders or granules, emulsion, hard or soft capsules, or syrups or elixirs, solutions or solid dispersion. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients, which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

In certain embodiments, the pharmaceutical compositions provided herein are formulated as solutions, suspensions, gels, creams, ointments, liposomes, ocular inserts or other pharmaceutical compositions suitable, in particular embodiments, for topical administration to the ocular surface, the cornea, the eyelid, margins of the eye, eyelashes and/or eye lid margin in order to deliver the composition to the eye. In some embodiments, liquid (aqueous or non-aqeuous) solutions may be used. In certain embodiments the pharmaceutical compositions are formulated as eye drops for topical administration to the ocular surface, the cornea, the eyelid, eye lid margins, eyelashes and/or margins of the eye in order to deliver the composition to the eye. Application of the pharmaceutical composition may be performed with an applicator, such as the subject's finger, a Weck-Cel®, Q-Tip®, or other device capable of delivering a formulation to the eyelid, eyelashes and/or eyelid margin in order to deliver the formulation to the eye. The pharmaceutical compositions provided herein may be viscous or semi-viscous; liquid, solid, or semi-solid; aqueous or non-aqueous, depending on the site of application, dose, solubility of drug, and a variety of other factors that are considered by those of skill in the art.

Any of a variety of carriers may be used in a pharmaceutical composition provided herein. In one embodiment, the pharmaceutically acceptable carrier is a non-aqueous carrier (e.g., oil, or oil mixture) having a viscosity in a range from about 50 cps to about 1000 cps, about 50 cps to about 500 cps, about 50 cps to about 200 cps, or about 60 cps to about 120 cps. In certain embodiments, the non-aqueous carrier comprises an oil, e.g., vegetable oils, silicone oils, mineral oil or any combination thereof. In some embodiments, the carrier may be liquid paraffin, white petrolatum, purified lanolin, gelation hydrocarbon, polyethylene glycol, hydrophilic ointment base, white ointment base, absorptive ointment base, Macrogol ointment base, simple ointment base, and the like. In certain embodiments, the pharmaceutical composition may include a monomeric polyol such as, glycerol, propylene glycol, and ethylene glycol, polymeric polyols such as polyethylene glycol, cellulose esters such hydroxypropylmethyl cellulose, carboxy methylcellulose sodium and hydroxy propylcellulose; dextrans such as dextran 70; water soluble proteins such as gelatin, polymers such as polyvinyl alcohol, polyvinylpyrrolidone, and povidone; carbomers, such as carbomer 934P. carbomer 941, carbomer 940 and carbomer 974P; and gums such as HP-guar.

Additional excipients may optionally be included in the pharmaceutical compositions provided herein. Examples of additional excipients include, for example, tonicity enhancers, preservatives, solubilizers, non-toxic excipients, demulcents, sequestering agents, pH adjusting agents, co-solvents, viscosity building agents, and combinations thereof.

The pharmaceutical composition of the disclosure may be in the form of an aqueous suspension or an aqueous solution. In one embodiment, the aqueous pharmaceutical composition of the disclosure is in the form of an aqueous suspension.

Aqueous pharmaceutical compositions according to the disclosure can be prepared using standard procedures that are familiar to the person skilled in the art, e.g., by admixture of the various components, suitably at ambient temperature and atmospheric pressure. In one embodiment, the aqueous pharmaceutical compositions of the disclosure are suitable for ocular administration.

In a further embodiment, the pharmaceutical composition of the disclosure is in the form of eye ointment, eye gel, eye cream, or eye drops.

In a further embodiment, the pharmaceutical composition of the disclosure is administered to the subject topically in the eyes.

The compounds of formula (I), in free form or in pharmaceutically acceptable salt form, exhibit valuable pharmacological properties, e.g., complement Factor B modulating properties e.g., as indicated in the in vitro tests as provided in the examples, and are therefore indicated for therapy or for use as research chemicals, e.g., as tool compounds.

Additional properties of the disclosed compounds include having good potency in the biological assays described herein, favorable safety profile, and possess favorable pharmacokinetic properties.

### Diseases and Disorders and Methods of Use

In a further aspect, the disclosure provides a compound of formula (I), or a pharmaceutically acceptable salt thereof, for use in therapy.

In a further aspect, the disclosure provides a compound of formula (I), or a pharmaceutically acceptable salt thereof, for use in the treatment of a disease or disorder for which complement factor B is indicated. In one embodiment, the disease or disorder is affected by the inhibition of complement factor B activity.

Compounds of formula (I) and their pharmaceutically acceptable salts have complement factor B modulating and/or inhibitory activity and are believed to be of potential use for the treatment or prophylaxis of certain diseases or disorders, such as age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

Having regard to their activity as complement factor B modulator or inhibitors, compounds of formula (I) and sub-formulae thereof, in free or pharmaceutically acceptable salt form, are useful in the treatment of conditions which may be treated by inhibition of complement factor B activity. In one aspect, the disclosure provides a method of treating or preventing a disease or disorder in a subject in need thereof, the method comprising administering to the subject a therapeutically effective amount of a compound of formula (I) or sub-formula thereof, or a pharmaceutically acceptable salt thereof.

In another aspect, the disclosure provides a method of modulating the complement alternative pathway activity in a subject, the method comprising administering to the subject a therapeutically effective amount of a compound of formula (I) or sub-formula thereof, or a pharmaceutically acceptable salt thereof.

In another aspect, the disclosure provides a method of treating a disease or disorder mediated by complement activation, in particular mediated by activation of the complement alternative pathway, in a subject in need thereof, the method comprising administering to the subject a therapeutically effective amount of a compound of formula (I) or sub-formula thereof, or a pharmaceutically acceptable salt thereof.

In another aspect, the disclosure provides a method of treating a disease or disorder that is affected by the modulation of complement alternative pathway comprising administering to the subject a therapeutically effective amount of a compound of formula (I) or sub-formula thereof, or a pharmaceutically acceptable salt thereof.

In another aspect, the disclosure provides a method of treating a disease or disorder associated with dysregulation of the complement alternative pathway comprising administering to the subject a therapeutically effective amount of a compound of formula (I) or sub-formula thereof, or a pharmaceutically acceptable salt thereof.

In another aspect, the disclosure provides a method of inhibiting the expression or activity of complement factor B, the method comprising administering to the subject a compound of formula (I) or sub-formula thereof, or a pharmaceutically acceptable salt thereof.

In another aspect, the disclosure provides a method of treating disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

In certain aspects, methods are provided for the treatment of diseases associated with increased activity of the C3 amplification loop of the complement pathway. In certain embodiments, methods of treating or preventing complement mediated diseases are provided in which the complement activation is induced by antibody-antigen interactions, by a component of an autoimmune disease, or by ischemic damage.

In a specific embodiment, the present disclosure provides a method of treating or preventing age-related macular degeneration (AMD) by administering to a subject in need thereof an effective amount of the compound of Formula (I) of the disclosure. In certain embodiments, patients who are currently asymptomatic but are at risk of developing a symptomatic macular degeneration related disorder are suitable for administration with a compound of the disclosure. The methods of treating or preventing AMD include, but are not limited to, methods of treating or preventing one or more symptoms or aspects of AMD selected from formation of ocular drusen, inflammation of the eye or eye tissue, loss of photoreceptor cells, loss of vision (including loss of visual acuity or visual field), neovascularization (including CNV), retinal detachment, photoreceptor degeneration, RPE degeneration, retinal degeneration, chorioretinal degeneration, cone degeneration, retinal dysfunction, retinal damage in response to light exposure, damage of the Bruch's membrane, and/or loss of RPE function.

The compound of Formula (I) of the disclosure can be used, inter alia, to prevent the onset of AMD, to prevent the progression of early AMD to advanced forms of AMD including neovascular AMD or geographic atrophy, to slow and/or prevent progression of geographic atrophy, to treat or prevent macular edema from AMD or other conditions (such as diabetic retinopathy, uveitis, or post surgical or non-surgical trauma), to prevent or reduce the loss of vision from AMD, and to improve vision lost due to pre-existing early or advanced AMD. It can also be used in combination with anti-VEGF therapies for the treatment of neovascular AMD patients or for the prevention of neovascular AMD.

All the aforementioned embodiments relating to the methods of treatment of the aforementioned diseases are equally applicable to:
a compound of formula (I) or sub-formula thereof, or a pharmaceutically acceptable salt thereof, for use in the treatment of the aforementioned diseases according to the present disclosure;
use of a compound of formula (I) or sub-formula thereof, or a pharmaceutically acceptable salt thereof, in the manufacture of a medicament for the treatment of the aforementioned diseases according to the present disclosure;
use of a compound of formula (I) or sub-formula thereof, or a pharmaceutically acceptable salt thereof, for the treatment of the aforementioned diseases according to the present disclosure; and
a pharmaceutical composition comprising a compound of formula (I) or sub-formula thereof, or a pharmaceutically acceptable salt thereof, and one or more pharmaceutically acceptable carriers, for use in the treatment of the aforementioned diseases according to the present disclosure.

### Dosage

The pharmaceutical composition or combination of the disclosure can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated.

The above-cited dosage properties are demonstrable in vitro and in vivo tests using advantageously mammals, e.g., mice, rats, dogs, monkeys or isolated organs, tissues and preparations thereof. The compounds of the disclosure can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, e.g., between about 0.1-500 mg/kg.

The therapeutically effective dosage of a compound, the pharmaceutical composition, or the combinations thereof, is dependent on the species of the subject, the body weight, age and individual condition, the disorder or disease or the severity thereof being treated.

The activity of a compound according to the disclosure can be assessed by the in vitro methods described in the Examples.

### Combination Therapy

In another aspect, the disclosure provides a pharmaceutical combination comprising a compound of formula (I), or subformulae thereof, or a pharmaceutically acceptable salt thereof, and one or more additional therapeutic agent(s) for simultaneous, separate or sequential use in therapy.

The compound of the disclosure may be administered either simultaneously with, or before or after, one or more other therapeutic agent. The compound of the disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the disclosure. Thus, in one embodiment, the disclosure provides a combination comprising a therapeutically effective amount of a compound of formula or subformulae thereof, or a pharmaceutically acceptable salt thereof, and one or more therapeutically active agents.

In certain embodiments, a compound of Formula (I), or subformulae thereof, or a pharmaceutically acceptable salt thereof, may be administered with an additional therapeutic agent.

The pharmaceutical compositions of the disclosure, e.g., a pharmaceutical composition comprising a compound of formula (I), can be administered alone or in combination with other molecules known to have a beneficial effect on retinal attachment or damaged retinal tissue, including molecules capable of tissue repair and regeneration and/or inhibiting inflammation. Examples of useful, cofactors include complement inhibitors (such as inhibitors of Factor D, C5a receptor and antibody or Fabs against C5, C3, properidin, factor H, and the like), anti-VEGF agents (such as an antibody or FAB against VEGF, e.g., Lucentis or Avastin), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics. Suitable agents for combination treatment with the compounds of the disclosure include agents known in the art that are able to modulate the activities of complement components.

In some embodiments, the present disclosure provide a combination therapy for preventing and/or treating AMD or another complement related ocular disease as described above with a compound of the disclosure and an anti-angiogenic, such as anti-VEGF agent (including Lucentis Avastin and VEGF-R2 inhibitors including pazopanib, sutent, inifanib, and VEGF-R2 inhibitors disclosed in WO2010/066684) or photodynamic therapy (such as as verteporfin).

In some embodiments, the present disclosure provide a combination therapy for preventing and/or treating autoimmune disease as described above with a compound of the disclosure and a B-Cell or T-Cell modulating agent (for example cyclosporine or analogs thereof, rapamycin, RAD001 or analogs thereof, and the like). In particular, for multiple sclerosis therapy may include the combination of a compound of the disclosure and a second MS agent selected from fingolimod, cladribine, tysarbi, laquinimod, rebif, avonex and the like. A non-limiting list of such agents incudes pharmaceutical agents effective in the treatment of diseases and conditions in which vanilloid receptor activation plays a role or is implicated, including cyclooxygenase-2 (COX-2) inhibitors, such as specific COX-2 inhibitors, e.g., celecoxib and rofecoxib; and non-steroidal anti-inflammatory drugs (NSAIDs), e.g., acetylsalicylic acid and propionic acid derivatives; tricyclic anti-depressants, e.g., Anafranil®, Asendin®, Aventyl®, Elavil®, Endep®, Norfranil®, Norpramin®, Pamelor®, Sinequan®, Surmontil®, Tipramine®, Tofranil®, Vivactil®, Tofranil-PM®; anti-convulsants, e.g., carbamazepine, oxcarbazepine and gabapentin; bradykinin B1 or B2 antagonists; and GABA_{B} agonists, e.g., L-baclofen.

In certain embodiments, further therapeutic agents may include, for instance, other compounds and antibodies useful for treating ocular disorders. A non-limiting list of such agents incudes retinoid X receptor agonists, such as vitamin A, retinoic acid, phytanic acid, Iithocholic acid, bexarotene, docosahexaenoic acid, or flurobexarotene. Other additional therapeutic agents include ophthalmic steroids such as, dexamethasone, fluocinolone, loteprednol, difluprednate, fluorometholone, prednisolone, prednisone, medrysone, triamcinolone, betamethasone, rimexolone, or pharmaceutically acceptable salts thereof. In addition, other additional therapeutic agents include those used to target ocular surface disease disorders, such as dry eye disease. Non-limiting example of such additional therapeutic agents include Xiidra® (lifitegrast), Restasis® (cyclosporine), minocycline, doxycycline, or other tetracycline antibiotics. Other examples include keratolytic agents such as selenium disulfide, salicylic acid, glycolic acid etc., or pharmaceutically acceptable salts thereof.

In certain embodiments, further therapeutic agents may include, for instance, other compounds useful in the treatment of pain. In an embodiment, a compound of Formula (I), or subformula thereof, or a pharmaceutically acceptable salt thereof, may be administered with an additional analgesic agent. Such anagelsic agent may be an NSAID (e.g., acetylsalicylic acid and propionic acid derivatives, e.g., Aleve®), opioid or steroid.

### Preparation of Compounds

It is understood that in the following description, combinations of substituents and/or variables of the depicted formulae are permissible only if such combinations result in stable compounds.

It will also be appreciated by those skilled in the art that in the processes described below, the functional groups of intermediate compounds may need to be protected by suitable protecting groups. Such functional groups include hydroxy, phenol, amino and carboxylic acid. Suitable protecting groups for hydroxy or phenol include trialkylsilyl or diarylalkylsilyl (e.g., tert-butyldimethylsilyl, tert-butyldiphenylsilyl or trimethylsilyl), tetrahydropyranyl, benzyl, substituted benzyl, methyl, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters.

Protecting groups may be added or removed in accordance with standard techniques, which are well-known to those skilled in the art and as described herein. The use of protecting groups is described in detail in J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; P. J. Kocienski, “Protecting Groups”, Third Edition, Georg Thieme Verlag, Stuttgart and New York 2005; and in “Methoden der organischen Chemie” (Methods of Organic Chemistry), Houben Weyl, 4th edition, Volume 15/I, Georg Thieme Verlag, Stuttgart 1974.

The protecting group may also be a polymer resin, such as a Wang resin or a 2-chlorotrityl-chloride resin.

The following reaction Examples illustrate methods to make compounds of this disclosure. It is understood that one skilled in the art would be able to make these compounds by similar methods or by methods known to one skilled in the art. In general, starting components and reagents may be obtained from sources such as Sigma Aldrich, Lancaster Synthesis, Inc., Maybridge, Matrix Scientific, TCI, and Fluorochem USA, Strem, other commercial vendors, or synthesized according to sources known to those skilled in the art, or prepared as described in this disclosure.

### Analytical Methods, Materials, and Instrumentation

Unless otherwise noted, reagents and solvents were used as received from commercial suppliers. Proton nuclear magnetic resonance (NMR) spectra were obtained on either Bruker Avance, Avance III or Avance Neo spectrometer 400 MHz, Varian Oxford 400 MHz, or Varian Mercury 300 MHz spectrometer unless otherwise noted. Spectra are given in ppm (δ) and coupling constants, J, are reported in Hertz. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2-5 mg) is dissolved in an appropriate deuterated solvent (1 mL). The chemical names were generated using ChemBioDraw Ultra v19 from CambridgeSoft.

Mass spectra (ESI-MS) were collected using a Waters System (Acquity UPLC and a Micromass ZQ mass spectrometer) or Agilent-1260 Infinity (6120 Quadrupole); all masses reported are the m/z of the protonated parent ions unless recorded otherwise. The sample was dissolved in a suitable solvent such as MeCN, DMSO, or MeOH and was injected directly into the column using an automated sample handler.

### Abbreviations

ACN acetonitrile
aq. aqueous
Boc tertiary butyl carboxy
br broad
c concentration
CO₂ carbon dioxide
d doublet
DAD diode-array detection
DBU 1,8-diazabicyclo[5.4.0]undec-7-ene
DCE 1,2-dichloroethane
DCM dichloromethane
dd doublet of doublets
de diastereomeric excess
DEA diethanolamine
DIPEA diisopropylethylamine
DMAP 4-N,N-dimethylaminopyridine
DMA N,N-dimethyl acetamide
DMF N,N-dimethylformamide
DMSO dimethylsulfoxide
dppf 1,1′-ferrocendiylbis(diphenylphosphine)
ds diastereomer
ELSD evaporative light scattering detector
ee enantiomeric excess
ent enantiopure
eq equivalent
EtOAc ethyl acetate
EtOH ethanol
h hour(s)
H₂ hydrogen
HCl hydrochloric acid
HMDS 1,1,1,3,3,3-hexamethyldisilazane
HPLC high performance liquid chromatography
IPA isopropanol
K₂CO₃ potassium carbonate
KI potassium iodide
KOtBu potassium tert-butoxide
K₃PO₄ potassium phosphate, tribasic
LC-MS liquid chromatography and mass spectrometry
m multiplet
m/z mass to charge ratio
MeOH methanol
Me₃SiCF₃ trimethyl(trifluoromethyl)silane
2-MeTHF 2-methyltetrahydrofuran
MgSO₄ magnesium sulfate
min minutes
MnO₂ manganese dioxide
MS mass spectrometry
MTBE methyl tert-butyl ether
n-BuLi n-Butyllithium
N₂ nitrogen
NaBH(OAc)₃ sodium triacetoxyborohydride
NaBH₄ sodium borohydride
Na₂CO₃ sodium carbonate
NaHCO₃ sodium bicarbonate
NaI sodium iodide
NaIO₄ sodium periodate
NaOH sodium hydroxide
NaN₃ sodium azide
Na₂SO₄ sodium sulfate
Na₂S₂O₃ sodium thiosulfate
NH₄Cl ammonium chloride
NH₄OH ammonium hydroxide
NH₄(CH₃CO₂) ammonium acetate
NMR nuclear magnetic resonance
NP normal phase
org. organic
p.a. pro analysis
Pd/C palladium on carbon
PdCl₂(PPh₃)₂ bis(triphenylphosphine)palladium(II) dichloride
PdCl₂(dtbpf) [1,1′-bis(di-tert-butylphosphino)ferrocene]palladium
(II) dichloride
Pd₂(dba)₃ bis(dibenzylideneacetone)dipalladium
Pd(dppf)Cl₂ [1,1′-bis(diphenylphosphino)ferrocene]palladium (II)
dichloride
PE petroleum ether
ppm parts per million
rac racemic
RM reaction mixture
RP reversed phase
Rt retention time
RT room temperature
RuCl₃ ruthenium trichloride
s singlet
sat. saturated
scCO₂ super critical carbon dioxide
SFC Supercritical Fluid Chromatography
SiO₂ silica gel
soln. solution
t triplet
TBAF tetra-n-butylammonium fluoride
TBS tert-butyldimethylsilyl
TBSCI tert-butyldimethylsilyl chloride
tert-Bu tertiary butyl
TFA trifluoroacetic acid
THE tetrahydrofuran
TLC thin layer chromatography
UPLC ultra performance liquid chromatography
wt % weight percent
Zn(CN)₂ zinc cyanide

The absolute configuration (S) at the 2-position of the piperidine ring was consistently and tentatively assigned to the biologically more active enantiomer. The absolute configuration of the compounds is further confirmed by X-ray crystallography of select compounds. Stereochemical nomenclature was assigned using ChemDraw. Compounds with Chemical Abstracts Registry numbers were either purchased from commercial suppliers or prepared by known methods.

### INTERMEDIATES

### Intermediate I-1

### tert-butyl 4-formyl-5-methoxy-7-methyl-1H-indole-1-carboxylate

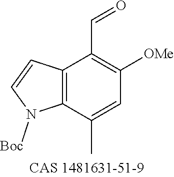 [SMILES: COc1cc(C)c2c(ccn2C(=O)OC(C)(C)C)c1C=O]

### Intermediate I-2

### tert-butyl 4-formyl-5,7-dimethyl-1H-indole-1-carboxylate

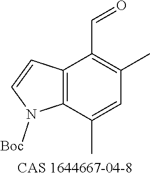 [SMILES: Cc1cc(C)c2c(ccn2C(=O)OC(C)(C)C)c1C=O]

### Intermediate I-3

### tert-butyl 5-cyclopropyl-4-formyl-7-methyl-1H-indole-1-carboxylate

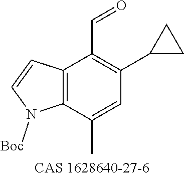 [SMILES: Cc1cc(C2CC2)c(C=O)c2ccn(C(=O)OC(C)(C)C)c12]

### Intermediate I-4 and Intermediate I-5

### tert-butyl (R)-5-(2,2-difluorocyclopropyl)-4-formyl-7-methyl-1H-indole-1-carboxylate and tert-butyl (S)-5-(2,2-difluorocyclopropyl)-4-formyl-7-methyl-1H-indole-1-carboxylate

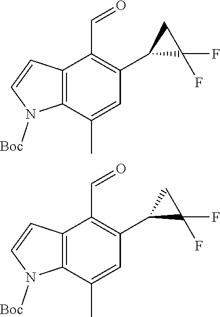

### Step 1: tert-butyl 4-formyl-7-methyl-5-vinyl-1H-indole-1-carboxylate (Intermediate I-4-1)

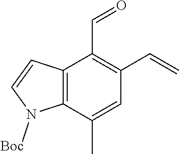 [SMILES: C=Cc1cc(C)c2c(ccn2C(=O)OC(C)(C)C)c1C=O]

To a degassed soln. of tert-butyl 5-bromo-4-formyl-7-methyl-1H-indole-1-carboxylate (CAS 1628640-26-5, 2.0 g, 5.913 mmol) in IPA (20 mL) was added added potassium vinyltetrafluoroborate (1.58 g, 11.82 mmol). The soln. was sparged with N₂ for 10 min, and then TEA (1.5 mL, 11.82 mmol) was added under N₂ atmosphere. To this soln. Pd(dppf)Cl₂-DCM (0.24 g, 0.295 mmol) was added. The mixture was sparged with N₂ for 10 min and heated to 90° C. for 16 h. The RM was concentrated, diluted with water, and extracted with EtOAc (2×). The combined org. layers were washed with water, brine, dried over Na₂SO₄, and concentrated under reduced pressure. The crude residue was purified by column chromatography (NP, 6 to 8% EtOAc in hexane). Fractions containing product were combined and concentrated under reduced pressure to yield the title compound as a brown solid (950 mg). ¹H NMR (300 MHz, CDCl₃) δ [ppm]10.6 (s, 1H), 7.65 (d, J=4.0 Hz, 1H), 7.49 (d, J=11.0 Hz, 1H), 7.52-7.42 (m, 1H), 7.24 (d, J=7.2 Hz, 1H), 5.65 (d, J=17.4 Hz, 1H), 5.54 (dd, J=11.0, 1.0 Hz, 1H), 2.69, (s, 3H), 1.64 (s, 9H).

### Step 2: tert-butyl 4-(hydroxymethyl)-7-methyl-5-vinyl-1H-indole-1-carboxylate (Intermediate I-4-2)

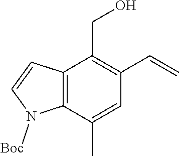 [SMILES: C=Cc1cc(C)c2c(ccn2C(=O)OC(C)(C)C)c1CO]

To a white suspension of tert-butyl 4-formyl-7-methyl-5-vinyl-1H-indole-1-carboxylate (Intermediate I-4-1, 950 mg, 3.329 mmol) in MeOH (5.0 mL) was added NaBH₄ (252 mg, 6.658 mmol) at 0° C., and the mixture stirred at RT for 1 h. The mixture was concentrated under reduced pressure, and the the residue was diluted with water. The mixture was extracted with EtOAc (3×). The combined extracts were concentrated under reduced pressure to yield the title compound as a white solid (920 mg). ¹H NMR (300 MHz, CDCl₃) δ [ppm]7.54 (d, J=4.0 Hz, 1H), 7.31 (s, 1H), 7.18, (dd, J=17.4, 11.0 Hz, 1H), 6.72 (d, J=4.0 Hz, 1H), 5.71 (dd, J=17.4, 1.0 Hz, 1H), 5.35 (dd, J=11.0, 1.0 Hz, 1H), 4.96 (s, 2H), 2.63 (s, 3H), 1.64 (s, 9H).

### Step 3: tert-butyl 4-(((tert-butyldimethylsilyl)oxy)methyl)-7-methyl-5-vinyl-1H-indole-1-carboxylate (Intermediate I-4-3)

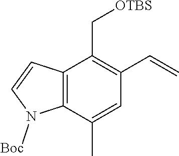 [SMILES: C=Cc1cc(C)c2c(ccn2C(=O)OC(C)(C)C)c1CO[Si](C)(C)C(C)(C)C]

To a stirred soln. of tert-butyl 4-(hydroxymethyl)-7-methyl-5-vinyl-1H-indole-1-carboxylate (Intermediate I-4-2, 900 mg, 3.131 mmol) in DCM (10 mL) were added TEA (1.21 mL, 9.395 mmol) and DMAP (0.11 g, 0.939 mmol). The mixture was stirred for 30 min at RT, TBSCI (1.41 g, 9.395 mmol) was added slowly, and the mixture was stirred at RT for 16 h. The RM was quenched with water and resulting mixture was extracted with EtOAc. The org. layer was dried over Na₂SO₄, filtered and concentrated under reduced pressure. The crude material was purified by chromatography (NP, 0 to 5% EtOAc in hexane) to obtain the title product as a yellow solid (900 mg). ¹H NMR (400 MHz, CDCl₃) δ [ppm]7.51 (d, J=4.0 Hz, 1H), 7.27 (s, 1H), 7.17, (dd, J=17.4, 11.0 Hz, 1H), 6.7 (d, 4.0 Hz, 1H), 5.63 (d, J=11.0 Hz, 1H), 5.30 (m, 1H), 4.96 (s, 2H), 2.62, (s, 3H), 1.63 (s, 9H), 0.89 (s, 9H), 0.05 (s, 6H).

### Step 4: tert-butyl 4-(((tert-butyldimethylsilyl)oxy)methyl)-5-(2,2-difluorocyclopropyl)-7-methyl-1H-indole-1-carboxylate (Intermediate I-4-4)

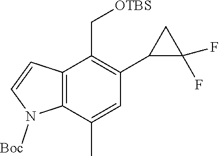 [SMILES: Cc1cc(C2CC2(F)F)c(CO[Si](C)(C)C(C)(C)C)c2ccn(C(=O)OC(C)(C)C)c12]

To a soln. of tert-butyl 4-(((tert-butyldimethylsilyl)oxy)methyl)-7-methyl-5-vinyl-1H-indole-1-carboxylate (Intermediate I-4-3, 900 mg, 2.240 mmol) in THE (15 mL) were added Me₃SiCF₃ (4.77 mg, 33.61 mmol) and NaI (0.743 g, 4.48 mmol) at RT. The RM was irradiated at 120° C. for 1 h in a microwave. The mixture was diluted with water and extracted with EtOAc (3×). The combined org. phases were washed with water, brine, dried over Na₂SO₄, and concentrated under reduced pressure to yield the title compound as a pale brown oil (950 mg). ¹H NMR (400 MHz, CDCl₃) δ [ppm]7.53 (d, J=4.0 Hz, 1H), 6.92 (s, 1H), 6.71, (d, J=4.0 Hz, 1H), 5.02 (d, J=15.0 Hz, 1H), 4.97 (d, J=15.0 Hz, 1H), 3.00-2.95 (m, 1H), 2.59, (s, 3H), 1.86-1.80 (m, 1H), 1.72-1.68 (m, 1H), 1.66 (s, 9H), 0.91 (s, 9H), 0.10 (s, 3H), 0.09 (s, 3H).

### Step-5: tert-butyl 5-(2,2-difluorocyclopropyl)-4-(hydroxymethyl)-7-methyl-1H-indole-1-carboxylate (Intermediate I-4-5)

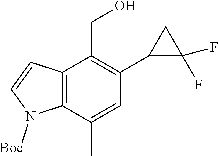 [SMILES: Cc1cc(C2CC2(F)F)c(CO)c2ccn(C(=O)OC(C)(C)C)c12]

To a soln. of tert-butyl 4-(((tert-butyldimethylsilyl)oxy)methyl)-5-(2,2-difluorocyclopropyl)-7-methyl-1H-indole-1-carboxylate (Intermediate I-4-4, 950 mg, 2.103 mmol) in THF (10 mL) was added TBAF (1.0 M in THF) (10 mL, 10.51 mmol) at RT. The RM was stirred at RT for 16 h. The mixture was diluted with water and extracted with EtOAc (3×). The combined org. layers were washed with water, brine soln., dried over Na₂SO₄, filtered, and concentrated under reduced pressure to provide the racemic title compound. LC-MS Method A-1: Rt=1.61 min; MS m/z [M−H]⁻=335.9.

### Step-6: tert-butyl (S)-5-(2,2-difluorocyclopropyl)-4-formyl-7-methyl-1H-indole-1-carboxylate and tert-butyl (R)-5-(2,2-difluorocyclopropyl)-4-formyl-7-methyl-1H-indole-1-carboxylate (Intermediate I-4-6 and Intermediate I-4-7)

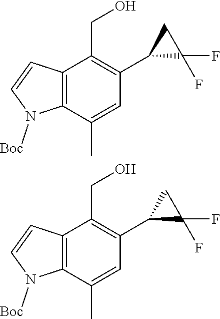

For chiral separation of Intermediate I-4-6 and Intermediate I-4-7 (1.03 g): Instrument: Agilent 1200; Column: Chiralpak IG 250 mm×20 mm×5 μm; flow rate: 15 mL/min; Mobile Phase: 90% A=hexane, 10% B=EtOH:MeOH (1:1) (isocratic).

Peak 1: Intermediate I-4-6. Isolated: 400 mg. Method SFC-16: Rt=5.40/30 min, ee 96%.

Peak 2: Intermediate I-4-7. Isolated: 420 mg. Method SFC-16: Rt=5.93/30 min, ee 96%.

### Step 7: tert-butyl 5-(2,2-difluorocyclopropyl)-4-formyl-7-methyl-1H-indole-1-carboxylate (Intermediate I-4)

To a soln. of tert-butyl 5-(2,2-difluorocyclopropyl)-4-(hydroxymethyl)-7-methyl-1H-indole-1-carboxylate (Intermediate I-4-6, 400 mg, 1.185 mmol) in 1,2 DCE (10 mL) were added MnO₂ (4.12 g, 47.42 mmol) at RT. The RM was stirred at 50° C. for 2 h, allowed to cool to RT and then filtered through Celite® and concentrated under reduced pressure. The crude material was purified by column chromatography over silica gel (2 to 4% EtOAc in hexane). Fractions containing product were combined and concentrated under reduced pressure to yield the title compound as an off-white solid (313 mg). ¹H NMR (400 MHz, CDCl₃) δ [ppm]10.61 (s, 1H), 7.78 (d, J=4.0 Hz, 1H), 7.42 (d, J=4.0 Hz, 1H), 7.15 (s, 1H), 3.42-3.36 (m, 1H), 2.66, (s, 3H), 2.08-2.01 (m, 1H), 1.88-1.85 (m, 1H), 1.66 (s, 9H). LC-MS Method A-1: Rt=1.70 min; MS m/z [M+H]⁺=336.1.

Intermediate I-5 was prepared according to the method described for Intermediate I-4 herein above, using Intermediate I-4-7 as starting material. ¹H NMR (400 MHz, CDCl₃) δ [ppm]10.61 (s, 1H), 7.78 (d, J=4.0 Hz, 1H), 7.42 (d, J=4.0 Hz, 1H), 7.15 (s, 1H), 3.42-3.34 (m, 1H), 2.66, (s, 3H), 2.08-2.00 (m, 1H), 1.88-1.85 (m, 1H), 1.66 (s, 9H).

### Intermediate I-6

### tert-butyl 4-(chloromethyl)-5-cyclopropyl-7-methyl-1H-indole-1-carboxylate

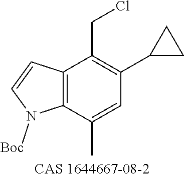 [SMILES: Cc1cc(C2CC2)c(CCl)c2ccn(C(=O)OC(C)(C)C)c12]

### Intermediate I-7

### tert-butyl 4-(chloromethyl)-5-methoxy-7-methyl-1H-indole-1-carboxylate

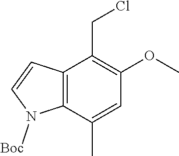 [SMILES: COc1cc(C)c2c(ccn2C(=O)OC(C)(C)C)c1CCl]

Cyanuric chloride (189.8 mg, 1.029 mmol) was added to DCM (20 mL) and DMSO (0.1 mL), and the soln. was stirred for 10 min. Tert-butyl 4-(hydroxymethyl)-5-methoxy-7-methyl-1H-indole-1-carboxylate (CAS 1644667-10-6, 500 mg, 1.716 mmol) was added slowly and the RM was stirred at RT for 2 h. The reaction was quenched with water and extracted with DCM three times. The combined extracts were dried over Na₂SO₄, and the solvents were removed under reduced pressure to give the title compound (550 mg). ¹H NMR (400 MHz, CDCl₃) δ [ppm]7.57 (d, J=4.0 Hz, 1H), 6.72 (s, 1H), 6.62 (d, J=4.0 Hz, 1H), 4.92 (s, 2H), 3.91 (s, 3H), 2.63 (s, 3), 1.62 (s, 9H).

### Intermediate I-8

### 7-methyl-1H-indole-4-carbaldehyde

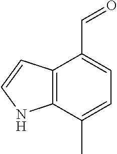

A mixture of 1H-indol-4-yl-methanol (CAS #1074-85-7, 100 mg, 0.62 mmol) and MnO₂ (216 mg, 2.48 mmol) in EtOAc (3 mL) was heated to 75° C. overnight. The RM was allowed to cool to RT and then filtered through Celite®, eluting with DCM. The filtrate was concentrated under reduced pressure and the residue was purified by chromatography over silica gel (NP, 0 to 70% EtOAc in heptane) to provide the title compound (79 mg) as a white solid. LC-MS Method C-2: Rt=0.75 min, MS m/z [M+H]⁺=160.1.

### Intermediate O-1:

### tert-butyl 2,2-dimethoxy-6-(((trifluoromethyl)sulfonyl)oxy)-7-azaspiro[3.5]non-5-ene-7-carboxylate

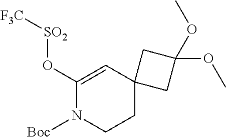 [SMILES: COC1(OC)CC2(C=C(OS(=O)(=O)C(F)(F)F)N(C(=O)OC(C)(C)C)CC2)C1]

### Step1: tert-butyl 2,2-dimethoxy-7-azaspiro[3.5]nonane-7-carboxylate (Intermediate O-1-1)

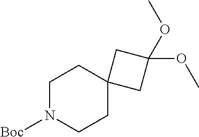 [SMILES: COC1(OC)CC2(CCN(C(=O)OC(C)(C)C)CC2)C1]

Camphorsulfonic acid (970 mg, 4.2 mmol) was added to a soln. of tert-butyl 2-oxo-7-azaspiro[3.5]nonane-7-carboxylate (20 g, 84 mmol) and trimethyl orthoformate (27 g, 27 mL, 0.25 mol) in MeOH. The RM was stirred at RT for 60 min. NaHCO₃ (7.0 g, 84 mmol) was added and most of the MeOH was evaporated under reduced pressure at 50° C. The residue was partitioned between MTBE and water. The layers were separated and washed with brine and MTBE. The combined org. layers were dried over MgSO₄, treated with activated charcoal and filtered over Celite®. The volatiles of the filtrate were removed under reduced pressure at 50° C. to give a colorless oil that was treated with hexane, the volatiles were again removed under reduced pressure at 50° C. to give the title compound as a colorless oil (23.58 g). ¹H NMR (400 MHz, DMSO-d₆) δ [ppm]3.25-3.19 (m, 4H), 3.02 (s, 6H), 1.86 (s, 4H), 1.46-1.41 (m, 4H), 1.38 (s, 9H).

### Step 2: tert-butyl 2,2-dimethoxy-6-oxo-7-azaspiro[3.5]nonane-7-carboxylate (Intermediate O-1-2)

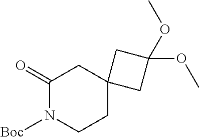 [SMILES: COC1(OC)CC2(CCN(C(=O)OC(C)(C)C)C(=O)C2)C1]

NaIO₄ (38.83 g, 181.5 mmol) was dissolved in water and RuCl₃ (1.712 g, 550 μL, 8.252 mmol) was added under mechanical stirring to give an orange soln.; NaHCO₃ (6.933 g, 82.52 mmol) was added in portions (gas development). The mixture was vigorously stirred while a soln. of tert-butyl 2,2-dimethoxy-7-azaspiro[3.5]nonane-7-carboxylate (23.55 g, 82.52 mmol) in EtOAc was added in a dropwise manner. The temperature was kept below 31° C. using a water bath. The RM was vigorously stirred at RT for 3 h 50 min, stirring was continued overnight. The RM was diluted with EtOAc and aq. Na₂S₂O₃ soln. (10 wt %) was added. The mixture was filtered over Celite®, and the black solid was washed with EtOAc. The layers of the filtrate were separated and washed with brine and EtOAc. The combined org. layers were dried over MgSO₄, filtered and the volatiles were removed under reduced pressure at 50° C.

The crude product was purified over silica gel (220 g) using an automated purification system (NP; Teledyne ISCO®; flow 150 mL/min, eluent: heptane+5 to 35% EtOAc in 35 min). Product-containing fractions were combined and the volatiles were removed under reduced pressure at 50° C. to give the title compound as a slightly yellow oil (17.26 g). LC-MS Method B-2: Rt=0.87 min; MS 300.3 [M+H]⁺.

### Step 3: tert-butyl 2,2-dimethoxy-6-(((trifluoromethyl)sulfonyl)oxy)-7-azaspiro[3.5]non-5-ene-7-carboxylate (Intermediate O-1)

A soln. of HMDS (12.10 g, 15.7 mL, 74.95 mmol) in 300 mL THE was evacuated/back-filled with N₂ twice. The solution was cooled to −75° C. and n-BuLi (1.6M in hexane, 46.84 mL, 74.95 mmol) was added in a dropwise manner (exothermic, the temperature was not allowed to exceed −65° C.). The soln. was then allowed to re-cool to −75° C. A soln. of Intermediate O-1-2 (17.26 g, 57.65 mmol) in THE (40 mL) was added in a dropwise, but continuous manner. The resulting yellow soln. was stirred at −75° C. for 150 min. A soln. of 1,1,1-trifluoro-N-phenyl-N-((trifluoromethyl)sulfonyl)methanesulfonamide (27.81 g, 77.83 mmol) in THE (50 m) was added in a dropwise manner. The cooling bath was removed, and the RM was stirred for 40 min. The RM was poured into a well-stirred emulsion of 1M aq. NaOH soln. and MTBE. The layers were separated and washed with 1M aq. NaOH soln., brine and MTBE. The combined org. layers were dried over K₂CO₃, filtered, and the volatiles were removed under reduced pressure at 50° C. to give a slightly yellow oil.

The crude product was purified over silica gel (330 g) using an automated purification system (NP; Teledyne ISCO®; collected at 245 nm+ELSD detector (flow 200 mL/min, eluent: heptane/Et₃N 99:1+10 to 47.2% DCM/Et₃N 99:1). Fractions were combined and the volatiles were removed under reduced pressure at 50° C. to give the title compound as a slightly yellow oil (22.37 g). LC-MS Method B-2: Rt=1.25 min; MS m/z [M-tertBu+H]⁺=376.2.

### Intermediate O-2:

### tert-butyl 2,2-difluoro-6-(((trifluoromethyl)sulfonyl)oxy)-7-azaspiro[3.5]non-5-ene-7-carboxylate

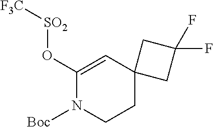 [SMILES: CC(C)(C)OC(=O)N1CCC2(C=C1OS(=O)(=O)C(F)(F)F)CC(F)(F)C2]

### Step 1: tert-butyl 2,2-difluoro-7-azaspiro[3.5]nonane-7-carboxylate (Intermediate O-2-1)

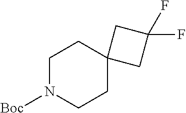 [SMILES: CC(C)(C)OC(=O)N1CCC2(CC1)CC(F)(F)C2]

To a soln. of tert-butyl 2-oxo-7-azaspiro[3.5]nonane-7-carboxylate (350 g, 1.46 mol) in CH₂Cl₂ (3.00 L) was added DAST (471 g, 2.93 mol, 386 mL) dropwise at 0° C. The RM was stirred at 25° C. for 48 h. The mixture was diluted with MeOH (0.70 L) at 0° C. and then concentrated under reduced pressure. The residue was purified by column chromatography over silica gel (PE/EtOAc=100/1 to 100/5) providing the title compound (280 g) as a yellow liquid. ¹H NMR (400 MHz, CDCl₃) δ [ppm]3.39-3.24 (m, 4H), 2.33 (t, J=12.6 Hz, 4H), 1.65-1.56 (m, 4H), 1.44 (s, 9H).

### Step 2: tert-butyl 2,2-difluoro-6-oxo-7-azaspiro[3.5]nonane-7-carboxylate (Intermediate O-2-2)

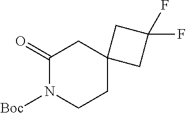 [SMILES: CC(C)(C)OC(=O)N1CCC2(CC1=O)CC(F)(F)C2]

To a soln. of NaIO₄ (504 g, 2.36 mol) in H₂O (1.40 L) was added RuCl₃ (22.2 g, 107 mmol, 7.15 mL) at 0° C. Then a soln. of tert-butyl 2,2-difluoro-7-azaspiro[3.5]nonane-7-carboxylate (Intermediate O-2-1, 280 g, 1.07 mol) in EtOAc (1.40 L) was added to the above mixture dropwise at 0° C. The mixture was exposed to air and stirred for 16 h at 18° C. The RM was filtered and extracted with EtOAc (3×). The combined org. layers were washed with 10% aq. Na₂S₂O₃ soln. (3×), dried over Na₂SO₄, filtered, and concentrated under reduced pressure. The resulting residue was diluted with MTBE and stirred for 1 h at 0° C. The precipitated solids were filtered off and dried under reduced pressure to give the title compound (90.0 g) as a white solid. ¹H NMR (400 MHz, CDCl₃) δ [ppm]3.69-3.58 (m, 2H), 2.64 (s, 2H), 2.47 (tt, J=12.2, 2.0 Hz, 4H), 1.96 (t, J=6.0 Hz, 2H), 1.52 (s, 9H).

Alternative preparation of tert-butyl 2,2-difluoro-6-oxo-7-azaspiro[3.5]nonane-7-carboxylate (Intermediate O-2-2):

To a soln. of NaIO₄ (504 g, 2.36 mol) in H₂O (14.7 mL) was added RuCl₃ (99 mg, 0.478 mmol). The orange soln., containing dark solid material, was vigorously stirred and cooled by a water bath while a soln. of tert-butyl 2,2-difluoro-7-azaspiro[3.5]nonane-7-carboxylate (Intermediate O-2-1, 1.25 g, 4.78 mmol) in EtOAc (14.7 mL) was added in a dropwise manner. The RM was vigorously stirred overnight and then diluted with EtOAc and 10% aq. Na₂S₂O₃ soln.; the mixture was filtered over Celite® and the solids were washed with EtOAc. The layers of the filtrate were separated and washed with brine and extracted with EtOAc. The combined org. layers were dried over MgSO₄, filtered, and concentrated under reduced pressure at 50° C. providing the crude title compound (1.28 g) as white solid which was directly used in the next reaction without further purification. LC-MS Method B-1: Rt=0.81 min; MS m/z [M-tertBu+H]⁺=220.3.

### Step 3: tert-butyl 2,2-difluoro-6-(((trifluoromethyl)sulfonyl)oxy)-7-azaspiro[3.5]non-5-ene-7-carboxylate (Intermediate O-2)

To a soln. of HMDS (39.5 g, 245 mmol, 51.3 mL) in THE (712 mL) was added n-BuLi (2.5M in hexane, 94.4 mL) at −78° C. under N₂ atmosphere. Then a soln. of tert-butyl 2,2-difluoro-6-oxo-7-azaspiro[3.5]nonane-7-carboxylate (Intermediate O-2-2, 50.0 g, 181 mmol) in THE (142 mL) was added dropwise to the above mixture. After stirring for 2 h at −78° C. a soln. of N-phenyl-bis(trifluoromethanesulfonimide) (87.6 g, 245 mmol) in THE (258 mL) was added dropwise. The cooling bath was removed, and the mixture was stirred for additional 8 h at 25° C. The RM was poured into a well-stirred 1M aq. NaOH soln. (300 mL) at 0° C. and then extracted with EtOAc (3×1.0 L). The combined org. layers were washed with brine, dried over Na₂SO₄, filtered, and concentrated under reduced pressure. The resulting residue was purified by RP-HPLC (ACN/water, 0.1% FA condition) providing the title compound (100 g) as a yellow liquid. ¹H NMR (400 MHz, CDCl₃) δ [ppm]5.25 (s, 1H), 3.64-3.60 (m, 2H), 2.74-2.62 (m, 2H), 2.58-2.46 (m, 2H), 1.93 (td, J=2.8, 5.2 Hz, 2H), 1.50 (s, 9H).

Note: Alternatively, crude material of tert-butyl 2,2-difluoro-6-(((trifluoromethyl)sulfonyl)oxy)-7-azaspiro[3.5]non-5-ene-7-carboxylate (Intermediate O-2) can be used directly in the next reaction without further purification.

To a soln. of HMDS (83.1 g, 514 mmol, 107 mL) in 2-MeTHF (0.60 L) was added n-BuLi (2.5M in hexane, 79.2 mmol, 198 mL) at −78° C. under N₂ atmosphere. Then a soln. of tert-butyl 2,2-difluoro-6-oxo-7-azaspiro[3.5]nonane-7-carboxylate (Intermediate O-2-2, 105 g, 381 mmol) in 2-MeTHF (0.40 L) was added dropwise to above mixture. After stirring for 2 h at −78° C. a soln. of N-phenyl-bis(trifluoromethanesulfonimide) (183 g, 514 mmol) in 2-MeTHF (0.40 L) was added dropwise. The cooling bath was removed, and the mixture was stirred for additional 18 h at 18° C. The RM was poured into a well-stirred 1M aq. NaOH soln. at 0° C. and then extracted with EtOAc (3×). The combined org. layers were washed with brine, dried over Na₂SO₄, filtered, and concentrated under reduced pressure to give crude title compound (315 g) as a yellow oil which was used directly in the next reaction without further purification.

### Intermediate O-3:

### tert-butyl 2,2-difluoro-6-(4,4,5,5-tetramethyl-1,3,2-dioxaborolan-2-yl)-7-azaspiro[3.5]non-5-ene-7-carboxylate

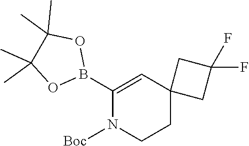 [SMILES: CC(C)(C)OC(=O)N1CCC2(C=C1B1OC(C)(C)C(C)(C)O1)CC(F)(F)C2]

A soln. of tert-butyl 2,2-difluoro-6-(((trifluoromethyl)sulfonyl)oxy)-7-azaspiro[3.5]non-5-ene-7-carboxylate (Intermediate O-2, 7.970 g, 19.57 mmol), triphenylphosphine (307.9 mg, 1.174 mmol) and 4,4,4′,4′,5,5,5′,5′-octamethyl-2,2′-bi(1,3,2-dioxaborolane) (7.453 g, 29.35 mmol) was evacuated and back-filled with N₂. K₂CO₃ (325 mesh, 4.056 g, 29.35 mmol) and PdCl₂(PPh₃)₂ (412 mg, 587 μmol) were added. The mixture was evacuated and back-filled with N₂ again, heated to 90° C. and stirred under a N₂ atmosphere for 4 h. The RM was cooled to RT. The RM was diluted with MTBE and the mixture was filtered. The solids were washed with MTBE. Water was added to the filtrate and the layers were separated. They were washed with brine and MTBE. The combined org. layers were dried over MgSO₄, filtered, and the volatiles were removed under reduced pressure at 50° C. to give a yellow oil. The crude material was purified over silica gel (220 g) using heptane/EtOAc. Fractions were combined and the volatiles were removed under reduced pressure at 50° C. to give the title compound as an off-white solid (5.40 g, containing bis(pinacolato)diboron in an unquantified amount due to overlapping NMR signals) that was used in the next step without further purification. LC-MS Method B-1: Rt=1.38 min; MS m/z [M-Boc+H]⁺=286.3.

### Intermediate O-4:

### tert-butyl 6-(((trifluoromethyl)sulfonyl)oxy)-2-oxa-7-azaspiro[3.5]non-5-ene-7-carboxylate

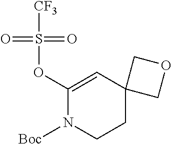 [SMILES: CC(C)(C)OC(=O)N1CCC2(C=C1OS(=O)(=O)C(F)(F)F)COC2]

### Step 1: tert-butyl 6-oxo-2-oxa-7-azaspiro[3.5]nonane-7-carboxylate (Intermediate O-4-1)

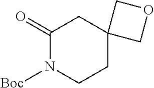 [SMILES: CC(C)(C)OC(=O)N1CCC2(COC2)CC1=O]

NaIO₄ (5.18 g, 24.2 mmol) was dissolved in water (26 mL) and RuCl₃ (228 mg, 1.1 mmol) was added. The mixture was vigorously stirred and cooled by a water bath while a soln. of tert-butyl 2-oxa-7-azaspiro[3.5]nonane-7-carboxylate (2.50 g, 11.0 mmol) in EtOAc (26 mL) was added in a dropwise manner. The RM was vigorously stirred overnight, then diluted with EtOAc and aq. Na₂S₂O₃ soln. (10 wt %) was added. The mixture was filtered over Celite®, and the black solid was washed with EtOAc. The layers of the filtrate were separated and washed with brine and EtOAc. The combined org. layers were dried over MgSO₄, filtered, and the volatiles were removed under reduced pressure at 50° C. to give an off-white solid that was triturated with hexane/MTBE 95/5. The suspension was treated with ultrasound and filtered. The solid was washed with hexane/MTBE 95/5 and dried under reduced pressure at 50° C. to give the title compound (1.86 g). LC-MS Method B-1: Rt=0.46 min; MS m/z [M-tertBu+H]⁺=186.1.

### Step 2: tert-butyl 6-(((trifluoromethyl)sulfonyl)oxy)-2-oxa-7-azaspiro[3.5]non-5-ene-7-carboxylate (Intermediate O-4)

A soln. of HMDS (261 mg, 1.616 mmol) in THE (2.6 mL) was evacuated/backfilled with N₂ twice and cooled to −78° C. n-BuLi (1.6M in hexane, 104 mL, 1.616 mmol) was added in dropwise manner. The resulting clear and colorless soln. was stirred in the dry ice bath for 5 to 10 min. This freshly prepared LiHMDS soln. was then cannulated into a pre-cooled (−78° C.) and degassed soln. of tert-butyl 6-oxo-2-oxa-7-azaspiro[3.5]nonane-7-carboxylate (Intermediate O-4-1, 300 mg, 1.243 mmol) in THE (3.7 mL). The RM was stirred for 120 min. A soln. of 1,1,1-trifluoro-N-phenyl-N-((trifluoromethyl)sulfonyl)-methanesulfonamide (600 mg, 1.678 mmol) in THE (1.6 mL) was added dropwise. The cooling bath was removed and the RM was stirred for 30 min, then poured under stirring on a mixture of aq. NaOH (c=1.0 mol/L) and MTBE. The layers were separated and washed with 0.5M aq. NaOH solution, brine and MTBE. The combined org. layers were dried over K₂CO₃, filtered, and the volatiles were removed under reduced pressure at 50° C. to give a yellow oil which was purified over silica gel (12 g) by eluting with heptane/EtOAc (9/1 to 6/1) containing 1% Et₃N. The pure fractions were combined and the volatiles were removed under reduced pressure at 50° C. to give the title compound as a colorless oil (464 mg). LC-MS Method B-1: Rt=1.15 min; MS m/z [M−H]⁻=372.2.

### Intermediate O-5:

### tert-butyl 2,2-dimethoxy-6-(4-(methoxycarbonyl)phenyl)-7-azaspiro[3.5]non-5-ene-7-carboxylate

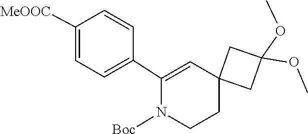 [SMILES: COC(=O)c1ccc(C2=CC3(CCN2C(=O)OC(C)(C)C)CC(OC)(OC)C3)cc1]

A soln. of K₃PO₄ (33.02 g, 12.88 mL, 155.6 mmol) in water was added to a soln. of tert-butyl 2,2-dimethoxy-6-(((trifluoromethyl)sulfonyl)oxy)-7-azaspiro[3.5]non-5-ene-7-carboxylate (Intermediate O-1, 22.37 g, 51.85 mmol) and (4-(methoxycarbonyl)phenyl)boronic acid (12.13 g, 67.41 mmol) in dioxane. The vigorously stirred emulsion was evacuated/back-filled with N₂ twice. PdCl₂(dtbpf) (1.690 g, 2.593 mmol) was added, and the capped vial was stirred at 55° C. (bath temperature) for 65 min. Stirring was continued for another 30 min, then another 0.1 eq (933 mg) (4-(methoxycarbonyl)phenyl)boronic acid and 0.01 eq (338 mg) PdCl₂(dtbpf) were added and stirring was continued for additional 30 min.

The RM was partitioned between water and EtOAc. The layers were separated and washed with sat. aq. NaHCO₃ soln., brine, and EtOAc. The combined org. layers were dried over MgSO₄, filtered, and the volatiles were removed under reduced pressure at 50° C. to give an amber oil that was purified over silica gel (330 g) using an automated purification system (NP; Teledyne ISCO®; collected at 254/280 nm+ELSD detector; flow 200 mL/min; eluent: heptane:Et₃N 99:1+5 to 22.2% EtOAc in 17.2 min). Fractions were combined and the volatiles were removed under reduced pressure at 50° C. to give an amber oil. The material was dissolved in hexane/MTBE 9/1 and treated with activated charcoal. The mixture was filtered over Celite®, and the coal was washed with hexane/MTBE 9/1 (3×). The volatiles were removed under reduced pressure at 50° C. to give the title compound as a slightly amber oil (18.82 g) which solidified upon standing. LC-MS Method B-2: Rt=1.20 min; MS m/z [M+H]⁺=418.4.

### Intermediate O-6:

### tert-butyl 2,2-difluoro-6-(4-(methoxycarbonyl)phenyl)-7-azaspiro[3.5]non-5-ene-7-carboxylate

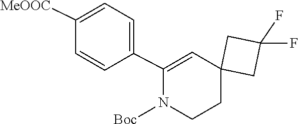 [SMILES: COC(=O)c1ccc(C2=CC3(CCN2C(=O)OC(C)(C)C)CC(F)(F)C3)cc1]

To crude tert-butyl 2,2-difluoro-6-(((trifluoromethyl)sulfonyl)oxy)-7-azaspiro[3.5]non-5-ene-7-carboxylate (Intermediate O-2, 155 g, 380 mmol, assumed 100% conversion rate) and (4-(methoxycarbonyl)phenyl)boronic acid (82.1 g, 456 mmol) in dioxane (1.20 L) were added the soln. of K₃PO₄ (242 g, 1.14 mol) in H₂O (0.30 L) and PdCl₂(dtbpf) (12.4 g, 19.0 mmol). The mixture was degassed under reduced pressure and purged 3× with N₂, then stirred at 50° C. for 12 h. The RM was concentrated under reduced pressure to remove dioxane. The residue was diluted with water and extracted with EtOAc (3×). The combined org. layers were concentrated under reduced pressure. The resulting residue was purified by column chromatography over silica gel (PE/EtOAc=100/1 to 0/100). The crude product was purified by RP-HPLC (ACN/water, 0.1% FA condition) providing the title compound (80.0 g) as a white solid. ¹H NMR (400 MHz, CDCl₃) δ [ppm]7.99 (d, J=8.4 Hz, 2H), 7.36 (d, J=8.4 Hz, 2H), 5.33 (s, 1H), 3.93 (s, 3H), 3.73 (td, J=2.8, 5.2 Hz, 2H), 2.74-2.62 (m, 2H), 2.56-2.46 (m, 2H), 2.10-1.98 (m, 2H), 1.07 (s, 9H).

### Intermediate O-7:

### tert-butyl 6-(4-(methoxycarbonyl)phenyl)-2-oxa-7-azaspiro[3.5]non-5-ene-7-carboxylate

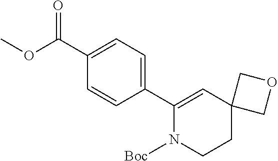 [SMILES: COC(=O)c1ccc(C2=CC3(CCN2C(=O)OC(C)(C)C)COC3)cc1]

tert-Butyl 6-(((trifluoromethyl)sulfonyl)oxy)-2-oxa-7-azaspiro[3.5]non-5-ene-7-carboxylate (Intermediate O-4, 315 mg, 0.844 mmol) and (4-(methoxycarbonyl)phenyl)boronic acid (197 mg, 1.097 mmol) were dissolved in dioxane (5.6 mL). A soln. of K₃PO₄ (537 mg, 2.53 mmol) in water (1.5 mL) was added. The mixture was evacuated/backfilled with N₂. PdCl₂(dtbpf (27.5 mg, 0.042 mg) was added and the mixture was evacuated/backfilled with N₂ twice. The RM was then heated to 50° C. and vigorously stirred for 80 min. The RM was partitioned between water and EtAc. The layers were separated and washed with sat. aq. NaHCO₃ soln., brine, and EtOAc. The combined org. layers were dried over MgSO₄, filtered, and the volatiles were removed under reduced pressure at 50° C. to give an amber oil that was purified over silica gel (24 g) using an automated purification system (NP; Teledyne ISCO®; collected at 280 nm; eluent: heptane+10 to 26.3% EtOAc in 16.3 min). Pure fractions were combined and the volatiles were removed under reduced pressure at 50° C. to give the title compound as a colorless oil (242 mg). LC-MS Method B-1: Rt=1.04 min; MS m/z [M-tertBu+H]⁺=304.3.

Intermediate rac-O-8:

### tert-butyl 2,2-difluoro-7-(4-(methoxycarbonyl)phenyl)-8-azaspiro[4.5]dec-6-ene-8-carboxylate

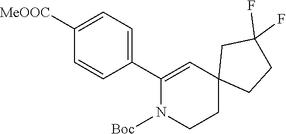 [SMILES: COC(=O)c1ccc(C2=CC3(CCN2C(=O)OC(C)(C)C)CCC(F)(F)C3)cc1]

Intermediate rac-O-8 was prepared according to the method described for Intermediate O-6 herein above, using tert-butyl 2-oxo-8-azaspiro[4.5]decane-8-carboxylate (CAS #1250994-14-9) as starting material. LC-MS Method C-2: Rt=3.09 min; MS m/z [M-tertBu+H]⁺=352.1.

### Intermediate (+)-O-9:

### (+)-tert-butyl 2,2-difluoro-7-(4-(methoxycarbonyl)phenyl)-8-azaspiro[4.5]dec-6-ene-8-carboxylate

### and Intermediate (−)-O-10:

### (−)-tert-butyl 2,2-difluoro-7-(4-methoxycarbonyl)phenyl)-8-azaspiro[4.5]dec-6-ene-8-carboxylate

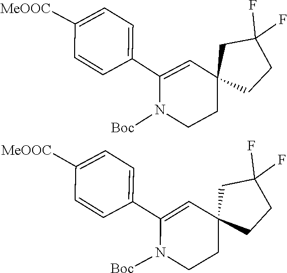

For SFC chiral purification of Intermediate rac-O-8 (2.5 g): Instrument Thar 80 preparative SFC; column: Chiralpak IE 21×250 mm 5 μm; flow rate: 80 g per min; co-solvent: 15% 1:1 MeOH:IPA in CO₂; detection: 300 nm; back pressure set point: 125 bar; injection size: 17.04 mg (14.2 mg/mL in MeOH).

Peak 1: Intermediate (+)-O-9, isolated: 1.716 g. Method SFC-1: Rt=1.14 min/5.0 min, ee>99%. Specific rotation Method OR1: [α]²⁵ @589 nm=+5.57° (10.53 mg of Intermediate (+)-O-9 in 1 mL DCM).

Peak 2: Intermediate (−)-O-10, isolated: 1.748 g. Method SFC-1: Rt=1.36 min/5.0 min, ee 97%. Specific rotation Method OR1: [α]²⁵ @589 nm=−1.10° (10.018 mg of Intermediate (−)-O-10 in 1 mL DCM).

### Intermediate O-11:

### tert-butyl 2,2-difluoro-6-(4-(3-hydroxyoxetan-3-yl)phenyl)-7-azaspiro[3.5]non-5-ene-7-carboxylate

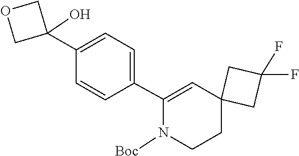 [SMILES: CC(C)(C)OC(=O)N1CCC2(C=C1c1ccc(C3(O)COC3)cc1)CC(F)(F)C2]

A soln. of K₃PO₄ (945 mg, 369 μL, 4.454 mmol) in water (2.63 mL) was added to a soln. of tert-butyl 2,2-difluoro-6-(4,4,5,5-tetramethyl-1,3,2-dioxaborolan-2-yl)-7-azaspiro[3.5]non-5-ene-7-carboxylate (Intermediate O-3, 572 mg, 1.485 mmol) and 3-(4-bromophenyl)oxetan-3-ol (510.2 mg, 2.227 mmol) in dioxane (9.90 mL). The well-stirred emulsion was evacuated/back-filled with N₂ twice. PdCl₂(dtbpf (48.4 mg, 74.2 μmol) was added, the mixture was evacuated/back-filled with N₂ once more and then stirred at 50° C. for 40 min. The RM was cooled to RT, then partitioned between water and EtOAc. The layers were separated and washed with sat. aq. NaHCO₃ soln., brine, and EtOAc. The combined org. layers were dried over MgSO₄, filtered, and the volatiles were removed under reduced pressure at 50° C. An amber solid was obtained. The crude product was taken up in DCM and suspended in an ultrasound bath. The suspension was filtered, and the filtrate was purified over silica gel (24 g) using an automated purification system (NP; Teledyne ISCO®; collected at 257/275 nm; flow 35 mL/min; eluent: heptane+5 to 26.4% (EtOAc:MeOH 95:5) in 18.1 min.; the gradient was paused when the product eluted). Fractions were combined and the volatiles were removed under reduced pressure at 50° C. to give the title compound as an amber oil (480 mg, containing ˜17 wt % pinacol). LC-MS Method B-1: Rt=1.10 min; MS m/z [M+H]⁺=408.4. The product was used in the next step without further purification.

### Intermediate O-12:

### tert-butyl 2,2-difluoro-6-(4-(2-hydroxypropan-2-yl)phenyl)-7-azaspiro[3.5]non-5-ene-7-carboxylate

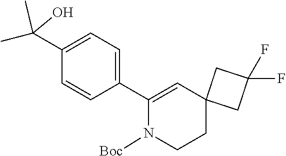 [SMILES: CC(C)(C)OC(=O)N1CCC2(C=C1c1ccc(C(C)(C)O)cc1)CC(F)(F)C2]

A soln. of K₃PO₄ (945.4 mg, 368.7 μL, 4.454 mmol) in water (2.63 mL) was added to a soln. of tert-butyl 2,2-difluoro-6-(4,4,5,5-tetramethyl-1,3,2-dioxaborolan-2-yl)-7-azaspiro[3.5]non-5-ene-7-carboxylate (Intermediate O-3, 572 mg, 1.485 mmol) and 2-(4-bromophenyl)propan-2-ol (479 mg, 2.227 mmol) in dioxane (9.90 mL). The well-stirred emulsion was evacuated/back-filled with N₂ twice. PdCl₂(dtbpf) (48.38 mg, 74.24 μmol) was added, the mixture was evacuated/back-filled with N₂ once more and then stirred at 50° C. for 50 min. The RM was cooled to RT, then partitioned between water and EtOAc. The layers were separated and washed with sat. aq. NaHCO₃ soln., brine, and EtOAc. The combined org. layers were dried over MgSO₄, filtered, and the volatiles were removed under reduced pressure at 50° C. An amber solid was obtained that was dissolved in DCM and purified over silica gel (24 g) using an automated purification system (NP; Teledyne ISCO®; collected at 255/270 nm; flow 35 mL/min; eluent: heptane+5 to 20.5% EtOAc in 16.1 min; the gradient was paused when the product eluted). Purest fractions were combined and the volatiles were removed under reduced pressure at 50° C. to give the title compound as a slightly amber oil (344 mg). LC-MS Method B-1: Rt=1.25 min; MS m/z [M−H₂O+H]⁺=376.3; MS m/z [M-tertBu-H₂O+H]⁺=320.2.

### Intermediate O-13:

### tert-butyl 8-(4-(1H-pyrazol-5-yl)phenyl)-2,2-difluoro-7-azaspiro[3.5]non-5-ene-7-carboxylate

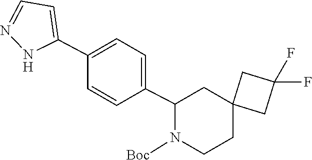 [SMILES: CC(C)(C)OC(=O)N1CCC2(CC1c1ccc(-c3ccn[nH]3)cc1)CC(F)(F)C2]

A soln. of K₃PO₄ (826.4 mg, 322.3 μL, 3.893 mmol) in water (2.30 mL) was added to a soln. of tert-butyl 2,2-difluoro-6-(4,4,5,5-tetramethyl-1,3,2-dioxaborolan-2-yl)-7-azaspiro[3.5]non-5-ene-7-carboxylate (Intermediate O-3, 500 mg, 1.298 mmol) and 5-(4-bromophenyl)-1H-pyrazole (434.3 mg, 1.947 mmol) in dioxane (8.65 mL). The well-stirred emulsion was evacuated/back-filled with N₂ twice. PdCl₂(dtbpf (42.29 mg, 64.89 μmol) was added, the mixture was evacuated/back-filled with N₂ once more and then stirred at 50° C. for 40 min. The RM was cooled to RT, then partitioned between water and MTBE. The slurry was filtered over Celite®, and the filtrate was transferred to a separation funnel. The layers were separated and washed with sat. aq. NaHCO₃ soln., brine and MTBE. The combined org. layers were dried over MgSO₄, filtered, and the volatiles were removed under reduced pressure at 50° C. An amber solid was obtained that was purified over silica gel (40 g) using an automated purification system (NP; Teledyne ISCO®; collected at 254/280 nm; flow 40 mL/min; eluent: heptane+10 to 40% EtOAc in 24.0 min). Fractions were combined and the volatiles were removed under reduced pressure at 50° C. to give the title compound as a slightly yellow oil (293 mg, containing −17 wt % pinacol). LC-MS Method B-1: Rt=1.25 min; MS m/z [M-tertBu+H]⁺=346.2.

### Intermediate O-14:

### tert-butyl 6-(4-(1H-pyrazol-1-yl)phenyl)-2,2-difluoro-7-azaspiro[3.5]non-5-ene-7-carboxylate

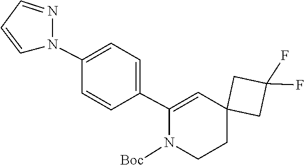 [SMILES: CC(C)(C)OC(=O)N1CCC2(C=C1c1ccc(-n3cccn3)cc1)CC(F)(F)C2]

A soln. of K₃PO₄ (661.1 mg, 257.9 μL, 3.115 mmol) in water (1.84 mL) was added to a soln. of tert-butyl 2,2-difluoro-6-(4,4,5,5-tetramethyl-1,3,2-dioxaborolan-2-yl)-7-azaspiro[3.5]non-5-ene-7-carboxylate (Intermediate O-3, 400 mg, 1.038 mmol) and 1-(4-bromophenyl)-1H-pyrazole (347.4 mg, 1.557 mmol) in dioxane (6.92 mL). The well-stirred emulsion was evacuated/back-filled with N₂ twice. PdCl₂(dtbpf) (33.83 mg, 51.91 μmol) was added, the mixture was evacuated/back-filled with N₂ once more and then stirred at 50° C. for 50 min. The RM was cooled to RT, then partitioned between water and MTBE. The slurry was filtered over Celite®, and the filtrate was transferred to a separation funnel. The layers were separated and washed with sat. aq. NaHCO₃ soln., brine and MTBE. The combined org. layers were dried over MgSO₄, filtered, and the volatiles were removed under reduced pressure at 50° C. An amber solid was obtained that was dissolved in DCM and purified over silica gel (24 g) using an automated purification system (NP; Teledyne ISCO®; collected at 278/254 nm; flow 35 mL/min; eluent: heptane+5 to 12.7% EtOAc in 15.4 min). Pure fractions were combined and the volatiles were removed under reduced pressure at 50° C. to give the title compound as slightly yellow oil (234 mg). LC-MS Method B-1: Rt=1.35 min; MS m/z [M-tertBu+H]⁺=346.2.

### Intermediate O-15:

### tert-butyl 6-(4-cyanophenyl)-2,2-difluoro-7-azaspiro[3.5]non-5-ene-7-carboxylate

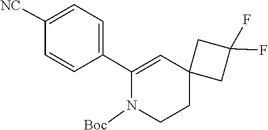 [SMILES: CC(C)(C)OC(=O)N1CCC2(C=C1c1ccc(C#N)cc1)CC(F)(F)C2]

A soln. of K₃PO₄ (1.157 g, 451.2 μL, 5.451 mmol) in water (3.22 mL) was added to a soln. of tert-butyl 2,2-difluoro-6-(4,4,5,5-tetramethyl-1,3,2-dioxaborolan-2-yl)-7-azaspiro[3.5]non-5-ene-7-carboxylate (Intermediate O-3, 700 mg, 1.817 mmol) and 4-bromobenzonitrile (496.1 mg, 2.725 mmol) in dioxane (12.11 mL). The well-stirred emulsion was evacuated/back-filled with N₂ twice. PdCl₂(dtbpf) (59.21 mg, 90.85 μmol) was added, the mixture was evacuated/back-filled with N₂ once more and then stirred at 50° C. for 50 min. The RM was cooled to RT, then partitioned between water and MTBE. The slurry was filtered over Celite® and the filtrate was transferred to a separation funnel. The layers were separated and washed with sat. aq. NaHCO₃ soln., brine and MTBE. The combined org. layers were dried over MgSO₄, filtered, and the volatiles were removed under reduced pressure at 50° C. An amber solid was obtained. The material was taken up in hexane and filtered. The volatiles of the filtrate were removed under reduced pressure at 50° C. to give an amber oil that was purified over silica gel (40 g) using an automated purification system (NP; Teledyne ISCO®; collected at 245/284 nm; flow 40 mL/min; eluent: heptane+5 to 16.6% EtOAc in 15.5 min). Pure fractions were combined and the volatiles were removed under reduced pressure at 50° C. to give the title compound as slightly yellow oil (363 mg) which solidified upon standing. ¹H NMR (400 MHz, DMSO-d₆) δ [ppm]7.84-7.77 (m, 2H), 7.50-7.43 (m, 2H), 5.55 (s, 1H), 3.67-3.59 (m, 2H), 2.82-2.69 (m, 2H), 2.56 (m, 2H), 1.96-1.90 (m, 2H), 1.02 (s, 9H).

### Intermediate O-16:

### tert-butyl 2,2-difluoro-6-phenyl-7-azaspiro[3.5]non-5-ene-7-carboxylate

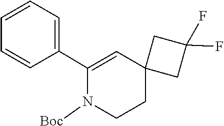 [SMILES: CC(C)(C)OC(=O)N1CCC2(C=C1c1ccccc1)CC(F)(F)C2]

A soln. of K₃PO₄ (843 mg, 328.8 μL, 3.971 mmol) in water (2.30 mL) was added to a soln. of Intermediate O-3 (510 mg, 1.324 mmol) and bromobenzene (353.3 mg, 2.250 mmol) in dioxane (8.65 mL). The well-stirred emulsion was evacuated/back-filled with N₂ twice. PdCl₂(dtbpf) (43.14 mg, 66.19 μmol) was added, the mixture was evacuated/back-filled with N₂ once more and then stirred at 50° C. for 90 min. The RM was cooled to RT, then partitioned between water and MTBE. The slurry was filtered over Celite®, and the filtrate was transferred to a separation funnel. The layers were separated and washed with sat. aq. NaHCO₃ soln., brine and MTBE. The combined org. layers were dried over MgSO₄, filtered, and the volatiles were removed under reduced pressure at 50° C. An amber oil was obtained. The crude product was dissolved in DCM and purified over silica gel (24 g) using an automated purification system (NP; Teledyne ISCO®; collected at 245/282 nm; flow 35 mL/min; eluent: heptane+0 to 6.4% EtOAc in 12.7 min). Pure fractions were combined and the volatiles were removed under reduced pressure at 50° C. to give the title compound (274 mg) as a slightly yellow oil. LC-MS Method B-1: Rt=1.47 min; MS m/z [M-tertBu+H]⁺=280.1.

### Intermediate O-17:

### tert-butyl 2,2-difluoro-6-(1-methyl-1H-pyrazol-4-yl)-7-azaspiro[3.5]non-5-ene-7-carboxylate

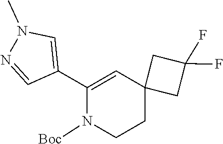 [SMILES: Cn1cc(C2=CC3(CCN2C(=O)OC(C)(C)C)CC(F)(F)C3)cn1]

A soln. of K₃PO₄ (763.6 mg, 297.8 μL, 3.598 mmol) in water (2.13 mL) was added to a soln. of Intermediate O-3 (462 mg, 1.199 mmol) and 4-bromo-1-methyl-1H-pyrazole (290 mg, 1.799 mmol) in dioxane (7.99 mL). The well-stirred emulsion was evacuated/back-filled with N₂ twice. PdCl₂(dtbpf) (39.08 mg, 59.96 μmol) was added, the mixture was evacuated/back-filled with N₂ once more and then stirred at 50° C. for 90 min. The RM was cooled to RT, then partitioned between water and MTBE. The slurry was filtered over Celite®, and the filtrate was transferred to a separation funnel. The layers were separated and washed with sat. aq. NaHCO₃ soln., brine and MTBE. The combined org. layers were dried over MgSO₄, filtered, and the volatiles were removed under reduced pressure at 50° C. An amber solid was obtained that was dissolved in DCM/hexane and purified over silica gel (24 g) using an automated purification system (NP; Teledyne ISCO®; collected at 278/254 nm; flow 35 mL/min; eluent: heptane+5-12.7% EtOAc in 15.4 min). Purest fractions were combined and the volatiles were removed under reduced pressure at 50° C. The residue was triturated with hexane and DCM, volatiles were again removed under reduced pressure at 50° C. to give the title compound as amber crystals (138 mg). LC-MS Method B-1: Rt=1.06 min; MS m/z [M+H]⁺=340.3.

Intermediate rac-B-1:

### (RS)-tert-butyl 2,2-dimethoxy-6-(4-(methoxycarbonyl)phenyl)-7-azaspiro[3.5]nonane-7-carboxylate

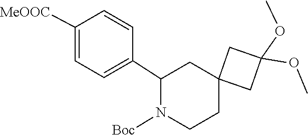 [SMILES: COC(=O)c1ccc(C2CC3(CCN2C(=O)OC(C)(C)C)CC(OC)(OC)C3)cc1]

A soln. of tert-butyl 2,2-dimethoxy-6-(4-(methoxycarbonyl)phenyl)-7-azaspiro[3.5]non-5-ene-7-carboxylate (Intermediate O-5, 18.8 g, 45.0 mmol) in EtOH/THF was brought under a N₂ atmosphere. Pd/C (2.40 g, 10 wt %, 2.25 mmol) was added and the RM was stirred under a H₂ atmosphere (balloon) for 120 min. The RM was flushed with N₂ and diluted with DCM. The suspension was filtered over Celite®, and rinsed with DCM (3×). The volatiles of the filtrate were removed under reduced pressure at 50° C. to obtain a colorless oil that was purified over silica gel (330 g) using an automated purification system (NP; Teledyne ISCO®; collected at 240/254 nm; flow 200 mL/min; eluent: heptane:Et₃N (99:1)+5 to 16.8% EtOAc in 19.6 min). The fractions were combined and the volatiles were removed under reduced pressure at 50° C. to give the title compound as a colorless, sticky oil (15.68 g). LC-MS Method B-2: Rt=1.18 min; MS m/z [M+H]⁺=420.4.

Intermediate rac-B-2:

### (RS)-tert-butyl 6-(4-(methoxycarbonyl)phenyl)-2-oxo-7-azaspiro[3.5]nonane-7-carboxylate

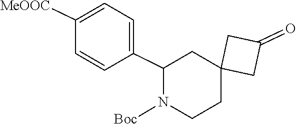 [SMILES: COC(=O)c1ccc(C2CC3(CCN2C(=O)OC(C)(C)C)CC(=O)C3)cc1]

2M aq. HCl soln. (46.72 mL, 93.44 mmol) was added to a soln. of tert-butyl 2,2-dimethoxy-6-(4-(methoxycarbonyl)phenyl)-7-azaspiro[3.5]nonane-7-carboxylate (Intermediate rac-B-1, 15.68 g, 37.38 mmol) in THF. The RM was stirred at RT for 90 min. Stirring was continued for 3 h. The RM was diluted with MTBE and brine. The separated layers were washed with brine and MTBE. The combined org. layers were dried over MgSO₄, filtered, and the volatiles were removed under reduced pressure at 50° C. to give the title compound (12.81 g) as a white, sticky foam that was used without further purification. LC-MS Method B-2: Rt=1.03 min; MS m/z [M+H]⁺=374.3.

Intermediate ds-rac-B-3:

### tert-butyl 2-hydroxy-6-(4-(methoxycarbonyl)phenyl)-7-azaspiro[3.5]nonane-7-carboxylate

(racemic mixtures; 4 stereoisomers)

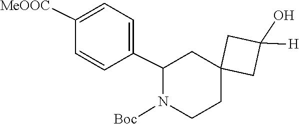 [SMILES: COC(=O)c1ccc(C2CC3(CCN2C(=O)OC(C)(C)C)CC(O)C3)cc1]

NaBH₄ (243 mg, 6.43 mmol) was added to a to 0° C. cooled soln. of tert-butyl 6-(4-(methoxycarbonyl)phenyl)-2-oxo-7-azaspiro[3.5]nonane-7-carboxylate (Intermediate rac-B-2, 4.80 g, 12.9 mmol) in MeOH. The RM was stirred at RT for 20 min in the ice bath. The RM was quenched with 1M aq. HCl soln., EtOAc and additional water were added and the layers were separated. The layers were washed with 1M aq. HCl soln., brine and EtOAc. The combined org. layers were dried over MgSO₄, filtered, and the volatiles were removed under reduced pressure at 50° C. to give a colorless oil that was purified over silica gel (120 g) using an automated purification system (NP; Teledyne ISCO®; collected at 246/254 nm; flow 85 mL/min; eluent: heptane+10 to 60% EtOAc in 20 min). Pure fractions were combined and the volatiles were removed under reduced pressure at 50° C. to give the title compound as a white foam (4.55 g, 55:45 diastereomeric mixture). LC-MS Method B-1: Rt=1.04 min; MS m/z [M-Boc+H]⁺=276.3.

Intermediate ds-rac-B-4:

### tert-butyl 2-hydroxy-6-(4-(methoxycarbonyl)phenyl)-2-methyl-7-azaspiro[3.5]nonane-7-carboxylate (racemic mixtures; 4 stereoisomers)

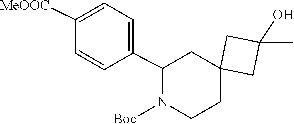 [SMILES: COC(=O)c1ccc(C2CC3(CCN2C(=O)OC(C)(C)C)CC(C)(O)C3)cc1]

A soln. of tert-butyl 6-(4-(methoxycarbonyl)phenyl)-2-oxo-7-azaspiro[3.5]nonane-7-carboxylate (Intermediate rac-B-2, 1.57 g, 4.20 mmol) in THE was brought under a N₂ atmosphere and cooled to −78° C. Methyl magnesium chloride (3M in THF, 3.08 mL, 9.25 mmol) was added in a dropwise manner. The RM was then allowed to reach −20° C., then the RM was poured into a well-stirred emulsion of 1M aq. HCl soln. and MTBE. The layers were then separated and washed with brine and MTBE. The combined org. layers were dried over MgSO₄, filtered, and the volatiles were removed under reduced pressure at 50° C. to give a colorless oil. The crude product was purified over silica gel (40 g) using an automated purification system (NP; Teledyne ISCO®; collected at 243/254 nm; flow 30 mL/min, eluent: DCM+0 to 32.2% MTBE in 20.2 min). Pure fractions were combined and the volatiles were removed under reduced pressure at 50° C. to give the title compound as a white foam (1.54 g, 68:32 diastereomer mixture). LC-MS Method B-3: Rt=5.19/5.29 min (68 and 32% area); MS m/z [M-tertBu+H]⁺=334.2.

Intermediate ds-rac-B-5:

### tert-butyl 2-fluoro-6-(4-(methoxycarbonyl)phenyl)-2-methyl-7-azaspiro[3.5]nonane-7-carboxylate

(racemic mixtures; 4 stereoisomers)

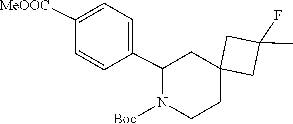 [SMILES: COC(=O)c1ccc(C2CC3(CCN2C(=O)OC(C)(C)C)CC(C)(F)C3)cc1]

A soln. of tert-butyl 2-hydroxy-6-(4-(methoxycarbonyl)phenyl)-2-methyl-7-azaspiro[3.5]nonane-7-carboxylate (Intermediate ds-rac-B-4, 402 mg, 1.03 mmol) in DCM was cooled to −78° C. DAST (266 mg, 218 μL, 1.65 mmol) was added in a dropwise manner. The RM was allowed to warm up to 0° C. and the mixture was stirred for 30 min. The RM was carefully and in portions poured into a well-stirred sat. aq. NaHCO₃ soln.; the layers were separated and the aq. layer was washed with DCM (2×). The combined org. layers were dried over K₂CO₃, filtered, and the volatiles were removed under reduced pressure at 50° C. to give a colorless oil. The crude product was purified over silica gel (12 g) using an automated purification system (NP; Teledyne ISCO®; collected at 246/254 nm; flow 35 mL/min; eluent: heptane+1 to 12% EtOAc in 15.6 min). Pure fractions were combined and the volatiles were removed under reduced pressure at 50° C. to give the title compound (diastereomeric mixture with unknown composition) as a colorless oil (362 mg). LC-MS Method B-1: Rt=1.30 min, MS m/z [M+H]⁺=392.5, [M-tertBu+H]⁺=336.5.

Intermediate rac-B-6:

### (RS)-tert-butyl 2,2-difluoro-6-(4-(methoxycarbonyl)phenyl)-7-azaspiro[3.5]nonane-7-carboxylate

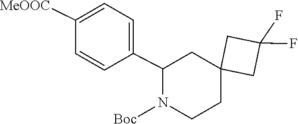 [SMILES: COC(=O)c1ccc(C2CC3(CCN2C(=O)OC(C)(C)C)CC(F)(F)C3)cc1]

### Achiral Hydrogenation:

To a soln. of compound tert-butyl 2,2-difluoro-6-(4-(methoxycarbonyl)phenyl)-7-azaspiro[3.5]non-5-ene-7-carboxylate (Intermediate O-6, 26.6 g, 67.6 mmol) in THE (130 mL) and EtOH (130 mL) was added Pd/C (2.70 g, 10 wt %) under N₂ atmosphere. The suspension was degassed under reduced pressure and purged with H₂ several times. The mixture was stirred under H₂ (15 psi) at 25° C. for 1.5 h. The RM was filtered and the filtrate was concentrated under reduced pressure providing the crude title compound (26.3 g) as a white solid. ¹H NMR (400 MHz, CDCl₃) δ [ppm]8.00 (d, J=8.4 Hz, 2H), 7.24 (d, J=8.0 Hz, 2H), 5.42 (br d, J=5.2 Hz, 1H), 4.24-4.15 (m, 1H), 3.90 (s, 3H), 3.07-2.97 (m, 1H), 2.40-2.27 (m, 3H), 2.10 (dd, J=6.4, 14.0 Hz, 1H), 1.96-1.81 (m, 1H), 1.77-1.68 (m, 3H), 1.39 (s, 9H).

Chiral Hydrogenation with (S,S)-iPr-DuPhos:

A round-bottom flask in the glovebox under N₂ atmosphere was charged with Rh(COD)₂BF₄ (331 mg; 4 mol %, CAS 35138-22-8) and (S,S)-iPr-DuPhos (344 mg; 4 mol %, CAS 147253-69-8), followed by hexafluoro-IPA (8 mL; CAS 920-66-1, degassed with N₂, dried over 4 Å molecular sieves overnight). The mixture was stirred for 1 h at RT and then added to tert-butyl 2,2-difluoro-6-(4-(methoxycarbonyl)phenyl)-7-azaspiro[3.5]non-5-ene-7-carboxylate (Intermediate O-6, 8.0 g, 20.33 mmol) in an autoclave in the glovebox, followed by hexafluoro-IPA (32 mL). The RM was degassed with N₂ and then hydrogenated under H₂ atmosphere (15 bar) at 60° C. for 16-18 h. The RM was cooled to ambient temperature providing a soln. of crude tert-butyl (S)-2,2-difluoro-6-(4-(methoxycarbonyl)phenyl)-7-azaspiro[3.5]nonane-7-carboxylate (70 and 87% ee; two different batches).

### Intermediate (+)-B-7:

### tert-butyl (R)-2,2-difluoro-6-(4-(methoxycarbonyl)phenyl)-7-azaspiro[3.5]nonane-7-carboxylate

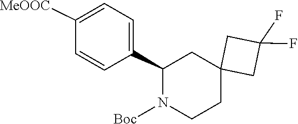 [SMILES: COC(=O)c1ccc([C@H]2CC3(CCN2C(=O)OC(C)(C)C)CC(F)(F)C3)cc1]

### and Intermediate (−)-B-8:

### tert-butyl (S)-2,2-difluoro-6-(4-(methoxycarbonyl)phenyl)-7-azaspiro[3.5]nonane-7-carboxylate

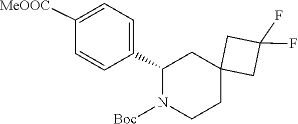 [SMILES: COC(=O)c1ccc([C@@H]2CC3(CCN2C(=O)OC(C)(C)C)CC(F)(F)C3)cc1]

Chiral separation of crude racemic tert-butyl 2,2-difluoro-6-(4-(methoxycarbonyl)phenyl)-7-azaspiro[3.5]nonane-7-carboxylate (Intermediate rac-B-6, 32 g) was carried out by chiral preparative SFC:

### Preparative Separation Method:

Instrument: Waters SFC prep 350; Column: REGIS (S,S) WHELK-01 (250 mm×50 mm, 10 μm); Mobile phase: A for CO₂ and B for EtOH (0.1% NH₄OH); Gradient: B 35%; Flow rate: 200 mL/min; Back pressure: 100 bar; Column temperature: 35° C.; Wavelength: 220 nm; Cycle time: ˜3.85 min; Sample preparation: Compound was dissolved in −300 mL MeOH and DCM; Injection: 18 mL per injection.

Peak 1: Intermediate (+)-B-7 eluting first from the column. Recovered amount 12.5 g.

Peak 2: Intermediate (−)-B-8 eluting second from the column. Recovered amount 13.1 g.

### Analytical Method:

Instrument: Agilent-1260; Column: (S,S)Whelk-O1 50×4.6 mm I.D., 3.5 μm; Mobile phase: Phase A for CO₂, and Phase B for MeOH (0.05% DEA); Gradient elution: B in A from 5% to 40%; Flow rate: 3 mL/min; Detector: DAD; Column temperature: 35° C.; Back pressure: 100 bar

Peak 1: Intermediate (+)-B-7 Rt=0.887 min; >99% ee. Specific rotation Method OR2: [α]²⁵ @589 nm=+49.1° (11.5 mg of Intermediate (+)-B-7 in 1.1 mL DCM).

¹H NMR (400 MHz, MeOD-d₄) δ [ppm]8.01 (d, J=8.44 Hz, 2H), 7.35 (d, J=8.08 Hz, 2H), 5.42 (br d, J=4.52 Hz, 1H), 4.17 (dt, J=13.88, 3.56 Hz, 1H), 3.90 (s, 3H), 3.12 (ddd, J=14.00, 10.44, 5.36 Hz, 1H), 2.48-2.27 (m, 3H), 2.12 (dd, J=14.04, 6.48 Hz, 1H), 2.02-1.91 (m, 1H), 1.79-1.68 (m, 3H), 1.40 (s, 9H).

Peak 2: Intermediate (−)-B-8 Rt=1.205 min; >99% ee. Specific rotation Method OR2: [α]²⁵ @589 nm=−49.1° (11.1 mg of Intermediate (−)-B-8 in 1.1 mL DCM).

¹H NMR (400 MHz, MeOD-d₄) δ [ppm]8.01 (d, J=8.44 Hz, 2H), 7.34 (d, J=8.20 Hz, 2H), 5.42 (br d, J=4.64 Hz, 1H), 4.17 (dt, J=13.92, 3.56 Hz, 1H), 3.90 (s, 3H), 3.12 (ddd, J=14.00, 10.40, 5.44 Hz, 1H), 2.49-2.28 (m, 3H), 2.12 (dd, J=14.08, 6.48 Hz, 1H), 1.97 (tdd, J=13.68, 13.68, 10.00, 3.68 Hz, 1H), 1.40 (s, 9H), 1.78-1.67 (m, 3H).

Intermediate ds-rac-B-9:

### tert-butyl 2-fluoro-6-(4-(methoxycarbonyl)phenyl)-7-azaspiro[3.5]nonane-7-carboxylate (Racemic Mixtures; 4 Stereoisomers)

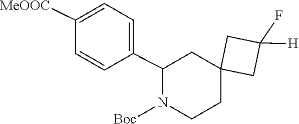 [SMILES: COC(=O)c1ccc(C2CC3(CCN2C(=O)OC(C)(C)C)CC(F)C3)cc1]

A soln. of tert-butyl 2-hydroxy-6-(4-(methoxycarbonyl)phenyl)-7-azaspiro[3.5]nonane-7-carboxylate (Intermediate ds-rac-B-3, 556 mg, 1.48 mmol) and 2,6-lutidine (793 mg, 858 μL, 7.40 mmol) in dry DCM (5.5 mL) was cooled to −78° C. Trifluoromethanesulfonic anhydride (836 mg, 500 μL, 2.96 mmol) was added in a dropwise manner. The cooling bath was removed and the RM was stirred for 20 min. The RM was partitioned between MTBE and 1M aq. HCl soln.; the layers were separated and washed with 1M aq. HCl soln., brine and MTBE. The combined org. layers were dried over MgSO₄, filtered, and the volatiles were removed under reduced pressure at 50° C. to give an orange oil. The material was dissolved in DCM (5 mL) and TBAF (2.96 mL of a 1M soln. in THF, 2.96 mmol) was added. The RM was stirred at RT for 60 min. The RM was partitioned between water and MTBE. The layers were separated and washed with water (2×), brine and MTBE. The combined org. layers were dried over MgSO₄, filtered, and the volatiles were removed under reduced pressure at 50° C. to give a yellow oil. The crude product was purified over silica gel (24 g) using an automated purification system (NP; Teledyne ISCO®; collected at 241/254 nm; flow 35 mL/min, eluent: heptane+1 to 15% EtOAc in 21.4 min). Pure fractions were combined and the volatiles were removed under reduced pressure at 50° C. to give the title compound as a 55:45 diastereomer mixture (284 mg). LC-MS Method B-1: Rt=1.30 min; MS m/z [M+H]⁺=378.3.

Intermediate rac-B-10:

### (RS)-tert-butyl 6-(4-(methoxycarbonyl)phenyl)-2-oxa-7-azaspiro[3.5]nonane-7-carboxylate

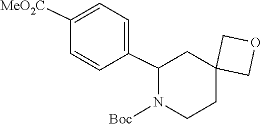 [SMILES: COC(=O)c1ccc(C2CC3(CCN2C(=O)OC(C)(C)C)COC3)cc1]

A soln. of tert-butyl 6-(4-(methoxycarbonyl)phenyl)-2-oxa-7-azaspiro[3.5]non-5-ene-7-carboxylate (Intermediate O-7, 236 mg, 0.657 mmol) in EtOH (4 mL)/THF(0.5 mL) was brought under a N₂ atmosphere. Pd/C (69.9 mg, 10 wt %, 0.066 mmol) was added and the RM was stirred under a H₂ atmosphere (balloon) for 60 min. The RM was purged with N₂ and filtered over Celite®. The catalyst was washed with DCM (3×). The filtrate was evaporated to dryness under reduced pressure at 50° C. to give the title compound as a colorless oil (228 mg) which solidified upon standing. LC-MS Method B-1: Rt=1.02 min; MS m/z [M+H]⁺=362.2.

### Intermediate B-11:

### benzyl (S)-2-(4-(methoxycarbonyl)phenyl)-4-methylenepiperidine-1-carboxylate

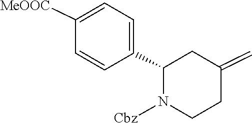 [SMILES: C=C1CCN(C(=O)OCc2ccccc2)[C@H](c2ccc(C(=O)OC)cc2)C1]

To the stirred white suspension of methyl triphenylphosphoniumilodide (990 mg, 2.45 mmol) in dry THE (5 mL) was added 1.0 M soln. KOtBu in THE (2.45 mL, 2.45 mmol) at 0° C. and the resulting yellow mixture was stirred for 30 min at 0° C. Then a soln. of benzyl (S)-2-(4-(methoxycarbonyl)phenyl)-4-oxopiperidine-1-carboxylate (CAS 2408761-20-4, 300 mg, 0.816 mmol) in THE (5 mL) was added to the mixture at 0° C. The ice bath was removed, and the mixture was stirred at RT for 2 h. The mixture was quenched with water (20 mL) and extracted with EtOAc (2×25 mL). The combined org. phases were combined, dried over Na₂SO₄, and concentrated under reduced pressure. The crude residue was purified over silica gel (12 g) using an automated purification system (NP; heptane/EtOAc). Fractions containing product were combined and concentrated to provide the title compound (130 mg) as a sticky yellow solid. LC-MS Method A-1: Rt=1.65 min; MS m/z [M+H]⁺=366.0.

### Intermediate B-12 and Intermediate B-13:

### benzyl (3S,5S)-1,1-difluoro-5-(4-(methoxycarbonyl)phenyl)-6-azaspiro[2.5]octane-6-carboxylate and benzyl (3R,5S)-1,1-difluoro-5-(4-(methoxycarbonyl)phenyl)-6-azaspiro[2.5]octane-6-carboxylate

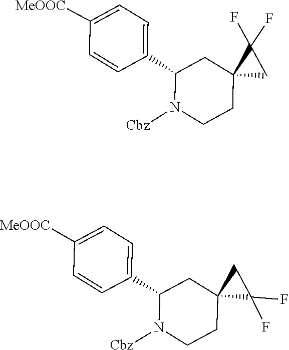 [SMILES: COC(=O)c1ccc([C@@H]2C[C@@]3(CCN2C(=O)OCc2ccccc2)CC3(F)F)cc1 ; COC(=O)c1ccc([C@@H]2C[C@]3(CCN2C(=O)OCc2ccccc2)CC3(F)F)cc1]

To a stirred soln. of Intermediate B-11 (130 mg, 0.355 mmol) in dry THE (1 mL) were added trimethyl(trifluoromethyl)silane (253 mg, 1.778 mmol) and NaI (27 mg, 0.177 mmol), and the mixture was heated at 120° C. for 1 h. The reaction was quenched with water (10 mL) and extracted with EtOAc (2×25 mL). The org. phases were combined, dried over Na₂SO₄, and the solvents were removed under reduced pressure. The crude mixture of diastereomers was purified over silica gel using an automated purification system (NP; heptane/EtOAc). Pure fractions were combined and the volatiles were removed under reduced pressure to provide the title compounds (105 mg) as a ˜10:1 mixture. LC-MS Method A-1: Rt=1.64 min; MS m/z=416.0 [M+H]⁺.

### Intermediate (+)-B-14(+):

### tert-butyl (5S,7R)-2,2-difluoro-7-(4-(methoxycarbonyl)phenyl)-8-azaspiro[4.5]decane-8-carboxylate or tert-butyl (5R,7R)-2,2-difluoro-7-(4-(methoxycarbonyl)phenyl)-8-azaspiro[4.5]decane-8-carboxylate

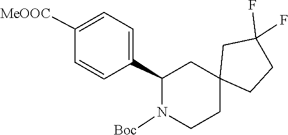 [SMILES: COC(=O)c1ccc([C@H]2CC3(CCN2C(=O)OC(C)(C)C)CCC(F)(F)C3)cc1]

### and Intermediate (−)-B-15(+):

### tert-butyl (5S,7S)-2,2-difluoro-7-(4-(methoxycarbonyl)phenyl)-8-azaspiro[4.5]decane-8-carboxylate or tert-butyl (5R,7S)-2,2-difluoro-7-(4-(methoxycarbonyl)phenyl)-8-azaspiro[4.5]decane-8-carboxylate

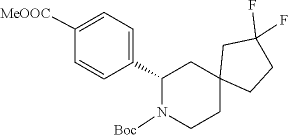 [SMILES: COC(=O)c1ccc([C@@H]2CC3(CCN2C(=O)OC(C)(C)C)CCC(F)(F)C3)cc1]

Hydrogenation of (+)-tert-butyl 2,2-difluoro-7-(4-(methoxycarbonyl)phenyl)-8-azaspiro[4.5]dec-6-ene-8-carboxylate (Intermediate (+)-O-9) provided Intermediate (+)-B-14(+) and Intermediate (−)-B-15(+): To a soln. of Intermediate (+)-O-9 (510 mg, 1.25 mmol) in THE (10 mL) and EtOH (15 mL) in a Parr pressure bottle was added Pd/C (wet form 50 wt %, 66.6 mg, 10 wt %, 0.0626 mmol). Hydrogenation was carried out under H₂ atmosphere at 45 psi for 2 to 3 h. The solids were filtered off and rinsed/washed with EtOH (containing ˜5% water). The filtrate was concentrated under reduced pressure and dried under reduced pressure providing a crude diastereomeric mixture of the title compounds as a white solid. LC-MS Method C-3: Rt=1.28 min; MS m/z [M-tertBu+H]⁺=354.3.

Diastereomer separation using SFC chiral separation method. Instrument: Thar 80 preparative SFC; column: (S,S) Whelk-O1 21×250 mm 5 μm; flow rate: 80 g per min; co-solvent: 20% 1:1 MeOH:IPA in CO₂; detection: 270 nm; back pressure set point: 125 bar; injection size: 78 mg (15.6 mg/mL in 9:1 MeOH/DCM).

Peak 1: Intermediate (+)-B-14(+) Isolated: 320 mg. White solid. Method SFC-2: Rt=1.32 min, ee>99%. Specific rotation Method OR-1: [α]²⁵ @589 nm=+49.1° (10.914 mg of Intermediate (+)-B-14(+) in 1 mL MeOH).

Peak 2: Intermediate (−)-B-15(+) Isolated: 146 mg. White solid. Method SFC-2: Rt=1.71 min, ee>99%. Specific rotation Method OR-1: [α]²⁵ @589 nm=−44.2° (10.696 mg of Intermediate (−)-B-15(+) in 1 mL MeOH).

### Intermediate (+)-B-16(−):

### tert-butyl (5S,7R)-2,2-difluoro-7-(4-(methoxycarbonyl)phenyl)-8-azaspiro[4.5]decane-8-carboxylate or tert-butyl (5R,7R)-2,2-difluoro-7-(4-(methoxycarbonyl)phenyl)-8-azaspiro[4.5]decane-8-carboxylate

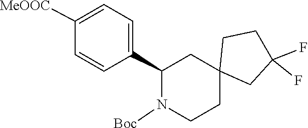 [SMILES: COC(=O)c1ccc([C@H]2CC3(CCN2C(=O)OC(C)(C)C)CCC(F)(F)C3)cc1]

### and Intermediate (−)-B-17(−):

### tert-butyl (5S,7S)-2,2-difluoro-7-(4-(methoxycarbonyl)phenyl)-8-azaspiro[4.5]decane-8-carboxylate or tert-butyl (5R,7S)-2,2-difluoro-7-(4-(methoxycarbonyl)phenyl)-8-azaspiro[4.5]decane-8-carboxylate

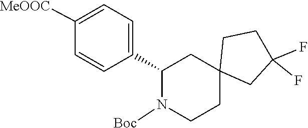 [SMILES: COC(=O)c1ccc([C@@H]2CC3(CCN2C(=O)OC(C)(C)C)CCC(F)(F)C3)cc1]

Hydrogenation of (−)-tert-butyl 2,2-difluoro-7-(4-(methoxycarbonyl)phenyl)-8-azaspiro[4.5]dec-6-ene-8-carboxylate (Intermediate (−)-O-10, 500 mg, 1.23 mmol) was carried out according to the method described for Intermediate (+)-O-9 herein above, providing a mixture of Intermediate (+)-B-16(−) and Intermediate (−)-B-17(−). LC-MS Method C-3: Rt=1.28 min; MS m/z [M-tertBu+H]⁺=354.3.

Diastereomer separation using SFC chiral separation method. Instrument: Thar 80 preparative SFC; column: (S,S) Whelk-O1 21×250 mm 5 μm; flow rate: 80 g per min; co-solvent: 25% 1:1 MeOH:IPA in CO₂; detection: 270 nm; back pressure set point: 125 bar; injection size: 88.2 mg (14.7 mg/mL in 9:1 MeOH/DCM).

Peak 1: Intermediate (+)-B-16(−) Isolated: 149 mg. White solid. Method SFC-2: Rt=1.30 min, ee>99%. Specific rotation Method OR-1: [α]²⁵ @589 nm=+44.5° (8.550 mg of Intermediate (+)-B-16(−) in 1 mL MeOH).

Peak 2: Intermediate (−)-B-17(−) Isolated: 330 mg. White solid. Method SFC-2: Rt=1.73 min/5.0 min, ee>99%. Specific rotation Method OR-1: [α]²⁵ @589 nm=−51.3° (10.230 mg of Intermediate (−)-Boc-P-17(−) in 1 mL MeOH).

In analogy to the tert-butyl (S)-2,2-difluoro-6-(4-(methoxycarbonyl)phenyl)-7-azaspiro[3.5]nonane-7-carboxylate (Intermediate (−)-B-8), here the Boc-piperidine derivatives with negative optical rotation were tentatively assigned with the (S)-configurated benzyl-amine.

The Boc-piperidine derivatives with positive optical rotation of were assigned with the (R)-configurated benzyl-amine.

Intermediate rac-B-18:

### (RS)-tert-butyl 2,2-difluoro-6-(4-(3-hydroxyoxetan-3-yl)phenyl)-7-azaspiro[3.5]nonane-7-carboxylate

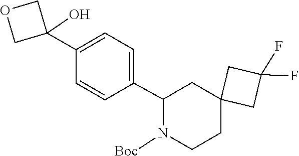 [SMILES: CC(C)(C)OC(=O)N1CCC2(CC1c1ccc(C3(O)COC3)cc1)CC(F)(F)C2]

A soln. of tert-butyl 2,2-difluoro-6-(4-(3-hydroxyoxetan-3-yl)phenyl)-7-azaspiro[3.5]non-5-ene-7-carboxylate (Intermediate O-11, 480 mg, 83 wt %, 978 μmol) in EtOH (10 mL) and THE (0.5 mL) was brought under a N₂ atmosphere. Pd/C (104.1 mg, 10 wt %, 97.78 μmol) was added and the RM was then stirred under a H₂ atmosphere (balloon) for 60 min. The RM was purged with N₂ and filtered over Celite®. The catalyst was washed with DCM (3×). The volatiles of the filtrate were removed under reduced pressure at 50° C. to give a colorless oil. The crude product was purified over silica gel (24 g) using an automated purification system (NP; Teledyne ISCO®; collected at 238/221, later 354 nm+ELSD detector used; flow 35 mL/min; eluent: heptane+5-24% (EtOAc:MeOH 95:5) in 22.1 min). Fractions were combined and the volatiles were removed under reduced pressure at 50° C. to give the title compound as a colorless oil (370 mg). LC-MS Method B-1: Rt=1.12 min. MS m/z [M+H]⁺=410.4, [M-tertBu+H]⁺=354.3.

Intermediate rac-B-19:

### (RS)-tert-butyl 2,2-difluoro-6-(4-(2-hydroxypropan-2-yl)phenyl)-7-azaspiro[3.5]nonane-7-carboxylate

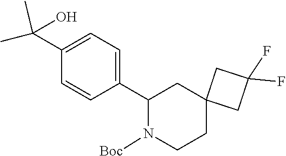 [SMILES: CC(C)(C)OC(=O)N1CCC2(CC1c1ccc(C(C)(C)O)cc1)CC(F)(F)C2]

A soln. of tert-butyl 2,2-difluoro-6-(4-(2-hydroxypropan-2-yl)phenyl)-7-azaspiro[3.5]non-5-ene-7-carboxylate (Intermediate O-12, 344.0 mg, 874.3 μmol) in EtOH (10.0 mL) and THE (0.50 mL) was brought under a N₂ atmosphere. Pd/C (93.04 mg, 10 wt %, 87.43 μmol) was added and the RM was then stirred under a H₂ atmosphere (balloon) for 60 min.

The RM was purged with N₂ and filtered over Celite®. The catalyst was 3× washed with DCM. The volatiles of the filtrate were removed under reduced pressure at 50° C. to give a colorless oil, which was purified over silica gel (12 g) using an automated purification system (NP; Teledyne ISCO®; collected at 235/256 nm+ELSD detector used; flow 30 mL/min; eluent: heptane+2 to 22.6% EtOAc in 17.9 min). Purest fractions were combined and the volatiles were removed under reduced pressure at 50° C. to give the title compound (258 mg) as a colorless oil. LC-MS Method B-1: Rt=1.24 min; MS m/z [M-tertBu-H₂O+H]⁺=322.3.

Intermediate rac-B-20:

### (RS)-tert-butyl 6-(4-(1H-pyrazol-5-yl)phenyl)-2,2-difluoro-7-azaspiro[3.5]nonane-7-carboxylate

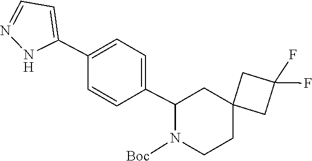 [SMILES: CC(C)(C)OC(=O)N1CCC2(CC1c1ccc(-c3ccn[nH]3)cc1)CC(F)(F)C2]

A soln. of tert-butyl 6-(4-(1H-pyrazol-1-yl)phenyl)-2,2-difluoro-7-azaspiro[3.5]non-5-ene-7-carboxylate (Intermediate O-13, 293.0 mg, 83 wt %, 605.8 μmol) in EtOH (7.0 mL) was brought under a N₂ atmosphere. Pd/C (64.47 mg, 10 wt %, 60.58 μmol) was added and the RM was then stirred under a H₂ atmosphere (balloon) for 75 min, another Pd/C (65 mg, 10 wt %) were added. Stirring was continued for another 4 h. The RM was purged with N₂ and filtered over Celite®. The solids were rinsed with DCM (3×). The volatiles of the filtrate were removed under reduced pressure at 50° C. to give a colorless oil that was treated with hexane. The volatiles were removed under reduced pressure at 50° C. to give a yellow oil. The crude product was purified over silica gel (12 g) using an automated purification system (NP; Teledyne ISCO®; collected at 257/280 nm; flow 30 mL/min; eluent: heptane+5 to 34.7% EtOAc in 20.4 min). Pure fractions were combined and the volatiles were removed under reduced pressure at 50° C. to give the title compound as a colorless oil (249 mg). LC-MS Method B-1: Rt=1.24 min; MS m/z [M+H]⁺=404.2, [M-tertBu+H]⁺=348.2.

Intermediate rac-B-21:

### (RS)-tert-butyl 6-(4-(1H-pyrazol-1-yl)phenyl)-2,2-difluoro-7-azaspiro[3.5]nonane-7-carboxylate

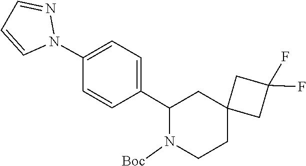 [SMILES: CC(C)(C)OC(=O)N1CCC2(CC1c1ccc(-n3cccn3)cc1)CC(F)(F)C2]

A soln. of tert-butyl 6-(4-(1H-pyrazol-1-yl)phenyl)-2,2-difluoro-7-azaspiro[3.5]non-5-ene-7-carboxylate (Intermediate O-14, 233.0 mg, 580.4 μmol) in EtOH (7.0 mL) was brought under a N₂ atmosphere. Pd/C (61.76 mg, 10 wt %, 58.04 μmol) was added and the RM was then stirred under a H₂ atmosphere (balloon) for 75 min. The RM was purged with N₂ and filtered over Celite®. The solids were rinsed with DCM (3×), the volatiles of the filtrate were removed under reduced pressure at 50° C., the resulting crude oil was treated with hexane and the solvent was removed again to give the title compound as a white solid (370 mg). LC-MS Method B-1: Rt=1.28 min; MS m/z [M-tertBu+H]⁺=348.3.

Intermediate rac-B-22:

### (RS)-tert-butyl 6-(4-cyanophenyl)-2,2-difluoro-7-azaspiro[3.5]nonane-7-carboxylate

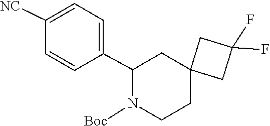 [SMILES: CC(C)(C)OC(=O)N1CCC2(CC1c1ccc(C#N)cc1)CC(F)(F)C2]

A soln. of tert-butyl 6-(4-cyanophenyl)-2,2-difluoro-7-azaspiro[3.5]non-5-ene-7-carboxylate (Intermediate O-15, 335.0 mg, 929.5 μmol) in EtOAc (15.0 mL) was brought under a N₂ atmosphere. Pd/C (98.92 mg, 10 wt %, 92.95 μmol) was added and the RM was then stirred under a H₂ atmosphere (balloon) for 60 min. The RM was purged with N₂, filtered over Celite®, the solids were rinsed with DCM (3×). The volatiles of the filtrate were removed under reduced pressure at 50° C. to give a colorless oil that was purified over silica gel (12 g) using an automated purification system (NP; Teledyne ISCO®; collected at 245/254 nm; flow 30 mL/min; eluent: heptane+5 to 19% EtOAc in 11.2 min). Pure fractions were combined and the volatiles were removed under reduced pressure at 50° C. to give the title compound as a colorless oil (259 mg). LC-MS Method B-1: Rt=1.24 min; MS m/z [M-Boc+H]⁺=263.3.

Intermediate rac-B-23:

### (RS)-tert-butyl 2,2-difluoro-6-phenyl-7-azaspiro[3.5]nonane-7-carboxylate

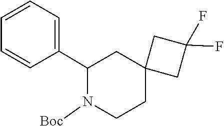 [SMILES: CC(C)(C)OC(=O)N1CCC2(CC1c1ccccc1)CC(F)(F)C2]

A soln. of tert-butyl 2,2-difluoro-6-phenyl-7-azaspiro[3.5]non-5-ene-7-carboxylate (Intermediate O-16, 274.0 mg, 817.0 μmol) in EtOH (7.0 mL) was brought under a N₂ atmosphere. Pd/C (86.94 mg, 10 wt %, 81.70 μmol) was added and the RM was then stirred under a H₂ atmosphere (balloon) for 90 min. The RM was purged with N₂ and filtered over Celite®. The solids were rinsed with DCM (3×). The volatiles of the filtrate were removed under reduced pressure at 50° C. to give a colorless oil that was triturated with hexane and dried. The crude product was purified over silica gel (12 g) using an automated purification system (NP; Teledyne ISCO®; collected at 254 nm+ELSD detector; flow 30 mL/min; eluent: heptane+0 to 7.4% EtOAc in 12.3 min). Pure fractions were combined and the volatiles were removed under reduced pressure at 50° C. to give the title compound as a colorless oil (242 mg). LC-MS Method B-1: Rt=1.44 min; MS m/z [M+H]⁺=338.3.

Intermediate rac-B-24:

### (RS)-tert-butyl 2,2-difluoro-6-(1-methyl-1H-pyrazol-4-yl)-7-azaspiro[3.5]nonane-7-carboxylate

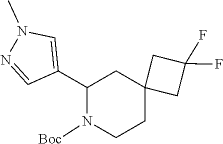 [SMILES: Cn1cc(C2CC3(CCN2C(=O)OC(C)(C)C)CC(F)(F)C3)cn1]

A soln. of tert-butyl 2,2-difluoro-6-(1-methyl-1H-pyrazol-4-yl)-7-azaspiro[3.5]non-5-ene-7-carboxylate (Intermediate O-17, 137.0 mg, 403.7 μmol) in EtOH (5.0 mL) was brought under a N₂ atmosphere. Pd/C (42.96 mg, 10 wt %, 40.37 μmol) was added and the RM was then stirred under a H₂ atmosphere (balloon) for overnight, the RM was purged with N₂ and filtered over Celite®. The solids were rinsed with EtOH (3×). The volatiles of the filtrate were removed under reduced pressure at 50° C. to give a grey oil. The residue was dissolved in EtOH (4 mL), brought under a N₂ atmosphere and Pd/C (80 mg, 10 wt %) were added. The RM was then stirred under a H₂ atmosphere (balloon) for 24 h. The RM was purged with N₂ and filtered over Celite®. The solids were rinsed with DCM (3×). The volatiles of the filtrate were removed under reduced pressure at 50° C. The residue was treated with hexane, and the volatiles were removed under reduced pressure at 50° C. to give a colorless oil that was was purified over silica gel (12 g) using an automated purification system (NP; Teledyne ISCO®; collected at 257/280 nm; flow 30 mL/min; eluent: heptane+10 to 90% EtOAc in 33.3 min). Pure fractions were combined and the volatiles were removed under reduced pressure at 50° C., treated with hexane/DCM, solvents removed again at 50° C. to give the title compound as a colorless oil. LC-MS Method B-1: Rt=1.08 min; MS m/z [M+H]⁺=342.4.

Intermediate rac-B-25:

### tert-butyl (RS)-6-(6-bromopyridin-3-yl)-2,2-difluoro-7-azaspiro[3.5]nonane-7-carboxylate

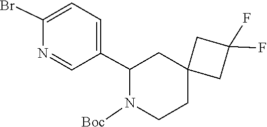 [SMILES: CC(C)(C)OC(=O)N1CCC2(CC1c1ccc(Br)nc1)CC(F)(F)C2]

### Step 1: tert-butyl (2-(1-(2-(6-bromopyridin-3-yl)-2-oxoethyl)-3,3-difluoro-cyclobutyl)ethyl)carbamate (Intermediate rac-B-25-1)

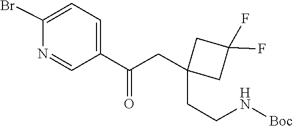 [SMILES: CC(C)(C)OC(=O)NCCC1(CC(=O)c2ccc(Br)nc2)CC(F)(F)C1]

A soln. of 2-bromo-5-iodopyridine (3.403 g, 11.99 mmol) in THE (30.0 mL) was evacuated/back-filled with N₂ twice and cooled to −30° C. Isopropylmagnesium chloride lithium chloride complex solution in THE (1.30 M, 9.40 mL, 12.26 mmol) was added dropwise and the RM was then stirred between −30° C. and −25° C. for 90 min. In a separate flask, tert-butyl 2,2-difluoro-6-oxo-7-azaspiro[3.5]nonane-7-carboxylate (Intermediate O-2-2, 1.50 g, 5.45 mmol) was dissolved in THE (15.0 mL), evacuated and back-filled with N₂ twice, and cooled to −78° C. The freshly prepared Grignard soln. above was added via cannula using a positive pressure of N₂. The resulting yellow soln. was stirred at −78° C. for 20 min. The temperature was allowed to rise to −25° C., then the RM was poured into a well-stirred mixture of aq. NH₄Cl soln. (10 wt %) and TBME. The layers were separated and washed with brine and TBME. The combined org. layers were dried over MgSO₄, filtered, and the volatiles were removed under reduced pressure at 50° C. The residue was purified over silica gel (80 g) using an automated purification system (NP; Teledyne ISCO®; collected at 245/275 nm; flow 60 mL/min; eluent: heptane+5 to 30% EtOAc in 27.4 min). Pure fractions were combined and the volatiles were removed under reduced pressure at 50° C. to give the title compound (1.88 g) as a yellow oil. LC-MS Method B-6: Rt=1.23 min; MS m/z [M−H]⁻=430.9/433.0.

### Step 2: 6-(6-bromopyridin-3-yl)-2,2-difluoro-7-azaspiro[3.5]nonane (Intermediate rac-B-25-2)

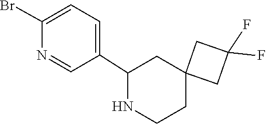 [SMILES: FC1(F)CC2(CCNC(c3ccc(Br)nc3)C2)C1]

TFA (9.0 mL) was added to a soln. of tert-butyl (2-(1-(2-(6-bromopyridin-3-yl)-2-oxoethyl)-3,3-difluoro-cyclobutyl)ethyl)carbamate (Intermediate rac-B-25-1, 1.880 g, 87.5 wt %, 3.797 mmol) in DCM (27.0 mL). The RM was stirred at RT for 1 h. The volatiles were removed under reduced pressure at 50° C. The residue was partitioned between DCM and sat. aq. NaHCO₃ soln. The separated aq. layer was washed twice with DCM. The combined org. layers were dried over MgSO₄, filtered, and the volatiles were removed under reduced pressure at 50° C. The residue was dissolved in EtOH (15.0 mL) and the resulting soln. was cooled to 0-5° C. NaBH₄ (287.2 mg, 7.593 mmol) was added in two portions over a period of 10 min. The RM was stirred in the ice bath for an additional 50 min. The RM was carefully poured into a well-stirred mixture of EtOAc and water. The separated layers were washed with sat. aq. NaHCO₃ soln., brine and EtOAc. The combined org. layers were dried over MgSO₄, filtered, and the volatiles were removed under reduced pressure at 50° C. The residue was dissolved in DCM and purified over silica gel (40 g) using an automated purification system (NP; Teledyne ISCO®; collected at 254/268 nm; flow 40 mL/min; eluent: heptane+10 to 77.5% {EtOAc/MeOH=95/5} in 18.8 min). Pure fractions were combined and the volatiles were removed under reduced pressure at 50° C. to give the title compound (1.02 g) as white crystals. LC-MS Method B-6: Rt=0.31 min; MS m/z [M+H]⁺=317.0/318.9.

### Step 3: tert-butyl 6-(6-bromopyridin-3-yl)-2,2-difluoro-7-azaspiro[3.5]nonane-7-carboxylate (Intermediate rac-B-25)

Triethylamine (493.1 mg, 679 μL, 4.873 mmol) and di-tert-butyl dicarbonate (930.6 mg, 4.264 mmol) were added to a soln. of 6-(6-bromopyridin-3-yl)-2,2-difluoro-7-azaspiro[3.5]nonane (Intermediate rac-B-25-2, 966.0 mg, 3.046 mmol) in DCM (10.0 mL) and the RM was stirred overnight. The volatiles were removed under reduced pressure at 50° C. to give a yellow oil that was purified over silica gel (40 g) using an automated purification system (NP; Teledyne ISCO®; collected at 254/272 nm; flow 40 mL/min; eluent: heptane+3.0 to 20.4% EtOAc in 18.3 min). Pure fractions were combined and the volatiles were removed under reduced pressure at 50° C. to give the title compound (1.27 g) as a colorless oil. LC-MS Method B-6: Rt=1.30 min; MS m/z [M+H]⁺=417.0/419.1.

Intermediate rac-B-26:

### tert-butyl (RS)-6-(6-cyanopyridin-3-yl)-2,2-difluoro-7-azaspiro[3.5]nonane-7-carboxylate

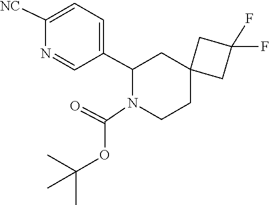 [SMILES: CC(C)(C)OC(=O)N1CCC2(CC1c1ccc(C#N)nc1)CC(F)(F)C2]

A mixture of tert-butyl 6-(6-bromopyridin-3-yl)-2,2-difluoro-7-azaspiro[3.5]nonane-7-carboxylate (Intermediate rac-B-25, 300 mg, 718.9 μmol), Zn(CN)₂ (50.65 mg, 431.4 μmol), zinc powder (5.640 mg, 1.231 μL, 86.27 μmol) and dppf (15.94 mg, 28.76 μmol) in DMA (2.3 mL) was placed in a vial and evacuated/back-filled with N₂ twice. Pd₂(dba)₃ (12.99 mg, 14.38 μmol) was added and the mixture was evacuated/backfilled with N₂ once more. The capped vial was stirred for 70 min at 105° C. The RM was cooled to RT and partitioned between TBME and water. The layers were separated and washed with water, brine and TBME. The combined org. layers were dried over MgSO₄, filtered, and the volatiles were removed under reduced pressure at 50° C. The residue was purified over silica gel (24 g) using an automated purification system (NP; Teledyne ISCO®; collected at 245/270 nm; flow 35 mL/min; eluent: heptane+5 to 30% EtOAc in 17.8 min). Pure fractions were combined and the volatiles were removed under reduced pressure at 50° C. to give the title compound (250 mg) as a colorless oil. LC-MS Method B-6: Rt=1.10 min; MS m/z [M+H]⁺=364.1.

Intermediate rac-B-27:

### tert-butyl (RS)-2,2-difluoro-6-(6-(methoxycarbonyl)pyridin-3-yl)-7-azaspiro[3.5]nonane-7-carboxylate

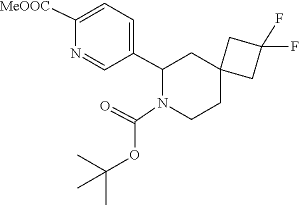 [SMILES: COC(=O)c1ccc(C2CC3(CCN2C(=O)OC(C)(C)C)CC(F)(F)C3)cn1]

A mixture of tert-butyl 6-(6-bromopyridin-3-yl)-2,2-difluoro-7-azaspiro[3.5]nonane-7-carboxylate (Intermediate rac-B-25, 504.0 mg, 1.208 mmol), carbon monoxide-molybdenum (6:1; 318.9 mg, 1.208 mmol) and tri-tert-butyl phosphonium tetrafluoroborate (70.08 mg, 241.6 μmol) in ACN (4.90 mL) and MeOH (1.23 mL) was placed in a vial and evacuated/back-filled with N₂ twice. Trans-bis(acetato)bis[2-[bis(2-methylphenyl)phosphino]benzyl]dipalladium(II) (56.62 mg, 60.39 μmol) and DBU (275.8 mg, 273.1 μL, 1.812 mmol) were added and the mixture was evacuated/backfilled with N₂ once more. The capped vial was heated at 75° C. for 21 h, then stirring was continued at RT for ˜2 days. The RM was partitioned between TBME and water, the layers were separated and washed with water, brine and TBME. The combined org. layers were dried over MgSO₄, filtered, and the volatiles were removed under reduced pressure at 50° C. The residue was purified over silica gel (24 g) using an automated purification system (NP; Teledyne ISCO®; collected at 245/270 nm; flow 35 mL/min; eluent: heptane+10 to 50% EtOAc in 21.7 min). Pure fractions were combined and the volatiles were removed under reduced pressure at 50° C. to give the title compound (345.0 mg) as an off-white solid. LC-MS Method B-6: Rt=1.08 min; MS m/z [M+H]⁺=397.2.

Intermediate rac-P-1 and Intermediate rac-P-2:

### methyl 4-(2-hydroxy-2-methyl-7-azaspiro[3.5]nonan-6-yl)benzoate

(separated racemic diastereomers; 2 mixtures of 2 stereoisomers each)

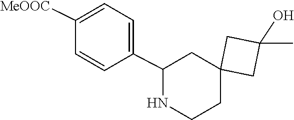 [SMILES: COC(=O)c1ccc(C2CC3(CCN2)CC(C)(O)C3)cc1]

tert-Butyl 2-hydroxy-6-(4-(methoxycarbonyl)phenyl)-2-methyl-7-azaspiro[3.5]nonane-7-carboxylate (Intermediate ds-rac-B-4, 400 mg, 1.03 mmol) was dissolved in 4M HCl in dioxane (5 mL). The RM was stirred at RT for 50 min, then poured into a well-stirred mixture of 4M aq. NaOH soln. (5.5 mL), ice and DCM. The layers were separated, and the aq. layer was washed with DCM (3×). The combined org. layers were dried over K₂CO₃, filtered, and the volatiles were removed under reduced pressure at 50° C. The crude product was purified over silica gel (24 g) using an automated purification system (NP; Teledyne ISCO®; collected at 235/254 nm; flow 35 mL/min; eluent: DCM+0 to 7.6 (MeOH:aq. NH₄OH 25% 9:1) in 21.4 min). The pure fractions of the separated racemic diastereomers were combined and the volatiles were removed under reduced pressure at 50° C. to give the title compounds:

Intermediate rac-P-1 eluting first from the column as a white solid (62 mg). LC-MS Method B-4: Rt=0.59 min; MS m/z [M+H]⁺=290.1.

Intermediate rac-P-2 eluting second from the column as a white solid (144 mg). LC-MS Method B-4: Rt=0.75 min; MS m/z [M+H]⁺=290.1.

Intermediate rac-P-3 and Intermediate rac-P-4:

### methyl 4-(2-fluoro-2-methyl-7-azaspiro[3.5]nonan-6-yl)benzoate

(separated racemic diastereomers; 2 mixtures of 2 stereoisomers each)

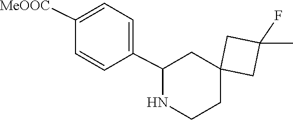 [SMILES: COC(=O)c1ccc(C2CC3(CCN2)CC(C)(F)C3)cc1]

TFA (1.5 mL) was added to a soln. of tert-butyl 2-fluoro-6-(4-(methoxycarbonyl)phenyl)-2-methyl-7-azaspiro[3.5]nonane-7-carboxylate (Intermediate ds-rac-B-5, 535 mg, 1.37 mmol) in DCM (4.5 mL). The RM was stirred at RT for 45 min, diluted with DCE, and the volatiles were removed under reduced pressure at 50° C. The residue was partitioned between DCM and aq. Na₂CO₃ soln. (10 wt %), the layers were separated and the aq. layer was washed with DCM (2×). The combined org. layers were dried over K₂CO₃, filtered, and the volatiles were removed under reduced pressure at 50° C. to give a yellow oil. The crude product was purified over silica gel (24 g) using an automated purification system (NP; Teledyne ISCO®; collected at 235/254 nm; flow 35 mL/min; eluent: heptane+25 to 80% (EtOAc:MeOH 95:5) in 25 min). The pure fractions of the separated racemic diastereomers were combined and the volatiles were removed under reduced pressure at 50° C. to give the title compounds:

Intermediate rac-P-3 eluting first from the column as a slightly yellow oil (119 mg). LC-MS Method B-4: Rt=0.82 min; MS m/z [M+H]⁺=292.2.

Intermediate rac-P-4 eluting second from the column as a white solid (227 mg). LC-MS Method B-4: Rt=0.86 min; MS m/z [M+H]⁺=292.2.

Intermediate rac-P-5:

### (RS)-methyl 4-(2,2-difluoro-7-azaspiro[3.5]nonan-6-yl)benzoate

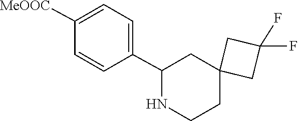 [SMILES: COC(=O)c1ccc(C2CC3(CCN2)CC(F)(F)C3)cc1]

TFA (3 mL) was added to a soln. of tert-butyl 2,2-difluoro-6-(4-(methoxycarbonyl)phenyl)-7-azaspiro[3.5]nonane-7-carboxylate (Intermediate rac-B-6, 917 mg, 2.32 mmol) in DCM (9 mL). The RM was stirred at RT for 30 min and then diluted with DCE. The volatiles were removed under reduced pressure. The residue was partioned between DCM and sat aq. NaHCO₃ soln. The separated aq. layer was extracted with DCM (3×), and the combined org. layers were dried over Na₂SO₄, filtered off, and concentrated under reduced pressure. The residue was dissolved in hexane and concentrated under reduced pressure at 50° C. to give the title compound (684 mg) as a colorless solid which was directly used without further purification. LC-MS Method B-1: Rt=0.47 min; MS m/z [M+H]⁺=296.3.

### Intermediate P-6:

### methyl (S)-4-(2,2-difluoro-7-azaspiro[3.5]nonan-6-yl)benzoate

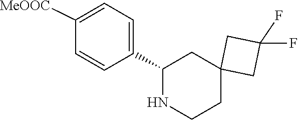 [SMILES: COC(=O)c1ccc([C@@H]2CC3(CCN2)CC(F)(F)C3)cc1]

TFA (70.7 mL) was added to a soln. of tert-butyl (S)-2,2-difluoro-6-(4-(methoxycarbonyl)phenyl)-7-azaspiro[3.5]nonane-7-carboxylate (Intermediate (−)-B-8, 20.8 g, 52.6 mmol) in DCM (212 mL). The RM was stirred at RT for 60 min. The volatiles were removed under reduced pressure at 50° C. The residue was dissolved in DCM and sat aq. NaHCO₃ soln. was added portion-wise to the well-stirred mixture.

The layers were separated and the aq. layer was washed with DCM (3×). The combined org. layers were dried over Na₂SO₄, filtered, and the volatiles were removed under reduced pressure at 500 to give a sticky, dark yellow solid that was purified over silica gel (220 g) using an automated purification system (NP; Teledyne ISCO®; collected at 234/254 nm; flow 150 mL/min; eluent: DCM+0 to 8% MeOH in 33.7 min). Pure fractions were combined, and the volatiles were removed under reduced pressure at 50° C., triturated with DCM/hexane and solvents were again removed under reduced pressure at 50° C. to give the title compound (12.79 g) as a light yellow solid. LC-MS Method B-1: Rt=0.45 min; MS m/z [M+H]⁺=296.3.

Intermediate rac-P-7 and Intermediate rac-P-8:

### methyl 4-(2-fluoro-7-azaspiro[3.5]nonan-6-yl)benzoate

(separated racemic diastereomers; 2 mixtures of 2 stereoisomers each)

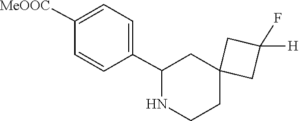 [SMILES: COC(=O)c1ccc(C2CC3(CCN2)CC(F)C3)cc1]

TFA (1 mL) was added to a soln. of tert-butyl 2-fluoro-6-(4-(methoxycarbonyl)phenyl)-7-azaspiro-[3.5]nonane-7-carboxylate (Intermediate ds-rac-B-9, 370 mg, 980 μmol) in DCM (3 mL). The RM was stirred at RT for 30 min, diluted with DCE and the volatiles were removed under reduced pressure at 50° C. The residue was partitioned between DCM and aq. Na₂CO₃ soln. (10 wt %). The layers were separated and the aq. layer was washed with DCM (2×). The combined org. layers were dried over K₂CO₃, filtered, and the volatiles were removed under reduced pressure at 50° C. to give a yellow oil that was purified over silica gel (24 g) using an automated purification system (NP; Teledyne ISCO®; collected at 235/254 nm; flow 35 mL/min; eluent: heptane+25-77.3% (EtOAc:MeOH/95:5) in 22.6 min). The pure fractions of the separated racemic diastereomers were combined, and the volatiles were removed under reduced pressure at 50° C. to give the title compounds:

Intermediate rac-P-7 eluting first from the column as colorless oil (112 mg). LC-MS Method B-1: Rt=0.34 min; MS m/z [M+H]⁺=278.2.

Intermediate rac-P-8 eluting second from the column as slightly yellow oil (139 mg). LC-MS Method B-1: Rt=0.44 min; MS m/z [M+H]⁺=278.3.

Intermediate rac-P-9:

### (RS)-methyl 4-(2-oxa-7-azaspiro[3.5]nonan-6-yl)benzoate

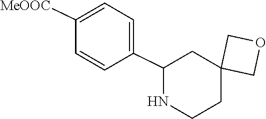 [SMILES: COC(=O)c1ccc(C2CC3(CCN2)COC3)cc1]

TFA (0.6 mL) was added to a soln. of tert-butyl 6-(4-(methoxycarbonyl)phenyl)-2-oxa-7-azaspiro[3.5]nonane-7-carboxylate (Intermediate rac-B-10, 226 mg, 0.625 mmol) in DCM (1.8 mL) and the RM was stirred for 50 min. The RM was diluted with DCE and evaporated to dryness. The residue was partitioned between DCM and sat. aq. NaHCO₃ soln.; the layers were separated and the aq. layer was washed with DCM (3×). The combined org. layers were dried over Na₂SO₄, filtered, and the volatiles were removed under reduced pressure at 50° C. to give an amber oil that was dissolved and purified by preparative TLC (MERCK 1.05744.0001, PLC silica gel 60 F254, 0.5 mm; eluent: DCM/(MeOH:NH₄OH 25% 9:1) 88/12. The main bands were scratched off the plates. The silica gel/compound mixture was suspended in DCM/(MeOH:NH₄OH 25% 9:1) 85/15, treated with ultrasound and filtered. The residue was washed with DCM/(MeOH:NH₄OH 25% 9:1) 85/15. The volatiles of the filtrate were removed under reduced pressure at 50° C. to give the title compound as a white fluffy solid (153 mg). LC-MS Method B-5: Rt=0.47 min; MS m/z [M+H]⁺=262.1.

### Intermediate P-10:

### methyl 4-((5S,7R)-2,2-difluoro-8-azaspiro[4.5]decan-7-yl)benzoate or methyl 4-((5R,7R)-2,2-difluoro-8-azaspiro[4.5]decan-7-yl)benzoate

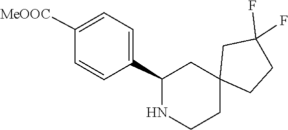 [SMILES: COC(=O)c1ccc([C@H]2CC3(CCN2)CCC(F)(F)C3)cc1]

Intermediate P-10 as its HCl salt was prepared according to the method described for Intermediate P-11 herein below, using Intermediate (+)-B-14(+). LC-MS Method C-1: Rt=1.02 min; MS m/z [M+H]⁺=310.1.

### Intermediate P-11:

### methyl 4-((5S,7S)-2,2-difluoro-8-azaspiro[4.5]decan-7-yl)benzoate or methyl 4-((5R,7S)-2,2-difluoro-8-azaspiro[4.5]decan-7-yl)benzoate

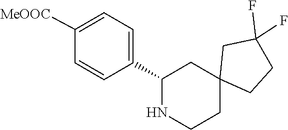 [SMILES: COC(=O)c1ccc([C@@H]2CC3(CCN2)CCC(F)(F)C3)cc1]

To Intermediate (−)-B-15(+) (65 mg, 0.159 mmo) in 1,4-dioxane (0.5 mL) was added slowly a HCl soln. (4M in 1,4-dioxane, 0.79 mL, 3.18 mmol). The mixture was stirred ˜2 to 3 h and then concentrated under reduced pressure. The residue was kept under reduced pressure overnight providing the crude title compound as its HCl salt as a white solid which was directly used in next step without further purification. LC-MS Method C-1: Rt=1.03 min; MS m/z [M+H]⁺=310.1.

### Intermediate P-12:

### methyl 4-((5S,7R)-2,2-difluoro-8-azaspiro[4.5]decan-7-yl)benzoate or methyl 4-((5R,7R)-2,2-difluoro-8-azaspiro[4.5]decan-7-yl)benzoate

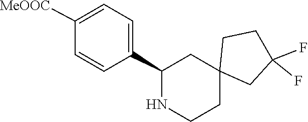 [SMILES: COC(=O)c1ccc([C@H]2CC3(CCN2)CCC(F)(F)C3)cc1]

Intermediate P-12 as its HCl salt was prepared according to the method described for Intermediate P-11 herein above, using Intermediate (+)-B-16(−). LC-MS Method C-1: Rt=1.03 min; MS m/z [M+H]⁺=310.1.

### Intermediate P-13:

### methyl 4-((5S,7S)-2,2-difluoro-8-azaspiro[4.5]decan-7-yl)benzoate or methyl 4-((5R,7S)-2,2-difluoro-8-azaspiro[4.5]decan-7-yl)benzoate

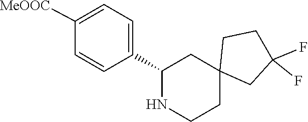 [SMILES: COC(=O)c1ccc([C@@H]2CC3(CCN2)CCC(F)(F)C3)cc1]

Intermediate P-13 as its HCl salt was prepared according to the method described for Intermediate P-11 herein above, using Intermediate (−)-B-17(−). LC-MS Method C-1: Rt=1.02 min; MS m/z [M+H]⁺=310.1.

Intermediate rac-P-14:

### (RS)-3-(4-(2,2-difluoro-7-azaspiro[3.5]nonan-6-yl)phenyl)oxetan-3-ol

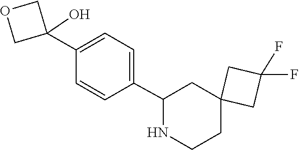 [SMILES: OC1(c2ccc(C3CC4(CCN3)CC(F)(F)C4)cc2)COC1]

TFA (1.50 mL) was added to a soln. of tert-butyl 2,2-difluoro-6-(4-(3-hydroxyoxetan-3-yl)phenyl)-7-azaspiro[3.5]nonane-7-carboxylate (Intermediate rac-B-18, 370.0 mg, 903.6 μmol) in DCM (6.0 mL). The RM was stirred at RT for 15 min. The RM was poured into a well-stirred aq. Na₂CO₃ soln. (10 wt %), the layers were separated, and the aq. layer was washed with DCM (3×). The combined org. layers were dried over K₂CO₃, filtered, and the volatiles were removed under reduced pressure at 50° C. to give an off-white solid that was purified over silica gel (12 g) using an automated purification system (NP; Teledyne ISCO®; collected at 218/262 nm+ELSD detector; flow 30 mL/min; eluent: DCM+0 to 9.1 (EtOH:NH₄OH 25% 9:1) in 18.1 min. Pure fractions were combined, and the volatiles were removed under reduced pressure at 50° C. to give the title compound as a white solid (235 mg). LC-MS Method B-1: Rt=0.21 min; MS m/z [M+H]⁺=310.3.

Intermediate rac-P-15:

### (RS)-2-(4-(2,2-difluoro-7-azaspiro[3.5]nonan-6-yl)phenyl)propan-2-ol

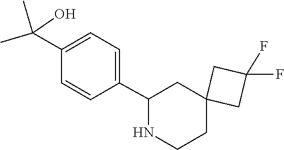 [SMILES: CC(C)(O)c1ccc(C2CC3(CCN2)CC(F)(F)C3)cc1]

tert-Butyl 2,2-difluoro-6-(4-(2-hydroxypropan-2-yl)phenyl)-7-azaspiro[3.5]nonane-7-carboxylate (Intermediate rac-B-19, 256.0 mg, 647.3 μmol) was dissolved in 4M HCl in dioxane (3.0 mL). The RM was stirred at RT for 15 min. The RM was poured into a well-stirred emulsion of water and DCM. Subsequently the mixture was carefully basified with solid Na₂CO₃ and layers were separated. The aq. layer was washed with DCM (3×). The combined org. layers were dried over K₂CO₃, filtered, and the volatiles were removed under reduced pressure at 50° C. to give a yellow oil that was purified over silica gel (12 g) using an automated purification system (NP; Teledyne ISCO®; collected at 220/254 nm; flow 30 mL/min; eluent: DCM+0 to 9.9% (EtOH:NH₄OH 25% 9:1) in 19.7 min). Pure fractions were combined, and the volatiles were removed under reduced pressure at 50° C. to give the title compound as a colorless oil (149 mg). LC-MS Method B-1: Rt=0.39 min; MS m/z [M+H]⁺=296.2.

Intermediate rac-P-16:

### (RS)-6-(4-(1H-pyrazol-5-yl)phenyl)-2,2-difluoro-7-azaspiro[3.5]nonane

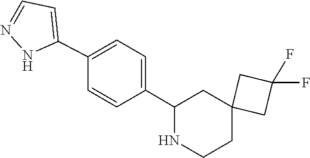 [SMILES: FC1(F)CC2(CCNC(c3ccc(-c4ccn[nH]4)cc3)C2)C1]

TFA (1.0 mL) was added to a soln. of tert-butyl 6-(4-(1H-pyrazol-5-yl)phenyl)-2,2-difluoro-7-azaspiro[3.5]nonane-7-carboxylate (Intermediate rac-B-20, 249.0 mg, ˜95 wt %, 586.3 μmol) in DCM (3.0 mL). The RM was stirred at RT for 15 min. and then poured into a well-stirred aq. Na₂CO₃ soln. (10 wt %). The layers were separated and the aq. layer was washed with DCM (3×). The combined org. layers were dried over K₂CO₃, filtered, and the volatiles were removed under reduced pressure at 50° C. to give a colorless oil that was purified over silica gel (12 g) using an automated purification system (NP; Teledyne ISCO®; collected at 254/280 nm; flow 30 mL/min; eluent: DCM+0 to 12.3% (EtOH:NH₄OH 25% 9:1) in 17.3 min). Pure fractions were combined, and the volatiles were removed under reduced pressure at 50° C. to give the title compound as a white foam (163 mg) LC-MS Method B-1: Rt=0.42 min; MS m/z [M+H]⁺=304.2.

Intermediate rac-P-17:

### (RS)-6-(4-(1H-pyrazol-1-yl)phenyl)-2,2-difluoro-7-azaspiro[3.5]nonane

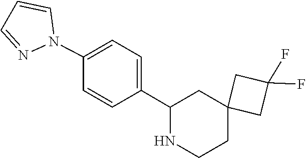 [SMILES: FC1(F)CC2(CCNC(c3ccc(-n4cccn4)cc3)C2)C1]

TFA (1.0 mL) was added to a soln. of tert-butyl 6-(4-(1H-pyrazol-1-yl)phenyl)-2,2-difluoro-7-azaspiro[3.5]nonane-7-carboxylate (Intermediate rac-B-21, 226.0 mg, 560.1 μmol) in DCM (3.0 mL). The RM was stirred at RT for 30 min, then poured into a well-stirred aq. Na₂CO₃ soln. (10 wt %) The layers were separated and the aq. layer was washed with DCM (3×). The combined org. layers were dried over K₂CO₃, filtered, and the volatiles were removed under reduced pressure at 50° C. to give a colorless oil that was purified over silica gel (12 g) using an automated purification system (NP; Teledyne ISCO®; collected at 256/280 nm; flow 30 mL/min; eluent: DCM+0 to 10% (EtOH:NH₄OH 25% 9:1) in 10 min). Pure fractions were combined, and the volatiles were removed under reduced pressure at 50° C. to give the title compound as a white solid (159 mg). LC-MS Method B-1: Rt=0.46 min; MS m/z [M+H]⁺=304.3.

Intermediate rac-P-18:

### (RS)-4-(2,2-difluoro-7-azaspiro[3.5]nonan-6-yl)benzonitrile

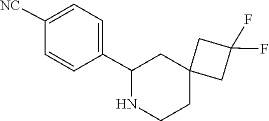 [SMILES: N#Cc1ccc(C2CC3(CCN2)CC(F)(F)C3)cc1]

TFA (1.0 mL) was added to a soln. of tert-butyl 6-(4-cyanophenyl)-2,2-difluoro-7-azaspiro[3.5]nonane-7-carboxylate (Intermediate rac-B-22, 276.0 mg, 95 wt %, 723.5 μmol) in DCM (3.0 mL). The RM was stirred at RT for 30 min. The RM was poured into a well-stirred aq. Na₂CO₃ soln. (10 wt %). Subsequently the layers were separated and the aq. layer was washed with DCM (3×). The combined org. layers were dried over K₂CO₃, filtered, and the volatiles were removed under reduced pressure at 50° C., triturated with DCM/hexane, the solvents were again removed under reduced pressure at 50° C. to give the title compound as a white solid (188 mg). LC-MS Method B-1: Rt=0.27 min; MS m/z [M+H]⁺=263.1.

Intermediate rac-P-19:

### (RS)-2,2-difluoro-6-phenyl-7-azaspiro[3.5]nonane

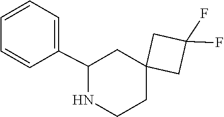 [SMILES: FC1(F)CC2(CCNC(c3ccccc3)C2)C1]

TFA (0.75 mL) was added to a soln. of tert-butyl 2,2-difluoro-6-phenyl-7-azaspiro[3.5]nonane-7-carboxylate (Intermediate rac-B-23, 242.0 mg, 95 wt %, 681.4 μmol) in DCM (2.25 mL). The RM was stirred at RT for 30 min, then poured into a well-stirred aq. Na₂CO₃ soln. (10 wt %). Subsequently the layers were separated and the aq. layer was washed with DCM (3×). The combined org. layers were dried over K₂CO₃, filtered, and the volatiles were removed under reduced pressure at 50° C. to give a colorless oil that was purified over silica gel (12 g) using an automated purification system (NP; Teledyne ISCO®; collected at 215/254 nm, but all fractions collected due to low UV activity; flow 30 mL/min; eluent: DCM+0 to 6.6% (EtOH:NH₄OH 25% 9:1) in 9.9 min). Pure fractions were combined, and the volatiles were removed under reduced pressure at 50° C., treated with hexane and the solvents were again removed under reduced pressure at 50° C. to give the title compound as light yellow liquid (137 mg). LC-MS Method B-1: Rt=0.28 min; MS m/z [M+H]⁺=238.3.

Intermediate rac-P-20:

### (RS)-2,2-difluoro-6-(1-methyl-1H-pyrazol-4-yl)-7-azaspiro[3.5]nonane

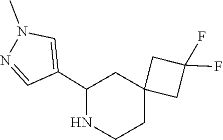 [SMILES: Cn1cc(C2CC3(CCN2)CC(F)(F)C3)cn1]

TFA (0.50 mL) was added to a soln. of tert-butyl 2,2-difluoro-6-(1-methyl-1H-pyrazol-4-yl)-7-azaspiro[3.5]nonane-7-carboxylate (Intermediate rac-B-24, 127.0 mg, 372.0 μmol) in DCM (1.5 mL). The RM was stirred at RT for 40 min. The RM was poured into a well-stirred aq. Na₂CO₃ soln. (10 wt %). Subsequently the layers were separated, and the aq. layer was washed with DCM (3×). The combined org. layers were dried over K₂CO₃, filtered, and the volatiles were removed under reduced pressure at 50° C. to give the title compound as a light yellow oil (90 mg) that was used in the next step without further purification. ¹H NMR (400 MHz, DMSO-d₆) δ [ppm]7.51 (s, 1H), 7.30 (s, 1H), 3.76 (s, 3H), 3.44 (dd, J=11.4, 2.5 Hz, 1H), 2.88 (m, 1H), 2.61-2.51 (m, 1H), 2.48-2.36 (m, 2H), 2.30 (m, 2H), 2.12-2.02 (br s, 1H), 1.78-1.72 (m, 1H), 1.58-1.41 (m, 3H).

The following Intermediates rac-P 21 to rac P-26 were synthesized from Intermediate O-3 and the appropriate aryl-bromides and by applying the synthetic sequence of coupling, catalytic hydrogenation, and Boc-deprotection utilizing similar methods described for the intermediates above.

Intermediate rac-P-21:

### (RS)-6-(1-(difluoromethyl)-1H-pyrazol-4-yl)-2,2-difluoro-7-azaspiro[3.5]nonane

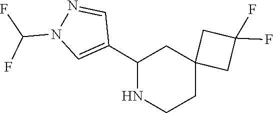 [SMILES: FC(F)n1cc(C2CC3(CCN2)CC(F)(F)C3)cn1]

LC-MS Method B-7: Rt=1.26 min; MS m/z [M+H]⁺=278.1.

Intermediate rac-P-22:

### (RS)-2,2-difluoro-6-(1-(2-methoxyethyl)-1H-pyrazol-4-yl)-7-azaspiro[3.5]nonane

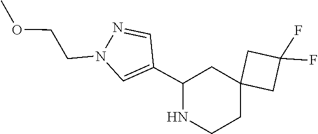 [SMILES: COCCn1cc(C2CC3(CCN2)CC(F)(F)C3)cn1]

¹H NMR (400 MHz, DMSO-d₆) δ [ppm]7.54 (s, 1H), 7.34 (s, 1H), 4.17 (t, J=5.4 Hz, 2H), 3.64 (t, J=5.4 Hz, 2H), 3.45 (d, J=11.3 Hz, 1H), 3.21 (s, 3H), 2.92-2.85 (m, 1H), 2.61-2.51 (m, 1H), 2.49-2.36 (m, 2H), 2.37-2.26 (m, 2H), 2.06 (s, 1H), 1.76 (d, J=12.6 Hz, 1H), 1.58-1.41 (m, 3H).

Intermediate rac-P-23:

### ethyl (RS)-2-(4-(2,2-difluoro-7-azaspiro[3.5]nonan-6-yl)-1H-pyrazol-1-yl)acetate

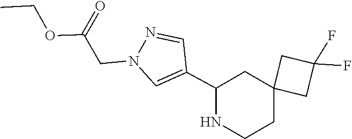 [SMILES: CCOC(=O)Cn1cc(C2CC3(CCN2)CC(F)(F)C3)cn1]

¹H NMR (400 MHz, DMSO-d₆) δ [ppm]7.59 (s, 1H), 7.40 (s, 1H), 4.98 (s, 2H), 4.17-4.09 (m, 2H), 3.53 (dd, J=11.4, 2.6 Hz, 1H), 2.99-2.88 (m, 2H), 2.64-2.56 (m, 1H), 2.48-2.38 (m, 2H), 2.37-2.29 (m, 2H), 1.83-1.76 (m, 1H), 1.58-1.51 (m, 2H), 1.20 (t, J=7.1 Hz, 4H).

Intermediate rac-P-24:

### (RS)-6-(4-(difluoromethyl)phenyl)-2,2-difluoro-7-azaspiro[3.5]nonane

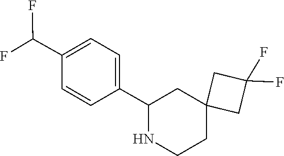 [SMILES: FC(F)c1ccc(C2CC3(CCN2)CC(F)(F)C3)cc1]

LC-MS Method B-1: Rt=0.43 min; MS m/z [M+H]⁺=288.3.

Intermediate rac-P-25:

### methyl (RS)-5-(2,2-difluoro-7-azaspiro[3.5]nonan-6-yl)picolinate

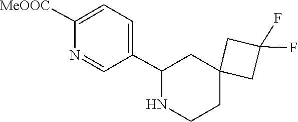 [SMILES: COC(=O)c1ccc(C2CC3(CCN2)CC(F)(F)C3)cn1]

LC-MS Method B-6: Rt=0.24 min; MS m/z [M+H]⁺=297.0.

Intermediate rac-P-26:

### (RS)-5-(2,2-difluoro-7-azaspiro[3.5]nonan-6-yl)picolinonitrile

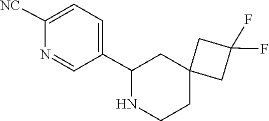 [SMILES: N#Cc1ccc(C2CC3(CCN2)CC(F)(F)C3)cn1]

LC-MS Method B-6: Rt=0.18 min; MS m/z [M+H]⁺=264.3.

### Intermediate P-27:

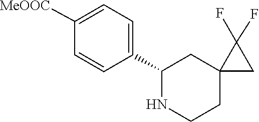 [SMILES: COC(=O)c1ccc([C@@H]2CC3(CCN2)CC3(F)F)cc1]

To a solution of benzyl (5S)-1,1-difluoro-5-(4-(methoxycarbonyl)phenyl)-6-azaspiro[2.5]octane-6-carboxylate (Intermediate B-12/B-13, 100 mg, 0.24 mmol) in MeOH (3 mL) and under N₂ atmosphere was added Pd/C (40 mg, 10 wt %). The solution was stirred for 3 h under H₂ atmosphere (balloon). The RM was purged with N₂, filtered through a pad of Celite® and rinsed with MeOH (20 mL). The filtrate was concentrated under reduced pressure to give the title compound (70 mg). LC-MS Method A-1: Rt=0.31 min; MS m/z [M+H]⁺=282.1.

Intermediate rac-A-1, Intermediate rac-A-2 and Intermediate A-15, Intermediate A-16:

### tert-butyl 4-((2-hydroxy-6-(4-(methoxycarbonyl)phenyl)-2-methyl-7-azaspiro[3.5]nonan-7-yl)methyl)-5-methoxy-7-methyl-1H-indole-1-carboxylate

(separated racemic diastereomers; mixtures of 2 stereoisomers each, one of them separated into 2 single stereoisomers)

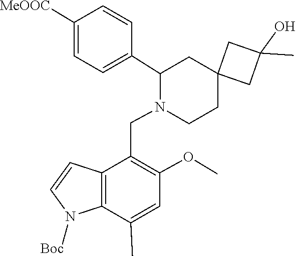 [SMILES: COC(=O)c1ccc(C2CC3(CCN2Cc2c(OC)cc(C)c4c2ccn4C(=O)OC(C)(C)C)CC(C)(O)C3)cc1]

Intermediate rac-A-2: Intermediate rac-P-2 (142 mg, 491 μmol) and tert-butyl 4-formyl-5-methoxy-7-methyl-1H-indole-1-carboxylate (Intermediate I-1, 177 mg, 613 μmol) were dissolved in 3.5 mL DCE. NaBH(OAc)₃ (182 mg, 859 μmol) was added in 2 portions over the course of 60 min. The RM was stirred at RT for a total of 36 h with addition of another 2×0.5 eq (52 mg each) NaBH(OAc)₃ every 8-14 h, then partitioned between EtOAc and sat. aq. NaHCO₃ soln.; the layers were separated and washed with sat. aq. NaHCO₃ soln., brine, and EtOAc. The combined org. layers were dried over MgSO₄, filtered, and the volatiles were removed under reduced pressure at 50° C. to give a colorless oil. The oil was re-dissolved in 1.5 mL DCE and the RM was then again treated with NaBH(OAc)₃ (182 mg, 859 μmol). After 7 h, 0.5 eq (52 mg) NaBH(OAc)₃ were added and the RM was stirred for additional 24 h, diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a colorless oil. The crude product was purified over silica gel (12 g) using an automated purification system (NP; Teledyne ISCO®; collected at 242/254 nm; flow 30 mL/min; eluent: heptane+10 to 35.9% EtOAc in 18.6 min). Pure fractions were combined, and the volatiles were removed under reduced pressure at 50° C. to give to title compound as a colorless oil that solidified upon standing (171 mg). LC-MS Method B-1: Rt=1.03 min; MS m/z [M+H]⁺=563.5.

Enantiomer separation and analytics according to Method SFC-7, using 122 mg of the racemic Intermediate rac-A-2, followed by removal of solvents under reduced pressure at 50° C., trituration with hexane and drying (under reduced pressure, 50° C.):

Peak 1: (99.5% ee), Intermediate A-15, 48 mg, colorless oil. LC-MS Method B-1: Rt=0.98 min. MS m/z [M+H]⁺=535.2. Analytical chiral HPLC (Method SFC-7): Rt=1.42 min.

Peak 2: (99.0% ee), Intermediate A-16, 47 mg, colorless oil. LC-MS Method B-1: Rt=0.95 min. MS m/z [M+H]⁺=535.4. Analytical chiral HPLC (Method SFC-7): Rt=2.01 min.

Intermediate rac-A-1 was prepared according to the method described for Intermediate rac-A-2 herein above, using Intermediate rac-P-1(60 mg, 210 μmol) and tert-butyl 4-formyl-5-methoxy-7-methyl-1H-indole-1-carboxylate (Intermediate I-1, 75 mg, 260 μmol) providing racemic Intermediate rac-A-1 (62 mg) as s colorless oil. LC-MS Method B-1: Rt=0.90 min; MS m/z [M+H]⁺=563.8. Racemate, not separated.

### Intermediate A-3, Intermediate A-4 and Intermediate A-5, Intermediate A-6:

### tert-butyl 4-((2-fluoro-6-(4-(methoxycarbonyl)phenyl)-2-methyl-7-azaspiro[3.5]nonan-7-yl)methyl)-5-methoxy-7-methyl-1H-indole-1-carboxylate

(separated racemic diastereomers, mixture of 2 stereoisomers each, both separated into two enantiomers each)

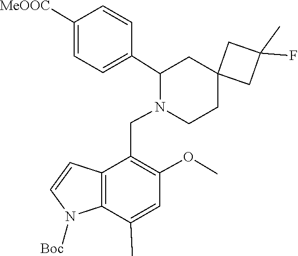 [SMILES: COC(=O)c1ccc(C2CC3(CCN2Cc2c(OC)cc(C)c4c2ccn4C(=O)OC(C)(C)C)CC(C)(F)C3)cc1]

A mixture of Intermediate rac-P-3 (105 mg, 360 μmol) and tert-butyl 4-formyl-5-methoxy-7-methyl-1H-indole-1-carboxylate (Intermediate I-1, 130 mg, 450 μmol) were dissolved in DCE. NaBH(OAc)₃ (134 mg, 631 μmol) was added in 2 portions over the course of 40 min. The RM was stirred for 5 d, 4×0.5 eq (38 mg each) NaBH(OAc)₃ were subsequently added. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed, under reduced pressure at 50° C. to give a slightly yellow oil. In order to remove unreacted aldehyde, the crude product was dissolved in 3 mL EtOH and NaBH₄ (13.6 mg, 360 μmol) was added and the RM was stirred for 15 min. The RM was partitioned between MTBE, water, and brine. The layers were separated and washed with brine and MTBE. The combined org. layers were dried over MgSO₄, filtered, and the volatiles were removed under reduced pressure at 50° C. to give a colorless oil.

The crude product was purified over silica gel (12 g) using an automated purification system (NP; Teledyne ISCO®; collected at 243/254 nm; flow 30 mL/min; eluent: heptane+2-11.7% EtOAc in 11.8 min). Pure fractions were combined, and the volatiles were removed under reduced pressure at 50° C. to give the title compound (single diastereoisomer, racemic) as a white foam (167 mg). LC-MS Method B-1: Rt=1.12 min; MS m/z [M+H]⁺=565.8.

Enantiomer separation and analytics according to Method SFC-3, followed by removal of solvents under reduced pressure at 50° C., trituration with hexane and drying (under reduced pressure, 50° C.):

Peak 1: (99.5% ee), Intermediate A-3, 52.7 mg, white foam. LC-MS Method B-1: Rt=1.23 min. MS m/z [M+H]⁺=565.4. Analytical chiral HPLC (Method SFC-3): Rt=1.29 min.

Peak 2: (99.5% ee), Intermediate A-4, 52.9 mg, white foam. LC-MS Method B-1: Rt=1.12 min. MS m/z [M+H]⁺=565.8. Analytical chiral HPLC (Method SFC-3): Rt=1.29 min.

Intermediate A-5/Intermediate A-6 were prepared according the method as described for Intermediate A-3/Intermediate A-4 above from Intermediate rac-P-4 (172 mg, 590 μmol) and tert-butyl 4-formyl-5-methoxy-7-methyl-1H-indole-1-carboxylate (Intermediate I-1, 213 mg, 738 μmol) yielding the title compound as a white foam (295 mg). LC-MS Method B-1: Rt=1.16 min; MS m/z [M+H]⁺=565.3.

Enantiomer separation and analytics according to Method SFC-4, followed by removal of solvents under reduced pressure at 50° C., trituration with hexane and drying (under reduced pressure, 50° C.):

Peak 1: (99.5% ee), Intermediate A-5, 112 mg, white foam. LC-MS Method B-1: Rt=1.14 min. MS m/z [M+H]⁺=565.5. Analytical chiral HPLC (Method SFC-4): Rt=1.46 min.

Peak 2: (99.5% ee), Intermediate A-6, 116 mg, white foam. LC-MS Method B-1: Rt=1.18 min. MS m/z [M+H]⁺=565.3. Analytical chiral HPLC (Method SFC-4): Rt=2.29 min.

Intermediate rac-A-78, Intermediate A-7, and Intermediate A-8:

### tert-butyl 4-((2,2-difluoro-6-(4-(methoxycarbonyl)phenyl)-7-azaspiro[3.5]nonan-7-yl)methyl)-5-methoxy-7-methyl-1H-indole-1-carboxylate, tert-butyl (S)-4-((2,2-difluoro-6-(4-(methoxycarbonyl)phenyl)-7-azaspiro[3.5]nonan-7-yl)methyl)-5-methoxy-7-methyl-1H-indole-1-carboxylate, and tert-butyl (R)-4-((2,2-difluoro-6-(4-(methoxycarbonyl)phenyl)-7-azaspiro[3.5]nonan-7-yl)methyl)-5-methoxy-7-methyl-1H-indole-1-carboxylate

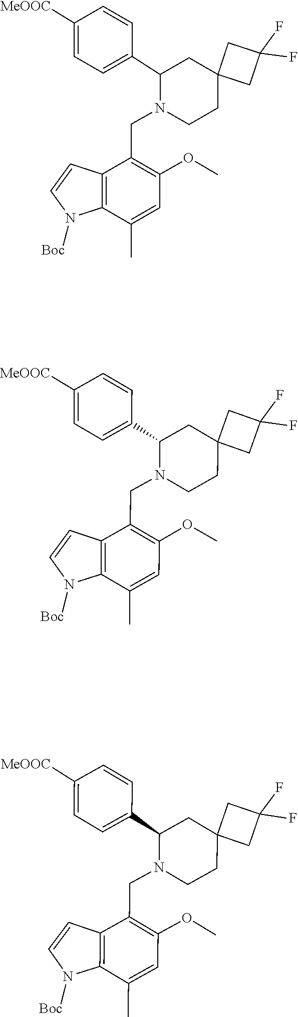 [SMILES: COC(=O)c1ccc(C2CC3(CCN2Cc2c(OC)cc(C)c4c2ccn4C(=O)OC(C)(C)C)CC(F)(F)C3)cc1 ; COC(=O)c1ccc([C@@H]2CC3(CCN2Cc2c(OC)cc(C)c4c2ccn4C(=O)OC(C)(C)C)CC(F)(F)C3)cc1 ; COC(=O)c1ccc([C@H]2CC3(CCN2Cc2c(OC)cc(C)c4c2ccn4C(=O)OC(C)(C)C)CC(F)(F)C3)cc1]

Racemic tert-butyl 4-((2,2-difluoro-6-(4-(methoxycarbonyl)phenyl)-7-azaspiro[3.5]nonan-7-yl)methyl)-5-methoxy-7-methyl-1H-indole-1-carboxylate was prepared according the method as described for Intermediate A-21 below, methyl 4-(2,2-difluoro-7-azaspiro[3.5]nonan-6-yl)benzoate (Intermediate rac-P-5, 667 mg, 2.26 mmol) and tert-butyl 4-formyl-5-methoxy-7-methyl-1H-indole-1-carboxylate (Intermediate I-1, 817 mg, 2.82 mmol) yielding the racemic mixture of the title compound as a white foam (1.19 g). LC-MS Method B-1: Rt=1.18 min; MS m/z [M+H]⁺=569.4.

Enantiomer separation and analytics according to Method SFC-15, using 1.16 g racemic tert-butyl 4-((2,2-difluoro-6-(4-(methoxycarbonyl)phenyl)-7-azaspiro[3.5]nonan-7-yl)methyl)-5-methoxy-7-methyl-1H-indole-1-carboxylate, followed by removal of solvents under reduced pressure at 50° C., trituration with hexane and drying under reduced pressure (50° C.):

Peak 1: ((+)-isomer, 99.5% ee), Intermediate A-7, 541 mg, white foam. LC-MS Method B-1: Rt=1.20 min. MS m/z [M+H]⁺=569.6; analytical chiral HPLC (Method SFC-15): Rt=1.19 min. Specific rotation Method OR2: [α]²⁵ @589 nm=+38.4° (in hexane).

Peak 2: ((−)-isomer, 99.5% ee), Intermediate A-8, 554 mg, white foam. LC-MS Method B-1: Rt=1.21 min. MS m/z [M+H]⁺=569.6. analytical chiral HPLC (Method SFC-15): Rt=2.24 min. Specific rotation Method OR2: [α]²⁵ @589 nm=−41.2° (in hexane).

Alternative preparation of tert-butyl (S)-4-((2,2-difluoro-6-(4-(methoxycarbonyl)phenyl)-7-azaspiro[3.5]nonan-7-yl)methyl)-5-methoxy-7-methyl-1H-indole-1-carboxylate (Intermediate A-7):

Methyl (S)-4-(2,2-difluoro-7-azaspiro[3.5]nonan-6-yl)benzoate (Intermediate P-6, 4.85 g, 16.4 mmol) and tert-butyl 4-formyl-5-methoxy-7-methyl-1H-indole-1-carboxylate (Intermediate I-1, 5.94 g, 20.5 mmol) were dissolved in DCE (73 mL). NaBH(OAc)₃ (6.09 g, 28.7 mmol) was added in 4 portions over the course of 4 hours. The RM was stirred for 1.5 h, and additional NaBH(OAc)₃ (870 mg) was added. The RM was stirred overnight, but as the reaction was still incomplete, every 2 hours additional NaBH(OAc)₃ were added (total: 5×870 mg). The RM was stirred overnight. The RM was carefully poured into a well-stirred sat. aq. NaHCO₃ soln.; after the evolution of CO₂ had ceased, the mixture was transferred to a separation funnel. The layers were separated and the aq. layer was washed with DCM (2×). The combined org. layers were dried over MgSO₄, filtered, and the volatiles were removed under reduced pressure at 50° C. to give a yellow oil. The material was re-dissolved in 73 mL DCE and every 2 h NaBH(OAc)₃ was added (total: 4×870 mg). The RM was eventually stirred overnight. The RM was carefully poured into a vigorously stirred sat. aq. NaHCO₃ soln; after the evolution of CO₂ had ceased, MTBE was added and the layers were separated. The layers were washed with sat. aq. NaHCO₃ soln., brine and MTBE. The combined org. layers were dried over MgSO₄, filtered, and the volatiles were removed under reduced pressure at 50° C. to give a slightly yellow oil that was dissolved in EtOH (50 mL) and NaBH₄ (311 mg, 8.21 mmol) was added to reduce remaining aldehyde. The RM was stirred at ambient temperature for 20 min, then partitioned between MTBE, water, and brine. The layers were separated and washed with brine and MTBE. The combined org. layers were dried over MgSO₄, filtered, and the volatiles were removed under reduced pressure at 50° C. to give a slightly amber foam (containing pure crude and minor trans-esterification to the ethyl ester). The crude was dissolved in 100 mL MTBE and 10 g SiliaMetS Thiol (PN R51030B by Silicycle Inc.; 40-63 μm, 1.40 mmol/g) were added. The vigorously stirred mixture was stirred at 50° C. for 4 h. The RM was then cooled to RT and filtered. The scavenger was washed with MTBE (2×), and the volatiles of the filtrate were removed under reduced pressure at 50° C. to give a light yellow foam. The crude product was purified over silica gel (330 g) using an automated purification system (NP; Teledyne ISCO®; collected at 254/275 nm; flow 200 mL/min, eluent: heptane+2 to 42.7% EtOAc using a step-wise gradient in 28.3 min). The pure fractions were combined, and the volatiles were removed under reduced pressure at 50° C. to give the title compound as a white foam (7.85 g) partially contaminated with the corresponding ethyl ester. LC-MS Method B-1: Rt=1.23 min; MS m/z [M+H]⁺=569.4. Corresponding ethyl ester: Rt=1.32 min [M+H]⁺583.4.

### Intermediate A-9, Intermediate A-10 and Intermediate A-11, Intermediate A-12:

### tert-butyl 4-((2-fluoro-6-(4-(methoxycarbonyl)phenyl)-7-azaspiro[3.5]nonan-7-yl)methyl)-5-methoxy-7-methyl-1H-indole-1-carboxylate

(separated racemic diastereomers, then separated into single stereoisomers)

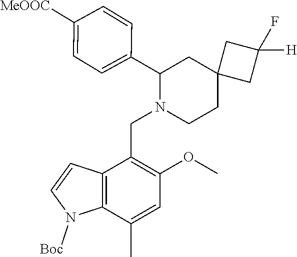 [SMILES: COC(=O)c1ccc(C2CC3(CCN2Cc2c(OC)cc(C)c4c2ccn4C(=O)OC(C)(C)C)CC(F)C3)cc1]

Intermediate rac-P-8 (137 mg, 494 μmol) and tert-butyl 4-formyl-5-methoxy-7-methyl-1H-indole-1-carboxylate (Intermediate I-1, 179 mg, 617 μmol) were dissolved in DCE. NaBH(OAc)₃ (183 mg, 864 μmol) was added in 3 portions over the course of 180 min. The RM was stirred for a total of 58 h with addition of another 4×0.5 eq (52 mg each) NaBH(OAc)₃ every 8 to 14 h. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow oil. To remove excess aldehyde, the crude product was dissolved in 3 mL EtOH and NaBH₄ (18.7 mg, 494 μmol) was added. The RM was stirred for 15 min at RT, then the RM was partitioned between MTBE, water, and brine. The layers were separated and washed with brine and MTBE. The combined org. layers were dried over MgSO₄, filtered, and the volatiles were removed under reduced pressure at 50° C. to give a colorless oil. The crude product was purified over silica gel (12 g) using an automated purification system (NP; Teledyne ISCO®; collected at 240/254 nm; flow 30 mL/min; eluent: heptane+2 to 12.9% EtOAc in 12.5 min). Pure fractions were combined, and the volatiles were removed under reduced pressure at 50° C. to give the title compound as a white foam (217 mg). LC-MS Method B-1: Rt=1.08 min; MS m/z [M+H]⁺=551.5.

Enantiomer separation and analytics according to method Method SFC-12, using 215 mg of the racemic mixture, followed by removal of solvents under reduced pressure at 50° C., trituration with hexane and drying (under reduced pressure, 50° C.):

Peak 1: (99.5% ee), Intermediate A-9, 64 mg, white foam. LC-MS Method B-1: Rt=1.05 min. MS m/z [M+H]⁺=551.4. Analytical chiral HPLC (Method SFC-12): Rt=1.65 min.

Peak 2: (99.5% ee), Intermediate A-10, 64 mg, white foam. LC-MS Method B-1: Rt=1.10 min. MS m/z [M+H]⁺=551.5. Analytical chiral HPLC (Method SFC-12): Rt=2.37 min.

Intermediate rac-P-7 (110 mg, 397 μmol) and tert-butyl 4-formyl-5-methoxy-7-methyl-1H-indole-1-carboxylate (Intermediate I-1, 143 mg, 496 μmol) were dissolved in DCE. NaBH(OAc)₃ (147 mg, 694 μmol) was added in 3 portions over the course of 180 min. The RM was stirred for a total of 58 h with addition of another 4×0.5 eq (42 mg each) NaBH(OAc)₃ every 8 to 14 h. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow solid. To remove unreacted Intermediate I-1, the oil was dissolved in 2 mL EtOH and NaBH₄ (15.0 mg, 397 μmol) was added and the RM stirred at RT for 10 min. The RM was partitioned between MTBE, water and some brine. The layers were separated and washed with brine and MTBE. The combined org. layers were dried over MgSO₄, filtered, and the volatiles were removed under reduced pressure at 50° C. to give a colorless oil. The crude product was purified over silica gel (12 g) using an automated purification system (NP; Teledyne ISCO®; collected at 240/254 nm; flow 30 mL/min; eluent: heptane+1 to 13.5% EtOAc in 13.2 min). Pure fractions were combined, and the volatiles were removed under reduced pressure at 50° C. to give the title compound as a white foam (172 mg). LC-MS Method B-1: Rt=1.14 min; MS m/z [M+H]⁺=551.4.

Enantiomer separation and analytics according to Method SFC-5, using 170 mg of the racemate, followed by removal of solvents under reduced pressure at 50° C., trituration with hexane and drying under reduced pressure (50° C.):

Peak 1: (99.5% ee), Intermediate A-11, 48 mg, white foam. LC-MS Method B-1: Rt=1.07 min. MS m/z [M+H]⁺=551.8. Analytical chiral HPLC (Method SFC-5): Rt=2.24 min.

Peak 2: (99.5% ee), Intermediate A-12, 48 mg, white foam. LC-MS Method B-1: Rt=1.08 min. MS m/z [M+H]⁺=551.8. Analytical chiral HPLC (Method SFC-5): Rt=2.90 min.

### Intermediate A-13 and Intermediate A-14:

### tert-butyl (R)-5-methoxy-4-((6-(4-(methoxycarbonyl)phenyl)-2-oxa-7-azaspiro[3.5]nonan-7-yl)methyl)-7-methyl-1H-indole-1-carboxylate and tert-butyl (S)-5-methoxy-4-((6-(4-(methoxycarbonyl)phenyl)-2-oxa-7-azaspiro[3.5]nonan-7-yl)methyl)-7-methyl-1H-indole-1-carboxylate

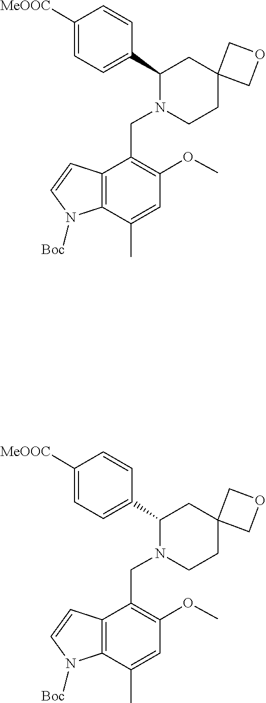 [SMILES: COC(=O)c1ccc([C@H]2CC3(CCN2Cc2c(OC)cc(C)c4c2ccn4C(=O)OC(C)(C)C)COC3)cc1 ; COC(=O)c1ccc([C@@H]2CC3(CCN2Cc2c(OC)cc(C)c4c2ccn4C(=O)OC(C)(C)C)COC3)cc1]

A soln. of methyl 4-(2-oxa-7-azaspiro[3.5]nonan-6-yl)benzoate (Intermediate rac-P-9, 77 mg, 295 μmol) and tert-butyl 4-formyl-5-methoxy-7-methyl-1H-indole-1-carboxylate (Intermediate I-1, 102 mg, 354 μmol) in DCE (2 mL) was stirred at RT for 40 min. NaBH(OAc)₃ (109 mg, 516 μmol) was added and the RM was stirred at RT for a total of 4 days with addition of another 3×0.5 eq (31 mg each) NaBH(OAc)₃ every 8-48 h. The RM was partitioned between sat. aq. NaHCO₃ soln. and MTBE. The layers were separated and washed with sat. aq. NaHCO₃ soln., brine and MTBE. The combined org. layers were dried over MgSO₄, filtered, and the volatiles were removed under reduced pressure at 50° C. to give a yellow oil. The crude product was purified over silica gel (12 g) using an automated purification system (NP; Teledyne ISCO®; collected at 229/254 nm; flow 30 mL/min; eluent: heptane+5 to 20.6% (EtOAc:MeOH 95:5) in 15.6 min. Pure fractions were combined, and the volatiles were removed under reduced pressure at 50° C. to give racemic tert-butyl 5-methoxy-4-((6-(4-(methoxycarbonyl)phenyl)-2-oxa-7-azaspiro[3.5]nonan-7-yl)methyl)-7-methyl-1H-indole-1-carboxylate compound as a white foam (125 mg). LC-MS Method B-1: Rt=1.00 min; MS m/z [M+H]⁺=535.3.

Enantiomer separation and analytics according to Method SFC-6, using 125 mg of the racemic mixture followed by removal of solvents under reduced pressure at 50° C., trituration with hexane and drying (under reduced pressure, 50° C.):

Peak 1: (99.5% ee), Intermediate A-13, 52 mg, white foam. LC-MS Method B-1: Rt=0.98 min. MS m/z [M+H]⁺=535.2. Analytical chiral HPLC (Method SFC-6): Rt=1.87 min.

Peak 2: (99.5% ee), Intermediate A-14, 51 mg, white foam. LC-MS Method B-1: Rt=0.95 min. MS m/z [M+H]⁺=535.4. Analytical chiral HPLC (Method SFC-6): Rt=3.89 min.

### Intermediate A-17:

### tert-butyl 4-(((5S,7R)-2,2-difluoro-7-(4-(methoxycarbonyl)phenyl)-8-azaspiro[4.5]decan-8-yl)methyl)-5-methoxy-7-methyl-1H-indole-1-carboxylate or tert-butyl 4-(((5R,7R)-2,2-difluoro-7-(4-(methoxycarbonyl)phenyl)-8-azaspiro[4.5]decan-8-yl)methyl)-5-methoxy-7-methyl-1H-indole-1-carboxylate

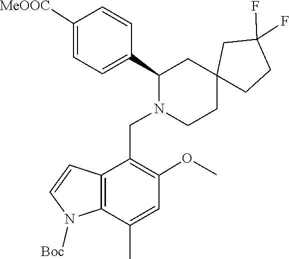 [SMILES: COC(=O)c1ccc([C@H]2CC3(CCN2Cc2c(OC)cc(C)c4c2ccn4C(=O)OC(C)(C)C)CCC(F)(F)C3)cc1]

Intermediate A-17 was prepared according to the method described for Intermediate A-18 herein below, using Intermediate P-10. LC-MS Method C-1: Rt=1.58 min; MS m/z [M-tertBu+Na+H]²⁺=274.1.

### Intermediate A-18:

### tert-butyl 4-(((5S,7S)-2,2-difluoro-7-(4-(methoxycarbonyl)phenyl)-8-azaspiro[4.5]decan-8-yl)methyl)-5-methoxy-7-methyl-1H-indole-1-carboxylate or tert-butyl 4-(((5R,7S)-2,2-difluoro-7-(4-(methoxycarbonyl)phenyl)-8-azaspiro[4.5]decan-8-yl)methyl)-5-methoxy-7-methyl-1H-indole-1-carboxylate

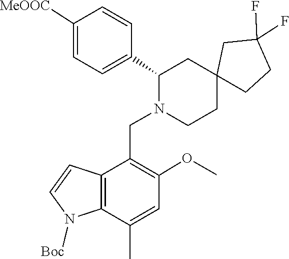 [SMILES: COC(=O)c1ccc([C@@H]2CC3(CCN2Cc2c(OC)cc(C)c4c2ccn4C(=O)OC(C)(C)C)CCC(F)(F)C3)cc1]

To a mixture of tert-butyl 4-formyl-5-methoxy-7-methyl-1H-indole-1-carboxylate (Intermediate I-1, 45.9 mg, 0.159 mmol) and crude Intermediate P-11 hydrochloride salt (0.159 mmol) in DCM (1.58 mL) and under N₂ atmosphere was added triethylamine (24.1 mg, 0.238 mmol). The mixture was stirred for ˜15 min and NaBH(OAc)₃ (50.5 mg, 0.238 mmol) was added in one portion. Stirring was continued for ˜2 days. The RM was diluted with DCM and washed with sat. aq. NaHCO₃ soln.; the separated org. layer was washed with brine, dried over Na₂SO₄, filtered off, and concentrated under reduced pressure providing the crude title compound. The crude material was directly used in the next reaction without further purification. LC-MS Method C-1: Rt=1.58 min; MS m/z [M-tertBu+Na+H]²⁺=274.1.

### Intermediate A-19:

### tert-butyl 4-(((5S,7R)-2,2-difluoro-7-(4-(methoxycarbonyl)phenyl)-8-azaspiro[4.5]decan-8-yl)methyl)-5-methoxy-7-methyl-1H-indole-1-carboxylate or tert-butyl 4-(((5R,7R)-2,2-difluoro-7-(4-(methoxycarbonyl)phenyl)-8-azaspiro[4.5]decan-8-yl)methyl)-5-methoxy-7-methyl-1H-indole-1-carboxylate

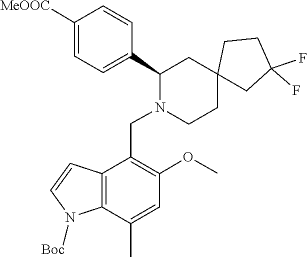 [SMILES: COC(=O)c1ccc([C@H]2CC3(CCN2Cc2c(OC)cc(C)c4c2ccn4C(=O)OC(C)(C)C)CCC(F)(F)C3)cc1]

Intermediate A-19 was prepared according to the method described for Intermediate A-18 herein above, using Intermediate P-12. LC-MS Method C-1: Rt=1.58 min; MS m/z [M-tertBu+Na+H]²⁺=274.1.

### Intermediate A-20:

### tert-butyl 4-(((5S,7S)-2,2-difluoro-7-(4-(methoxycarbonyl)phenyl)-8-azaspiro[4.5]decan-8-yl)methyl)-5-methoxy-7-methyl-1H-indole-1-carboxylate or tert-butyl 4-(((5R,7S)-2,2-difluoro-7-(4-(methoxycarbonyl)phenyl)-8-azaspiro[4.5]decan-8-yl)methyl)-5-methoxy-7-methyl-1H-indole-1-carboxylate

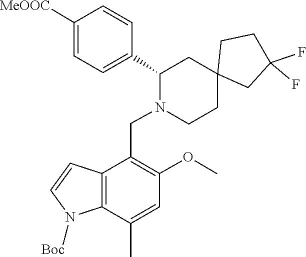 [SMILES: COC(=O)c1ccc([C@@H]2CC3(CCN2Cc2c(OC)cc(C)c4c2ccn4C(=O)OC(C)(C)C)CCC(F)(F)C3)cc1]

Intermediate A-20 was prepared according to the method described for Intermediate A-18 herein above, using Intermediate P-13. LC-MS Method C-1: Rt=1.57 min; MS m/z [M-tertBu+Na+H]²⁺=274.1.

### Intermediate A-21:

### tert-butyl (S)-5-cyclopropyl-4-((2,2-difluoro-6-(4-(methoxycarbonyl)phenyl)-7-azaspiro[3.5]nonan-7-yl)methyl)-7-methyl-1H-indole-1-carboxylate

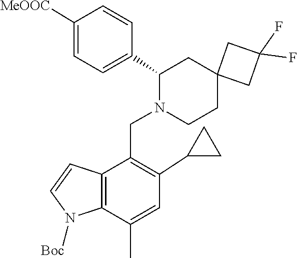 [SMILES: COC(=O)c1ccc([C@@H]2CC3(CCN2Cc2c(C4CC4)cc(C)c4c2ccn4C(=O)OC(C)(C)C)CC(F)(F)C3)cc1]

Methyl (S)-4-(2,2-difluoro-7-azaspiro[3.5]nonan-6-yl)benzoate (Intermediate P-6, 519 mg, 1.76 mmol) and tert-butyl 5-cyclopropyl-4-formyl-7-methyl-1H-indole-1-carboxylate (Intermediate I-3, 631 mg, 2.11 mmol) were dissolved in DCE. NaBH(OAc)₃ (652 mg, 3.08 mmol) was added in 4 portions over the course of 4 h. The RM was stirred for a total of 51 h with the addition of 4×0.5 eq (186 mg each) after 4 to 14 h. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow oil that was dissolved in 8.9 mL DCE and reacted with 0.5 eq (186 mg) NaBH(OAc)₃ for another 50 h with addition of another 0.5 eq (186 mg) NaBH(OAc)₃. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow oil. To remove unreacted aldehyde, the RM was dissolved in EtOH (10 mL) and NaBH₄ (33.2 mg, 879 μmol) was added. The RM was stirred for 30 min at RT, diluted with MTBE and water and brine was added. The layers were separated and washed with brine and MTBE. The combined org. layers were dried over MgSO₄, filtered, and the volatiles were removed under reduced pressure at 50° C. to give a yellow oil. The crude product was purified over silica gel (40 g) using an automated purification system (NP; Teledyne ISCO®; collected at 246/254 nm; flow 40 mL/min; eluent: heptane+1 to 23% EtOAc in 23 min). The pure fractions were combined, and the volatiles were removed under reduced pressure at 50° C. to give the title compound as a white foam (767 mg). LC-MS Method B-3: Rt=8.34 min; MS m/z [M+H]⁺=580.4 containing minor amounts of the corresponding ethyl ester Rt=8.70 min; MS m/z [M+H]⁺=593.5.

### Intermediate A-22:

### tert-butyl (S)-4-((2,2-difluoro-6-(4-(methoxycarbonyl)phenyl)-7-azaspiro[3.5]nonan-7-yl)methyl)-5,7-dimethyl-1H-indole-1-carboxylate

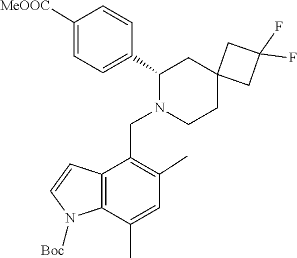 [SMILES: COC(=O)c1ccc([C@@H]2CC3(CCN2Cc2c(C)cc(C)c4c2ccn4C(=O)OC(C)(C)C)CC(F)(F)C3)cc1]

Methyl (S)-4-(2,2-difluoro-7-azaspiro[3.5]nonan-6-yl)benzoate (Intermediate P-6, 70 mg, 0.24 mmol) and tert-butyl 4-formyl-5,7-dimethyl-1H-indole-1-carboxylate (Intermediate I-2, 78 mg, 0.28 mmol) were dissolved in DCE. NaBH(OAc)₃ (88 mg, 0.41 mmol) was added in 2 portions over the course of 60 min. The RM was stirred for a total of 64 h with addition of 7×0.5 eq (25 mg each) of triacetoxyborohydride after 4 to 14 h. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow oil. To reduce unreacted Intermediate I-2 and facilitate separation, the RM was dissolved in 1.5 mL EtOH and NaBH₄ (4.5 mg, 0.12 mmol) was added The RM was stirred for 10 min at RT, then diluted with MTBE, water, and brine. The layers were separated and washed with brine and MTBE. The combined org. layers were dried over MgSO₄, filtered, and the volatiles were removed under reduced pressure at 50° C. to give a yellow oil that was purified over silica gel (12 g) using an automated purification system (NP; Teledyne ISCO®; collected at 238/254 nm; flow 30 mL/min; eluent: heptane+1 to 8.5% EtOAc in 11.6 min). The pure fractions were combined, and the volatiles were removed under reduced pressure at 50° C. to give the title compound as a white foam (93 mg). LC-MS Method B-1: Rt=1.69 min; MS m/z [M+H]⁺=553.5.

### Intermediate A-23:

### tert-butyl 4-(((S)-2,2-difluoro-6-(4-(methoxycarbonyl)phenyl)-7-azaspiro[3.5]nonan-7-yl)methyl)-5-((R)-2,2-difluorocyclopropyl)-7-methyl-1H-indole-1-carboxylate or tert-butyl 4-(((S)-2,2-difluoro-6-(4-(methoxycarbonyl)phenyl)-7-azaspiro[3.5]nonan-7-yl)methyl)-5-((S)-2,2-difluorocyclopropyl)-7-methyl-1H-indole-1-carboxylate

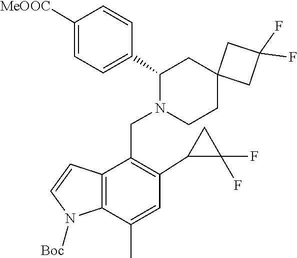 [SMILES: COC(=O)c1ccc([C@@H]2CC3(CCN2Cc2c(C4CC4(F)F)cc(C)c4c2ccn4C(=O)OC(C)(C)C)CC(F)(F)C3)cc1]

Methyl (S)-4-(2,2-difluoro-7-azaspiro[3.5]nonan-6-yl)benzoate (Intermediate P-6, 70.0 mg, 237.0 μmol) and Intermediate I-4 (5.38 mg, 284.4 μmol) were dissolved in DCE. NaBH(OAc)₃ (87.91 mg, 414.8 μmol) was added in 4 portions over the course of 4 hours. The RM was stirred for a total of 6 d with addition of 7×0.5 eq (25 mg each) NaBH(OAc)₃ after 4 to 48 h. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow oil. The crude product was dissolved in EtOH (1.0 mL) and in order to reduce excess aldehyde, NaBH₄ (6.73 mg, 177.8 μmol) was added. The RM was stirred for min, then diluted with MTBE, water, and brine. The layers were separated and washed with brine and MTBE. The combined org. layers were dried over MgSO₄, filtered, and the volatiles were removed under reduced pressure at 50° C. to give a colorless oil that was purified over silica gel (12 g) using an automated purification system (NP; Teledyne ISCO®; collected at 250/254 nm; flow 30 mL/min; eluent: heptane+1 to 10.2% EtOAc in 13.1 min). The pure fractions were combined, and the volatiles were removed under reduced pressure at 50° C. to give the title compound as a white foam (95 mg). LC-MS Method B-1: Rt=1.73 min; MS m/z [M+H]⁺=615.4.

### Intermediate A-24:

### tert-butyl 4-(((S)-2,2-difluoro-6-(4-(methoxycarbonyl)phenyl)-7-azaspiro[3.5]nonan-7-yl)methyl)-5-((R)-2,2-difluorocyclopropyl)-7-methyl-1H-indole-1-carboxylate or tert-butyl 4-(((S)-2,2-difluoro-6-(4-(methoxycarbonyl)phenyl)-7-azaspiro[3.5]nonan-7-yl)methyl)-5-((S)-2,2-difluorocyclopropyl)-7-methyl-1H-indole-1-carboxylate

Intermediate A-24 was prepared similar to its diastereomer Intermediate A-23 as described above, using methyl (S)-4-(2,2-difluoro-7-azaspiro[3.5]nonan-6-yl)benzoate (Intermediate P-6, 70.0 mg, 237 μmol) and Intermediate I-5, (87.4 mg, 260.7 μmol) yielding Intermediate A-24 as a white foam (83 mg). LC-MS Method B-1: Rt=1.70 min; MS m/z [M+H]⁺=615.5.

Intermediate rac-A-25:

### (RS)-tert-butyl 5-cyclopropyl-4-((2,2-difluoro-6-(4-(3-hydroxyoxetan-3-yl)phenyl)-7-azaspiro[3.5]nonan-7-yl)methyl)-7-methyl-1H-indole-1-carboxylate

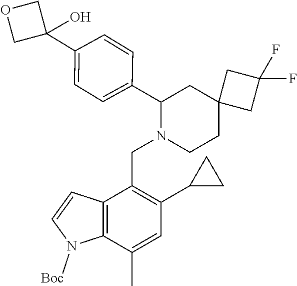 [SMILES: Cc1cc(C2CC2)c(CN2CCC3(CC2c2ccc(C4(O)COC4)cc2)CC(F)(F)C3)c2ccn(C(=O)OC(C)(C)C)c12]

A soln. of 3-(4-(2,2-difluoro-7-azaspiro[3.5]nonan-6-yl)phenyl)oxetan-3-ol (Intermediate rac-P-14, 109.2 mg, 353.0 μmol), tert-butyl 4-(chloromethyl)-5-cyclopropyl-7-methyl-1H-indole-1-carboxylate (Intermediate I-6, 129.8 mg, 405.9 μmol) and DIPEA (91.3 mg, 123 μL, 706 μmol) was placed in a capped vial and the RM was stirred at 50° C. for 20 h. The RM was distributed between water and MTBE. The layers were separated and washed with brine and MTBE. The combined org. layers were dried over MgSO₄, filtered, and the volatiles were removed under reduced pressure at 50° C. The residue was purified over silica gel (12 g) using an automated purification system (NP; Teledyne ISCO®; collected at 250/272 nm; flow 30 mL/min; eluent: heptane+2 to 32% EtOAc in 20 min). Pure fractions were combined, and the volatiles were removed under reduced pressure at 50° C. to give the title compound as a white foam (199 mg). LC-MS Method B-1: Rt=1.21 min; MS m/z [M+H]⁺=593.5.

Intermediate rac-A-26:

### (RS)-tert-butyl 5-cyclopropyl-4-((2,2-difluoro-6-(4-(2-hydroxypropan-2-yl)phenyl)-7-azaspiro[3.5]nonan-7-yl)methyl)-7-methyl-1H-indole-1-carboxylate

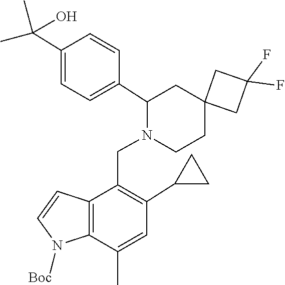 [SMILES: Cc1cc(C2CC2)c(CN2CCC3(CC2c2ccc(C(C)(C)O)cc2)CC(F)(F)C3)c2ccn(C(=O)OC(C)(C)C)c12]

A soln. of 2-(4-(2,2-difluoro-7-azaspiro[3.5]nonan-6-yl)phenyl)propan-2-ol (Intermediate rac-P-15, 112.0 mg, 379.2 μmol), tert-butyl 4-(chloromethyl)-5-cyclopropyl-7-methyl-1H-indole-1-carboxylate (Intermediate I-6, 139.5 mg, 436.1 μmol) and DIPEA (98.02 mg, 132 μL, 758.4 μmol) was placed in a capped vial and the RM was stirred at 50° C. overnight. The RM was distributed between water and MTBE. The layers were separated and washed with brine and MTBE. The combined org. layers were dried over MgSO₄, filtered, and the volatiles were removed under reduced pressure at 50° C. to give a colorless oil that was purified over silica gel (12 g) using an automated purification system (NP; Teledyne ISCO®; collected at 250/272 nm; flow 30 mL/min; eluent: heptane+2 to 20.5% EtOAc in 13.2 min). Pure fractions were combined, and the volatiles were removed under reduced pressure at 50° C. to give the title compound as a white foam (218 mg). LC-MS Method B-1: Rt=1.33 min; MS m/z [M+H]⁺=579.5.

Intermediate rac-A-27:

### (RS)-tert-butyl 4-((6-(4-(1H-pyrazol-5-yl)phenyl)-2,2-difluoro-7-azaspiro[3.5]nonan-7-yl)methyl)-5-cyclopropyl-7-methyl-1H-indole-1-carboxylate

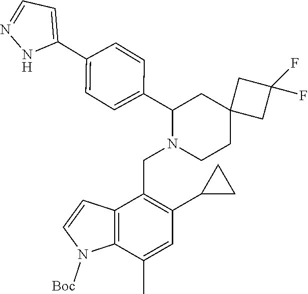 [SMILES: Cc1cc(C2CC2)c(CN2CCC3(CC2c2ccc(-c4ccn[nH]4)cc2)CC(F)(F)C3)c2ccn(C(=O)OC(C)(C)C)c12]

A soln. of 6-(4-(1H-pyrazol-5-yl)phenyl)-2,2-difluoro-7-azaspiro[3.5]nonane (Intermediate rac-P-16, 110.0 mg, 362.6 μmol), tert-butyl 4-(chloromethyl)-5-cyclopropyl-7-methyl-1H-indole-1-carboxylate (Intermediate I-6, 133.4 mg, 417.0 μmol) and DIPEA (93.73 mg, 126 μL, 725.2 μmol) was placed in a capped vial and the RM was stirred at 50° C. overnight. The RM was distributed between water and MTBE. The layers were separated and washed with brine and MTBE. The combined org. layers were dried over MgSO₄, filtered, and the volatiles were removed under reduced pressure at 50° C. to give a colorless oil. The crude product was purified over silica gel (12 g) using an automated purification system (NP; Teledyne ISCO®; collected at 254/275 nm; flow 30 mL/min; eluent: heptane+5 to 39.3% EtOAc in 15.2 min). Pure fractions were combined, and the volatiles were removed under reduced pressure at 50° C. to give the title compound as a white foam (190 mg). LC-MS Method B-1: Rt=1.37 min; MS m/z [M+H]⁺=587.4.

Intermediate rac-A-28:

### (RS)-tert-butyl 4-((6-(4-(1H-pyrazol-1-yl)phenyl)-2,2-difluoro-7-azaspiro[3.5]nonan-7-yl)methyl)-5-cyclopropyl-7-methyl-1H-indole-1-carboxylate

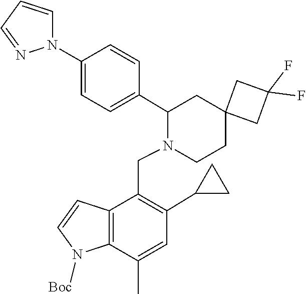 [SMILES: Cc1cc(C2CC2)c(CN2CCC3(CC2c2ccc(-n4cccn4)cc2)CC(F)(F)C3)c2ccn(C(=O)OC(C)(C)C)c12]

A soln. of 6-(4-(1H-pyrazol-1-yl)phenyl)-2,2-difluoro-7-azaspiro[3.5]nonane (Intermediate rac-P-17, 110.0 mg, 362.6 μmol), tert-butyl 4-(chloromethyl)-5-cyclopropyl-7-methyl-1H-indole-1-carboxylate (Intermediate I-6, 133.4 mg, 417.0 μmol) and DIPEA (93.73 mg, 126 μL, 725.2 μmol) was placed in a capped vial and the RM was stirred at 50° C. overnight. The RM was diluted with water and MTBE. The layers were separated and washed with brine and MTBE. The combined org. layers were dried over MgSO₄, filtered, and the volatiles were removed under reduced pressure at 50° C. to give a colorless oil. The crude product was purified over silica gel (12 g) using an automated purification system (NP; Teledyne ISCO®; collected at 254/275 nm; flow 30 mL/min; eluent: heptane+0 to 7.8% EtOAc in 20.1 min). Pure fractions were combined and the volatiles were removed under reduced pressure at 50° C. to give the title compound as a white foam (209 mg). LC-MS Method B-1: Rt=1.53 min; MS m/z [M+H]⁺=587.5.

Intermediate rac-A-29:

### (RS)-tert-butyl 4-((6-(4-cyanophenyl)-2,2-difluoro-7-azaspiro[3.5]nonan-7-yl)methyl)-5-cyclopropyl-7-methyl-1H-indole-1-carboxylate

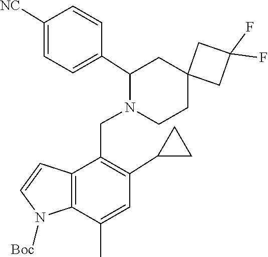 [SMILES: Cc1cc(C2CC2)c(CN2CCC3(CC2c2ccc(C#N)cc2)CC(F)(F)C3)c2ccn(C(=O)OC(C)(C)C)c12]

A soln. of 4-(2,2-difluoro-7-azaspiro[3.5]nonan-6-yl)benzonitrile (Intermediate rac-P-18, 137.0 mg, 522.3 μmol), tert-butyl 4-(chloromethyl)-5-cyclopropyl-7-methyl-1H-indole-1-carboxylate (Intermediate I-6, 192.1 mg, 600.6 μmol) and DIPEA (135.0 mg, 182 μL, 1.045 mmol) was placed in a capped vial and the RM was stirred at 50° C. overnight. The RM was distributed between water and MTBE. The layers were separated and washed with brine and MTBE. The combined org. layers were dried over MgSO₄, filtered, and the volatiles were removed under reduced pressure at 50° C. to give a colorless oil.

The crude product was purified over silica gel (24 g) using an automated purification system (NP; Teledyne ISCO®; collected at 254/275 nm; flow 35 mL/min; eluent: heptane+0 to 7.1% EtOAc in 17.7 min). Purest fractions were combined, and the volatiles were removed under reduced pressure at 50° C. to give the title compound as a white foam (273 mg). LC-MS Method B-1: Rt=1.69 min; MS m/z [M+H]⁺=546.3.

Intermediate rac-A-30:

### (RS)-tert-butyl 4-((2,2-difluoro-6-phenyl-7-azaspiro[3.5]nonan-7-yl)methyl)-5-methoxy-7-methyl-1H-indole-1-carboxylate

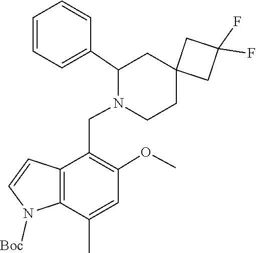 [SMILES: COc1cc(C)c2c(ccn2C(=O)OC(C)(C)C)c1CN1CCC2(CC1c1ccccc1)CC(F)(F)C2]

2,2-Difluoro-6-phenyl-7-azaspiro[3.5]nonane (Intermediate rac-P-19, 68.50 mg, 288.7 μmol) and tert-butyl 4-formyl-5-methoxy-7-methyl-1H-indole-1-carboxylate (Intermediate I-1, 108.6 mg, 375.3 μmol) were dissolved in DCE. NaBH(OAc)₃ (107.1 mg, 505.2 μmol) was added in portions over the course of 4 h. The RM was stirred for a total of 2.5 d with addition of 7×0.5 eq NaBH(OAc)₃ (31 mg each) after 2.5 to 14 h. The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a slightly yellow oil. In order to reduce excess Intermediate I-1 and facilitate separation, the crude material was dissolved in EtOH (2.0 mL) and NaBH₄ (10.92 mg, 288.7 μmol) was added. The RM was stirred for 10 min at RT and then partitioned between MTBE, water, and brine. The layers were separated and washed with brine and MTBE. The combined org. layers were dried over MgSO₄, filtered, and the volatiles were removed under reduced pressure at 50° C. to give a colorless oil that was purified over silica gel column (12 g) using an automated purification system (NP; Teledyne ISCO®; collected at 245/275 nm; flow 30 mL/min; eluent: heptane+1.0 to 6.5% EtOAc in 12.3 min). Pure fractions were combined, and the volatiles were removed under reduced pressure at 50° C. to give the title compound as a white foam (115 mg). LC-MS Method B-1: Rt=1.07 min; MS m/z [M+H]⁺=511.3.

Intermediate rac-A-31:

### (RS)-tert-butyl 4-((2,2-difluoro-6-(1-methyl-1H-pyrazol-4-yl)-7-azaspiro[3.5]nonan-7-yl)methyl)-5-methoxy-7-methyl-1H-indole-1-carboxylate

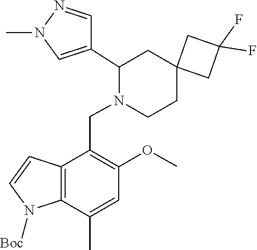 [SMILES: COc1cc(C)c2c(ccn2C(=O)OC(C)(C)C)c1CN1CCC2(CC1c1cnn(C)c1)CC(F)(F)C2]

2,2-Difluoro-6-(1-methyl-1H-pyrazol-4-yl)-7-azaspiro[3.5]nonane (Intermediate rac-P-20, 89.50 mg, 370.9 μmol) and tert-butyl 4-formyl-5-methoxy-7-methyl-1H-indole-1-carboxylate (Intermediate I-1, 150.2 mg, 519.3 μmol) were dissolved in DCE (1.50 mL). NaBH(OAc)₃ (137.6 mg, 649.1 μmol) was added in four portions over the course of 4 h. Another 0.5 eq (39 mg) NaBH(OAc)₃ were added and the RM was stirred for a total of 34 h with addition of additional 3×0.5 eq NaBH(OAc)₃ (39 mg each) after 4 to 14 h. The RM was diluted with DCE and sat. aq. Na₂CO₃ soln. (10 wt %). The layers were separated. The aq. layer was extracted with DCE (2×). The combined org. layers were dried over MgSO₄, filtered, and the volatiles were removed under reduced pressure at 50° C. to give a yellow oil. The material was re-dissolved in 1.5 mL DCE and 0.5 eq (39 mg) NaBH(OAc)₃ was added. The RM was stirred for a total of 21 h with addition of additional 2×0.5 eq NaBH(OAc)₃ (39 mg each). The RM was diluted with MTBE and sat. aq. NaHCO₃ soln. was added. The layers were separated and washed with sat. aq. NaHCO₃ soln., brine and MTBE. The combined org. layers were dried over MgSO₄, filtered, and the volatiles were removed under reduced pressure at 50° C. to give a yellow oil. The crude product was purified over silica gel (12 g) using an automated purification system (NP; Teledyne ISCO®; collected at 250/275 nm; flow 30 mL/min; eluent: heptane+5 to 44.8% (EtOAc:MeOH 95:5) in 14.5 min). Pure fractions were combined, and the volatiles were removed under reduced pressure at 50° C. to give the title compound as white crystals (167 mg). LC-MS Method B-1: Rt=0.87 min; MS m/z [M+H]⁺=515.2.

### Intermediate A-32 and Intermediate A-33:

### tert-butyl (S)-4-((6-(1-(2-ethoxy-2-oxoethyl)-1H-pyrazol-4-yl)-2,2-difluoro-7-azaspiro[3.5]nonan-7-yl)methyl)-5-methoxy-7-methyl-1H-indole-1-carboxylate and tert-butyl (R)-4-((6-(1-(2-ethoxy-2-oxoethyl)-1H-pyrazol-4-yl)-2,2-difluoro-7-azaspiro[3.5]nonan-7-yl)methyl)-5-methoxy-7-methyl-1H-indole-1-carboxylate

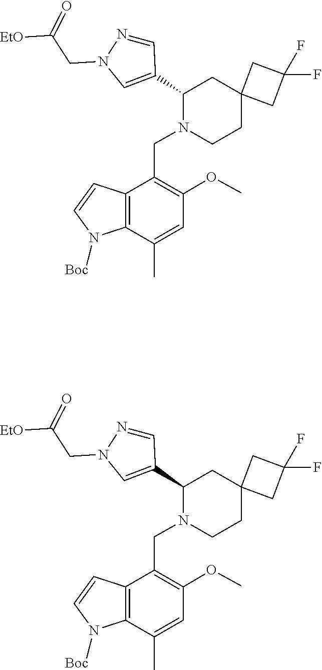 [SMILES: CCOC(=O)Cn1cc([C@@H]2CC3(CCN2Cc2c(OC)cc(C)c4c2ccn4C(=O)OC(C)(C)C)CC(F)(F)C3)cn1 ; CCOC(=O)Cn1cc([C@H]2CC3(CCN2Cc2c(OC)cc(C)c4c2ccn4C(=O)OC(C)(C)C)CC(F)(F)C3)cn1]

Racemic tert-butyl 4-((6-(1-(2-ethoxy-2-oxoethyl)-1H-pyrazol-4-yl)-2,2-difluoro-7-azaspiro[3.5]nonan-7-yl)methyl)-5-methoxy-7-methyl-1H-indole-1-carboxylate was prepared similar to the method described for Intermediate rac-A-31 herein above, using Intermediate rac-P-23 and Intermediate I-1. LC-MS Method B-6: Rt=0.88 min; MS m/z [M+H]⁺=587.2 Enantiomer separation and analytics according to Method SFC-22 of the racemic mixture provided:

Peak 1: (99.3% ee), Intermediate A-32, 35.4 mg, yellow oil. LC-MS Method B-6: Rt=0.86 min. MS m/z [M+H]⁺=587.4. Analytical chiral HPLC (Method SFC-22): Rt=1.59 min.

Peak 2: (99.3% ee), Intermediate A-33, 34.0 mg, yellow oil. LC-MS Method B-6: Rt=0.87 min. MS m/z [M+H]⁺=587.3. Analytical chiral HPLC (Method SFC-22): Rt=2.06 min.

### Intermediate A-34 and Intermediate A-35:

### tert-butyl (S)-4-((2,2-difluoro-6-(6-(methoxycarbonyl)pyridin-3-yl)-7-azaspiro[3.5]nonan-7-yl)methyl)-5-methoxy-7-methyl-1H-indole-1-carboxylate and tert-butyl (R)-4-((2,2-difluoro-6-(6-(methoxycarbonyl)pyridin-3-yl)-7-azaspiro[3.5]nonan-7-yl)methyl)-5-methoxy-7-methyl-1H-indole-1-carboxylate

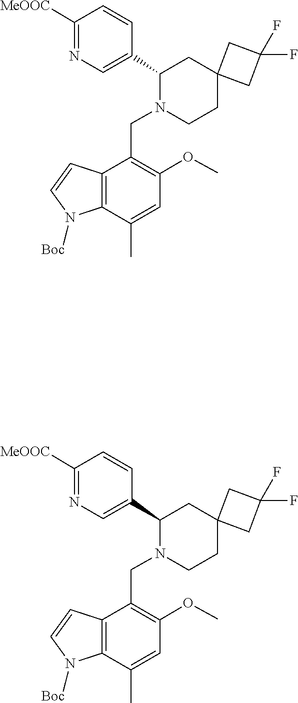 [SMILES: COC(=O)c1ccc([C@@H]2CC3(CCN2Cc2c(OC)cc(C)c4c2ccn4C(=O)OC(C)(C)C)CC(F)(F)C3)cn1 ; COC(=O)c1ccc([C@H]2CC3(CCN2Cc2c(OC)cc(C)c4c2ccn4C(=O)OC(C)(C)C)CC(F)(F)C3)cn1]

Racemic tert-butyl 4-((2,2-difluoro-6-(6-(methoxycarbonyl)pyridin-3-yl)-7-azaspiro[3.5]nonan-7-yl)methyl)-5-methoxy-7-methyl-1H-indole-1-carboxylate was prepared similar to the method described for Intermediate rac-A-78 herein above, using Intermediate rac-P-25 and Intermediate I-1. LC-MS Method B-6: Rt=1.19 min; MS m/z [M+H]⁺=570.2

Enantiomer separation and analytics according to Method SFC-26 of the racemic mixture provided:

Peak 1: (99.5% ee), Intermediate A-34, 80.4 mg, white. LC-MS Method B-6: Rt=1.19 min. MS m/z [M+H]⁺=570.2. Analytical chiral HPLC (Method SFC-26): Rt=1.19 min.

Peak 2: (98.5% ee), Intermediate A-35, 83.1 mg, white. LC-MS Method B-6: Rt=1.20 min. MS m/z [M+H]⁺=570.2. Analytical chiral HPLC (Method SFC-26): Rt=1.79 min.

### Intermediate A-36 and Intermediate A-37:

### tert-butyl (S)-5-cyclopropyl-4-((2,2-difluoro-6-(6-(methoxycarbonyl)pyridin-3-yl)-7-azaspiro[3.5]nonan-7-yl)methyl)-7-methyl-1H-indole-1-carboxylate and tert-butyl (R)-5-cyclopropyl-4-((2,2-difluoro-6-(6-(methoxycarbonyl)pyridin-3-yl)-7-azaspiro[3.5]nonan-7-yl)methyl)-7-methyl-1H-indole-1-carboxylate

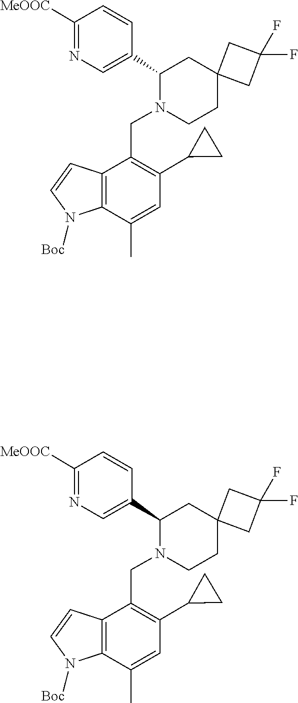 [SMILES: COC(=O)c1ccc([C@@H]2CC3(CCN2Cc2c(C4CC4)cc(C)c4c2ccn4C(=O)OC(C)(C)C)CC(F)(F)C3)cn1 ; COC(=O)c1ccc([C@H]2CC3(CCN2Cc2c(C4CC4)cc(C)c4c2ccn4C(=O)OC(C)(C)C)CC(F)(F)C3)cn1]

Racemic tert-butyl 5-cyclopropyl-4-((2,2-difluoro-6-(6-(methoxycarbonyl)pyridin-3-yl)-7-azaspiro[3.5]nonan-7-yl)methyl)-7-methyl-1H-indole-1-carboxylate was prepared similar to the method described for Intermediate rac-A-78 herein above, using Intermediate rac-P-25 and Intermediate I-3. LC-MS Method B-6: Rt=1.65 min; MS m/z [M+H]⁺=580.3 Enantiomer separation and analytics according to Method SFC-27 of the racemic mixture provided:

Peak 1: (99.5% ee), Intermediate A-36, 60.7 mg, white solid. LC-MS Method B-6: Rt=1.64 min. MS m/z [M+H]⁺=580.5. Analytical chiral HPLC (Method SFC-27): Rt=2.79 min.

Peak 2: (98.9% ee), Intermediate A-37, 63.1 mg, white solid. LC-MS Method B-6: Rt=1.64 min. MS m/z [M+H]⁺=580.3. Analytical chiral HPLC (Method SFC-27): Rt=3.87 min.

### Intermediate A-38 and Intermediate A-39:

### tert-butyl (S)-4-((2,2-difluoro-6-(6-(methoxycarbonyl)pyridin-3-yl)-7-azaspiro[3.5]nonan-7-yl)methyl)-5,7-dimethyl-1H-indole-1-carboxylate and tert-butyl (R)-4-((2,2-difluoro-6-(6-(methoxycarbonyl)pyridin-3-yl)-7-azaspiro[3.5]nonan-7-yl)methyl)-5,7-dimethyl-1H-indole-1-carboxylate

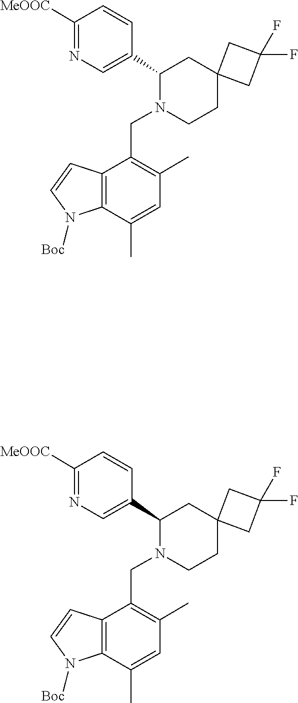 [SMILES: COC(=O)c1ccc([C@@H]2CC3(CCN2Cc2c(C)cc(C)c4c2ccn4C(=O)OC(C)(C)C)CC(F)(F)C3)cn1 ; COC(=O)c1ccc([C@H]2CC3(CCN2Cc2c(C)cc(C)c4c2ccn4C(=O)OC(C)(C)C)CC(F)(F)C3)cn1]

Racemic tert-butyl 4-((2,2-difluoro-6-(6-(methoxycarbonyl)pyridin-3-yl)-7-azaspiro[3.5]nonan-7-yl)methyl)-5,7-dimethyl-1H-indole-1-carboxylate was prepared similar to the method described for Intermediate rac-A-78 herein above, using Intermediate rac-P-25 and Intermediate I-2. LC-MS Method B-6: Rt=1.62 min; MS m/z [M+H]⁺=554.2

Enantiomer separation and analytics according to Method SFC-28 of the racemic mixture provided:

Peak 1: (99.5% ee), Intermediate A-38, 69.9 mg, white foam. LC-MS Method B-6: Rt=1.62 min. MS m/z [M+H]⁺=554.1. Analytical chiral HPLC (Method SFC-28): Rt=1.99 min.

Peak 2: (99.5% ee), Intermediate A-39, 68.9 mg, white foam. LC-MS Method B-6: Rt=1.62 min. MS m/z [M+H]⁺=554.1. Analytical chiral HPLC (Method SFC-28): Rt=2.72 min.

### COMPOUND EXAMPLES

### Example Ex-5

### (S)-4-(2,2-difluoro-7-((5-methoxy-7-methyl-1H-indol-4-yl)methyl)-7-azaspiro[3.5]nonan-6-yl)benzoic acid

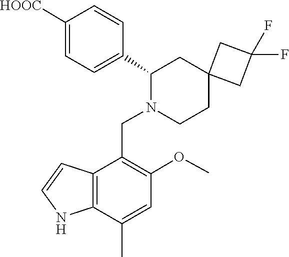 [SMILES: COc1cc(C)c2[nH]ccc2c1CN1CCC2(C[C@H]1c1ccc(C(=O)O)cc1)CC(F)(F)C2]

A soln. of tert-butyl (S)-4-((2,2-difluoro-6-(4-(methoxycarbonyl)phenyl)-7-azaspiro[3.5]nonan-7-yl)methyl)-5-methoxy-7-methyl-1H-indole-1-carboxylate (Intermediate A-7, 7.85 g, 13.8 mmol) in THE (60 mL) and MeOH (148 mL) was evacuated/back-filled with N₂. 4M aq. NaOH (34.5 mL, 138 mmol) was added. The mixture was evacuated/back-filled with N₂ once more. The well-stirred RM was kept under N₂ and heated to 50° C. to give a homogeneous soln. and stirred for 21 h. The RM was cooled to RT and quenched with 4M aq. HCl (5.03 g, 34.5 mL, 138 mmol). About 50% of the volatiles (MeOH, THF) were removed under reduced pressure at 50° C. EtOAc, some water, and brine were added. The layers were separated and washed with brine and EtOAc. The combined org. layers were dried over MgSO₄, filtered, and most of the volatiles were removed under reduced pressure at 50° C. to give a slightly amber foam. All solvents used in the following purification step were of p.a. quality. The crude product was dissolved in DCM and some drops of MeOH. The soln. was purified over silica gel (220 g) using an automated purification system (NP; Teledyne ISCO®; collected at 230/282 nm; flow 150 mL/min; eluent: DCM+2 to 17% MeOH in 29.2 min, isocratic gradient at the end). Pure fractions were combined, and the volatiles were removed under reduced pressure at 50° C. to give a slightly yellow foam that dissolved in EtOAc and as little MeOH as possible. The volatiles were removed again under reduced pressure at 50° C. The resulting semi-solid gum was redissolved in EtOAc and as little MeOH as possible. The volatiles were again removed under reduced pressure at 50° C.; this step was repeated 2×, then the residue was dissolved in EtOAc, and the mixture was concentrated under reduced pressure at 50° C. to a volume of about 40 mL. The mixture was cooled to RT and 100 mL hexane was gradually added via dropping funnel in order to precipitate the product. The resulting white suspension was treated with ultrasound and stirred for another 10 min. Subsequently, the suspension was filtered and the filter cake was washed with ice cold hexane/EtOAc 4/1. The filter cake was dried under reduced pressure at 50° C. to give the title compound as a white powder (5.96 g). LC-MS Method B-3: Rt=2.34 min; MS m/z [M+H]⁺=455.2.

### Example Ex-6

### (R)-4-(2,2-difluoro-7-((5-methoxy-7-methyl-1H-indol-4-yl)methyl)-7-azaspiro[3.5]nonan-6-yl)benzoic acid

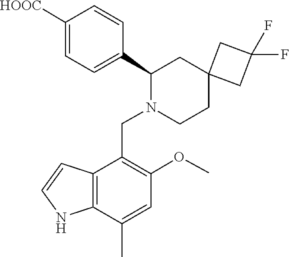 [SMILES: COc1cc(C)c2[nH]ccc2c1CN1CCC2(C[C@@H]1c1ccc(C(=O)O)cc1)CC(F)(F)C2]

An aq. 4M NaOH soln. (492 μL) was added to a soln. of tert-butyl (R)-4-((2,2-difluoro-6-(4-(methoxycarbonyl)phenyl)-7-azaspiro[3.5]nonan-7-yl)methyl)-5-methoxy-7-methyl-1H-indole-1-carboxylate (Intermediate A-8, 111 mg, 195 μmol) in MeOH/THF (2.0 mL/1.0 mL). The RM was stirred at 50° C. for 3 h 40 min. The RM was cooled to RT, quenched with 4M aq. HCl soln. (488 μL), EtOAc, some water, and a few drops of brine were added. The layers were separated and washed with brine and EtOAc. The combined org. layers were dried over MgSO₄, filtered, and the volatiles were removed under reduced pressure at 50° C. to give a milky oil. The mixture was dissolved in ACN/MeOH, the volatiles were removed again under reduced pressure at 50° C., and the residue was dried under reduced pressure at 50° C. to give the title compound as a grey foam that was purified on a preparative TLC plate (MERCK 1.05744.0001, PLC silica gel 60 F254, 0.5 mm; eluent: DCM/MeOH 88/12). The main band (by UV) was scratched off the plate. The silica gel/compound mixture was suspended in DCM/MeOH 85/15, treated with ultrasound and filtered. The silica gel was rinsed with DCM/MeOH 85/15. The volatiles of the filtrate were removed under reduced pressure at 50° C. to give the title compound as an off-white powder (80 mg). LC-MS Method B-3: Rt=2.30 min; MS m/z [M+H]⁺=455.4.

### Example Ex-9

### 4-(2-fluoro-7-((5-methoxy-7-methyl-1H-indol-4-yl)methyl)-7-azaspiro[3.5]nonan-6-yl)benzoic acid (single stereoisomer 3)

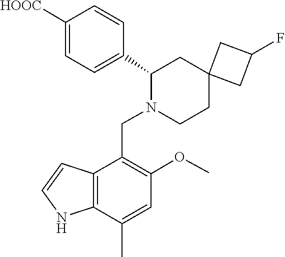 [SMILES: COc1cc(C)c2[nH]ccc2c1CN1CCC2(CC(F)C2)C[C@H]1c1ccc(C(=O)O)cc1]

An aq. 4M NaOH soln. (179 μL) was added to a soln. of Intermediate A-11 (49.2 mg, 89.3 μmol) in MeOH (1 mL) and THE (0.2 μL). The RM was stirred at 50° C. overnight, cooled to RT, and quenched with aq. 4M HCl soln. (179 μL). This soln. was directly purified by preparative HPLC (Waters Xbridge C18 Dimensions: 30 mm×100 mm 5 μm; flow 50 mL/min; eluent A=water+0.1% TFA; eluent B=ACN; gradient: 15 to 50% B in 12 min). Product containing fractions were combined, volatiles were removed under reduced pressure at 40° C. and the remaining aq. soln. was lyophilized to give the title compound as an off-white solid (44.3 mg). LC-MS Method B-1: Rt=0.48 min; MS m/z [M+H]⁺=437.4.

### Example Ex-18

### 4-((5S,7S)-2,2-difluoro-8-((5-methoxy-7-methyl-1H-indol-4-yl)methyl)-8-azaspiro[4.5]decan-7-yl)benzoic acid or 4-((5R,7S)-2,2-difluoro-8-((5-methoxy-7-methyl-1H-indol-4-yl)methyl)-8-azaspiro[4.5]decan-7-yl)benzoic acid

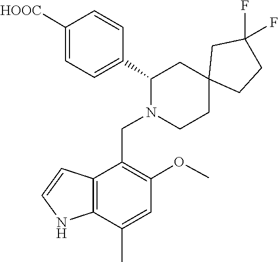 [SMILES: COc1cc(C)c2[nH]ccc2c1CN1CCC2(CCC(F)(F)C2)C[C@H]1c1ccc(C(=O)O)cc1]

To a mixture of crude Intermediate A-18 (61.8 mg, 0.106 mmol) in THF/MeOH (1.06 mL/1.06 mL) was added 2N aq. NaOH soln. (1.06 mL, 2.12 mmol). The mixture was stirred at RT for ˜15 min and then heated at 50° C. for ˜3 h. The volatile solvent was removed under reduced pressure to approximate % of the original volume. The residue was diluted with water and ACN and purified by preparative HPLC on an XBridge C18 OBD column (30×50 mm, 5 μm) eluting with 15% to 40% ACN in aq. NH₄OH (5 mM) to provide the title compound (24 mg) as a white solid after lyophilization. LC-MS Method C-1: Rt=0.74 min; MS m/z [M+H]⁺=469.4.

### Example Ex-21

### (S)-4-(7-((5-cyclopropyl-7-methyl-1H-indol-4-yl)methyl)-2,2-difluoro-7-azaspiro[3.5]nonan-6-yl)benzoic acid

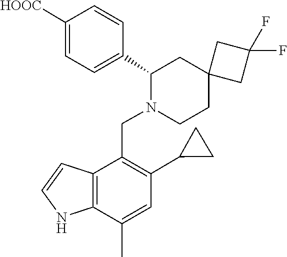 [SMILES: Cc1cc(C2CC2)c(CN2CCC3(C[C@H]2c2ccc(C(=O)O)cc2)CC(F)(F)C3)c2cc[nH]c12]

A 4M aq. NaOH soln. (3.31 mL, 13.3 mmol) was added to a soln. of tert-butyl (S)-5-cyclopropyl-4-((2,2-difluoro-6-(4-(methoxycarbonyl)phenyl)-7-azaspiro[3.5]nonan-7-yl)methyl)-7-methyl-1H-indole-1-carboxylate (Intermediate A-21, 767 mg, 1.33 mmol) in MeOH/THF (9 mL, 5.3 mL). The RM was stirred at 50° C. overnight, then quenched with 4M aq. HCl soln. (3.31 mL, 13.3 mmol). EtOAc, water, and a few drops of brine were added. The layers were separated and washed with brine and EtOAc. The combined org. layers were dried over MgSO₄, filtered, and the volatiles were removed under reduced pressure at 50° C. to give an off-white oil. The crude product was purified over silica gel (25 g) using an automated purification system (NP; Teledyne ISCO®; collected at 234/285 nm; flow 35 mL/min; eluent: DCM+0.5 to 13.9% MeOH in 18.5 min). Pure fractions were combined and the volatiles were removed under reduced pressure at 50° C., taken up in ACN, the volatiles were again removed under reduced pressure at 50° C. and the product was further dried under reduced pressure at 50° C. to give the title compound as an off-white powder (582 mg). LC-MS Method B-3: Rt=3.27 min; MS m/z=465.4.

### Example Ex-49, Example Ex-25, and Example Ex-26

### 3-(4-(7-((5-cyclopropyl-7-methyl-1H-indol-4-yl)methyl)-2,2-difluoro-7-azaspiro[3.5]nonan-6-yl)phenyl)oxetan-3-ol, (S)-3-(4-(7-((5-cyclopropyl-7-methyl-1H-indol-4-yl)methyl)-2,2-difluoro-7-azaspiro[3.5]nonan-6-yl)phenyl)oxetan-3-ol, and (R)-3-(4-(7-((5-cyclopropyl-7-methyl-1H-indol-4-yl)methyl)-2,2-difluoro-7-azaspiro[3.5]nonan-6-yl)phenyl)oxetan-3-ol

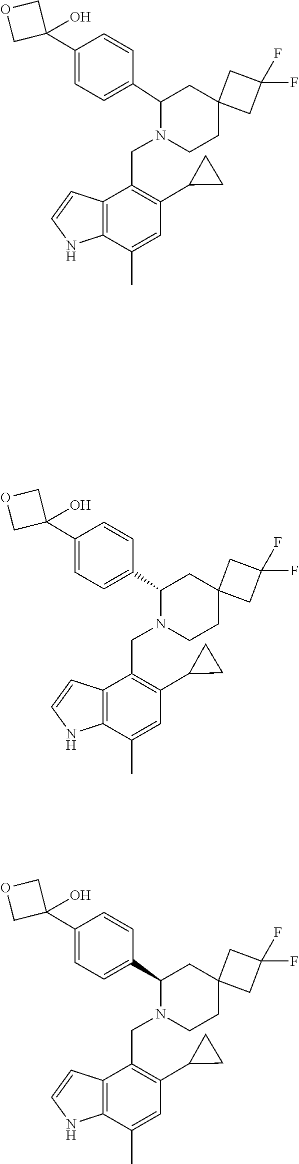 [SMILES: Cc1cc(C2CC2)c(CN2CCC3(CC2c2ccc(C4(O)COC4)cc2)CC(F)(F)C3)c2cc[nH]c12 ; Cc1cc(C2CC2)c(CN2CCC3(C[C@H]2c2ccc(C4(O)COC4)cc2)CC(F)(F)C3)c2cc[nH]c12 ; Cc1cc(C2CC2)c(CN2CCC3(C[C@@H]2c2ccc(C4(O)COC4)cc2)CC(F)(F)C3)c2cc[nH]c12]

Potassium carbonate 325 mesh (264.6 mg, 1.915 mmol) was added to a soln. of racemic tert-butyl 5-cyclopropyl-4-((2,2-difluoro-6-(4-(3-hydroxyoxetan-3-yl)phenyl)-7-azaspiro[3.5]nonan-7-yl)methyl)-7-methyl-1H-indole-1-carboxylate (Intermediate rac-A-25, 227.0 mg, 383.0 μmol) in MeOH (5.0 mL). The RM was stirred at 60° C. for ˜10 h, then kept without heating overnight. The MeOH was removed under reduced pressure at 50° C. The resulting residue was distributed between water and DCM. The layers were separated, and the aq. layer was washed with DCM (2×). The combined org. layers were dried over MgSO₄, filtered and the volatiles were removed under reduced pressure at 50° C. to give a white foam. The crude product was dissolved in DCM and equally distributed over two TLC plates (MERCK 1.05744.0001, PLC silica gel 60 F254, 0.5 mm; eluent: DCM/MeOH 92/8). The main bands (UV detection) were scratched off the plates. The silica gel/compound mixture was suspended in DCM/MeOH 85/15, treated with ultrasound, and filtered. The silica gel was rinsed with DCM/MeOH 85/15. The volatiles of the filtrate were removed under reduced pressure at 50° C., taken up in DCM/hexane, the volatiles were again removed under reduced pressure at 50° C. and the material was further dried under reduced pressure at 50° C. to give racemic 3-(4-(7-((5-cyclopropyl-7-methyl-1H-indol-4-yl)methyl)-2,2-difluoro-7-azaspiro[3.5]nonan-6-yl)phenyl)oxetan-3-ol (Example Ex-49) as an off-white solid. LC-MS Method B-1: Rt=0.58 min; MS m/z [M+H]⁺=493.4.

Enantiomer separation and analytics according to Method SFC-8 using 123 mg of the racemic 3-(4-(7-((5-cyclopropyl-7-methyl-1H-indol-4-yl)methyl)-2,2-difluoro-7-azaspiro[3.5]nonan-6-yl)phenyl)oxetan-3-ol, followed by removal of solvents under reduced pressure at 50° C., trituration with hexane+a few drops of DCM, followed by removal the solvents under reduced pressure at 50° C. (repeated 3×), and drying under reduced pressure at 50° C. yielded:

Peak 1: (99.5% ee), Example Ex-25, 54 mg, off-white powder. LC-MS Method B-1: Rt=0.60 min. MS m/z [M+H]⁺=493.3. Analytical chiral HPLC (Method SFC-8): Rt=1.65 min.

Peak 2: (99.5% ee), Example Ex-26, 52 mg, off-white powder. LC-MS Method B-1: Rt=0.62 min. MS m/z [M+H]⁺=493.3. Analytical chiral HPLC (Method SFC-8): Rt=3.15 min.

### Example Ex-57, Example Ex-33, and Example Ex-34

### 6-(4-(1H-tetrazol-5-yl)phenyl)-7-((5-cyclopropyl-7-methyl-1H-indol-4-yl)methyl)-2,2-difluoro-7-azaspiro[3.5]nonane, (S)-6-(4-(1H-tetrazol-5-yl)phenyl)-7-((5-cyclopropyl-7-methyl-1H-indol-4-yl)methyl)-2,2-difluoro-7-azaspiro[3.5]nonane, and (R)-6-(4-(1H-tetrazol-5-yl)phenyl)-7-((5-cyclopropyl-7-methyl-1H-indol-4-yl)methyl)-2,2-difluoro-7-azaspiro[3.5]nonane

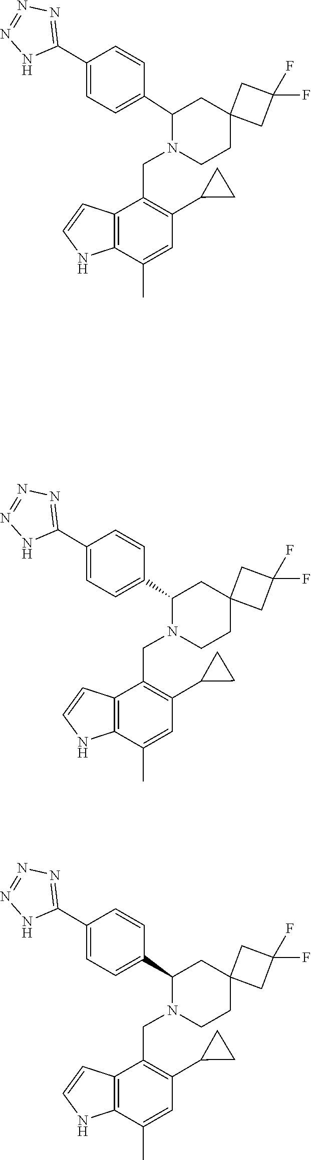 [SMILES: Cc1cc(C2CC2)c(CN2CCC3(CC2c2ccc(-c4nnn[nH]4)cc2)CC(F)(F)C3)c2cc[nH]c12 ; Cc1cc(C2CC2)c(CN2CCC3(C[C@H]2c2ccc(-c4nnn[nH]4)cc2)CC(F)(F)C3)c2cc[nH]c12 ; Cc1cc(C2CC2)c(CN2CCC3(C[C@@H]2c2ccc(-c4nnn[nH]4)cc2)CC(F)(F)C3)c2cc[nH]c12]

NaN₃ (42.20 mg, 649.1 μmol) and NH₄Cl (32.24 mg, 602.7 μmol) were added to a soln. of tert-butyl 4-((6-(4-cyanophenyl)-2,2-difluoro-7-azaspiro[3.5]nonan-7-yl)methyl)-5-cyclopropyl-7-methyl-1H-indole-1-carboxylate (Intermediate rac-A-29, 253.0 mg, 463.7 μmol) in DMF (2.50 mL). The capped vial was stirred at 100° C. overnight, then cooled to RT. The RM was diluted with MeOH (5.0 mL) and K₂CO₃ (325 mesh, 320.4 mg, 2.318 mmol) was added. The RM was stirred at 60° C. for 8 h, the RM was then kept without heating overnight. In order to quench the base, concentrated HCl (˜12M, 193.2 μL) was added. The MeOH was removed under reduced pressure at 50° C. The resulting suspension was filtered, and the solid was washed with MeOH. The filtrate soln. was directly purified in three runs by RP-HPLC (Waters Xbridge C18 Dimensions: 30 mm×100 mm 5 μm; flow 50 mL/min; eluent A=water+0.2% FA; eluent B=ACN; gradient: 15 to 39.7% B in 12 min; wavelength: 229/250 nm). Purest fractions were combined, and ACN was removed under reduced pressure at 45° C. The aq. soln. was lyophilized to give racemic 6-(4-(1H-tetrazol-5-yl)phenyl)-7-((5-cyclopropyl-7-methyl-1H-indol-4-yl)methyl)-2,2-difluoro-7-azaspiro[3.5]nonane (Example Ex-57, 107 mg) as a slightly red, fluffy solid. LC-MS Method B-1: Rt=0.66 min; MS m/z=489.3 [M+H]⁺.

Enantiomer separation and analytics according to Method SFC-13 using 96 mg of the racemic 6-(4-(1H-tetrazol-5-yl)phenyl)-7-((5-cyclopropyl-7-methyl-1H-indol-4-yl)methyl)-2,2-difluoro-7-azaspiro[3.5]nonane, followed by removal of solvents under reduced pressure at 50° C., trituration with hexane/DCM (2×), followed by removal the solvents under reduced pressure at 50° C., dissolving the sample in ACN/MeOH, followed by removal the solvents under reduced pressure at 50° C. and drying under reduced pressure at 50° C. yielded:

Peak 1: (99.5% ee), Example Ex-33, 38 mg, grey powder. LC-MS Method B-1: Rt=0.68 min. MS m/z [M+H]⁺=489.5. Analytical chiral HPLC (Method SFC-13): Rt=1.13 min.

Peak 2: (99.5% ee), Example Ex-34, 40 mg, grey, slightly green powder. LC-MS Method B-1: Rt=0.65 min. MS m/z [M+H]⁺=489.3. Analytical chiral HPLC (Method SFC-13): Rt=2.08 min.

The following examples were synthesized from the appropriate starting material by applying similar methods described in the examples above.

### Example Ex-37 and Example Ex-38

### (S)-2,2-difluoro-7-((5-methoxy-7-methyl-1H-indol-4-yl)methyl)-6-phenyl-7-azaspiro[3.5]nonane and (R)-2,2-difluoro-7-((5-methoxy-7-methyl-1H-indol-4-yl)methyl)-6-phenyl-7-azaspiro[3.5]nonane

Enantiomer separation and analytics according to Method SFC-18 of the racemic mixture Ex-provided:

Peak 1: (99.5% ee), Example Ex-37, 19.4 mg, off-white foam. LC-MS Method B-6: Rt=0.66 min. MS m/z [M+H]⁺=411.3. Analytical chiral HPLC (Method SFC-18): Rt=1.35 min.

Peak 2: (99.5% ee), Example Ex-38, 21.0 mg, off-white foam. LC-MS Method B-6: Rt=0.66 min. MS m/z [M+H]⁺=411.4. Analytical chiral HPLC (Method SFC-18): Rt=2.06 min.

### Example Ex-39 and Example Ex-40

### (S)-2,2-difluoro-7-((5-methoxy-7-methyl-1H-indol-4-yl)methyl)-6-(1-methyl-1H-pyrazol-4-yl)-7-azaspiro[3.5]nonane and (R)-2,2-difluoro-7-((5-methoxy-7-methyl-1H-indol-4-yl)methyl)-6-(1-methyl-1H-pyrazol-4-yl)-7-azaspiro[3.5]nonane

Enantiomer separation and analytics according to Method SFC-19 of the racemic mixture Ex-36 provided:

Peak 1: (99.5% ee), Example Ex-39, 35.2 mg, off-white foam. LC-MS Method B-6: Rt=0.49 min. MS m/z [M+H]⁺=415.1. Analytical chiral HPLC (Method SFC-19): Rt=1.31 min.

Peak 2: (99.5% ee), Example Ex-40, 34.6 mg, off-white foam. LC-MS Method B-6: Rt=0.49 min. MS m/z [M+H]⁺=415.1. Analytical chiral HPLC (Method SFC-19): Rt=1.82 min.

### Example Ex-41 and Example Ex-42

### (S)-6-(1-(difluoromethyl)-1H-pyrazol-4-yl)-2,2-difluoro-7-((5-methoxy-7-methyl-1H-indol-4-yl)methyl)-7-azaspiro[3.5]nonane and (R)-6-(1-(difluoromethyl)-1H-pyrazol-4-yl)-2,2-difluoro-7-((5-methoxy-7-methyl-1H-indol-4-yl)methyl)-7-azaspiro[3.5]nonane

### Intermediate 41-42: (RS)-6-(1-(difluoromethyl)-1H-pyrazol-4-yl)-2,2-difluoro-7-((5-methoxy-7-methyl-1H-indol-4-yl)methyl)-7-azaspiro[3.5]nonane

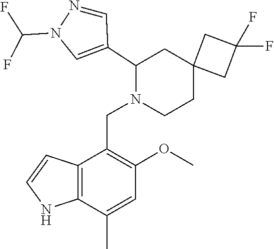 [SMILES: COc1cc(C)c2[nH]ccc2c1CN1CCC2(CC1c1cnn(C(F)F)c1)CC(F)(F)C2]

Racemic (RS)-6-(1-(difluoromethyl)-1H-pyrazol-4-yl)-2,2-difluoro-7-((5-methoxy-7-methyl-1H-indol-4-yl)methyl)-7-azaspiro[3.5]nonane was prepared similar to the method described for Intermediate rac-A-31 herein above, using Intermediate rac-P-21 and Intermediate I-1. LC-MS Method B-6: Rt=0.48 min; MS m/z [M+H]⁺=451.1

Enantiomer separation and analytics according to Method SFC-20 of the racemic Intermediate 41-42 provided:

Peak 1: (99.5% ee), Example Ex-41, 8.2 mg, yellow oil. LC-MS Method B-6: Rt=0.45 min. MS m/z [M+H]⁺=451.4. Analytical chiral HPLC (Method SFC-20): Rt=1.23 min.

Peak 2: (98.0% ee), Example Ex-42, 13.0 mg, yellow oil. LC-MS Method B-6: Rt=0.44 min. MS m/z [M+H]⁺=451.4. Analytical chiral HPLC (Method SFC-20): Rt=1.48 min.

### Example Ex-43 and Example Ex-44

### (R)-2,2-difluoro-7-((5-methoxy-7-methyl-1H-indol-4-yl)methyl)-6-(1-(2-methoxyethyl)-1H-pyrazol-4-yl)-7-azaspiro[3.5]nonane and (S)-2,2-difluoro-7-((5-methoxy-7-methyl-1H-indol-4-yl)methyl)-6-(1-(2-methoxyethyl)-1H-pyrazol-4-yl)-7-azaspiro[3.5]nonane

### Intermediate 43-44: (RS)-2,2-difluoro-7-((5-methoxy-7-methyl-1H-indol-4-yl)methyl)-6-(1-(2-methoxyethyl)-1H-pyrazol-4-yl)-7-azaspiro[3.5]nonane

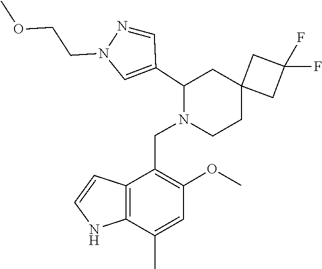 [SMILES: COCCn1cc(C2CC3(CCN2Cc2c(OC)cc(C)c4[nH]ccc24)CC(F)(F)C3)cn1]

Racemic (RS)-2,2-difluoro-7-((5-methoxy-7-methyl-1H-indol-4-yl)methyl)-6-(1-(2-methoxyethyl)-1H-pyrazol-4-yl)-7-azaspiro[3.5]nonane was prepared similar to the method described for Intermediate rac-A-31 herein above, using Intermediate rac-P-22 and Intermediate I-1. LC-MS Method B-6: Rt=0.46 min; MS m/z [M+H]⁺=459.4

Enantiomer separation and analytics according to Method SFC-21 of the racemic Intermediate 43-44 provided:

Peak 1: (99.5% ee), Example Ex-43, 71.2 mg, off-white foam. LC-MS Method B-6: Rt=0.48 min. MS m/z [M+H]⁺=459.5. Analytical chiral HPLC (Method SFC-21): Rt=1.05 min.

Peak 2: (99.4% ee), Example Ex-44, 72.9 mg, off-white foam. LC-MS Method B-6: Rt=0.46 min. MS m/z [M+H]⁺=459.2. Analytical chiral HPLC (Method SFC-21): Rt=1.59 min.

### Example Ex-54, Example Ex-55, and Example Ex-56

### (SR)-6-(4-(1H-tetrazol-5-yl)phenyl)-2,2-difluoro-7-((5-methoxy-7-methyl-1H-indol-4-yl)methyl)-7-azaspiro[3.5]nonane, (S)-6-(4-(1H-tetrazol-5-yl)phenyl)-2,2-difluoro-7-((5-methoxy-7-methyl-1H-indol-4-yl)methyl)-7-azaspiro[3.5]nonane, and (R)-6-(4-(1H-tetrazol-5-yl)phenyl)-2,2-difluoro-7-((5-methoxy-7-methyl-1H-indol-4-yl)methyl)-7-azaspiro[3.5]nonane

Racemic (SR)-6-(4-(1H-tetrazol-5-yl)phenyl)-2,2-difluoro-7-((5-methoxy-7-methyl-1H-indol-4-yl)methyl)-7-azaspiro[3.5]nonane was prepared similar to the method described for Example Ex-57 herein above, using Intermediate rac-P-18 and Intermediate I-1. Example Ex-54: LC-MS Method B-6: Rt=0.49 min; MS m/z [M−H]⁻=477.2

Enantiomer separation and analytics according to Method SFC-23 of the racemic mixture Ex-54 provided:

Peak 1: (99.5% ee), Example Ex-55, 61.9 mg, off-white solid. LC-MS Method B-6: Rt=0.52 min. MS m/z [M+H]⁺=479.5. Analytical chiral HPLC (Method SFC-23): Rt=1.03 min.

Peak 2: (99.5% ee), Example Ex-56, 63.6 mg, off-white solid. LC-MS Method B-6: Rt=0.55 min. MS m/z [M+H]⁺=479.1. Analytical chiral HPLC (Method SFC-23): Rt=1.60 min.

### Example 58, Example Ex-59, and Example Ex-60

### (SR)-6-(6-(1H-tetrazol-5-yl)pyridin-3-yl)-2,2-difluoro-7-((5-methoxy-7-methyl-1H-indol-4-yl)methyl)-7-azaspiro[3.5]nonane, (S)-6-(6-(1H-tetrazol-5-yl)pyridin-3-yl)-2,2-difluoro-7-((5-methoxy-7-methyl-1H-indol-4-yl)methyl)-7-azaspiro[3.5]nonane, and (R)-6-(6-(1H-tetrazol-5-yl)pyridin-3-yl)-2,2-difluoro-7-((5-methoxy-7-methyl-1H-indol-4-yl)methyl)-7-azaspiro[3.5]nonane

Racemic (SR)-6-(6-(1H-tetrazol-5-yl)pyridin-3-yl)-2,2-difluoro-7-((5-methoxy-7-methyl-1H-indol-4-yl)methyl)-7-azaspiro[3.5]nonane was prepared similar to the method described for Example Ex-57 herein above, using Intermediate rac-P-26 and Intermediate I-1. Example Ex-58: LC-MS Method B-6: Rt=0.43 min; MS m/z [M+H]⁺=480.2

Enantiomer separation and analytics according to Method SFC-24 of the racemic mixture Ex-58 provided:

Peak 1: (99.5% ee), Example Ex-59, 57.8 mg, off-white powder. LC-MS Method B-6: Rt=0.46 min. MS m/z [M+H]⁺=480.1. Analytical chiral HPLC (Method SFC-24): Rt=1.09 min.

Peak 2: (99.5% ee), Example Ex-60, 55.0 mg, off-white powder. LC-MS Method B-6: Rt=0.43 min. MS m/z [M+H]⁺=480.5. Analytical chiral HPLC (Method SFC-24): Rt=2.08 min.

### Example Ex-61, Example Ex-62, and Example Ex-63

### (SR)-6-(6-(1H-tetrazol-5-yl)pyridin-3-yl)-7-((5-cyclopropyl-7-methyl-1H-indol-4-yl)methyl)-2,2-difluoro-7-azaspiro[3.5]nonane, (S)-6-(6-(1H-tetrazol-5-yl)pyridin-3-yl)-7-((5-cyclopropyl-7-methyl-1H-indol-4-yl)methyl)-2,2-difluoro-7-azaspiro[3.5]nonane, and (R)-6-(6-(1H-tetrazol-5-yl)pyridin-3-yl)-7-((5-cyclopropyl-7-methyl-1H-indol-4-yl)methyl)-2,2-difluoro-7-azaspiro[3.5]nonane

Racemic (SR)-6-(6-(1H-tetrazol-5-yl)pyridin-3-yl)-7-((5-cyclopropyl-7-methyl-1H-indol-4-yl)methyl)-2,2-difluoro-7-azaspiro[3.5]nonane was prepared similar to the method described for Example Ex-57 herein above, using Intermediate rac-P-26 and Intermediate I-3. Example Ex-61: LC-MS Method B-6: Rt=0.73 min; MS m/z [M+H]⁺=490.2

Enantiomer separation and analytics according to Method SFC-25 of the racemic mixture Example Ex-61 provided:

Peak 1: (99.5% ee), Example Ex-62, 58.3 mg, amber solid. LC-MS Method B-6: Rt=0.73 m r. MS m/z [M+H]=490.2. Analytical chiral HPLC (Method SFC-25): Rt=1.29 m.

Peak 2: (99.5% ee), Example Ex-63, 57.3 mg, off-white solid. LC-MS Method B-6: Rt=0.74 mA. MS m z [M+H]=490.3. Analytical chiral HPLC (Method SFC-25): Rt=3.35 m.

The representative analytical data of exemplified compounds are summarized below. The reported coupling patterns are apparent coupling patterns as provided by MNova NMR software.

| | | | | | Stereo- |
|---|---|---|---|---|---|
| | Starting | | | LC-MS | chemical |
| | material | Chemical Structure | NMR | Method | Information |
| Ex-1 | Intermediate A-3 | 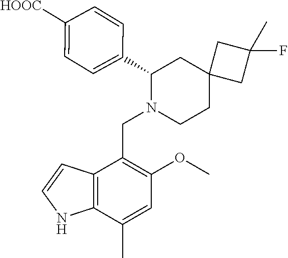 [SMILES: COc1cc(C)c2[nH]ccc2c1CN1CCC2(C[C@H]1c1ccc(C(=O)O)cc1)CC(C)(F)C2] | ¹H NMR (TFA salt, 400 MHz, DMSO-d₆) δ [ppm] 13.22 (s, 1H), 11.19 (t, J = 2.4 Hz, 1H), 8.93 (q, J = 8.2 Hz, 1H), 8.14 (d, J = 7.9 Hz, 2H), 7.82 (d, J = 7.9 Hz, 2H), 7.42 (t, J = 2.8 Hz, 1H), 6.77 (s, 1H), 6.36 (t, J = 2.5 Hz, 1H), 4.53 (t, J = 10.9 Hz, 1H), 4.21 (dd, J = 12.7, 8.6 Hz, 1H), 4.06 (d, J = 12.6 Hz, 1H), 3.68 (s, 3H), 3.44 − 3.28 (m, 1H), 3.22 (d, J = 11.3 Hz, 1H), 2.48 (s, 3H), 2.35 (ddd, J = 29.7, 13.6, 9.1 Hz, 2H), 2.05 (dq, J = 38.7, 13.8 Hz, 5H), 1.84 (d, J = | Method B-1: Rt = 0.57 min; MS [M + H]⁺ = 451.4 | *) single stereoisomer, (partially) unknown configuration |
| | | | 14.2 Hz, 1H), 1.44 (d, J = | | |
| | | | 22.7 Hz, 3H) | | |
| | | | 32 of 32H reported | | |
| Ex-2 | Intermediate A-6 | 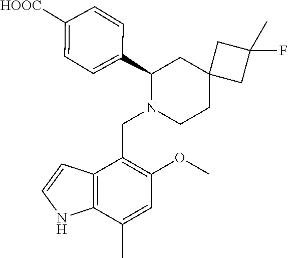 [SMILES: COc1cc(C)c2[nH]ccc2c1CN1CCC2(C[C@@H]1c1ccc(C(=O)O)cc1)CC(C)(F)C2] | ¹H NMR (TFA salt, 400 MHz, DMSO-d₆) δ [ppm] 13.22 (s, 1H), 11.19 (s, 1H), 8.94 (q, J = 8.1 Hz, 1H), 8.14 (d, J = 7.9 Hz, 2H), 7.83 (d, J = 8.5 Hz, 2H), 7.42 (t, J = 2.8 Hz, 1H), 6.77 (s, 1H), 6.38 (s, 1H), 4.58 (t, J = 10.7 Hz, 1H), 4.22 (dd, J = 12.7, 8.6 Hz, 1H), 4.07 (d, J = 12.5 Hz, 1H), 3.69 (s, 3H), 3.36 − 3.23 (m, 1H), 3.19 (d, J = 12.4 Hz, 1H), 2.48 (s, 3H), 2.46 − 2.40 (m, 1H), 2.31 (t, J = 14.8 Hz, 1H), 2.25 − 2.08 (m, 2H), 2.01 (t, J = 14.4 Hz, 2H), 1.95 − 1.85 | Method B-1: Rt = 0.57 min; MS m/z [M + H]⁺ = 451.4 | * |
| | | | (m, 1H), 1.79 (d, J = 14.2 Hz, | | |
| | | | 1H), 1.46 (d, J = 22.8 Hz, 3H) | | |
| | | | 32 of 32H reported | | |
| Ex-3 | Intermediate A-5 | 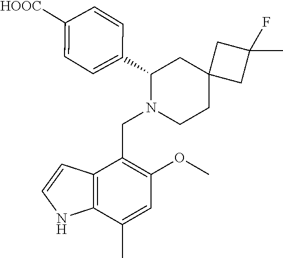 [SMILES: COc1cc(C)c2[nH]ccc2c1CN1CCC2(C[C@H]1c1ccc(C(=O)O)cc1)CC(C)(F)C2] | ¹H NMR (TFA salt, 400 MHz, DMSO-d₆) δ [ppm] 13.22 (s, 1H), 11.19 (t, J = 2.3 Hz, 1H), 9.36 − 8.74 (m, 1H), 8.14 (d, J = 7.9 Hz, 2H), 7.83 (d, J = 8.7 Hz, 2H), 7.42 (t, J = 2.8 Hz, 1H), 6.77 (s, 1H), 6.38 (t, J = 2.4 Hz, 1H), 4.58 (t, J = 10.9 Hz, 1H), 4.22 (dd, J = 12.7, 8.6 Hz, 1H), 4.07 (d, J = 12.5 Hz, 1H), 3.69 (s, 3H), 3.36 − 3.24 (m, 1H), 3.19 (d, J = 12.3 Hz, 1H), 2.48 (s, 3H), 2.46 − 2.39 (m, 1H), 2.31 (t, J = 14.9 Hz, 1H), 2.25 − 2.08 (m, 2H), 2.01 (t, J = 14.4 Hz, | Method B-1: Rt = 0.60 min; MS m/z [M + H]⁺ = 451.2 | *) |
| | | | 2H), 1.96 − 1.86 (m, 1H), | | |
| | | | 1.85 − 1.74 (m, 1H), 1.46 (d, | | |
| | | | J = 22.8 Hz, 3H) | | |
| | | | 32 of 32H reported | | |
| Ex-4 | Intermediate A-4 | 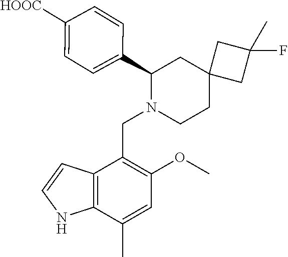 [SMILES: COc1cc(C)c2[nH]ccc2c1CN1CCC2(C[C@@H]1c1ccc(C(=O)O)cc1)CC(C)(F)C2] | ¹H NMR (TFA salt, 400 MHz, DMSO-d₆) δ [ppm] 13.19 (s, 1H), 11.19 (t, J = 2.3 Hz, 1H), 9.26 − 9.02 (m, 1H), 8.13 (d, J = 8.3 Hz, 2H), 7.85 (d, J = 8.6 Hz, 2H), 7.41 (t, J = 2.8 Hz, 1H), 6.76 (s, 1H), 6.37 (dd, J = 3.2, 1.8 Hz, 1H), 4.59 − 4.46 (m, 1H), 4.21 (dd, J = 12.7, 8.6 Hz, 1H), 4.06 (d, J = 12.5 Hz, 1H), 3.69 (s, 3H), 3.34 (q, J = 11.5 Hz, 1H), 3.21 (d, J = 12.5 Hz, 1H), 2.48 (s, 3H), 2.43 − 2.26 (m, 2H), 2.20 − 2.01 (m, 4H), 1.96 (d, J = 14.2 Hz, 1H), | Method B-1: Rt = 0.55 min; MS m/z [M + H]⁺ = 451.4 | *) |
| | | | 1.83 (d, J = 14.0 Hz, 1H), | | |
| | | | 1.43 (d, J = 22.7 Hz, 3H) | | |
| | | | 32 of 32H reported | | |
| Ex-5 | Intermediate A-7 | 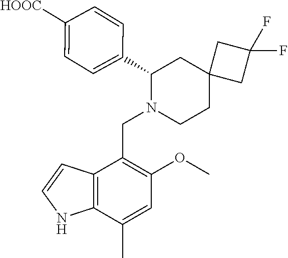 [SMILES: COc1cc(C)c2[nH]ccc2c1CN1CCC2(C[C@H]1c1ccc(C(=O)O)cc1)CC(F)(F)C2] | ¹H NMR (400 MHz, DMSO- d₆) δ [ppm] 12.83 (s, 1H), 10.81 (s, 1H), 7.97 (d, J = 8.2 Hz, 2H), 7.68 (d, J = 7.8 Hz, 2H), 7.25 (t, J = 2.8 Hz, 1H), 6.65 (s, 1H), 6.45 (dd, J = 3.1, 1.9 Hz, 1H), 3.70 (s, 3H), 3.53 (d, J = 11.8 Hz, 1H), 3.22 (dd, J = 9.6, 4.6 Hz, 1H), 3.16 (d, J = 11.9 Hz, 1H), 2.67 (d, J = 12.0 Hz, 1H), 2.58 (t, J = 13.2 Hz, 1H), 2.48 − 2.42 (m, 1H), 2.42 − 2.37 (m, 3H), 2.29 (t, J = 13.3 Hz, 2H), 2.07 − 1.92 (m, 1H), 1.81 − 1.62 (m, 2H), 1.59 − | Method B-3: Rt = 2.34 min; MS m/z [M + H]⁺ = 455.2 | |
| | | | 1.46 (m, 2H) | | |
| | | | 28 of 28H reported | | |
| Ex-6 | Intermediate A-8 | 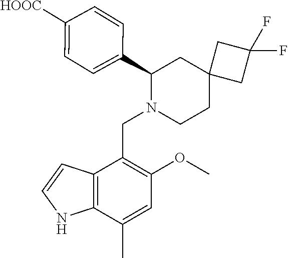 [SMILES: COc1cc(C)c2[nH]ccc2c1CN1CCC2(C[C@@H]1c1ccc(C(=O)O)cc1)CC(F)(F)C2] | ¹H NMR (400 MHz, DMSO- d₆) δ [ppm] 12.86 (s, 1H), 10.82 (s, 1H), 7.97 (d, J = 7.9 Hz, 2H), 7.68 (d, J = 7.8 Hz, 2H), 7.25 (t, J = 2.8 Hz, 1H), 6.65 (s, 1H), 6.45 (d, J = 2.6 Hz, 1H), 3.70 (s, 3H), 3.53 (d, J = 11.8 Hz, 1H), 3.21 (dd, J = 9.2, 4.6 Hz, 1H), 3.16 (d, J = 11.9 Hz, 1H), 2.67 (d, J = 11.9 Hz, 1H), 2.58 (t, J = 13.3 Hz, 1H), 2.49 − 2.45 (m, 1H), 2.41 (s, 3H), 2.29 (t, J = 13.3 Hz, 2H), 2.03 − 1.88 (m, 1H), 1.80 − 1.64 (m, 2H), 1.59 − 1.43 (m, 2H) | Method B-3: Rt = 2.30 min; MS m/z [M + H]⁺ = 455.4 | |
| | | | 28 of 28H reported | | |
| Ex-7 | Intermediate A-9 | 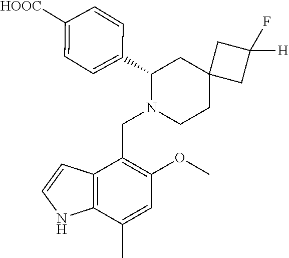 [SMILES: COc1cc(C)c2[nH]ccc2c1CN1CCC2(CC(F)C2)C[C@H]1c1ccc(C(=O)O)cc1] | ¹H NMR (TFA salt, 400 MHz, DMSO-d₆) δ [ppm] 13.23 (s, 1H), 11.19 (t, J = 2.3 Hz, 1H), 9.03 − 8.84 (m, 1H), 8.14 (d, J = 7.9 Hz, 2H), 7.82 (d, J = 7.7 Hz, 2H), 7.42 (t, J = 2.8 Hz, 1H), 6.77 (s, 1H), 6.44 − 6.35 (m, 1H), 5.28 − 4.96 (m, 1H), 4.55 (t, J = 10.9 Hz, 1H), 4.22 (dd, J = 12.7, 8.7 Hz, 1H), 4.06 (d, J = 12.5 Hz, 1H), 3.69 (s, 3H), 3.35 (q, J = 11.5 Hz, 1H), 3.19 (d, J = 12.6 Hz, 1H), 2.71 − 2.56 (m, 1H), 2.47 (s, 3H), 2.29 − 2.13 (m, 3H), 2.09 − 1.84 (m, 3H), | Method B-1: Rt = 0.48 min; MS m/z [M + H]⁺ = 437.6 | * |
| | | | 1.71 (d, J = 14.1 Hz, 1H) | | |
| | | | 30 of 30H reported | | |
| Ex-8 | Intermediate A-12 | 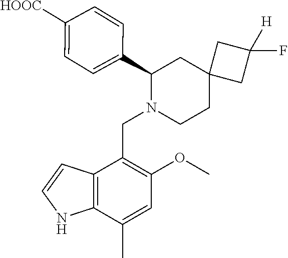 [SMILES: COc1cc(C)c2[nH]ccc2c1CN1CCC2(CC(F)C2)C[C@@H]1c1ccc(C(=O)O)cc1] | ¹H NMR (TFA salt, 400 MHz, DMSO-d₆) δ [ppm] 13.21 (s, 1H), 11.19 (t, J = 2.3 Hz, 1H), 9.07 − 8.74 (m, 1H), 8.14 (d, J = 8.0 Hz, 2H), 7.83 (d, J = 6.7 Hz, 2H), 7.42 (t, J = 2.8 Hz, 1H), 6.77 (s, 1H), 6.34 (t, J = 2.4 Hz, 1H), 5.13 (m, 1H), 4.60 (t, J = 10.6 Hz, 1H), 4.22 (dd, J = 12.8, 8.6 Hz, 1H), 4.06 (d, J = 12.5 Hz, 1H), 3.69 (s, 3H), 3.30 (q, J = 12.0 Hz, 1H), 3.19 (d, J = 12.3 Hz, 1H), 2.82 − 2.69 (m, 1H), 2.48 (s, 3H), 2.29 − 2.16 (m, 1H), 2.16 − 1.91 (m, 5H), | Method B-1: Rt = 0.44 min; MS m/z [M + H]⁺ = 437.5 | *) |
| | | | 1.77 (d, J = 13.9 Hz, 1H) | | |
| | | | 30 of 30H reported | | |
| Ex-9 | Intermediate A-11 | 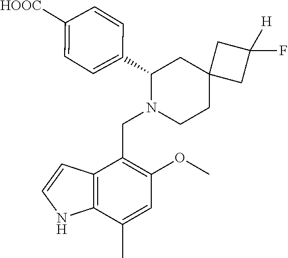 [SMILES: COc1cc(C)c2[nH]ccc2c1CN1CCC2(CC(F)C2)C[C@H]1c1ccc(C(=O)O)cc1] | ¹H NMR (TFA salt, 400 MHz, DMSO-d₆) δ [ppm] 13.22 (s, 1H), 11.19 (t, J = 2.2 Hz, 1H), 9.07 − 8.79 (m, 1H), 8.14 (d, J = 7.9 Hz, 2H), 7.82 (d, J = 7.8 Hz, 2H), 7.42 (t, J = 2.8 Hz, 1H), 6.77 (s, 1H), 6.41 − 6.27 (m, 1H), 5.13 (m, 1H), 4.60 (t, J = 10.2 Hz, 1H), 4.22 (dd, J = 12.8, 8.6 Hz, 1H), 4.06 (d, J = 12.5 Hz, 1H), 3.69 (s, 3H), 3.30 (q, J = 11.6 Hz, 1H), 3.20 (d, J = 12.3 Hz, 1H), 2.82 − 2.69 (m, 1H), 2.48 (s, 3H), 2.29 − 2.17 (m, 1H), 2.17 − 1.89 (m, 5H), | Method B-1: Rt = 0.48 min; MS m/z [M + H]⁺ = 437.4 | *) |
| | | | 1.77 (d, J = 14.1 Hz, 1H) | | |
| | | | 30 of 30H reported | | |
| Ex-10 | Intermediate A-10 | 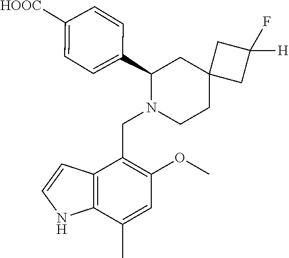 [SMILES: COc1cc(C)c2[nH]ccc2c1CN1CCC2(CC(F)C2)C[C@@H]1c1ccc(C(=O)O)cc1] | ¹H NMR (TFA salt, 400 MHz, DMSO-d₆) δ [ppm] 13.22 (s, 1H), 11.19 (t, J = 2.3 Hz, 1H), 9.13 − 8.84 (m, 1H), 8.14 (d, J = 8.0 Hz, 2H), 7.82 (d, J = 8.0 Hz, 2H), 7.42 (t, J = 2.8 Hz, 1H), 6.76 (s, 1H), 6.54 − 6.32 (m, 1H), 5.10 (m, 1H), 4.55 (t, J = 11.0 Hz, 1H), 4.22 (dd, J = 12.7, 8.8 Hz, 1H), 4.06 (d, J = 12.5 Hz, 1H), 3.69 (s, 3H), 3.36 − 3.27 (m, 1H), 3.19 (d, J = 12.6 Hz, 1H), 2.76 − 2.58 (m, 1H), 2.47 (s, 3H), 2.29 − 2.13 (m, 3H), 2.11 − 1.84 (m, 3H), 1.71 (d, | Method B-1: Rt = 0.54 min; MS m/z [M + H]⁺ = 437.4 | *) |
| | | | J = 14.1 Hz, 1H) | | |
| | | | 30 of 30H reported | | |
| Ex-11 | Intermediate A-14 | 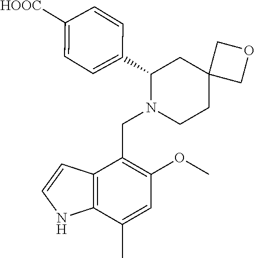 [SMILES: COc1cc(C)c2[nH]ccc2c1CN1CCC2(COC2)C[C@H]1c1ccc(C(=O)O)cc1] | ¹H NMR (400 MHz, DMSO- d₆) δ [ppm] 12.86 (s, 1H), 10.82 (d, J = 2.7 Hz, 1H), 7.98 (d, J = 8.5 Hz, 2H), 7.68 (d, J = 7.8 Hz, 2H), 7.24 (t, J = 2.8 Hz, 1H), 6.64 (s, 1H), 6.43 (dd, J = 3.0, 1.9 Hz, 1H), 4.53 (d, J = 5.8 Hz, 1H), 4.41 (d, J = 5.7 Hz, 1H), 4.24 − 4.13 (m, 2H), 3.69 (s, 3H), 3.52 (d, J = 11.9 Hz, 1H), 3.12 (t, J = 10.5 Hz, 2H), 2.73 − 2.62 (m, 1H), 2.41 (s, 3H), 2.13 − 2.03 (m, 1H), 1.94 − 1.78 (m, 2H), 1.65 (t, J = 12.3 Hz, 1H), 1.52 − 1.40 (m, 1H) | Method B-3: Rt = 1.55 min; MS m/z [M + H]⁺ = 421.5 | |
| | | | 28 of 28H reported | | |
| Ex-12 | Intermediate A-13 | 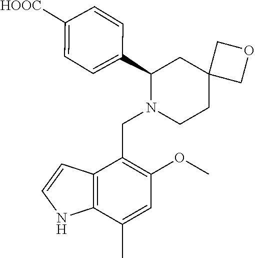 [SMILES: COc1cc(C)c2[nH]ccc2c1CN1CCC2(COC2)C[C@@H]1c1ccc(C(=O)O)cc1] | ¹H NMR (400 MHz, DMSO- d₆) δ [ppm] 12.86 (s, 1H), 10.82 (s, 1H), 7.98 (d, J = 7.9 Hz, 2H), 7.69 (d, J = 7.8 Hz, 2H), 7.24 (t, J = 2.8 Hz, 1H), 6.65 (s, 1H), 6.43 (t, J = 2.5 Hz, 1H), 4.53 (d, J = 5.7 Hz, 1H), 4.41 (d, J = 5.7 Hz, 1H), 4.19 (t, J = 4.4 Hz, 2H), 3.69 (s, 3H), 3.51 (d, J = 11.8 Hz, 1H), 3.12 (t, J = 10.3 Hz, 2H), 2.67 (d, J = 12.2 Hz, 1H), 2.41 (s, 3H), 2.08 (d, J = 9.5 Hz, 1H), 1.97 − 1.79 (m, 2H), 1.65 (t, J = 12.4 Hz, 1H), 1.46 (t, J = 12.7 Hz, 1H) | Method B-3: Rt = 1.52 min; MS m/z [M + H]⁺ = 421.3 | |
| | | | 28 of 28H reported | | |
| Ex-13 | Intermediate rac-A-1 | 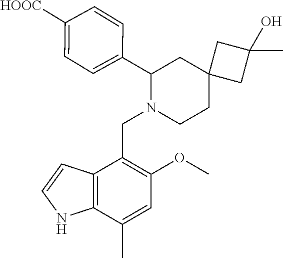 [SMILES: COc1cc(C)c2[nH]ccc2c1CN1CCC2(CC1c1ccc(C(=O)O)cc1)CC(C)(O)C2] | ¹H NMR (TFA salt, 400 MHz, DMSO-d₆) δ [ppm] 13.22 (s, 1H), 11.18 (t, J = 2.3 Hz, 1H), 9.12 − 8.80 (m, 1H), 8.14 (d, J = 7.8 Hz, 2H), 7.83 (d, J = 8.5 Hz, 2H), 7.42 (t, J = 2.8 Hz, 1H), 6.76 (s, 1H), 6.36 (dd, J = 3.1, 1.8 Hz, 1H), 4.75 (s, 1H), 4.61 − 4.50 (m, 1H), 4.21 (dd, J = 12.7, 8.7 Hz, 1H), 4.06 (d, J = 12.5 Hz, 1H), 3.69 (s, 3H), 3.32 − 3.14 (m, 2H), 2.48 (s, 3H), 2.20 − 1.99 (m, 4H), 1.96 − 1.84 (m, 1H), 1.83 (s, 2H), 1.74 (d, J = 13.9 Hz, 1H), 1.25 (s, 3H) | Method B-1: Rt = 0.66 min; MS m/z [M + H]⁺ = 449.2 | Racemate |
| | | | 33 of 33H reported | | |
| Ex- 14 | Intermediate A-15 | 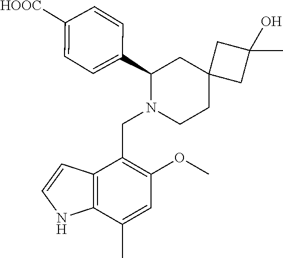 [SMILES: COc1cc(C)c2[nH]ccc2c1CN1CCC2(C[C@@H]1c1ccc(C(=O)O)cc1)CC(C)(O)C2] | ¹H NMR (TFA salt, 400 MHz, DMSO-d₆) δ [ppm] 13.20 (s, 1H), 11.18 (t, J = 2.3 Hz, 1H), 9.10 − 8.75 (m, 1H), 8.13 (d, J = 8.4 Hz, 2H), 7.82 (d, J = 8.3 Hz, 2H), 7.42 (t, J = 2.8 Hz, 1H), 6.76 (s, 1H), 6.36 (t, J = 2.2 Hz, 1H), 4.80 (s, 1H), 4.49 (t, J = 10.7 Hz, 1H), 4.21 (dd, J = 12.7, 8.7 Hz, 1H), 4.05 (d, J = 12.5 Hz, 1H), 3.69 (s, 3H), 3.36 − 3.25 (m, 1H), 3.20 (d, J = 11.9 Hz, 1H), 2.48 (s, 3H), 2.18 (d, J = 12.3 Hz, 1H), 2.04 (t, J = 13.5 Hz, 2H), 1.96 − 1.85 (m, 3H), | Method B-1: Rt = 0.38 min; MS m/z [M + H]⁺ = 449.4 | *) |
| | | | 1.85 − 1.74 (m, 2H), 1.23 (s, 3H) | | |
| | | | 33 of 33H reported | | |
| Ex-15 | Intermediate A-16 | 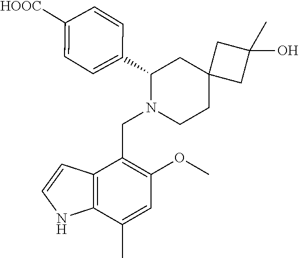 [SMILES: COc1cc(C)c2[nH]ccc2c1CN1CCC2(C[C@H]1c1ccc(C(=O)O)cc1)CC(C)(O)C2] | ¹H NMR (TFA salt, 400 MHz, DMSO-d₆) δ [ppm] 13.20 (s, 1H), 11.18 (t, J = 2.3 Hz, 1H), 8.89 (d, J = 9.4 Hz, 1H), 8.13 (d, J = 7.6 Hz, 2H), 7.82 (d, J = 8.4 Hz, 2H), 7.42 (t, J = 2.8 Hz, 1H), 6.76 (s, 1H), 6.36 (dd, J = 3.2, 1.8 Hz, 1H), 4.77 (s, 1H), 4.49 (t, J = 10.8 Hz, 1H), 4.21 (dd, J = 12.8, 8.7 Hz, 1H), 4.05 (d, J = 12.5 Hz, 1H), 3.69 (s, 3H), 3.36 − 3.24 (m, 1H), 3.20 (d, J = 12.0 Hz, 1H), 2.48 (s, 3H), 2.18 (d, J = 12.3 Hz, 1H), 2.04 (t, J = 13.3 Hz, 2H), 1.97 − 1.85 (m, 3H), | Method B-1: Rt = 0.45 min; MS m/z [M + H]⁺ = 449.2 | *) |
| | | | 1.85 − 1.80 (m, 2H), 1.23 (s, 3H) | | |
| | | | 33 of 33H reported | | |
| Ex-16 | Intermediate rac-A-2 | 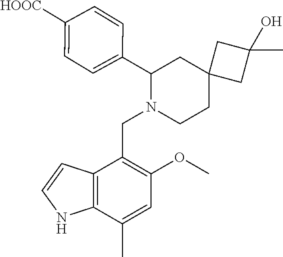 [SMILES: COc1cc(C)c2[nH]ccc2c1CN1CCC2(CC1c1ccc(C(=O)O)cc1)CC(C)(O)C2] | ¹H NMR (400 MHz, DMSO- d₆) δ [ppm] 12.83 (s, 1H), 10.81 (s, 1H), 7.96 (d, J = 7.8 Hz, 2H), 7.76 − 7.56 (m, 2H), 7.24 (s, 1H), 6.65 (s, 1H), 6.44 (s, 1H), 4.70 (s, 1H), 3.69 (s, 3H), 3.50 (d, J = 11.6 Hz, 1H), 3.22 − 3.01 (m, 2H), 2.65 − 2.55 (m, 1H), 2.41 (s, 3H), 2.04 (d, J = 11.9 Hz, 1H), 1.99 − 1.90 (m, 1H), 1.86 (d, J = 11.6 Hz, 1H), 1.81 − 1.68 (m, 2H), 1.68 − 1.47 (m, 3H), 1.47 − 1.30 (m, 1H), 1.20 (s, 3H) 32 of 32H reported | Method B-1: Rt = 0.45 min; MS m/z [M + H]⁺ = 449.4 | Racemate |
| Ex-17 | Intermediate A-17 | 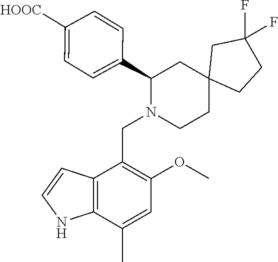 [SMILES: COc1cc(C)c2[nH]ccc2c1CN1CCC2(CCC(F)(F)C2)C[C@@H]1c1ccc(C(=O)O)cc1] | ¹H NMR (400 MHz, DMSO- d₆) δ [ppm] 10.80 (s, 1H), 7.89 (d, J = 7.9 Hz, 2H), 7.52 (d, J = 7.9 Hz, 2H), 7.28 − 7.20 (m, 1H), 6.64 (s, 1H), 6.46 (d, J = 2.9 Hz, 1H), 3.70 (s, 3H), 3.54 (d, J = 11.9 Hz, 1H), 3.30 − 3.25 (m, 1H* under water peak), 3.18 (d, J = 11.9 Hz, 1H), 2.64 − 2.55 (m, 1H), 2.41 (s, 3H), 2.23 − 2.06 (m, 3H), 1.95 − 1.75 (m, 4H), 1.66 − 1.58 (m, 2H), 1.51 − 1.41 (m, 2H) 29 of 30H reported | Method C-1: Rt = 0.74 min; MS m/z [M + H]⁺ = 469.3 | *) |
| Ex-18 | Intermediate A-18 | 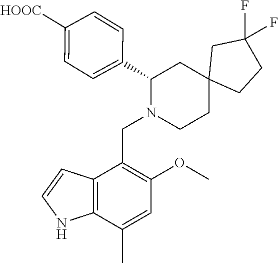 [SMILES: COc1cc(C)c2[nH]ccc2c1CN1CCC2(CCC(F)(F)C2)C[C@H]1c1ccc(C(=O)O)cc1] | ¹H NMR (400 MHz, DMSO- d₆) δ [ppm] 10.80 (s, 1H), 7.86 (d, J = 7.8 Hz, 2H), 7.46 (d, J = 7.7 Hz, 2H), 7.26 − 7.21 (m, 1H), 6.64 (s, 1H), 6.48 (d, J = 2.6 Hz, 1H), 3.70 (s, 3H), 3.55 (d, J = 11.9 Hz, 1H), 3.20 − 3.14 (m, 2H* partially under water peak), 2.70 − 2.60 (m, 1H), 2.41 (s, 3H), 2.29 − 1.95 (m, 5H), 1.66 − 1.51 (m, 4H), 1.49 − 1.32 (m, 2H) 29 of 30H reported | Method C-1: Rt = 0.74 min; MS m/z [M + H]⁺ = 469.4 | *) |
| Ex-19 | Intermediate A-19 | 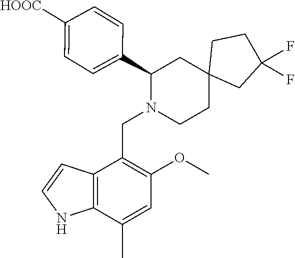 [SMILES: COc1cc(C)c2[nH]ccc2c1CN1CCC2(CCC(F)(F)C2)C[C@@H]1c1ccc(C(=O)O)cc1] | ¹H NMR (400 MHz, DMSO- d₆) δ [ppm] 10.80 (s, 1H), 7.86 (d, J = 7.8 Hz, 2H), 7.47 (d, J = 7.7 Hz, 2H), 7.32 − 7.19 (m, 1H), 6.64 (s, 1H), 6.54 − 6.42 (m, 1H), 3.70 (s, 3H), 3.55 (d, J = 11.9 Hz, 1H), 3.20 − 3.15 (m, 2H* under water peak), 2.69 − 2.60 (m, 1H), 2.41 (s, 3H), 2.28 − 1.95 (m, 5H), 1.65 − 1.51 (m, 4H), 1.49 − 1.32 (m, 2H) 29 of 30H reported | Method C-1: Rt = 0.76 min; MS m/z [M + H]⁺ = 469.3 | *) |
| Ex-20 | Intermediate A-20 | 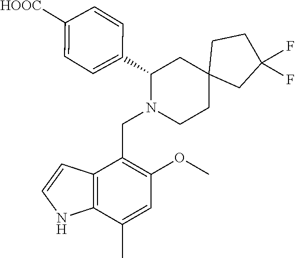 [SMILES: COc1cc(C)c2[nH]ccc2c1CN1CCC2(CCC(F)(F)C2)C[C@H]1c1ccc(C(=O)O)cc1] | ¹H NMR (400 MHz, DMSO- d₆) δ [ppm] 10.79 (s, 1H), 7.86 (d, J = 7.8 Hz, 2H), 7.45 (d, J = 7.7 Hz, 2H), 7.27 − 7.19 (m, 1H), 6.63 (s, 1H), 6.46 (d, J = 2.9 Hz, 1H), 3.70 (s, 3H), 3.54 (d, J = 11.9 Hz, 1H), 3.26 − 3.21 (m, 1H* partially under water/solvent peak), 3.17 (d, J = 11.9 Hz, 1H), 2.63 − 2.55 (m, 1H), 2.41 (s, 3H), 2.23 − 2.03 (m, 3H), 1.96 − 1.74 (m, 4H), 1.68 − 1.57 (m, 2H), 1.49 − 1.40 (m, 2H) 29 of 30H reported | Method C-1: Rt = 0.75 min; MS m/z [M + H]⁺ = 469.3 | *) |
| Ex-21 | Intermediate A-21 | 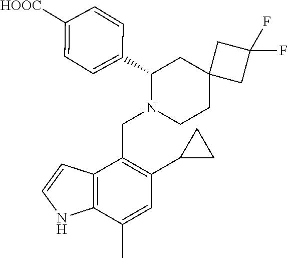 [SMILES: Cc1cc(C2CC2)c(CN2CCC3(C[C@H]2c2ccc(C(=O)O)cc2)CC(F)(F)C3)c2cc[nH]c12] | ¹H NMR (400 MHz, DMSO- d₆) δ [ppm] 12.87 (br s, 1H), 10.83 (br s, 1H), 7.94 (d, J = 8.4 Hz, 2H), 7.64 (d, J = 8.0 Hz, 2H), 7.23 (t, J = 2.8 Hz, 1H), 6.55 (dd, J = 1.9, 3.0 Hz, 1H), 6.45 (s, 1H), 3.72 (d, J = 12.0 Hz, 1H), 3.28 − 3.22 (m, 2H), 2.67 − 2.53 (m, 2H), 2.48 − 2.39 (m, 1H), 2.35 (s, 3H), 2.34 − 2.26 (m, 2H), 2.19 − 2.08 (m, 1H), 2.07 − 1.96 (m, 1H), 1.83 (dd, J = 11.5, 13.2 Hz, 1H), 1.70 (d, J = 13.9 Hz, 1H), 1.60 − 1.48 (m, 2H), 0.86 − 0.76 (m, 1H), | Method B-3: Rt = 3.27 min; MS m/z [M + H]⁺ = 465.4 | |
| | | | 0.72 − 0.62 (m, 1H), 0.56 − | | |
| | | | 0.46 (m, 1H), 0.20 − 0.11 (m, | | |
| | | | 1H) | | |
| | | | 30 of 30H reported | | |
| Ex-22 | Intermediate A-22 | 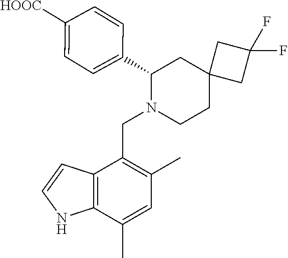 [SMILES: Cc1cc(C)c2[nH]ccc2c1CN1CCC2(C[C@H]1c1ccc(C(=O)O)cc1)CC(F)(F)C2] | ¹H NMR (400 MHz, DMSO- d₆) δ [ppm] 15.33 − 11.28 (m, 1H), 10.83 (s, 1H), 7.96 (d, J = 8.4 Hz, 2H), 7.64 (br d, J = 7.8 Hz, 2H), 7.22 (t, J = 2.8 Hz, 1H), 6.64 (s, 1H), 6.50 (dd, J = 1.9, 2.9 Hz, 1H), 3.53 (d, J = 12.1 Hz, 1H), 3.21 (br dd, J = 2.4, 11.3 Hz, 1H), 3.12 (d, J = 12.0 Hz, 1H), 2.68 − 2.52 (m, 2H), 2.45 (d, J = 12.8 Hz, 1H), 2.36 (s, 3H), 2.30 (t, J = 13.3 Hz, 2H), 2.22 (s, 3H), 1.99 − 1.89 (m, 1H), 1.81 (dd, J = 11.5, 12.6 Hz, 1H), 1.69 (d, J = 12.9 Hz, 1H), | Method B-1: Rt = 0.66 min; MS m/z [M + H]⁺ = 439.4 | |
| | | | 1.58 − 1.45 (m, 2H) | | |
| | | | 28 of 28H reported | | |
| Ex-23 | Intermediate A-24 | 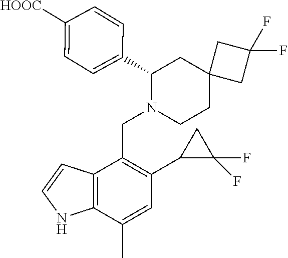 [SMILES: Cc1cc(C2CC2(F)F)c(CN2CCC3(C[C@H]2c2ccc(C(=O)O)cc2)CC(F)(F)C3)c2cc[nH]c12] | ¹H NMR (400 MHz, DMSO- d₆) δ [ppm] 13.47 − 12.45 (m, 1H), 10.99 (s, 1H), 7.96 (d, J = 8.4 Hz, 2H), 7.66 (br d, J = 7.8 Hz, 2H), 7.29 (t, J = 2.8 Hz, 1H), 6.68 (s, 1H), 6.63 (dd, J = 1.9, 3.0 Hz, 1H), 3.64 (d, J = 12.5 Hz, 1H), 3.26 − 3.18 (m, 2H), 2.99 − 2.88 (m, 1H), 2.67 − 2.52 (m, 2H), 2.39 (s, 3H), 2.37 − 2.18 (m, 2H), 2.07 (s, 1H), 1.96 − 1.88 (m, 1H), 1.84 (br t, J = 12.4 Hz, 1H), 1.74 − 1.65 (m, 1H), 1.79 − 1.64 (m, 2H), 1.59 (dt, J = 3.1, 12.6 Hz, | Method B-1: Rt = 1.01 min; MS m/z [M + H]⁺ = 501.3 | *) |
| | | | 1H), 1.49 (d, J = 12.3 Hz, 1H) | | |
| | | | 28 of 28H reported | | |
| Ex-24 | Intermediate A-23 | 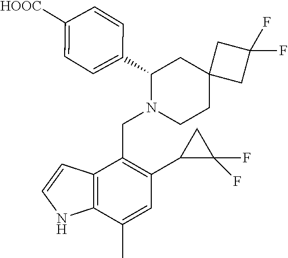 [SMILES: Cc1cc(C2CC2(F)F)c(CN2CCC3(C[C@H]2c2ccc(C(=O)O)cc2)CC(F)(F)C3)c2cc[nH]c12] | ¹H NMR (400 MHz, DMSO- d₆) δ [ppm] 13.93 − 12.04 (m, 1H), 11.00 (s, 1H), 7.92 (d, J = 7.7 Hz, 2H), 7.61 (d, J = 8.1 Hz, 2H), 7.30 (t, J = 2.8 Hz, 1H), 6.66 (dd, J = 1.9, 3.0 Hz, 1H), 6.65 (s, 1H), 3.68 (d, J = 12.3 Hz, 1H), 3.27 − 3.22 (m, 3H), 2.68 − 2.51 (m, 2H), 2.47 − 2.37 (m, 4H), 2.31 (br t, J = 13.4 Hz, 2H), 1.99 − 1.84 (m, 3H), 1.80 (br d, J = 11.8 Hz, 1H), 1.70 (d, J = 12.7 Hz, 1H), 1.63 − 1.53 (m, 1H), 1.50 (d, J = 12.2 Hz, 1H) 28 of 28H reported | Method B-1: Rt = 0.88 min; MS m/z [M + H]⁺ = 501.3 | *) |
| Ex-25 | Intermediate rac-A-25 (Ex-49) | 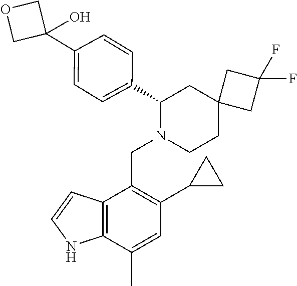 [SMILES: Cc1cc(C2CC2)c(CN2CCC3(C[C@H]2c2ccc(C4(O)COC4)cc2)CC(F)(F)C3)c2cc[nH]c12] | ¹H NMR (400 MHz, DMSO- d₆) δ [ppm] 10.81 (s, 1H), 7.59 (d, J = 8.3 Hz, 2H), 7.53 (d, J = 7.1 Hz, 2H), 7.23 (t, J = 2.8 Hz, 1H), 6.57 (dd, J = 1.9, 3.0 Hz, 1H), 6.44 (s, 1H), 6.30 (s, 1H), 4.77 (d, J = 6.3 Hz, 2H), 4.68 (dd, J = 6.3, 10.7 Hz, 2H), 3.79 (d, J = 12.1 Hz, 1H), 3.29 − 3.22 (m, 1H), 3.16 (dd, J = 2.4, 11.4 Hz, 1H), 2.67 − 2.52 (m, 2H), 2.48 − 2.39 (m, 1H), 2.35 (s, 3H), 2.30 (br t, J = 13.3 Hz, 2H), 2.21 − 2.08 (m, 1H), 1.99 (dt, J = 3.1, 11.8 Hz, 1H), 1.85 (t, J = 12.9 Hz, 1H), 1.68 (d, J = 13.3 Hz, 1H), | Method B-1: Rt = 0.60 min; MS m/z [M + H]⁺ = 493.3 | Peak 1 (SFC-8) Analytic al chiral HPLC: Rt = 1.65 min. 99.5% ee. |
| | | | 1.62 − 1.46 (m, 2H), 0.84 − | | |
| | | | 0.75 (m, 1H), 0.72 − 0.62 (m, | | |
| | | | 1H), 0.53 (ddt, J = 3.7, 5.6, | | |
| | | | 8.9 Hz, 1H), 0.20 − 0.10 (m, 1H) | | |
| | | | 34 of 34H reported | | |
| Ex-26 | Intermediate rac-A-25 (Ex-49) | 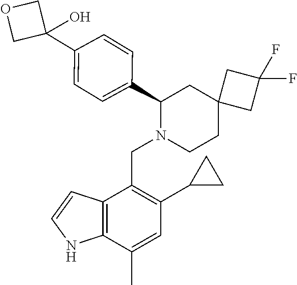 [SMILES: Cc1cc(C2CC2)c(CN2CCC3(C[C@@H]2c2ccc(C4(O)COC4)cc2)CC(F)(F)C3)c2cc[nH]c12] | ¹H NMR (400 MHz, DMSO- d₆) δ [ppm] 10.81 (s, 1H), 7.59 (d, J = 9.2 Hz, 2H), 7.53 (d, J = 8.1 Hz, 2H), 7.23 (t, J = 2.8 Hz, 1H), 6.57 (dd, J = 1.9, 3.0 Hz, 1H), 6.44 (s, 1H), 6.30 (s, 1H), 4.77 (d, J = 6.3 Hz, 2H), 4.68 (dd, J = 6.4, 10.6 Hz, 2H), 3.79 (d, J = 12.1 Hz, 1H), 3.25 (d, J = 11.8 Hz, 1H), 3.16 (dd, J = 2.3, 11.4 Hz, 1H), 2.67 − 2.52 (m, 2H), 2.48 − 2.39 (m, 1H), 2.35 (s, 3H), 2.30 (br t, J = 13.3 Hz, 2H), 2.20 − 2.08 (m, 1H), 1.99 (dt, J = 3.2, 11.8 Hz, 1H), 1.90 − 1.79 (m, 1H), 1.69 (br d, J = 13.0 Hz, 1H), | Method B-1: Rt = 0.63 min; MS m/z [M + H]⁺ = 493.3 | Peak 2 (SFC-8) Analytic al chiral HPLC: Rt = 3.15 min; 99.5% ee. |
| | | | 1.61 − 1.47 (m, 2H), 0.83 − | | |
| | | | 0.77 (m, 1H), 0.72 − 0.61 (m, | | |
| | | | 1H), 0.58 − 0.48 (m, 1H), | | |
| | | | 0.19 − 0.11 (m, 1H) | | |
| | | | 34 of 34H reported | | |
| Ex-27 | Intermediate rac-A-26 (Ex-48) | 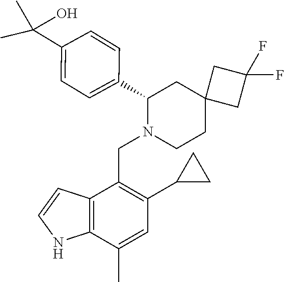 [SMILES: Cc1cc(C2CC2)c(CN2CCC3(C[C@H]2c2ccc(C(C)(C)O)cc2)CC(F)(F)C3)c2cc[nH]c12] | ¹H NMR (400 MHz, DMSO- d₆) δ [ppm] 10.81 (s, 1H), 7.46 − 7.39 (m, 4H), 7.22 (t, J = 2.8 Hz, 1H), 6.57 (dd, J = 1.9, 3.0 Hz, 1H), 6.43 (s, 1H), 4.95 (s, 1H), 3.78 (d, J = 12.1 Hz, 1H), 3.23 (d, J = 12.3 Hz, 1H), 3.11 (dd, J = 2.5, 11.4 Hz, 1H), 2.68 − 2.52 (m, 2H), 2.48 − 2.38 (m, 1H), 2.35 (s, 3H), 2.33 − 2.26 (m, 2H), 2.19 − 2.08 (m, 1H), 1.98 (dt, J = 3.2, 11.7 Hz, 1H), 1.84 (dd, J = 12.3, 12.8 Hz, 1H), 1.67 (br d, J = 13.1 Hz, 1H), 1.59 − 1.46 (m, 2H), 1.42 (d, J = 2.1 Hz, 6H), 0.82 − 0.74 (m, 1H), 0.70 − 0.63 (m, 1H), | Method B-1: Rt = 0.69 min; MS m/z [M + H]⁺ = 479.3 | Peak 1 (SFC-9) Analytic al chiral HPLC: Rt = 1.23 min; 99.5% ee. |
| | | | 0.56 − 0.45 (m, 1H), 0.15 − | | |
| | | | 0.08 (m, 1H) | | |
| | | | 36 of 36H reported | | |
| Ex-28 | Intermediate rac-A-26 (Ex-48) | 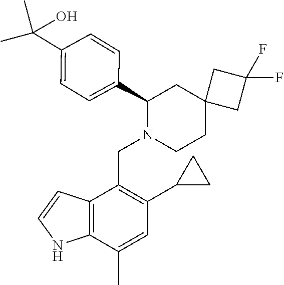 [SMILES: Cc1cc(C2CC2)c(CN2CCC3(C[C@@H]2c2ccc(C(C)(C)O)cc2)CC(F)(F)C3)c2cc[nH]c12] | ¹H NMR (400 MHz, DMSO- d₆) δ [ppm] 10.81 (br s, 1H), 7.47 − 7.39 (m, 4H), 7.22 (t, J = 2.8 Hz, 1H), 6.57 (dd, J = 1.9, 3.0 Hz, 1H), 6.43 (s, 1H), 4.95 (s, 1H), 3.78 (d, J = 12.1 Hz, 1H), 3.26 − 3.19 (m, 1H), 3.11 (dd, J = 2.4, 11.4 Hz, 1H), 2.68 − 2.52 (m, 2H), 2.48 − 2.43 (m, 1H), 2.35 (s, 3H), 2.30 (br t, J = 12.9 Hz, 2H), 2.19 − 2.08 (m, 1H), 1.98 (dt, J = 3.0, 11.7 Hz, 1H), 1.84 (t, J = 12.7 Hz, 1H), 1.67 (br d, J = 13.1 Hz, 1H), 1.59 − 1.46 (m, 2H), 1.44 − 1.39 (m, 6H), 0.82 − 0.74 (m, 1H), 0.70 − 0.63 (m, 1H), | Method B-1: Rt = 0.69 min; MS m/z [M + H]⁺ = 479.5 | Peak 2 (SFC-9) Analytic al chiral HPLC: Rt = 1.99 min; 99.0% ee. |
| | | | 0.55 − 0.46 (m, 1H), 0.16 − | | |
| | | | 0.08 (m, 1H) | | |
| | | | 36 of 36H reported | | |
| Ex-29 | Intermediate rac-A-27 (Ex-51) | 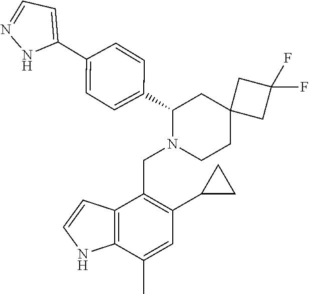 [SMILES: Cc1cc(C2CC2)c(CN2CCC3(C[C@H]2c2ccc(-c4ccn[nH]4)cc2)CC(F)(F)C3)c2cc[nH]c12] | ¹H NMR (400 MHz, DMSO- d₆) δ [ppm] 12.84 (br s, 1H), 10.82 (s, 1H), 7.79 (br d, J = 7.0 Hz, 3H), 7.54 (br d, J = 7.3 Hz, 2H), 7.23 (t, J = 2.8 Hz, 1H), 6.69 (d, J = 2.1 Hz, 1H), 6.57 (dd, J = 1.9, 3.0 Hz, 1H), 6.44 (s, 1H), 3.82 (d, J = 12.1 Hz, 1H), 3.30 − 3.26 (m, 1H), 3.16 (br d, J = 9.5 Hz, 1H), 2.67 − 2.53 (m, 2H), 2.48 − 2.39 (m, 1H), 2.35 (s, 3H), 2.34 − 2.24 (m, 2H), 2.22 − 2.14 (m, 1H), 2.00 (dt, J = 3.0, 11.8 Hz, 1H), 1.87 (dd, J = 12.0, 12.8 Hz, 1H), 1.72 (br d, J = 13.1 Hz, 1H), 1.61 − 1.48 (m, 2H), 0.84 − 0.77 (m, 1H), 0.71 − 0.62 (m, | Method B-1: Rt = 0.76 min; MS m/z [M + H]⁺ = 487.5 | Peak 1 (SFC-10) Analytic al chiral HPLC: Rt = 1.20 min; 99.5% ee. |
| | | | 1H), 0.60 − 0.51 (m, 1H), | | |
| | | | 0.20 − 0.12 (m, 1H) | | |
| | | | 32 of 32H reported | | |
| Ex-30 | Intermediate rac-A-27 (Ex-51) | 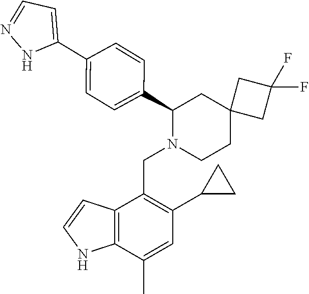 [SMILES: Cc1cc(C2CC2)c(CN2CCC3(C[C@@H]2c2ccc(-c4ccn[nH]4)cc2)CC(F)(F)C3)c2cc[nH]c12] | ¹H NMR (400 MHz, DMSO- d₆) δ [ppm] 12.83 (br s, 1H), 10.82 (s, 1H), 7.91 − 7.66 (m, 3H), 7.54 (br d, J = 7.0 Hz, 2H), 7.23 (t, J = 2.8 Hz, 1H), 6.69 (d, J = 2.1 Hz, 1H), 6.57 (dd, J = 2.0, 3.0 Hz, 1H), 6.44 (s, 1H), 3.82 (br d, J = 12.1 Hz, 1H), 3.30 − 3.26 (m, 1H), 3.16 (br d, J = 9.4 Hz, 1H), 2.67 − 2.53 (m, 2H), 2.48 − 2.39 (m, 1H), 2.35 (s, 3H), 2.34 − 2.24 (m, 2H), 2.22 − 2.14 (m, 1H), 2.00 (dt, J = 2.9, 11.8 Hz, 1H), 1.87 (t, J = 13.1 Hz, 1H), 1.72 (br d, J = 13.0 Hz, 1H), 1.61 − 1.48 (m, 2H), 0.84 − 0.77 (m, 1H), 0.72 − 0.62 (m, 1H), 0.61 − | Method B-1: Rt = 0.75 min; MS m/z [M + H]⁺ = 487.3 | Peak 2 (SFC-10) Analytic al chiral HPLC: Rt = 1.22 min; 99.5% ee. |
| | | | 0.50 (m, 1H), 0.21 − 0.11 (m, | | |
| | | | 1H) | | |
| | | | 32 of 32H reported | | |
| Ex-31 | Intermediate rac-A-28 (Ex-50) | 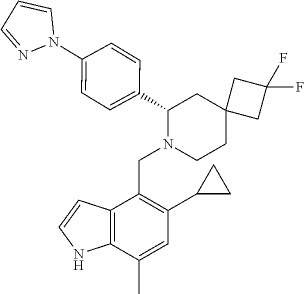 [SMILES: Cc1cc(C2CC2)c(CN2CCC3(C[C@H]2c2ccc(-n4cccn4)cc2)CC(F)(F)C3)c2cc[nH]c12] | ¹H NMR (400 MHz, DMSO- d₆) δ [ppm] 10.83 (br s, 1H), 8.48 (d, J = 2.4 Hz, 1H), 7.84 (d, J = 8.7 Hz, 2H), 7.73 (d, J = 1.6 Hz, 1H), 7.62 (d, J = 8.2 Hz, 2H), 7.23 (t, J = 2.8 Hz, 1H), 6.57 (dd, J = 1.9, 2.9 Hz, 1H), 6.53 (t, J = 2.3 Hz, 1H), 6.45 (s, 1H), 3.81 (d, J = 12.0 Hz, 1H), 3.29 − 3.26 (m, 1H), 3.20 (dd, J = 2.3, 11.3 Hz, 1H), 2.67 − 2.55 (m, 2H), 2.49 − 2.39 (m, 1H), 2.36 (s, 3H), 2.33 − 2.27 (m, 1H), 2.17 (tt, J = 5.5, 8.4 Hz, 1H), 2.01 (dt, J = 2.9, 11.8 Hz, 1H), 1.87 (t, J = 12.8 Hz, 1H), 1.71 (d, J = 13.6 Hz, 1H), | Method B-1: Rt = 0.85 min; MS m/z [M + H]⁺ = 487.5 | Peak 1 (SFC-11) Analytic al chiral HPLC: Rt = 1.33 min; 99.5% ee. |
| | | | 1.61 − 1.49 (m, 2H), 1.31 − | | |
| | | | 1.19 (m, 1H), 0.89 − 0.78 (m, | | |
| | | | 1H), 0.74 − 0.64 (m, 1H), | | |
| | | | 0.62 − 0.51 (m, 1H), 0.21 − | | |
| | | | 0.12 (m, 1H) | | |
| | | | 32 of 32H reported | | |
| Ex-32 | Intermediate rac-A-28 (Ex-50) | 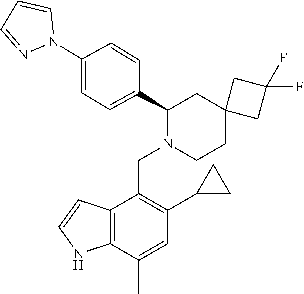 [SMILES: Cc1cc(C2CC2)c(CN2CCC3(C[C@@H]2c2ccc(-n4cccn4)cc2)CC(F)(F)C3)c2cc[nH]c12] | ¹H NMR (400 MHz, DMSO- d₆) δ [ppm] 10.82 (s, 1H), 8.48 (d, J = 2.2 Hz, 1H), 7.84 (d, J = 8.7 Hz, 2H), 7.73 (d, J = 1.5 Hz, 1H), 7.62 (d, J = 8.1 Hz, 2H), 7.23 (t, J = 2.8 Hz, 1H), 6.57 (dd, J = 2.0, 3.0 Hz, 1H), 6.53 (dd, J = 1.8, 2.4 Hz, 1H), 6.45 (s, 1H), 3.81 (d, J = 12.0 Hz, 1H), 3.31 − 3.26 (m, 1H), 3.20 (dd, J = 2.4, 11.4 Hz, 1H), 2.67 − 2.53 (m, 2H), 2.49 − 2.41 (m, 1H), 2.35 (s, 3H), 2.34 − 2.24 (m, 2H), 2.17 (tt, J = 5.5, 8.4 Hz, 1H), 2.01 (dt, J = 3.0, 11.8 Hz, 1H), 1.87 (t, J = 12.7 Hz, 1H), 1.72 (br d, J = 13.0 Hz, 1H), | Method B-1: Rt = 0.82 min; MS m/z [M + H]⁺ = 487.3 | Peak 2 (SFC-11) Analytic al chiral HPLC: Rt = 1.94 min; 99.5% ee. |
| | | | 1.63 − 1.47 (m, 2H), 0.85 − | | |
| | | | 0.77 (m, 1H), 0.72 − 0.63 (m, | | |
| | | | 1H), 0.62 − 0.51 (m, 1H), | | |
| | | | 0.21 − 0.12 (m, 1H) | | |
| | | | 32 of 32H reported | | |
| Ex-33 | Ex-57 | 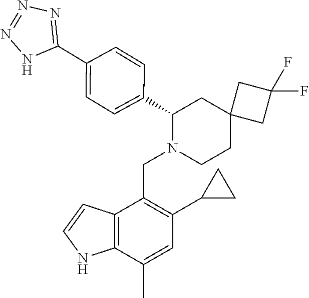 [SMILES: Cc1cc(C2CC2)c(CN2CCC3(C[C@H]2c2ccc(-c4nnn[nH]4)cc2)CC(F)(F)C3)c2cc[nH]c12] | ¹H NMR (400 MHz, DMSO- d₆) δ [ppm] 10.90 (br s, 1H), 8.06 (d, J = 8.2 Hz, 2H), 7.74 (br d, J = 7.7 Hz, 2H), 7.27 (br s, 1H), 6.54 (br s, 1H), 6.48 (s, 1H), 3.89 (br s, 1H), 3.74 − 3.55 (m, 2H), 2.78 − 2.52 (m, 3H), 2.43 − 2.28 (m, 6H), 2.17 − 2.07 (m, 1H), 2.01 − 1.87 (m, 1H), 1.79 (br d, J = 13.0 Hz, 1H), 1.70 − 1.54 (m, 2H), 0.88 − 0.77 (m, 1H), 0.70 (br dd, J = 4.4, 9.0 Hz, 1H), 0.48 (br s, 1H), 0.18 − 0.06 (m, 1H) 29 of 30H reported | Method B-1: Rt = 0.68 min; MS m/z [M + H]⁺ = 489.5 | Peak 1 (SFC-13) Analytic al chiral HPLC: Rt = 1.13 min; 99.5% ee. |
| Ex-34 | Ex-57 | 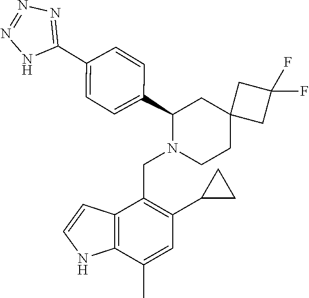 [SMILES: Cc1cc(C2CC2)c(CN2CCC3(C[C@@H]2c2ccc(-c4nnn[nH]4)cc2)CC(F)(F)C3)c2cc[nH]c12] | ¹H NMR (400 MHz, DMSO- d₆) δ [ppm] 10.90 (br s, 1H), 8.06 (d, J = 8.4 Hz, 2H), 7.74 (br d, J = 7.8 Hz, 2H), 7.27 (t, J = 2.5 Hz, 1H), 6.55 (br s, 1H), 6.48 (s, 1H), 3.94 − 3.81 (m, 1H), 3.49 (br s, 2H), 2.77 - 2.59 (m, 2H), 2.57 − 2.52 (m, 1H), 2.40 − 2.26 (m, 6H), 2.15 − 2.08 (m, 1H), 2.02 − 1.86 (m, 1H), 1.79 (br d, J = 12.9 Hz, 1H), 1.67 − 1.54 (m, 2H), 0.87 − 0.75 (m, 1H), 0.74 − 0.59 (m, 1H), 0.48 (br s, 1H), 0.18 − 0.06 (m, 1H) 29 of 30H reported | Method B-1: Rt = 0.65 min; MS m/z [M + H]⁺ = 489.3 | Peak 2 (SFC-13) Analytic al chiral HPLC: Rt = 2.08 min; 99.5% ee. |
| Ex-35 | Intermediate rac-A-30 | 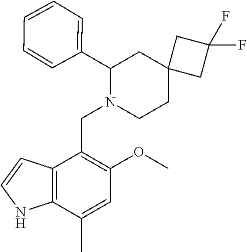 [SMILES: COc1cc(C)c2[nH]ccc2c1CN1CCC2(CC1c1ccccc1)CC(F)(F)C2] | ¹H NMR (400 MHz, DMSO- d₆) δ [ppm] 10.79 (br s, 1H), 7.55 (br d, J = 7.2 Hz, 2H), 7.39 (t, J = 7.7 Hz, 2H), 7.30 - 7.25 (m, 1H), 7.23 (t, J = 2.8 Hz, 1H), 6.64 (s, 1H), 6.48 (dd, J = 2.0, 2.9 Hz, 1H), 3.69 (s, 3H), 3.56 (d, J = 12.0 Hz, 1H), 3.15 (d, J = 12.0 Hz, 1H), 3.11 (dd, J = 3.0, 11.0 Hz, 1H), 2.69 − 2.62 (m, 1H), 2.62 − 2.52 (m, 1H), 2.46 − 2.39 (m, 4H), 2.29 (br t, J = 13.1 Hz, 2H), 2.02 − 1.87 (m, 1H), 1.79 − 1.65 (m, 2H), 1.58 − 1.47 (m, 2H) | Method B-1: Rt = 0.65 min; MS m/z [M + H]⁺ = 411.4 | Racemate |
| | | | 28 of 28H reported | | |
| Ex-36 | Intermediate rac-A-31 | 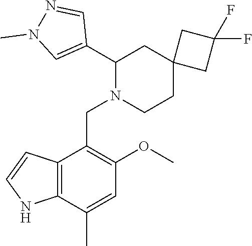 [SMILES: COc1cc(C)c2[nH]ccc2c1CN1CCC2(CC1c1cnn(C)c1)CC(F)(F)C2] | ¹H NMR (400 MHz, DMSO- d₆) δ [ppm] 10.76 (br s, 1H), 7.68 (s, 1H), 7.48 (s, 1H), 7.20 (t, J = 2.8 Hz, 1H), 6.64 (s, 1H), 6.38 (dd, J = 1.9, 3.0 Hz, 1H), 3.83 (s, 3H), 3.76 (d, J = 12.0 Hz, 1H), 3.70 (s, 3H), 3.14 (d, J = 11.9 Hz, 1H), 3.08 (dd, J = 2.7, 11.3 Hz, 1H), 2.65 − 2.56 (m, 1H), 2.48 − 2.37 (m, 5H), 2.30 (dd, J = 12.0, 12.8 Hz, 2H), 1.90 − 1.86 (m, 1H), 1.85 − 1.77 (m, 1H), 1.69 (d, J = 11.7 Hz, 1H), 1.53 − 1.40 (m, 2H) 28 of 28H reported | Method B-3: Rt = 1.94 min; MS m/z [M + H]⁺ = 415.4 | Racemate |
| Ex-37 | Ex-35 | 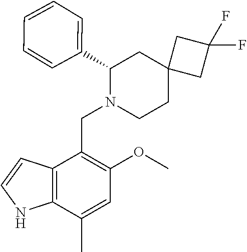 [SMILES: COc1cc(C)c2[nH]ccc2c1CN1CCC2(C[C@H]1c1ccccc1)CC(F)(F)C2] | ¹H NMR (400 MHz, DMSO- d₆) δ [ppm] 10.89 (br s, 1H), 7.55 (d, J = 7.5 Hz, 2H), 7.39 (t, J = 7.5 Hz, 2H), 7.31 − 7.25 (m, 1H), 7.23 (t, J = 2.8 Hz, 1H), 6.64 (s, 1H), 6.48 (dd, J = 3.1, 1.9 Hz, 1H), 3.69 (s, 3H), 3.56 (d, J = 12.0 Hz, 1H), 3.15 (d, J = 12.0 Hz, 1H), 3.11 (dd, J = 11.0, 3.2 Hz, 1H), 2.71 − 2.63 (m, 1H), 2.63 − 2.52 (m, 1H), 2.48 − 2.37 (m, 4H), 2.34 − 2.24 (m, 2H), 2.01 − 1.88 (m, 1H), 1.79 − 1.64 (m, 2H), 1.58 − 1.46 (m, 2H) | Method B-6: Rt = 0.66 min; MS m/z [M + H]⁺ = 411.3 | Peak 1 (SFC-18) |
| | | | 28 of 28H reported | | |
| Ex-38 | Ex-35 | 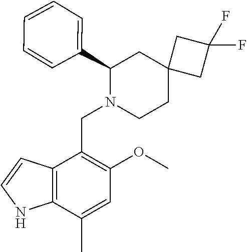 [SMILES: COc1cc(C)c2[nH]ccc2c1CN1CCC2(C[C@@H]1c1ccccc1)CC(F)(F)C2] | | Method B-6: Rt = 0.66 min; MS m/z [M + H]⁺ = 411.4 | Peak 2 (SFC-18) |
| Ex-39 | Ex-36 | 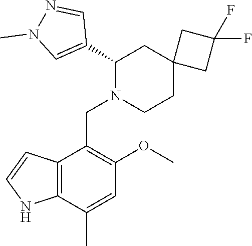 [SMILES: COc1cc(C)c2[nH]ccc2c1CN1CCC2(C[C@H]1c1cnn(C)c1)CC(F)(F)C2] | ¹H NMR (400 MHz, DMSO- d₆) δ [ppm] 10.76 (br s, 1H), 7.68 (d, J = 0.8 Hz, 1H), 7.48 (d, J = 0.8 Hz, 1H), 7.20 (t, J = 2.8 Hz, 1H), 6.64 (d, J = 1.0 Hz, 1H), 6.38 (dd, J = 3.1, 1.9 Hz, 1H), 3.83 (s, 3H), 3.77 (d, J = 12.0 Hz, 1H), 3.70 (s, 3H), 3.14 (d, J = 12.0 Hz, 1H), 3.08 (dd, J = 11.4, 2.8 Hz, 1H), 2.61 (m, 1H), 2.47 − 2.37 (m, 5H), 2.33 − 2.22 (m, 2H), 1.93 − 1.84 (m, 1H), 1.85 − 1.76 (m, 1H), 1.73 − 1.66 (m, 1H), 1.49 − 1.42 (m, 2H) | Method B-6: Rt = 0.49 min; MS m/z [M + H]⁺ = 415.1 | Peak 1 (SFC-19) |
| | | | 28 of 28H reported | | |
| Ex-40 | Ex-36 | 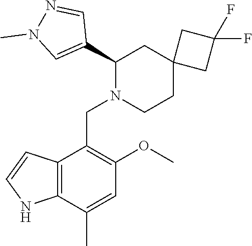 [SMILES: COc1cc(C)c2[nH]ccc2c1CN1CCC2(C[C@@H]1c1cnn(C)c1)CC(F)(F)C2] | | Method B-6: Rt = 0.49 min; MS m/z [M + H]⁺ = 415.1 | Peak 2 (SFC-19) |
| Ex-41 | Intermediate 41-42 | 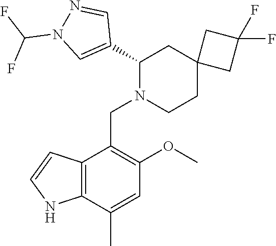 [SMILES: COc1cc(C)c2[nH]ccc2c1CN1CCC2(C[C@H]1c1cnn(C(F)F)c1)CC(F)(F)C2] | ¹H NMR (400 MHz, DMSO- d₆) δ [ppm] 10.80 (br s, 1H), 8.23 (s, 1H), 7.97 − 7.62 (m, 2H), 7.26 − 7.17 (m, 1H), 6.65 (s, 1H), 6.36 (dd, J = 3.1, 1.9 Hz, 1H), 3.74 − 3.64 (m, 4H), 3.26 − 3.12 (m, 2H), 2.69 − 2.59 (m, 1H), 2.54 − 2.47 (m, 1H* partially under solvent peak), 2.47 − 2.36 (m, 4H), 2.35 − 2.23 (m, 2H), 1.99 − 1.89 (m, 1H), 1.87 − 1.78 (m, 1H), 1.77 − 1.69 (m, 1H), 1.54 − 1.44 (m, 2H) 26 of 26H reported | Method B-6: Rt = 0.45 min; MS m/z [M + H]⁺ = 451.4 | Peak 1 (SFC-20) |
| Ex- 42 | Intermediate 41-42 | 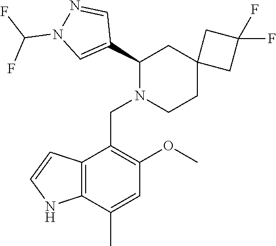 [SMILES: COc1cc(C)c2[nH]ccc2c1CN1CCC2(C[C@@H]1c1cnn(C(F)F)c1)CC(F)(F)C2] | | Method B-6: Rt = 0.41 min; MS m/z [M + H]⁺ = 451.4 | Peak 2 (SFC-20) |
| Ex- 43 | Intermediate 43-44 | 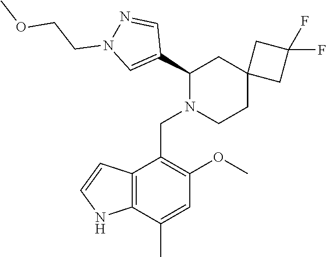 [SMILES: COCCn1cc([C@H]2CC3(CCN2Cc2c(OC)cc(C)c4[nH]ccc24)CC(F)(F)C3)cn1] | | Method B-6: Rt = 0.48 min; MS m/z [M + H]⁺ = 459.5 | Peak 1 (SFC-21) |
| Ex- 44 | Intermediate 43-44 | 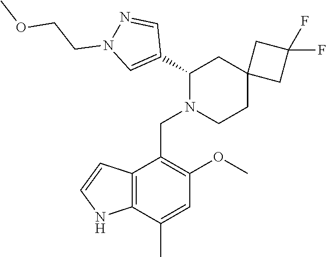 [SMILES: COCCn1cc([C@@H]2CC3(CCN2Cc2c(OC)cc(C)c4[nH]ccc24)CC(F)(F)C3)cn1] | ¹H NMR (400 MHz, DMSO- d₆) δ [ppm] 10.75 (br s, 1H), 7.72 (s, 1H), 7.51 (s, 1H), 7.19 (t, J = 2.8 Hz, 1H), 6.64 (s, 1H), 6.37 (dd, J = 3.1, 1.9 Hz, 1H), 4.27 − 4.24 (m, 2H), 3.75 (d, J = 11.9 Hz, 1H), 3.72 − 3.65 (m, 5H), 3.20 (s, 3H), 3.15 (d, J = 11.9 Hz, 1H), 3.09 (dd, J = 11.3, 2.8 Hz, 1H), 2.64 − 2.57 (m, 1H), 2.49 − 2.35 (m, 5H* partially under solvent peak), 2.35 − 2.22 (m, 2H), 1.92 − 1.84 (m, 1H), 1.74 − 1.67 (m, 2H), 1.49 − 1.42 (m, 2H) | Method B-6: Rt = 0.46 min; MS m/z [M + H]⁺ = 459.2 | Peak 2 (SFC-21) |
| | | | 32 of 32H reported | | |
| Ex-45 | Intermediate A-32 | 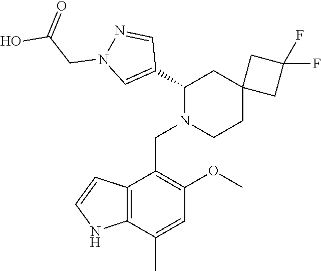 [SMILES: COc1cc(C)c2[nH]ccc2c1CN1CCC2(C[C@H]1c1cnn(CC(=O)O)c1)CC(F)(F)C2] | Formic acid salt ¹H NMR (400 MHz, DMSO- d₆) δ [ppm] 10.81 (br s, 1H), 7.75 (s, 1H), 7.54 (s, 1H), 7.22 (t, J = 2.8 Hz, 1H), 6.66 (s, 1H), 6.39 (dd, J = 3.1, 1.9 Hz, 1H), 4.90 − 4.80 (m, 2H), 3.92 − 3.83 (m, 1H), 3.70 (s, 3H), 3.43 − 3.19 (m, 2H), 2.73 − 2.64 (m, 1H), 2.59 − 2.48 (m, 1H* partially under solvent peak), 2.48 − 2.39 (m, 4H), 2.37 − 2.24 (m, 2H), 2.13 − 1.98 (m, 1H), 1.97 − 1.85 (m, 1H), 1.80 − 1.69 (m, 1H), 1.62 − 1.45 (m, 2H) 27 of 28H reported | Method B-6: Rt = 0.43 min; MS m/z [M + H]⁺ = 459.0 | |
| | | | (—COOH not observed) | | |
| Ex-46 | Intermediate A-33 | 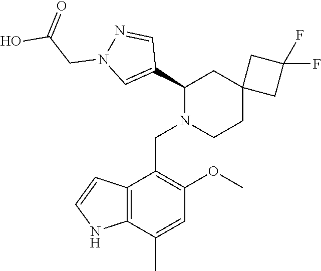 [SMILES: COc1cc(C)c2[nH]ccc2c1CN1CCC2(C[C@@H]1c1cnn(CC(=O)O)c1)CC(F)(F)C2] | | Method B-6: Rt = 0.43 min; MS m/z [M + H]⁺ = 459.0 | |
| Ex-47 | Intermediate rac-P-5 | 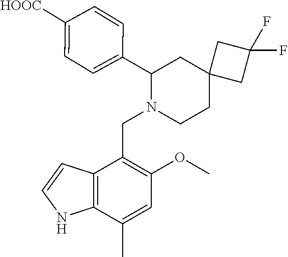 [SMILES: COc1cc(C)c2[nH]ccc2c1CN1CCC2(CC1c1ccc(C(=O)O)cc1)CC(F)(F)C2] | | Method B-1: Rt = 0.50 min; MS m/z [M + H]⁺ = 455.3 | Racemate |
| Ex-48 | Intermediate rac-P-15 | 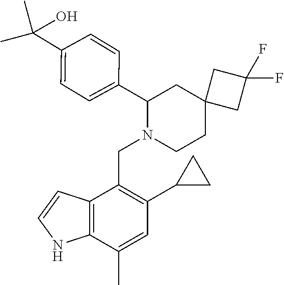 [SMILES: Cc1cc(C2CC2)c(CN2CCC3(CC2c2ccc(C(C)(C)O)cc2)CC(F)(F)C3)c2cc[nH]c12] | | Method B-1: Rt = 0.73 min; MS m/z [M + H]⁺ = 479.3 | Racemate |
| Ex-49 | Intermediate rac-P-14 | 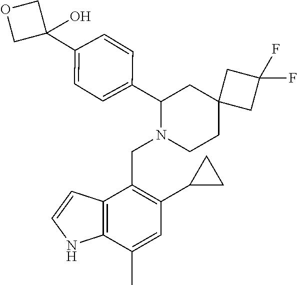 [SMILES: Cc1cc(C2CC2)c(CN2CCC3(CC2c2ccc(C4(O)COC4)cc2)CC(F)(F)C3)c2cc[nH]c12] | | Method B-1: Rt = 0.58 min; MS m/z [M + H]⁺ = 493.4 | Racemate |
| Ex-50 | Intermediate rac-P-17 | 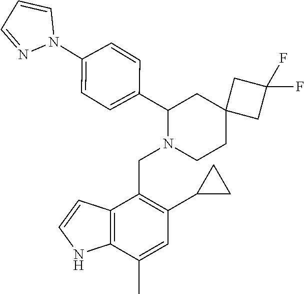 [SMILES: Cc1cc(C2CC2)c(CN2CCC3(CC2c2ccc(-n4cccn4)cc2)CC(F)(F)C3)c2cc[nH]c12] | | Method B-1: Rt = 0.82 min; MS m/z [M + H]⁺ = 487.3 | Racemate |
| Ex-51 | Intermediate rac-P-16 | 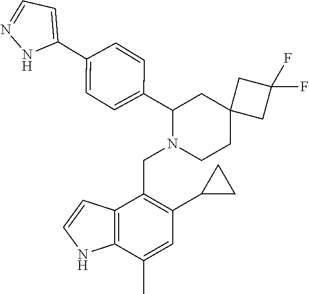 [SMILES: Cc1cc(C2CC2)c(CN2CCC3(CC2c2ccc(-c4ccn[nH]4)cc2)CC(F)(F)C3)c2cc[nH]c12] | | Method B-1: Rt = 0.75 min; MS m/z [M + H]⁺ = 487.5 | Racemate |
| Ex-52 | Intermediate rac-P-24 | 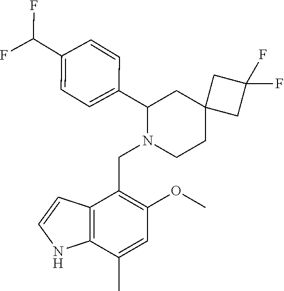 [SMILES: COc1cc(C)c2[nH]ccc2c1CN1CCC2(CC1c1ccc(C(F)F)cc1)CC(F)(F)C2] | | Method B-1: Rt = 0.75 min; MS m/z [M + H]⁺ = 461.3 | Racemate |
| Ex-53 | | 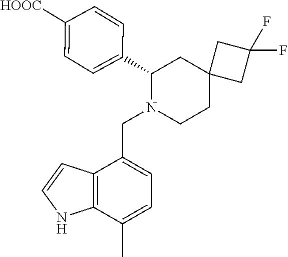 [SMILES: Cc1ccc(CN2CCC3(C[C@H]2c2ccc(C(=O)O)cc2)CC(F)(F)C3)c2cc[nH]c12] | ¹H NMR (400 MHz, DMSO) δ [ppm] 10.91 (br s, 1H), 7.94 − 7.84 (m, 2H), 7.58 (d, J = 7.9 Hz, 2H), 7.19 (t, J = 2.8 Hz, 1H), 6.76 (d, J = 7.3 Hz, 1H), 6.73 (d, J = 7.3 Hz, 1H), 6.39 (dd, J = 3.1, 1.9 Hz, 1H), 3.65 (d, J = 13.1 Hz, 1H), 3.21 (dd, J = 8.2, 6.0 Hz, 2H), 2.95 (d, J = 13.1 Hz, 1H), 2.69 (dt, J = 11.9, 3.4 Hz, 1H), 2.53 (q, J = 13.1 Hz, 1H), 2.34 (s, 3H), 2.32 − 2.21 (m, 2H), 1.87 (td, J = 12.1, 2.8 Hz, 1H), 1.67 (d, J = 7.9 Hz, 2H), 1.61 − 1.44 (m, 2H) 25 of 26H reported | Method C-3: Rt = 1.43 min; MS m/z [M + H]⁺ = 425.3 | |
| | | | (—COOH not observed) | | |
| Ex-54 | | 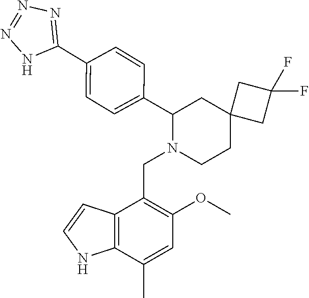 [SMILES: COc1cc(C)c2[nH]ccc2c1CN1CCC2(CC1c1ccc(-c3nnn[nH]3)cc1)CC(F)(F)C2] | Formic acid salt | Method B-6: Rt = 0.49 min; MS m/z [M-H] = 477.2 | Racemate |
| Ex-55 | Ex-54 | 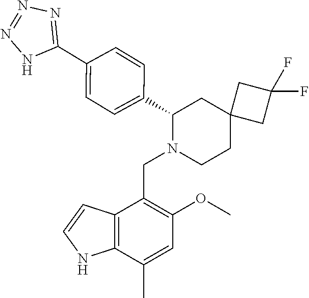 [SMILES: COc1cc(C)c2[nH]ccc2c1CN1CCC2(C[C@H]1c1ccc(-c3nnn[nH]3)cc1)CC(F)(F)C2] | ¹H NMR (400 MHz, DMSO- d₆) δ [ppm] 10.94 (br s, 1H), 8.10 (d, J = 8.2 Hz, 2H), 7.75 (d, J = 7.8 Hz, 2H), 7.30 (t, J = 2.8 Hz, 1H), 6.69 (s, 1H), 6.49 − 6.38 (m, 1H), 3.87 − 3.76 (m, 1H), 3.70 (s, 3H), 3.64 − 3.45 (m, 2H), 2.95 − 2.82 (m, 1H), 2.75 − 2.61 (m, 1H), 2.61 − 2.47 (m, 1H* partially under solvent peak), 2.43 (s, 3H), 2.42 − 2.31 (m, 2H), 2.05 − 1.89 (m, 1H), 1.88 − 1.80 (m, 1H), 1.78 − 1.67 (m, 1H), 1.67 − 1.57 (m, 1H), 1.30 − 1.20 (m, 1H) 27 of 28H reported (tetrazole-N-H not observed) | Method B-6: Rt = 0.52 min; MS m/z [M + H]⁺ = 479.5 | Peak 1 (SFC-23) |
| Ex-56 | Ex-54 | 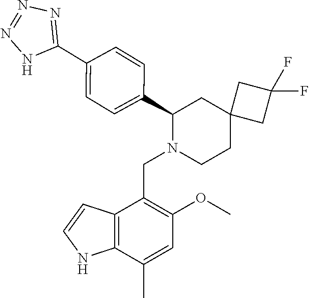 [SMILES: COc1cc(C)c2[nH]ccc2c1CN1CCC2(C[C@@H]1c1ccc(-c3nnn[nH]3)cc1)CC(F)(F)C2] | | Method B-6: Rt = 0.55 min; MS m/z [M + H]⁺ = 479.1 | Peak 2 (SFC-23) |
| Ex-57 | | 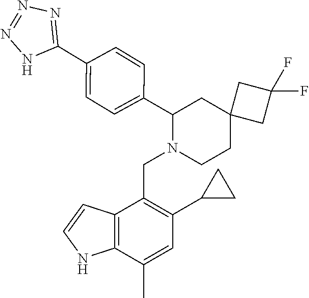 [SMILES: Cc1cc(C2CC2)c(CN2CCC3(CC2c2ccc(-c4nnn[nH]4)cc2)CC(F)(F)C3)c2cc[nH]c12] | Formic acid salt | Method B-1: Rt = 0.66 min; MS m/z [M + H]⁺ = 489.3 | Racemate |
| Ex-58 | | 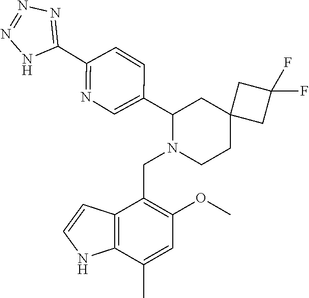 [SMILES: COc1cc(C)c2[nH]ccc2c1CN1CCC2(CC1c1ccc(-c3nnn[nH]3)nc1)CC(F)(F)C2] | Formic acid salt | Method B-6: Rt = 0.46 min; MS m/z [M + H]⁺ = 480.2 | Racemate |
| Ex-59 | Ex-58 | 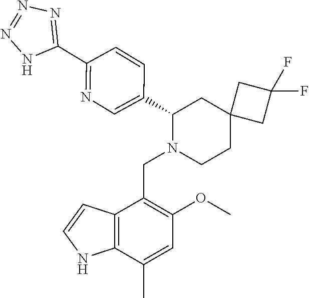 [SMILES: COc1cc(C)c2[nH]ccc2c1CN1CCC2(C[C@H]1c1ccc(-c3nnn[nH]3)nc1)CC(F)(F)C2] | ¹H NMR (400 MHz, DMSO- d₆) δ [ppm] 10.89 (br s, 1H), 8.92 (s, 1H), 8.29 − 8.16 (m, 2H), 7.28 (t, J = 2.8 Hz, 1H), 6.65 (s, 1H), 6.43 (t, J = 2.4 Hz, 1H), 3.73 − 3.61 (m, 4H), 3.59 − 3.43 (m, 1H), 3.43 − 3.28 (m, 1H), 2.87 − 2.76 (m, 1H), 2.75 − 2.62 (m, 1H), 2.59 − 2.44 (m, 1H* partially under solvent peak), 2.42 (s, 3H), 2.39 − 2.29 (m, 2H), 2.29 − 2.15 (m, 1H), 1.92 − 1.73 (m, 2H), 1.72 − 1.53 (m, 2H) 26 of 27H reported (tetrazole-N—H not observed) | Method B-6: Rt = 0.46 min; MS m/z [M + H]⁺ = 480.1 | Peak 1 (SFC-24) |
| Ex-60 | Ex-58 | 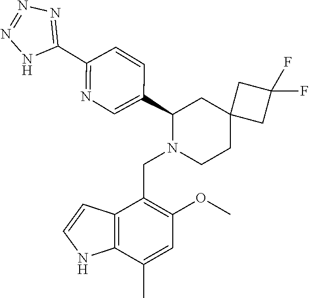 [SMILES: COc1cc(C)c2[nH]ccc2c1CN1CCC2(C[C@@H]1c1ccc(-c3nnn[nH]3)nc1)CC(F)(F)C2] | | Method B-6: Rt = 0.43 min; MS m/z [M + H]⁺ = 480.5 | Peak 2 (SFC-24) |
| Ex-61 | | 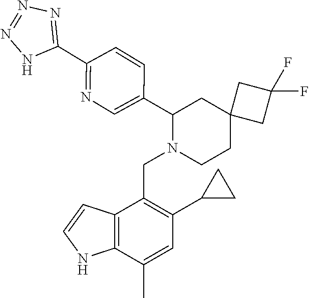 [SMILES: Cc1cc(C2CC2)c(CN2CCC3(CC2c2ccc(-c4nnn[nH]4)nc2)CC(F)(F)C3)c2cc[nH]c12] | | Method B-6: Rt = 0.72 min; MS m/z [M + H]⁺ = 490.2 | Racemate |
| Ex-62 | Ex-61 | 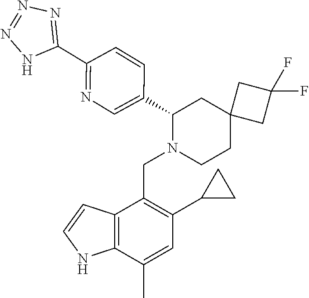 [SMILES: Cc1cc(C2CC2)c(CN2CCC3(C[C@H]2c2ccc(-c4nnn[nH]4)nc2)CC(F)(F)C3)c2cc[nH]c12] | ¹H NMR (400 MHz, DMSO- d₆) δ [ppm] 10.87 (br s, 1H), 8.92 (s, 1H), 8.27 − 8.15 (m, 2H), 7.26 (t, J = 2.8 Hz, 1H), 6.57 − 6.52 (m, 1H), 6.48 (s, 1H), 3.85 − 3.72 (m, 1H), 3.56 − 3.18 (m, 2H* partially under water peak), 2.77 − 2.61 (m, 2H), 2.57 − 2.44 (m, 1H* partially under solvent peak), 2.40 − 2.28 (m, 5H), 2.16 − 2.08 (m, 2H), 2.01 − 1.87 (m, 1H), 1.86 − 1.74 (m, 1H), 1.71 − 1.52 (m, 2H), 0.87 − 0.76 (m, 1H), 0.73 − 0.63 (m, 1H), 0.55 − 0.40 (m, 1H), 0.19 − 0.08 (m, 1H) 28 of 29H reported (tetrazole-N-H not | Method B-6: Rt = 0.73 min; MS m/z [M + H]⁺ = 490.2 | Peak 1 (SFC-25) |
| | | | observed) | | |
| Ex-63 | Ex-61 | 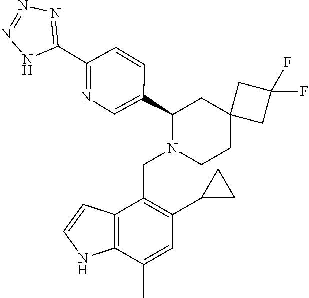 [SMILES: Cc1cc(C2CC2)c(CN2CCC3(C[C@@H]2c2ccc(-c4nnn[nH]4)nc2)CC(F)(F)C3)c2cc[nH]c12] | | Method B-6: Rt = 0.74 min; MS m/z [M + H]⁺ = 490.3 | Peak 2 (SFC-25) |
| Ex-64 | Intermediat A-34 | 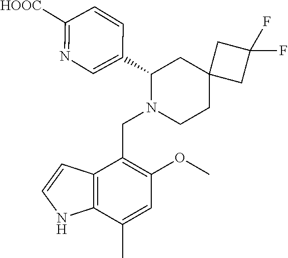 [SMILES: COc1cc(C)c2[nH]ccc2c1CN1CCC2(C[C@H]1c1ccc(C(=O)O)nc1)CC(F)(F)C2] | ¹H NMR (400 MHz, DMSO- d₆), measured with 1 drop of TFA δ [ppm] 11.20 (br s, 1H), 9.43 − 9.27 (m, 1H), 9.07 − 8.93 (m, 1H), 8.46 − 8.30 (m, 1H), 8.32 − 8.21 (m, 1H), 7.43 (t, J = 2.8 Hz, 1H), 6.76 (s, 1H), 6.43 (t, J = 2.4 Hz, 1H), 4.74 − 4.64 (m, 1H), 4.29 − 4.18 (m, 1H), 4.07 (d, J = 12.6 Hz, 1H), 3.67 (s, 3H), 3.48 − 3.35 (m, 1H), 3.34 − 3.25 (m, 1H), 2.96 − 2.82 (m, 1H), 2.76 − 2.62 (m, 1H), 2.50 − 2.39 (m, 5H), 2.34 − 2.25 (m, 1H), 2.17 − 1.99 (m, | Method B-7: Rt = 1.84 min; MS m/z [M + H]⁺ = 456.4 | |
| | | | 2H), 1.85 − 1.75 (m, 1H) | | |
| | | | 27 of 28H reported | | |
| | | | (—COOH not observed) | | |
| Ex-65 | Intermediate A-35 | 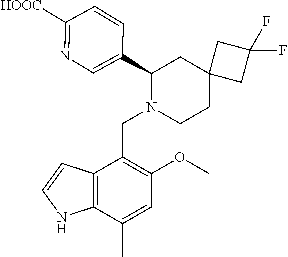 [SMILES: COc1cc(C)c2[nH]ccc2c1CN1CCC2(C[C@@H]1c1ccc(C(=O)O)nc1)CC(F)(F)C2] | | Method B-7: Rt = 1.88 min; MS m/z [M + H]⁺ = 456.2 | |
| Ex-66 | Intermediate A-36 | 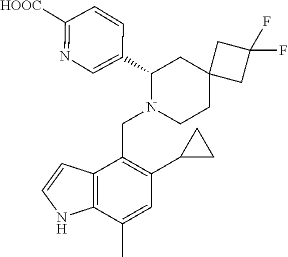 [SMILES: Cc1cc(C2CC2)c(CN2CCC3(C[C@H]2c2ccc(C(=O)O)nc2)CC(F)(F)C3)c2cc[nH]c12] | Partial formic acid salt ¹H NMR (400 MHz, DMSO- d₆) δ [ppm] 13.11 (br s, 1H), 10.85 (br s, 1H), 8.86 − 8.78 (m, 1H), 8.12 − 8.01 (m, 2H), 7.24 (t, J = 2.8 Hz, 1H), 6.56 − 6.51 (m, 1H), 6.47 (s, 1H), 3.67 (d, J = 12.0 Hz, 1H), 3.40 − 3.32 (m, 2H* partially under water peak), 2.70 − 2.58 (m, 2H), 2.50 − 2.43 (m, 1H), 2.39 − 2.27 (m, 5H* partially under solvent peak), 2.15 − 2.01 (m, 2H), 1.92 − 1.81 (m, 1H), 1.78 − 1.70 (m, 1H), 1.63 − 1.48 (m, | Method B-6: Rt = 0.81 min; MS m/z [M + H]⁺ = 466.2 | |
| | | | 2H), 0.86 − 0.76 (m, 1H), | | |
| | | | 0.72 − 0.64 (m, 1H), 0.54 − | | |
| | | | 0.44 (m, 1H), 0.19 − 0.10 (m, | | |
| | | | 1H) | | |
| | | | 27 of 27H reported | | |
| Ex-67 | Intermediate A-37 | 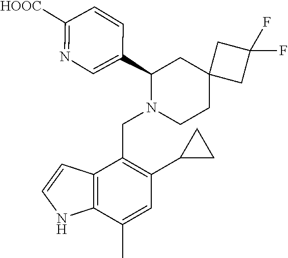 [SMILES: Cc1cc(C2CC2)c(CN2CCC3(C[C@@H]2c2ccc(C(=O)O)nc2)CC(F)(F)C3)c2cc[nH]c12] | | Method B-6: Rt = 0.82 min; MS m/z [M + H]⁺ = 466.4 | |
| Ex-68 | Intermediate A-38 | 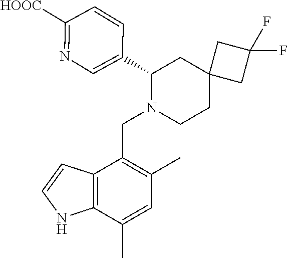 [SMILES: Cc1cc(C)c2[nH]ccc2c1CN1CCC2(C[C@H]1c1ccc(C(=O)O)nc1)CC(F)(F)C2] | Partial formic acid salt ¹H NMR (400 MHz, DMSO- d₆) δ [ppm] 13.08 (br s, 1H), 10.85 (br s, 1H), 8.91 − 8.81 (m, 1H), 8.11 − 8.03 (m, 2H), 7.22 (t, J = 2.8 Hz, 1H), 6.64 (s, 1H), 6.49 (dd, J = 3.1, 1.9 Hz, 1H), 3.47 (d, J = 12.1 Hz, 1H), 3.37 − 3.30 (m, 1H* partially under water peak), 3.20 (d, J = 12.1 Hz, 1H), 2.69 − 2.54 (m, 2H), 2.53 − 2.41 (m, 1H* partially under solvent peak), 2.39 − 2.27 (m, 5H), 2.21 (s, 3H), 2.04 − 1.94 (m, 1H), 1.90 − 1.79 (m, | Method B-6: Rt = 0.71 min; MS m/z [M + H]⁺ = 440.0 | |
| | | | 1H), 1.76 − 1.67 (m, 1H), | | |
| | | | 1.61 − 1.47 (m, 2H) | | |
| | | | 27 of 27H reported | | |
| Ex-69 | Intermediate A-39 | 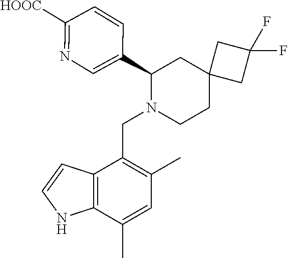 [SMILES: Cc1cc(C)c2[nH]ccc2c1CN1CCC2(C[C@@H]1c1ccc(C(=O)O)nc1)CC(F)(F)C2] | | Method B-6: Rt = 0.69 min; MS m/z [M + H]⁺ = 440.4 | |
| Ex-70 | Intermediate P-27 | 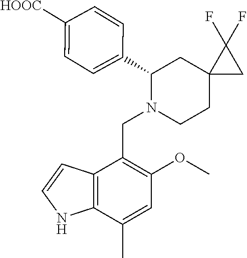 [SMILES: COc1cc(C)c2[nH]ccc2c1CN1CCC2(C[C@H]1c1ccc(C(=O)O)cc1)CC2(F)F] | ¹H NMR (400 MHz, DMSO- d₆) δ [ppm] 10.80 (s, 1H), 7.89 − 7.82 (m, 2H), 7.48 − 7.39 (m, 2H), 7.24 (t, J = 2.8 Hz, 1H), 6.65 (s, 1H), 6.43 (dd, J = 3.0, 1.9 Hz, 1H), 3.71 (s, 3H), 3.57 (d, J = 11.8 Hz, 1H), 3.17 (d, J = 11.8 Hz, 1H), 3.07 (d, J = 11.2 Hz, 1H), 2.82 (d, J = 11.5 Hz, 1H), 2.44 − 2.38 (m, 3H), 2.06 − 1.88 (m, 2H), 1.79 − 1.68 (m, 1H), 1.47 (d, J = 13.6 Hz, 1H), 1.34 (d, J = 13.3 Hz, 1H), 1.30 − 1.13 (m, 2H) | Method A-1: Rt = 1.32 min; MS m/z [M + H]⁺ = 441.2 | |
| | | | 25 of 26H reported | | |

### Measurement of Optical Rotations

Method OR1: Anton Paar MOP 200 Polarimeter, serial #82006136, 100-mm path-length cylindrical glass cell at 25° C. The wavelength of the light used was 589 nanometer (the sodium 0 line). Optical rotation of the same cell filled with solvent was subtracted as baseline. The final result was the average of ten measurements.

Method OR²: Perkin-Elmer 241 Polarimeter MC, serial #5126, 100-mm path-length cylindrical glass cell. The wavelength of the light used was 589 nanometer (the sodium 0 line). Optical rotation of the same cell filled with solvent was subtracted as baseline. The final result was the average of three measurements.

### LC-MS Methods

### LC-MS Method A-1:

Column: Synergi 2.5μ (20×4.0 mm), MAX-RP 100 A Mercury
Column temperature: 40° C.
Eluents: A: water+FA (0.1%)
   B: ACN
Flow rate: 2.0 mL/min
Gradient: Time/% B: 0.1/5; 0.5/5; 1.0/95; 1.5/95; 2.0/5; 3.0/5 (3 min method)

### LC-MS Method C-1:

Column: Acquity UPLC BEH C18, 130 Å 1.7 μm, 2.1 mm×30 mm
Column temperature: 50° C.
Eluents: A: water+NH₄OH (5 mM)
   B: ACN+NH₄OH (5 mM)
Flow rate: 1.0 mL/min
Gradient: 2% to 98% B in 2.0 min

### LC-MS Method C-2:

Column: Acquity UPLC BEH C18, 130 Å 1.7 μm, 2.1 mm×50 mm
Column temperature: 50° C.
Eluents: A: water+NH₄OH (5 mM)
   B: ACN+NH₄OH (5 mM)
Flow rate: 1.0 mL/min
Gradient: 2% to 98% B in 5.2 min

### LC-MS Method C-3:

Column: ACQUITY UPLC BEH C18, 130 Å, 1.7 μm, 2.1 mm×30 mm
Column temperature: 50° C.
Eluents: A: water+FA (0.1%)
   B: ACN+FA (0.1%)
Flow rate: 1.0 mL/min
Gradient: 2% to 98% B in 2.0 min

### LC-MS Method B-1:

Column: CORTECS C18+, 2.1×50 mm Column, 2.7 μm
Column temperature: 80° C.
Eluents: A: water+4.76% IPA+0.05% FA+3.75 mM NH₄(CH₃CO₂)
   B: IPA+0.05% FA
Flow rate: 1.0 mL/min
Gradient: initial 1% B; 1% to 50% B in 1.4 min, 50% to 98% B in 0.30 min; 0.10 min 98% B.

### LC-MS Method B-2:

Column: XBridge BEH, 2.1×50 mm Column, 2.5 μm
Column temperature: 80° C.
Eluents: A: water+5 mM NH₄OH
   B: ACN+5 mM NH₄OH:
Flow rate: 1.0 mL/min
Gradient: initial 2% B; 2% to 98% B in 1.40 min; 0.40 min 98% B.

### LC-MS Method B-3:

Column: Acquity UPLC BEH C18, 2.1×100 mm Column, 1.7 μm
Column temperature: 80° C.
Eluents: A: water+4.76% IPA+0.05% FA+3.75 mM NH₄(CH₃CO₂)
   B: IPA+0.05% FA
Flow rate: 0.4 mL/min
Gradient: Pre-run with 1% B for 0.50 min; 1% to 60% B in 8.4 min, 60% to 98% B in 1.00 min; 0.40 min 98% B.

### LC-MS Method B-4:

Column: CORTECS C18+, 2.1×50 mm Column, 2.7 μm
Column temperature: 80° C.
Eluents: A: water+0.05% FA+3.75 mM NH₄(CH₃CO₂)
   B: IPA+0.05% FA
Flow rate: 1.0 mL/min
Gradient: initial 1% B; from 1% to 98% B in 1.40 min; 0.40 min 98% B.

### LC-MS Method B-5:

Column: ACQUITY UPLC BEH C18, 2.1×50 mm Column, 1.7 μm
Column temperature: 80° C.
Eluents: A: water+0.01% TFA
   B: ACN
Flow rate: 1.0 mL/min
Gradient: initial 5% B; from 5 to 98% B in 1.4 min.

### LC-MS Method B-6:

Column: CORTECS C18+, 2.1×50 mm Column, 2.7 μm
Column temperature: 80° C.
Eluents: A: water+0.05% FA+3.75 mM NH₄(CH₃CO₂)
   B: IPA+0.05% FA
Flow rate: 1.0 mL/min
Gradient: initial 5% B; 5% to 50% B in 1.4 min, 50% to 98% B in 0.30 min; 0.10 min 98% B.

### LC-MS Method B-7:

Column: Acquity UPLC BEH C18, 2.1×100 mm Column, 1.7 μm
Column temperature: 80° C.
Eluents: A: water+0.05% FA+3.75 mM ammonium acetate
   B: IPA+0.05% FA
Flow rate: 0.4 mL/min
Gradient: Pre-run with 5% B for 0.50 min; 5% to 60% B in 8.4 min, 60% to 98% B in 1.00 min; 0.40 min 98% B.

### Achiral Preparative HPLC Methods

Waters AutoPurification System with PDA (photodiode array detector) and single quadrupole mass detector with ESI ionization.

### Instrument:

Pump: Waters 2545
Fraction Collector: Waters 2767
Modifier Pump: Waters 515
Detector: Waters 2998
Mass Spec: Waters SQD-2
Column: Waters XBridge BEH C18 OBD 5 μm 30×50 mm
Eluents: A: water
   B: ACN
Flow rate: 75 mL/min
Stop Time: 6.5 min
Column temperature: RT
UV 210-400 nm

### Chiral HPLC Methods

### Method SFC-1:

Instrument: Waters Acquity UPC2
Column: Chiralpak IE-3 (3 μm) 3 mm×100 mm
Column temperature: 40° C.
Eluents: A: 0.1% NH₃ in MeOH/IPA (1/1)
   B: scCO₂
Flow rate: 2.5 mL/min
Gradient: 5% B in 0.2 min, 5-55% in 3.0 min
Pressure: 1800 psi

### Method SFC-2:

Instrument: Waters Acquity UPC2
Column: (S,S) Whelk-O1 (3.5 μm) 3 mm×100 mm
Column temperature: 40° C.
Eluents: A: 0.1% NH₃ in MeOH/IPA (1/1)
   B: scCO₂
Flow rate: 2.5 mL/min
Gradient: 5% B in 0.2 min, 5-55% in 3.0 min
Pressure: 1800 psi

### Method SFC-3:

### Preparative Chiral HPLC

Instrument: Sepiatec prep SFC-100
Column: Chiralpak AD, 250 mm×30 mm 5 μm
Eluent: A: 20% 0.05% NH₃ in MeOH, B: 80% scCO₂ (isocratic)
Flow rate: 90.0 mL/min
Detection: UV 234 nm
Injection volume: 2.3 mL
Oven temperature: 40° C.
Pressure: 130 bar

### Analytical Chiral HPLC

Instrument: Analytical SFC-MS Waters UPC2
Injection: 5 μL Mobile phase: A: 20% 0.1% NH₃ in IPA, B: 80% scCO₂ (isocratic)
Flow rate: 3 mL/min
Column: Chiralpak AD (4.6 mm×100 mm 5 μm)
Detection UV: DAD
Oven temperature: 40° C.

Pressure: 1800 psi

### Method SFC-4:

### Preparative Chiral HPLC

Instrument: Sepiatec prep SFC-100
Column: Chiralpak AD, 250 mm×30 mm 5 μm
Eluent: A: 22% 0.05% NH₃ in MeOH, B: 78% scCO₂ (isocratic)
Flow rate: 90.0 mL/min
Detection: UV 234 nm
Injection volume: 2.5 mL
Oven temperature: 40° C.
Pressure: 130 bar

### Analytical Chiral HPLC

Instrument: Analytical SFC-MS Waters UPC2
Injection: 5 μL
Mobile phase: A: 20% 0.1% NH₃ in IPA, B: 80% scCO₂ (isocratic)
Flow rate: 3 mL/min
Column: Chiralpak AD (4.6 mm×100 mm 5 μm)
Detection UV: DAD
Oven Temperature: 40° C.
Pressure: 1800 psi

### Method SFC-5:

### Preparative Chiral HPLC

Instrument: Waters Prep SFC100 MS
Column: Chiralpak AD, 250 mm×30 mm 5 μm
Eluent: A: 18% 0.05% NH₃ in IPA, B: 82% scCO₂ (isocratic)
Flow rate: 80.0 mL/min
Detection: DAD
Injection volume: 2.0 mL
Oven temperature: 40° C.
Pressure: 120 bar

### Analytical Chiral HPLC

Instrument: Analytical SFC-MS Waters UPC2
Injection: 5 μL
Mobile phase: A: 18% 0.1% NH₃ in IPA, B: 82% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-6:

### Preparative Chiral HPLC

Instrument: Waters Prep SFC100 MS
Column: Chiralpak IG, 250 mm×30 mm 5 μm
Eluent: A: 50% 0.05% NH₃ in MeOH, B: 50% scCO₂ (isocratic)
Flow rate: 80.0 mL/min
Detection: DAD
Injection volume: 3.5 mL
Oven temperature: 40° C.
Pressure: 120 bar

### Analytical Chiral HPLC

Instrument: Analytical SFC-MS Waters UPC2
Injection: 5 μL
Mobile phase: A: 50% 0.05% NH₃ in MeOH, B: 50% scCO₂ (isocratic)
Flow rate: 3 mL/min
Column: Chiralpak IG (4.6 mm×100 mm 5 μm)
Detection UV: DAD
Oven Temperature: 40° C.
Pressure: 1800 psi

### Method SFC-7:

### Preparative Chiral HPLC

Instrument: Sepiatec prep SFC-100
Column: Chiralpak IC, 250 mm×30 mm 5 μm
Eluent: A: 30% 0.05% NH₃ in IPA, B: 70% scCO₂ (isocratic)
Flow rate: 80.0 mL/min
Detection: UV 237 nm
Injection volume: 0.8 mL
Oven temperature: 36° C.
Pressure: 130 bar

### Analytical Chiral HPLC

Instrument: Analytical SFC-MS Waters UPC2
Injection: 5 μL
Mobile phase: A: 30% 0.05% NH₃ in IPA, B: 70% scCO₂ (isocratic)
Flow rate: 3 mL/min
Column: Chiralpak IC (4.6 mm×100 mm 5 μm)
Detection UV: DAD
Oven temperature: 40° C.
Pressure: 1800 psi

### Method SFC-8:

### Preparative Chiral HPLC

Instrument: Waters Prep SFC100 MS
Column: Amylose-1 250×30 mm 5 μm
Eluent: A: 22% 0.05% NH₃ in MeOH B: 78% scCO₂ (isocratic)
Flow rate: 80.0 mL/min
Detection: DAD
Injection volume: 2.0 mL
Oven temperature: 40° C.
Pressure: 120 bar

### Analytical Chiral HPLC

Instrument: Analytical SFC-MS Waters UPC2
Injection: 5 μL
Mobile phase: A: 20% 0.05% NH₃ in MeOH, B: 80% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-9:

### Preparative Chiral HPLC

Instrument: Sepiatec SFC 100
Column: Chiralpak AD, 250 mm×30 mm 5 μm
Eluent: A: 18% 0.05% NH₃ in MeOH, B: 82% scCO₂ (isocratic)
Flow rate: 80.0 mL/min
Detection: UV 220 nm
Injection volume: 2.4 mL
Oven temperature: 40° C.
Pressure: 130 bar

### Analytical Chiral HPLC

Instrument: Analytical SFC-MS Waters UPC2
Injection: 5 μL
Mobile phase: A: 18% 0.05% NH₃ in MeOH, B: 82% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-10:

### Preparative Chiral HPLC

Instrument: Sepiatec SFC100
Column: Chiralpak IG, 250 mm×30 mm 5 μm
Eluent: A: 40% 0.05% NH₃ in MeOH, B: 60% scCO₂ (isocratic)
Flow rate: 80.0 mL/min
Detection: UV 220 nm
Injection volume: 2.2 mL
Oven temperature: 40° C.
Pressure: 130 bar

### Analytical Chiral HPLC

Instrument: Analytical SFC-MS Waters UPC2
Injection: 5 μL
Mobile phase: A: 40% 0.05% NH₃ in MeOH, B: 60% scCO₂ (isocratic)
Flow rate: 3 mL/min
Column: Chiralpak IG (4.6 mm×100 mm 5 μm)
Detection UV: DAD
Oven temperature: 40° C.
Pressure: 1800 psi

### Method SFC-11:

### Preparative Chiral HPLC

Instrument: Sepiatec SFC100
Column: Chiralpak AD, 250 mm×30 mm 5 μm
Eluent: A: 35% 0.05% NH₃ in MeOH, B: 65% scCO₂ (isocratic)
Flow rate: 80.0 mL/min
Detection: DAD
Injection volume: 2.8 mL
Oven temperature: 40° C.
Pressure: 130 bar

### Analytical Chiral HPLC

Instrument: Analytical SFC-MS Waters UPC2
Injection: 5 μL
Mobile phase: A: 30% 0.05% NH₃ in MeOH, B: 70% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-12:

### Preparative Chiral HPLC

Instrument: Waters Prep SFC100 MS
Column: Chiralpak IG, 250 mm×30 mm 5 μm
Eluent: A: 20% 0.05% NH₃ in IPA, B: 80% scCO₂ (isocratic)
Flow rate: 80.0 mL/min
Detection: DAD
Injection volume: 4.0 mL
Oven temperature: 40° C.
Pressure: 120 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak IG (4.6 mm×100 mm 5 μm)
Detection UV: DAD
Oven temperature: 40° C.
Pressure: 1800 psi

### Method SFC-13:

### Preparative Chiral HPLC

instrument: Waters Prep SFC100 MS
Column: Chiralpak IG, 250 mm×30 mm 5 μm
Eluent: A: 30% 0.05% NH₃ in IPA, B: 70% scCO₂ (isocratic)
Flow rate: 80.0 mL/min
Detection: DAD
Injection volume: 4 mL
Oven temperature: 40° C.
Pressure: 120 bar

### Analytical Chiral HPLC

Instrument: Analytical SFC-MS Waters UPC2
Injection: 5 μL
Mobile phase: A: 30% 0.05% NH₃ in IPA, B: 70% scCO₂ (isocratic)
Flow rate: 3 mL/min
Column: Chiralpak IG (4.6 mm×100 mm 5 μm)
Detection UV: DAD
Oven temperature: 40° C.
Pressure 1800 psi

### Method SFC-14:

### Preparative Chiral HPLC

Instrument: Sepiatec SFC100
Column: Chiralpak AD-H, 250 mm×30 mm 5 μm
Eluent: A: 23% 0.05% NH₃ in MeOH, B: 77% scCO₂ (isocratic)
Flow rate: 90.0 mL/min;
Detection: UV 234 nM
Injection volume: 2.30 mL
Oven temperature: 36° C.
Pressure: 130 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-15:

### Preparative Chiral HPLC

Instrument: Sepiatec SFC100
Column: Chiralpak AD, 250 mm×30 mm 5 μm
Eluent: A: 22% 0.05% NH₃ in IPA, B: 78% scCO₂ (isocratic)
Flow rate: 85.0 mL/min
Detection: UV 234 nm
Injection volume: 9 mL
Oven temperature: 40° C.
Pressure: 120 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-16:

Instrument: Agilent 1260 infinity II
Column: Chiralpak IJ (5 μm×4.6 mm×150 mm)
Column temperature: 25° C.
Eluent: A: 80% hexane, B: 20% EtOH/MeOH (1:1) (isocratic)
Flow rate: 1 mL/min
Detection UV: DAD

### Method SFC-17:

Instrument: Agilent 1260 infinity II
Column: Chiralpak IG (5 μm×4.6 mm×150 mm)
Column temperature: 25° C.
Eluent: A: 70% hexane, B: 30% EtOH/MeOH (1:1) (isocratic)
Flow rate: 1 mL/min
Detection UV: DAD

### Method SFC-18:

### Preparative Chiral HPLC

instrument: Waters Prep SFC100 MS
Column: Chiralpak AD, 250 mm×30 mm 5 μm
Eluent: A: 19% 0.05% NH₃ in IPA, B: 81% scCO₂ (isocratic)
Flow rate: 80.0 mL/min
Detection: DAD
Injection volume: 3 mL
Oven temperature: 40° C.
Pressure: 120 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak AD (4.6 mm×100 mm 5 μm)
Detection UV: DAD
Oven temperature: 40° C.
Pressure 1800 psi

### Method SFC-19:

### Preparative Chiral HPLC

instrument: Sepiatec prep SFC 100
Column: Lux Cellulose 2, 250 mm×30 mm 5 μm (OZ-like)
Eluent: A: 25% 0.05% NH₃ in IPA, B: 75% scCO₂ (isocratic)
Flow rate: 110.0 mL/min
Detection: UV 217 nm
Injection volume: 27×3 mL
Oven temperature: 40° C.
Pressure: 130 bar

### Analytical Chiral HPLC

Instrument: Analytical SFC-MS Waters UPC2
Injection: 5 μL
Mobile phase: A: 30% 0.05% NH₃ in IPA, B: 70% scCO₂ (isocratic)
Flow rate: 3 mL/min
Column: Chiralcel OZ (4.6 mm×100 mm 5 μm)
Detection UV: DAD
Oven temperature: 40° C.
Pressure 1800 psi

### Method SFC-20:

### Preparative Chiral HPLC

instrument: Sepiatec prep SFC 100
Column: Chiralpak AD-H, 250 mm×30 mm 5 μm
Eluent: A: 15% 0.05% NH₃ in IPA, B: 85% scCO₂ (isocratic)
Flow rate: 80 mL/min
Detection: UV 220 nm
Injection volume: 7×1.60 mL
Oven temperature: 40° C.
Pressure: 90 bar

### Analytical Chiral HPLC

Instrument: Analytical SFC-MS Waters UPC2
Injection: 5 μL
Mobile phase: A: 18% 0.05% NH₃ in IPA, B: 82% scCO₂ (isocratic)
Flow rate: 3 mL/min
Column: Chiralpak AD (4.6 mm×100 mm 5 μm)
Detection UV: DAD
Oven temperature: 40° C.
Pressure 1800 psi

### Method SFC-21:

### Preparative Chiral HPLC

instrument: Sepiatec prep SFC 100
Column: Chiralpak IG, 250 mm×30 mm 5 μm
Eluent: A: 30% 0.05% NH₃ in IPA, B: 70% scCO₂ (isocratic)
Flow rate: 80 mL/min
Detection: UV 220 nm
Injection volume: 21×0.60 mL
Oven temperature: 40° C.
Pressure: 130 bar

### Analytical Chiral HPLC

Instrument: Analytical SFC-MS Waters UPC2
Injection: 5 μL
Mobile phase: A: 30% 0.05% NH₃ in IPA, B: 70% scCO₂ (isocratic)
Flow rate: 3 mL/min
Column: Chiralpak IG (4.6 mm×100 mm 5 μm)
Detection UV: DAD
Oven temperature: 40° C.
Pressure 1800 psi

### Method SFC-22:

### Preparative Chiral HPLC

instrument: Sepiatec prep SFC 100
Column: Chiralpak IG, 250 mm×30 mm 5 μm
Eluent: A: 18% 0.05% NH₃ in IPA, B: 82% scCO₂ (isocratic)
Flow rate: 80 mL/min
Detection: UV 235 nm
Injection volume: 16×0.80 mL
Oven temperature: 40° C.
Pressure: 140 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak IG (4.6 mm×100 mm 5 μm)
Detection UV: DAD
Oven temperature: 40° C.
Pressure 1800 psi

### Method SFC-23:

### Preparative Chiral HPLC

instrument: Sepiatec prep SFC 100
Column: Chiralpak IG, 250 mm×30 mm 5 μm
Eluent: A: 40% 0.05% NH₃ in IPA, B: 60% scCO₂ (isocratic)
Flow rate: 130 mL/min
Detection: UV 245 nm
Injection volume: 15×3.00 mL
Oven temperature: 40° C.
Pressure: 130 bar

### Analytical Chiral HPLC

Instrument: Analytical SFC-MS Waters UPC2
Injection: 5 μL
Mobile phase: A: 35% 0.05% NH₃ in IPA, B: 65% scCO₂ (isocratic)
Flow rate: 3 mL/min
Column: Chiralpak IG (4.6 mm×100 mm 5 μm)
Detection UV: DAD
Oven temperature: 40° C.
Pressure 1800 psi

### Method SFC-24:

### Preparative Chiral HPLC

instrument: Sepiatec prep SFC 100
Column: Chiralpak AD-H, 250 mm×30 mm 5 μm
Eluent: A: 35% 0.05% NH₃ in IPA, B: 65% scCO₂ (isocratic)
Flow rate: 80 mL/min
Detection: UV 220 nm
Injection volume: 9×1.50 mL
Oven temperature: 40° C.
Pressure: 130 bar

### Analytical Chiral HPLC

Instrument: Analytical SFC-MS Waters UPC2
Injection: 5 μL
Mobile phase: A: 35% 0.05% NH₃ in IPA, B: 65% scCO₂ (isocratic)
Flow rate: 3 mL/min
Column: Chiralpak AD (4.6 mm×100 mm 5 μm)
Detection UV: DAD
Oven temperature: 40° C.
Pressure 1800 psi

### Method SFC-25:

### Preparative Chiral HPLC

instrument: Sepiatec prep SFC 100
Column: Chiralpak AD-H, 250 mm×30 mm 5 μm
Eluent: A: 38% 0.05% NH₃ in IPA, B: 62% scCO₂ (isocratic)
Flow rate: 80 mL/min
Detection: UV 220 nm
Injection volume: 10×2.00 mL
Oven temperature: 40° C.
Pressure: 130 bar

### Analytical Chiral HPLC

Instrument: Analytical SFC-MS Waters UPC2
Injection: 5 μL
Mobile phase: A: 30% 0.05% NH₃ in IPA, B: 70% scCO₂ (isocratic)
Flow rate: 3 mL/min
Column: Chiralpak AD (4.6 mm×100 mm 5 μm)
Detection UV: DAD
Oven temperature: 40° C.
Pressure 1800 psi

### Method SFC-26:

### Preparative Chiral HPLC

instrument: Waters Prep SFC100 MS
Column: Chiralpak IC, 250 mm×30 mm 5 μm
Eluent: A: 28% 0.1% NH₃ in MeOH, B: 72% scCO₂ (isocratic)
Flow rate: 80 mL/min
Detection: DAD
Injection volume: 1.0 mL
Oven temperature: 40° C.
Pressure: 120 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak AD (4.6 mm×100 mm 5 μm)
Detection UV: DAD
Oven temperature: 40° C.
Pressure 1800 psi

### Method SFC-27:

### Preparative Chiral HPLC

instrument: Sepiatec prep SFC 100
Column: Chiralpak IG, 250 mm×30 mm 5 μm
Eluent: A: 27% 0.05% NH₃ in IPA, B: 73% scCO₂ (isocratic)
Flow rate: 80 mL/min
Detection: UV 235 nm
Injection volume: 7×1.00 mL
Oven temperature: 40° C.
Pressure: 110 bar

### Analytical Chiral HPLC

Instrument: Analytical SFC-MS Waters UPC2
Injection: 5 μL
Mobile phase: A: 25% 0.05% NH₃ in IPA, B: 75% scCO₂ (isocratic)
Flow rate: 3 mL/min
Column: Chiralpak IG (4.6 mm×100 mm 5 μm)
Detection UV: DAD
Oven temperature: 40° C.
Pressure 1800 psi

### Method SFC-28:

### Preparative Chiral HPLC

instrument: Sepiatec prep SFC 100
Column: Chiralpak IG, 250 mm×30 mm 5 μm
Eluent: A: 25% 0.05% NH₃ in IPA, B: 75% scCO₂ (isocratic)
Flow rate: 80 mL/min
Detection: UV 230 nm
Injection volume: 13×1.00 mL
Oven temperature: 40° C.
Pressure: 130 bar

### Analytical Chiral HPLC

Instrument: Analytical SFC-MS Waters UPC2
Injection: 5 μL
Mobile phase: A: 25% 0.05% NH₃ in IPA, B: 75% scCO₂ (isocratic)
Flow rate: 3 mL/min
Column: Chiralpak IG (4.6 mm×100 mm 5 μm)
Detection UV: DAD
Oven temperature: 40° C.
Pressure 1800 psi

### Biological Analysis

### Determination of Complement Factor B Inhibition

### Biological Example 1: Human Complement Factor B ELISA Assay

CVF-Bb complex prepared from purified cobra venom factor (1 μM), recombinant human complement factor B (expressed in drosophila cells and purified using standard methods) and human complement factor D (expressed in E. Coli, refolded and purified using standard methods). CVF-Bb complex at 3 nM concentration was incubated with test compound at various concentrations for 1 h at RT in PBS pH 7.4 containing 10 mM MgCl₂ and 0.05% (w/v) CHAPS. Human complement C3 substrate purified from plasma was added to a final concentration of 1 μM. After 1 h incubation at RT, the enzyme reaction was stopped by addition of a cocktail of concentrated pan-protease inhibitors. The product of the reaction, C3a, was quantified by means of an enzyme-linked-immunosorbent assay. IC₅₀ values were calculated from percentage of inhibition of CVF-Bb activity as a function of test compound concentration.

### Biological Example 2: Human Complement Factor B TR-FRET Assay

Recombinant human factor B (expressed in drosophila cells and purified using standard methods) labeled with biotin (10 nM), europium-labeled streptavidin (5 nM) and (+) or (−)-2-((1E,3E,5E)-5-(1-(6-((2-(3-(4-((R)-3-amino-3-phenylpropanoyl)-1-(4-amino-6,7-dimethoxyquinazolin-2-yl)piperazin-2-yl)phenoxy)ethyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium (prepared as described in WO2015/009616, 240 nM activity agaist factor B when tested using the assay of Biological Example 1) (75 nM) were incubated with test compound at various concentrations up to 2 hours at RT in 20 mM Tris/HCl, pH 7.4, 0.005% (v/v) Tween20.

The time-gated decrease in fluorescence intensity related to the competition between labeled and unlabeled factor B ligands was recorded at both 620 nm and 665 nm, 70 μs after excitation at 337 nm using a microplate spectrofluorimeter. IC₅₀ values were calculated from percentage of inhibition of complement factor B-(+) or (−)-2-((1E,3E,5E)-5-(1-(6-((2-(3-(4-((R)-3-amino-3-phenylpropanoyl)-1-(4-amino-6,7-dimethoxyquinazolin-2-yl)piperazin-2-yl)phenoxy)ethyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium (Biological Example 2.6, 240 nM activity agaist factor B when tested using the assay of Biological Example 1) displacement as a function of test compound concentration.

Compounds of disclosure are active on factor B inhibition. Data in Table 1 were collected using the assay of Biological Example 2.

TABLE 1

| | hFactor-B TR- |
|---|---|
| Example | FRET Qualified IC₅₀ [μM] |
| Ex-1 | 0.0005 |
| Ex-2 | 6.8122 |
| Ex-3 | 0.0407 |
| Ex-4 | 0.4611 |
| Ex-5 | 0.0005 |
| Ex-6 | 0.7145 |
| Ex-7 | 0.0008 |
| Ex-8 | 0.2270 |
| Ex-9 | 0.0021 |
| Ex-10 | 0.2423 |
| Ex-11 | 0.0058 |
| Ex-12 | 5.3044 |
| Ex-13 | 0.0294 |
| Ex-14 | 9.9996 |
| Ex-15 | 0.1054 |
| Ex-16 | 0.4342 |
| Ex-17 | 0.3197 |
| Ex-18 | 0.0034 |
| Ex-19 | 1.4580 |
| Ex-20 | 0.0076 |
| Ex-21 | 0.0014 |
| Ex-22 | 0.0024 |
| Ex-23 | 0.0089 |
| Ex-24 | 0.0314 |
| Ex-25 | 0.0040 |
| Ex-26 | >0.4* |
| Ex-27 | 0.0069 |
| Ex-28 | >0.4* |
| Ex-29 | 0.0051 |
| Ex-30 | >0.1* |
| Ex-31 | 0.0038 |
| Ex-32 | >0.4* |
| Ex-33 | 0.0016 |
| Ex-34 | 0.1241 |
| Ex-35 | 0.0173 |
| Ex-36 | 0.0245 |
| Ex-37 | 0.0112 |
| Ex-38 | 5.5067 |
| Ex-39 | 0.0103 |
| Ex-40 | 0.8644 |
| Ex-41 | 0.0096 |
| Ex-42 | 0.8611 |
| Ex-43 | 2.7883 |
| Ex-44 | 0.0099 |
| Ex-45 | 0.0094 |
| Ex-46 | 1.4650 |
| Ex-47 | 0.0025 |
| Ex-48 | 0.0089 |
| Ex-49 | 0.0056 |
| Ex-50 | 0.0070 |
| Ex-51 | 0.0042 |
| Ex-52 | 0.1722 |
| Ex-53 | 0.0077 |
| Ex-54 | 0.0022 |
| Ex-55 | 0.0006 |
| Ex-56 | 0.0586 |
| Ex-57 | 0.0050 |
| Ex-58 | 0.0038 |
| Ex-59 | 0.0021 |
| Ex-60 | 0.1521 |
| Ex-61 | 0.0038 |
| Ex-62 | 0.0019 |
| Ex-63 | 0.4336 |
| Ex-64 | 0.0021 |
| Ex-65 | 0.2348 |
| Ex-66 | 0.0021 |
| Ex-67 | 0.3368 |
| Ex-68 | 0.0045 |
| Ex-69 | 1.2337 |
| Ex-70 | 0.0147 |

*Assay behavior at and above the stated concentration prevented the measurement of activity for determining an IC₅₀.

The compounds of the disclosure inhibit complement factor B activity at -micromolar IC₅₀ values. As such, the compounds of the disclosure may be useful in treating the diseases and/or disorders described herein, e.g., a disease/disorder mediated by complement factor B.

All publications and patent documents cited herein are incorporated herein by reference as if each such publication or document was specifically and individually indicated to be incorporated herein by reference. The present disclosure and its embodiments have been described in detail. However, the scope of the present disclosure is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present disclosure. Accordingly, one of ordinary skill in the art will readily appreciate from the disclosure that later modifications, substitutions, and/or variations performing substantially the same function or achieving substantially the same result as embodiments described herein may be utilized according to such related embodiments of the present disclosure. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

1. A compound of formula (I) or a pharmaceutically acceptable salt thereof, 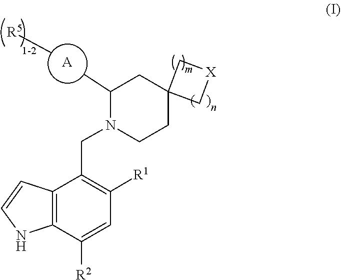
wherein:
X is O or CR^{X1}R^{X2};
R¹ is selected from H, C₁-C₆alkoxyl, C₃-C₅cycloalkoxyl, C₁-C₆alkyl, and C₃-C₅cycloalkyl, wherein the C₁-C₆alkoxyl, C₁-C₆alkyl, and C₃-C₅cycloalkyl are unsubstituted or substituted with 1 or 2 halogen substituents;
R² is C₁-C₃alkyl or C₃cycloalkyl wherein the C₁-C₃alkyl or C₃cycloalkyl are unsubstituted or substituted with 1 or 2 halogen substituents;
R^{X1} is selected from hydrogen, fluoro, C₁-C₆alkyl, and C₃-C₅cycloalkyl;
R^{X2} is selected from hydroxyl, fluoro, C₁-C₆alkyl, and C₃-C₅cycloalkyl;
with the proviso that R^{X1} is not fluoro when R^{X2} is hydroxyl;
or R^{X1} and R^{X2}, in combination with the carbon atom to which they are attached, form a spirocyclic carbocycle having 3-5 ring atoms;
A is a phenyl ring or a 5 or 6 membered heteroaryl ring having 1-4 heteroatoms independently selected from N, O, and S;
each R⁵ is independently selected from H, —CO₂R^{5b}, C₁-C₆alkyl, CH₂CO₂R^{5b}, C₁-C₆hydroxyalkyl, C₃-C₅cycloalkyl, 5- to 6-membered heteroaryl having 1-4 heteroatoms independently selected from N, O, and S, and 4- to 6-membered heterocyclyl having 1-2 heteroatoms independently selected from N, O, and S, wherein the C₁-C₆hydroxyalkyl, C₁-C₆alkyl, 5- to 6-membered heteroaryl and 4- to 6-membered heterocyclyl are unsubstituted or substituted with 1 or 2 R^{5a};
each R^{5a} is independently selected from fluoro, hydroxyl, and C₁-C₆alkyl, wherein the C₁-C₆alkyl is unsubstituted or substituted with 1, 2, or 3 fluoro;
wherein when R⁵ is a 4- to 6-membered heterocyclyl, two R^{5a} are not fluoro and hydroxyl substituted on the same position; and
R^{5b} is selected from H or C₁-C₅ alkyl;
m is 0 or 1;
n is 0, 1, or 2;
wherein both m and n are not 0; and
with the proviso that
when X is O, then m is 1 and n is 1 or 2.

2. The compound of formula (I) according to claim 1, or a pharmaceutically acceptable salt thereof, of formula (I-A) or (I-B)

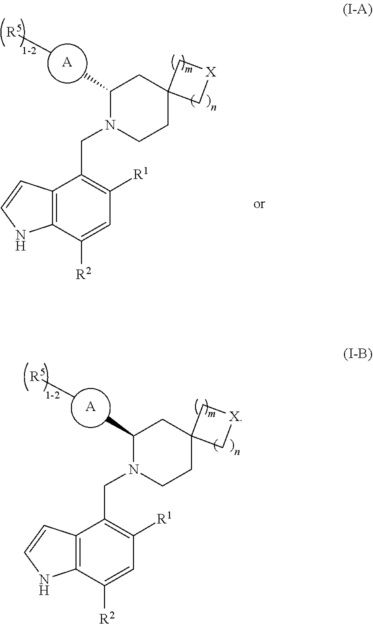

3. The compound I-A according to claim 2, or a pharmaceutically acceptable salt thereof.

4. The compound according to any one of the preceding claims, or a pharmaceutically acceptable salt thereof, wherein R² is selected from methyl, ethyl, and cyclopropyl.

5. The compound of according to claim 4, or a pharmaceutically acceptable salt thereof, wherein R² is methyl.

6. The compound according to any one of the preceding claims, or a pharmaceutically acceptable salt thereof, wherein A is a phenyl ring.

7. The compound of claim 6, or a pharmaceutically acceptable salt thereof, wherein R⁵ is substituted on the para position of the phenyl ring.

8. The compound according to any of claims 1-5, or a pharmaceutically acceptable salt thereof, wherein A is selected from furanyl, thiophenyl, pyrrolyl, oxazolyl, isoxazolyl, thiazolyl, pyrazolyl, imiadazolyl, pyridyl, triazolyl, tetrazolyl, oxadiazolyl, isoxadiazolyl, pyrimidinyl, pyrazinyl, and pyridazinyl.

9. The compound according to claim 8, or a pharmaceutically acceptable salt thereof, wherein A is selected from:

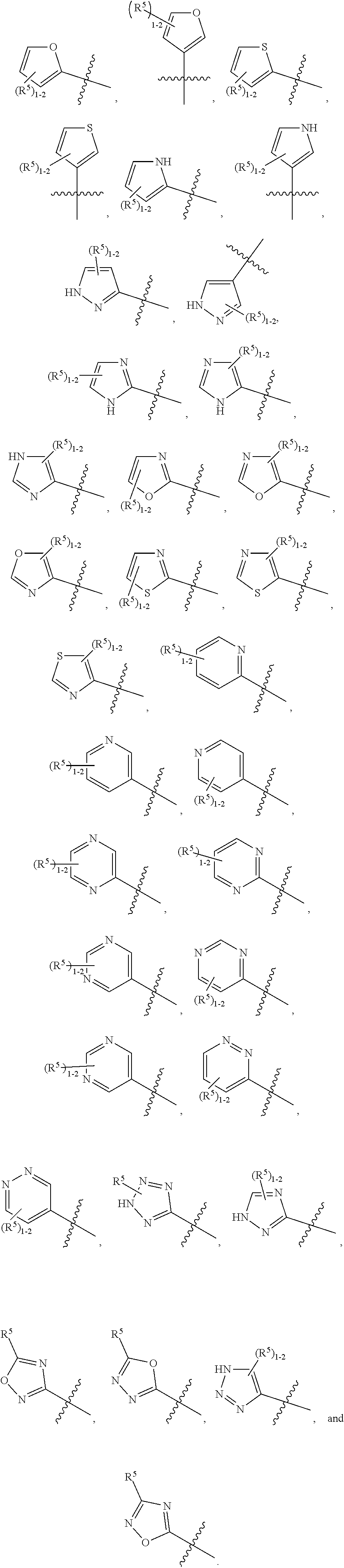

10. The compound according to any one of the preceding claims, or a pharmaceutically acceptable salt thereof, wherein R⁵ is selected from —CO₂H, C₁-C₆hydroxyalkyl, 5- to 6-membered heteroaryl having 1-4 heteroatoms independently selected from N, O, and S, and 4- to 6-membered heterocyclyl having 1 O heteroatom, wherein the 4- to 6-membered heterocyclyl is substituted with 0-1 R^{5a}.

11. The compound according to any one of the preceding claims, or a pharmaceutically acceptable salt thereof, wherein R⁵ is selected from CO₂R^{5b}, C₁-C₆hydroxyalkyl, 5-membered heteroaryl having 2 N heteroatoms, and 4- to 6-membered heterocyclyl having 1 O heteroatom, wherein 4- to 6-membered heterocyclyl is unsubstituted or substituted with 1 R^{5a}, wherein R^{5a} is hydroxyl and R^{5b} is H.

12. The compound according to any one of the preceding claims, or a pharmaceutically acceptable salt thereof, wherein R⁵ is selected from —CO₂H, tetrazole, and oxetane substituted with hydroxyl.

13. The compound according to any one of the preceding claims, or a pharmaceutically acceptable salt thereof, wherein R⁵ is —CO₂H.

14. The compound according to any one of the preceding claims, or a pharmaceutically acceptable salt thereof, wherein R⁵ is tetrazole.

15. The compound according to any one of the preceding claims, or a pharmaceutically acceptable salt thereof, wherein R¹ is selected from C₁-C₄alkoxyl, C₁-C₄alkyl, and C₃-C₆cycloalkyl, wherein the C₃-C₆cycloalkyl is unsubstituted or substituted with 1 or 2 fluoro substituents.

16. The compound according to any one of the preceding claims, or a pharmaceutically acceptable salt thereof, wherein R¹ is selected from methoxyl, methyl, and cyclopropyl, wherein the cyclopropyl is unsubstituted or substituted with 1 or 2 fluoro substituents.

17. The compound according to any one of the preceding claims, or a pharmaceutically acceptable salt thereof, wherein R¹ is selected from methoxyl, and cyclopropyl, wherein the cyclopropyl is unsubstituted or substituted with 1 or 2 fluoro substituents.

18. The compound according to any one of the preceding claims, or a pharmaceutically acceptable salt thereof, wherein R^{X1} and R^{X2} are both fluoro, fluoro and C₁-C₆alkyl, hydrogen and fluoro, C₁-C₆alkyl and hydroxyl, or hydrogen and hydroxyl.

19. The compound according to claim 18, or a pharmaceutically acceptable salt thereof, wherein R^{X1} and R^{X2} are both fluoro or fluoro and C₁-C₆alkyl, or fluoro and H.

20. The compound according to any one of the preceding claims, or a pharmaceutically acceptable salt thereof, wherein n is 1 or 2.

21. The compound according to any one of the preceding claims, or a pharmaceutically acceptable salt thereof, wherein X is O.

22. The compound according to any one of claims 1 to 20, or a pharmaceutically acceptable salt thereof, wherein X is CR^{X1}R^{X2}.

23. The compound according to claim 22, wherein R^{X1} is fluoro, and R^{X2} is selected from fluoro, and C₁-C₆alkyl.

24. The compound according to any one of the preceding claims, or a pharmaceutically acceptable salt thereof, wherein R^{5a} is hydroxyl.

25. The compound of formula (I) according to claim 1, or a pharmaceutically acceptable salt thereof, selected from:

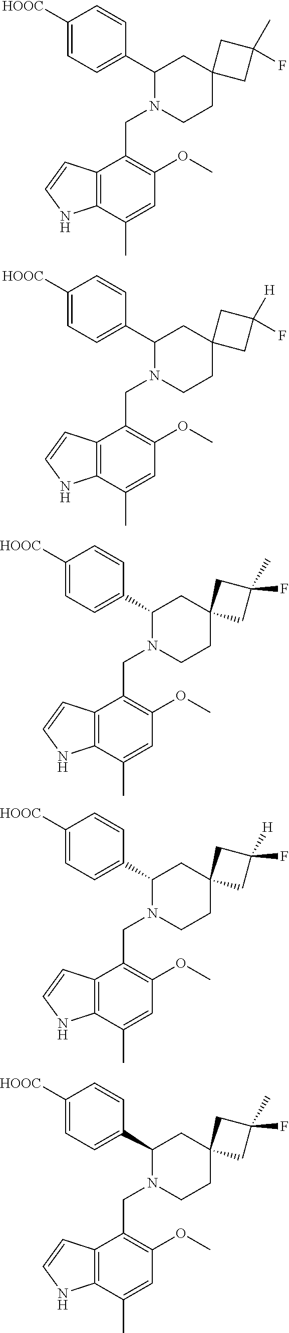

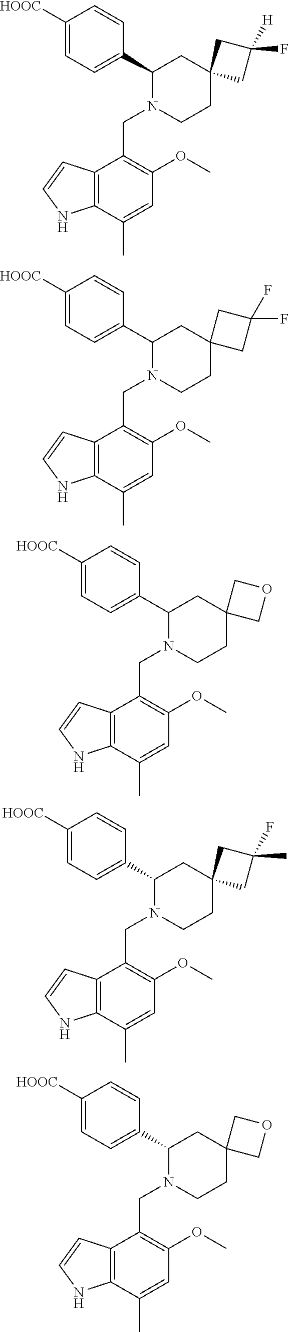

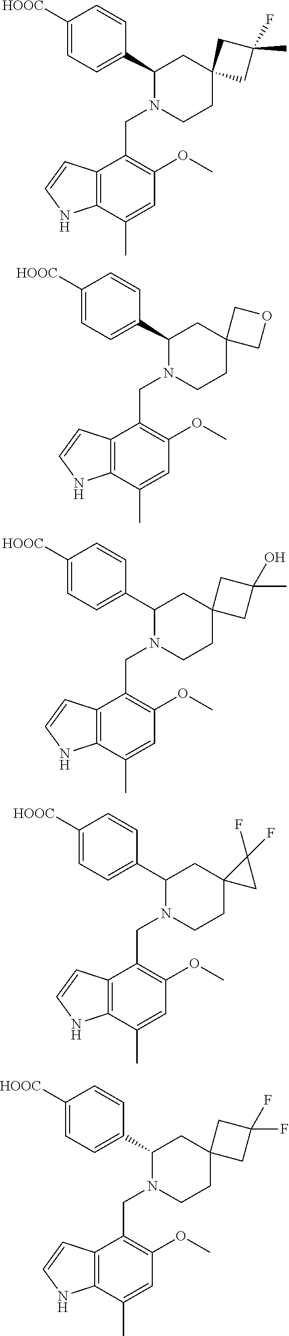

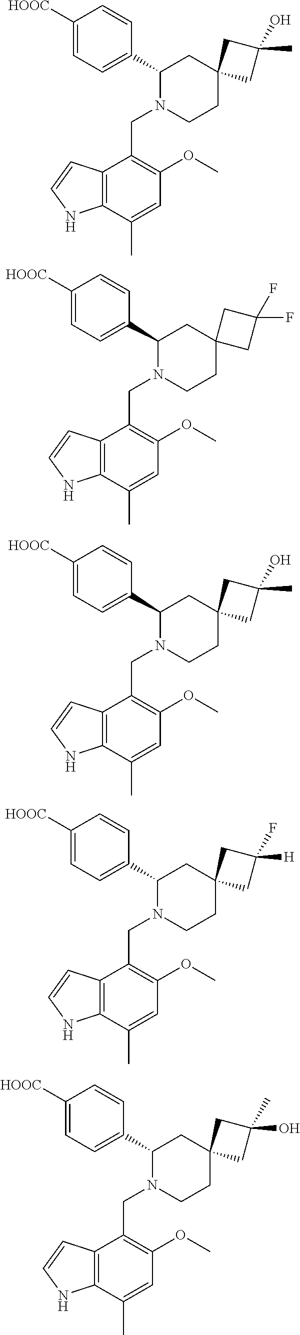

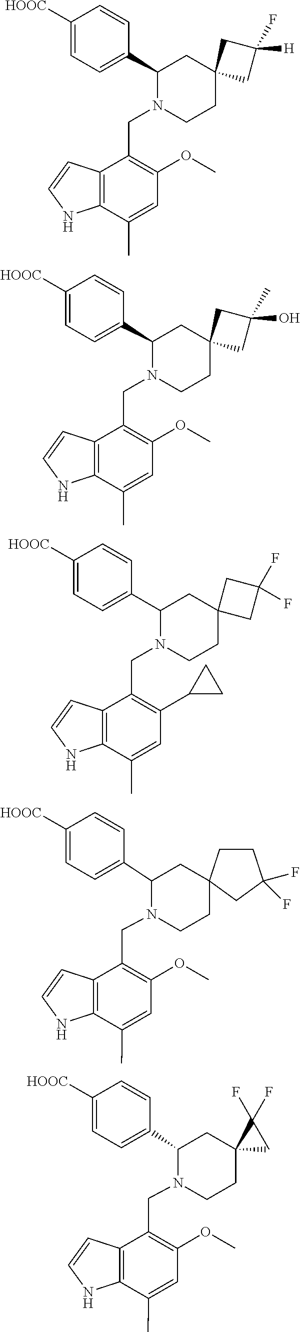

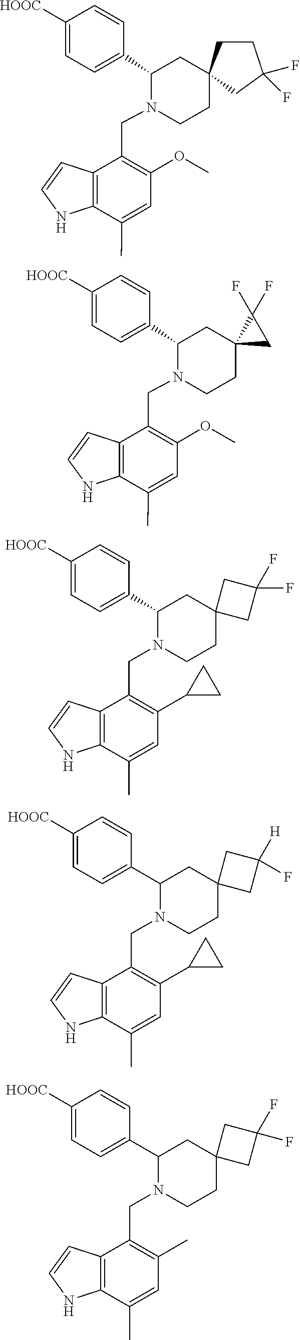

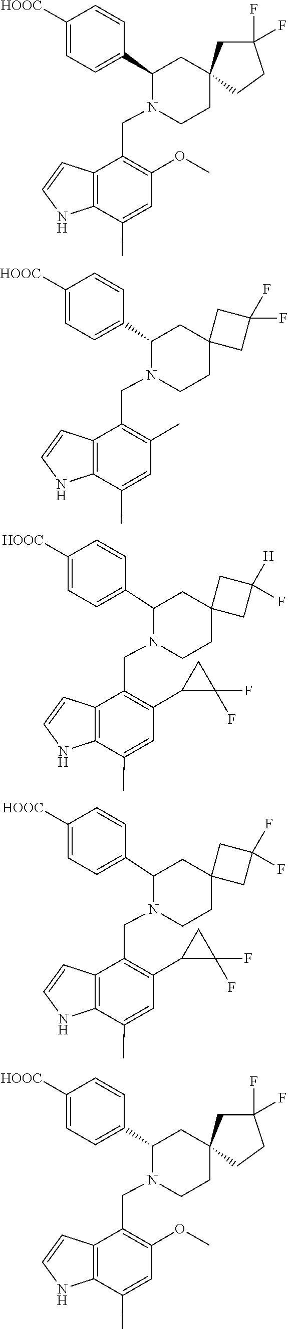

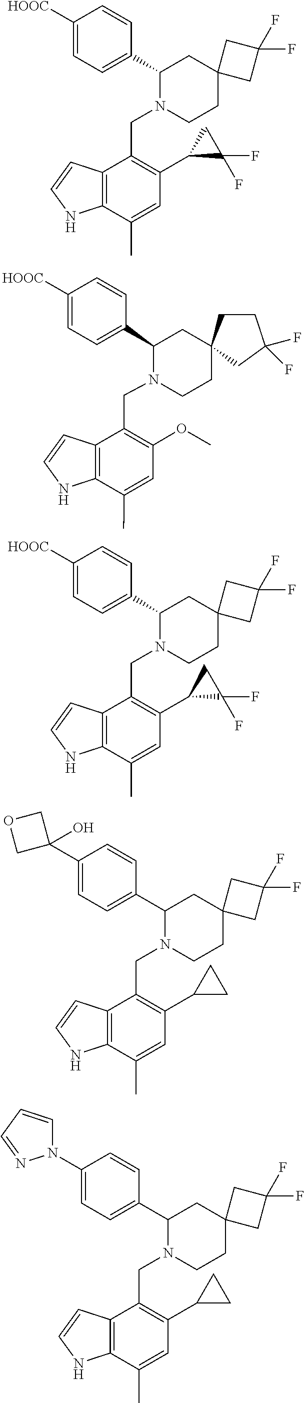

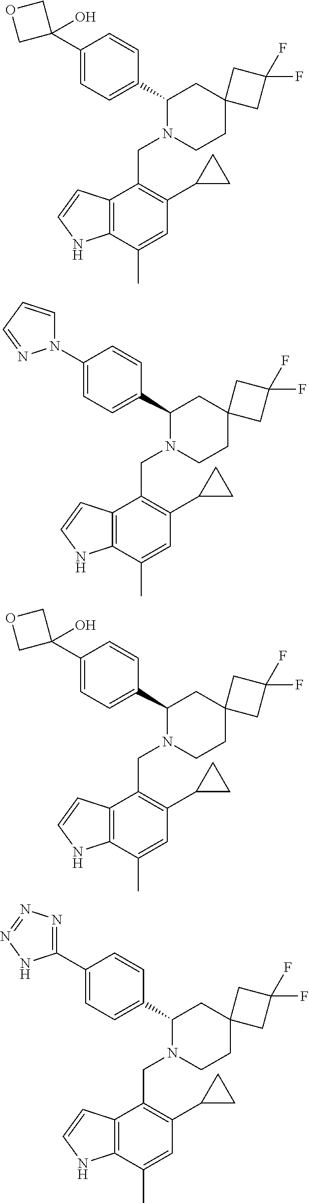 [SMILES: Cc1cc(C2CC2)c(CN2CCC3(C[C@H]2c2ccc(C4(O)COC4)cc2)CC(F)(F)C3)c2cc[nH]c12 ; Cc1cc(C2CC2)c(CN2CCC3(C[C@@H]2c2ccc(-n4cccn4)cc2)CC(F)(F)C3)c2cc[nH]c12 ; Cc1cc(C2CC2)c(CN2CCC3(C[C@@H]2c2ccc(C4(O)COC4)cc2)CC(F)(F)C3)c2cc[nH]c12 ; Cc1cc(C2CC2)c(CN2CCC3(C[C@H]2c2ccc(-c4nnn[nH]4)cc2)CC(F)(F)C3)c2cc[nH]c12]

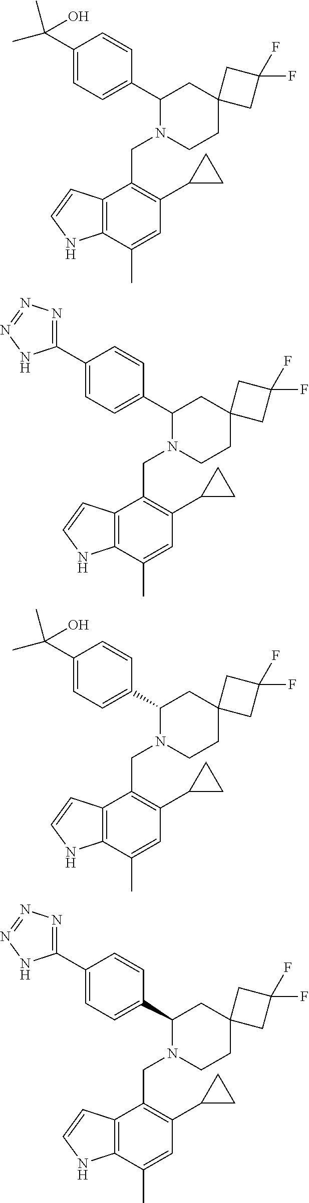 [SMILES: Cc1cc(C2CC2)c(CN2CCC3(CC2c2ccc(C(C)(C)O)cc2)CC(F)(F)C3)c2cc[nH]c12 ; Cc1cc(C2CC2)c(CN2CCC3(CC2c2ccc(-c4nnn[nH]4)cc2)CC(F)(F)C3)c2cc[nH]c12 ; Cc1cc(C2CC2)c(CN2CCC3(C[C@H]2c2ccc(C(C)(C)O)cc2)CC(F)(F)C3)c2cc[nH]c12 ; Cc1cc(C2CC2)c(CN2CCC3(C[C@@H]2c2ccc(-c4nnn[nH]4)cc2)CC(F)(F)C3)c2cc[nH]c12]

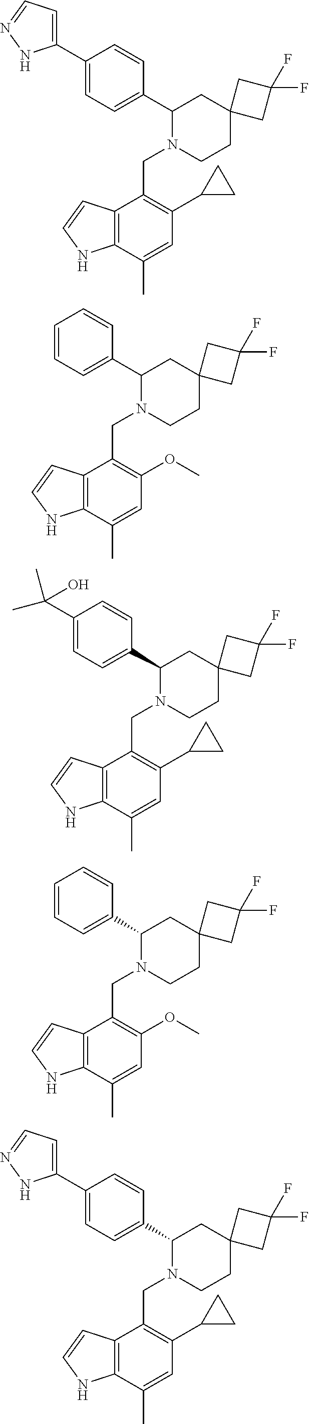

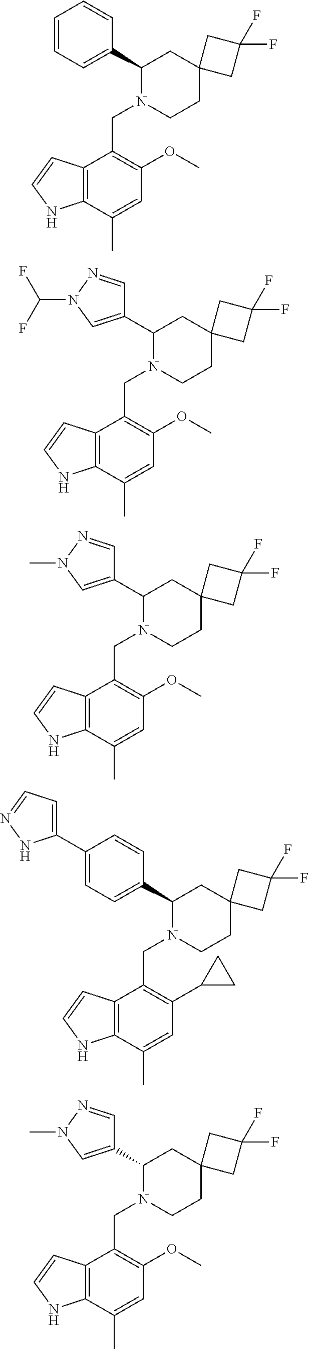

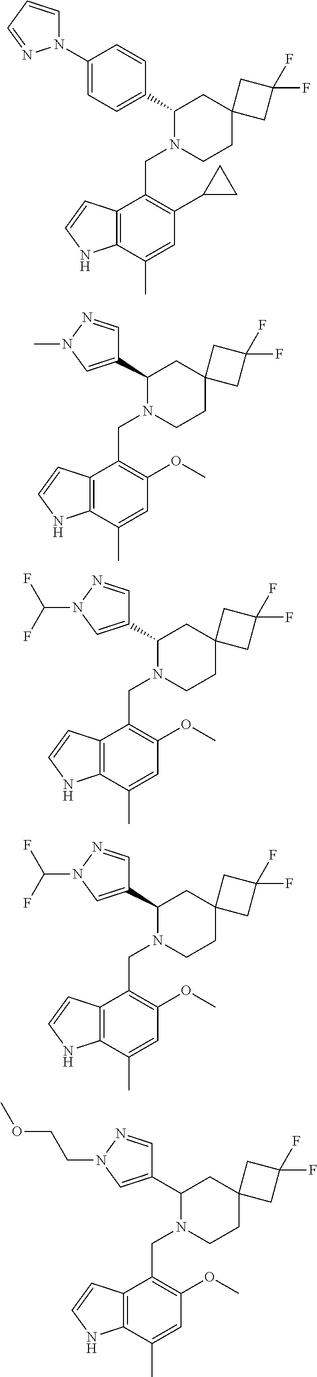

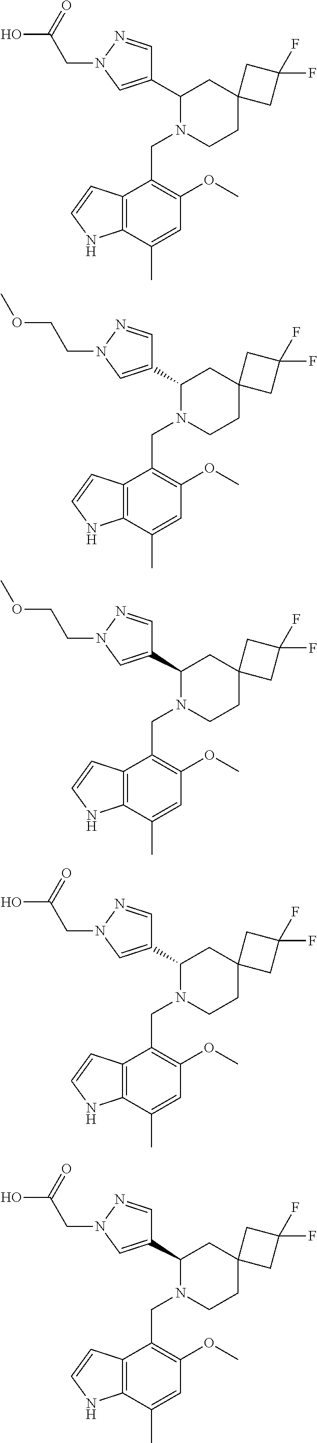 [SMILES: COc1cc(C)c2[nH]ccc2c1CN1CCC2(CC1c1cnn(CC(=O)O)c1)CC(F)(F)C2 ; COCCn1cc([C@@H]2CC3(CCN2Cc2c(OC)cc(C)c4[nH]ccc24)CC(F)(F)C3)cn1 ; COCCn1cc([C@H]2CC3(CCN2Cc2c(OC)cc(C)c4[nH]ccc24)CC(F)(F)C3)cn1 ; COc1cc(C)c2[nH]ccc2c1CN1CCC2(C[C@H]1c1cnn(CC(=O)O)c1)CC(F)(F)C2 ; COc1cc(C)c2[nH]ccc2c1CN1CCC2(C[C@@H]1c1cnn(CC(=O)O)c1)CC(F)(F)C2]

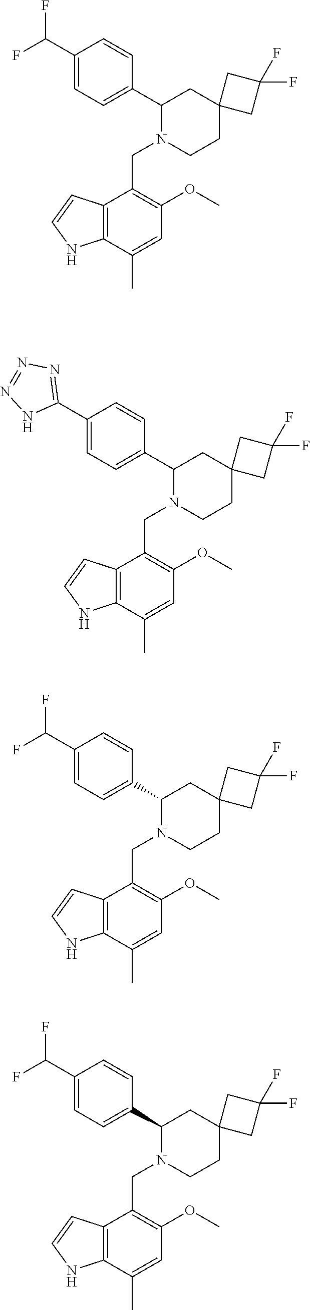 [SMILES: COc1cc(C)c2[nH]ccc2c1CN1CCC2(CC1c1ccc(C(F)F)cc1)CC(F)(F)C2 ; COc1cc(C)c2[nH]ccc2c1CN1CCC2(CC1c1ccc(-c3nnn[nH]3)cc1)CC(F)(F)C2 ; COc1cc(C)c2[nH]ccc2c1CN1CCC2(C[C@H]1c1ccc(C(F)F)cc1)CC(F)(F)C2 ; COc1cc(C)c2[nH]ccc2c1CN1CCC2(C[C@@H]1c1ccc(C(F)F)cc1)CC(F)(F)C2]

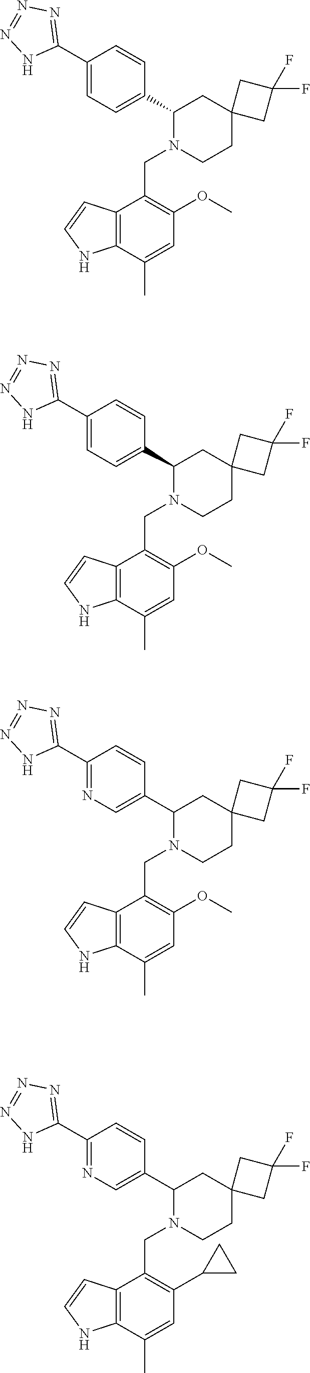 [SMILES: COc1cc(C)c2[nH]ccc2c1CN1CCC2(C[C@H]1c1ccc(-c3nnn[nH]3)cc1)CC(F)(F)C2 ; COc1cc(C)c2[nH]ccc2c1CN1CCC2(C[C@@H]1c1ccc(-c3nnn[nH]3)cc1)CC(F)(F)C2 ; COc1cc(C)c2[nH]ccc2c1CN1CCC2(CC1c1ccc(-c3nnn[nH]3)nc1)CC(F)(F)C2 ; Cc1cc(C2CC2)c(CN2CCC3(CC2c2ccc(-c4nnn[nH]4)nc2)CC(F)(F)C3)c2cc[nH]c12]

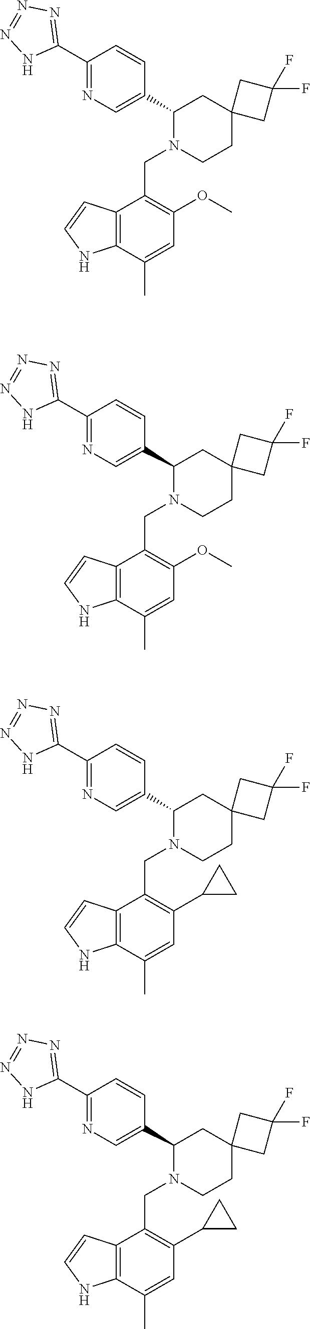 [SMILES: COc1cc(C)c2[nH]ccc2c1CN1CCC2(C[C@H]1c1ccc(-c3nnn[nH]3)nc1)CC(F)(F)C2 ; COc1cc(C)c2[nH]ccc2c1CN1CCC2(C[C@@H]1c1ccc(-c3nnn[nH]3)nc1)CC(F)(F)C2 ; Cc1cc(C2CC2)c(CN2CCC3(C[C@H]2c2ccc(-c4nnn[nH]4)nc2)CC(F)(F)C3)c2cc[nH]c12 ; Cc1cc(C2CC2)c(CN2CCC3(C[C@@H]2c2ccc(-c4nnn[nH]4)nc2)CC(F)(F)C3)c2cc[nH]c12]

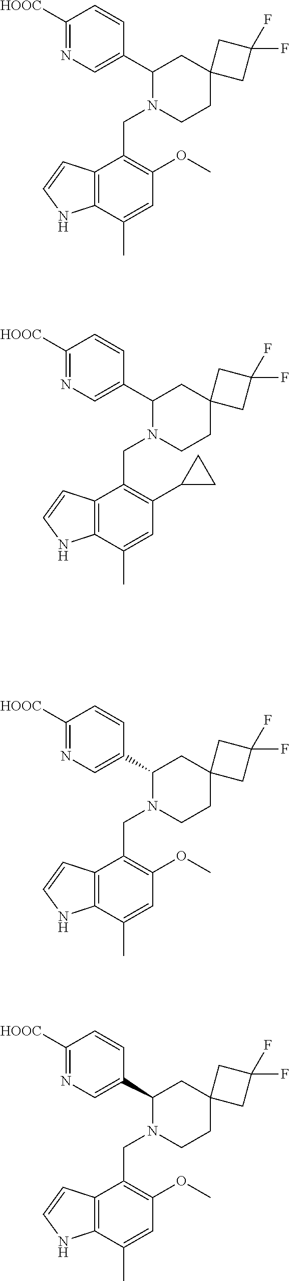 [SMILES: COc1cc(C)c2[nH]ccc2c1CN1CCC2(CC1c1ccc(C(=O)O)nc1)CC(F)(F)C2 ; Cc1cc(C2CC2)c(CN2CCC3(CC2c2ccc(C(=O)O)nc2)CC(F)(F)C3)c2cc[nH]c12 ; COc1cc(C)c2[nH]ccc2c1CN1CCC2(C[C@H]1c1ccc(C(=O)O)nc1)CC(F)(F)C2 ; COc1cc(C)c2[nH]ccc2c1CN1CCC2(C[C@@H]1c1ccc(C(=O)O)nc1)CC(F)(F)C2]

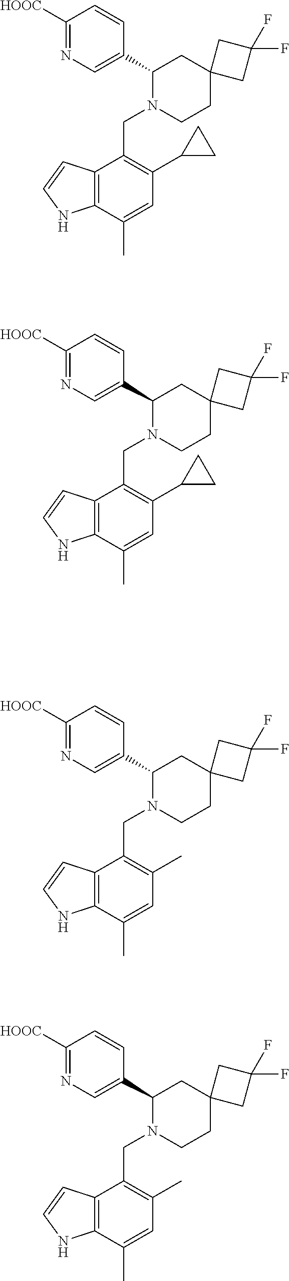 [SMILES: Cc1cc(C2CC2)c(CN2CCC3(C[C@H]2c2ccc(C(=O)O)nc2)CC(F)(F)C3)c2cc[nH]c12 ; Cc1cc(C2CC2)c(CN2CCC3(C[C@@H]2c2ccc(C(=O)O)nc2)CC(F)(F)C3)c2cc[nH]c12 ; Cc1cc(C)c2[nH]ccc2c1CN1CCC2(C[C@H]1c1ccc(C(=O)O)nc1)CC(F)(F)C2 ; Cc1cc(C)c2[nH]ccc2c1CN1CCC2(C[C@@H]1c1ccc(C(=O)O)nc1)CC(F)(F)C2]

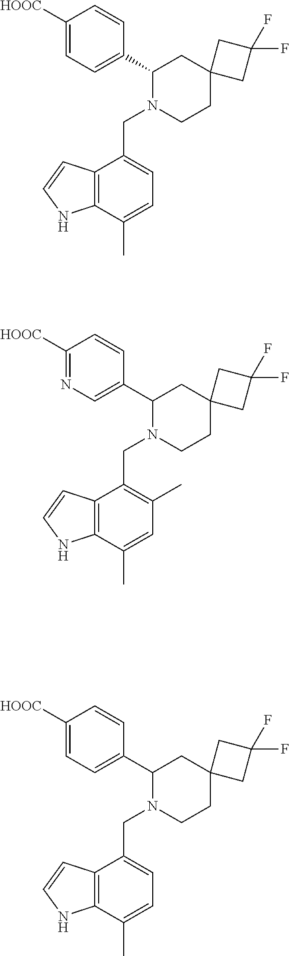 [SMILES: Cc1ccc(CN2CCC3(C[C@H]2c2ccc(C(=O)O)cc2)CC(F)(F)C3)c2cc[nH]c12 ; Cc1cc(C)c2[nH]ccc2c1CN1CCC2(CC1c1ccc(C(=O)O)nc1)CC(F)(F)C2 ; Cc1ccc(CN2CCC3(CC2c2ccc(C(=O)O)cc2)CC(F)(F)C3)c2cc[nH]c12]

26. The compound according to any one of the preceding claims, or a pharmaceutically acceptable salt thereof, wherein the compound is present in at least 90% enantiomeric excess, at least 95% enantiomeric excess, or at least 99% enantiomeric excess.

27. The compound according to any one of the preceding claims, or a pharmaceutically acceptable salt thereof, wherein the compound is present in at least 90% diastereomeric excess, at least 95% diastereomeric excess, or at least 99% diastereomeric excess.

28. A pharmaceutical composition comprising a therapeutically effective amount of a compound according to any one of the preceding claims, or a pharmaceutically acceptable salt thereof, and a pharmaceutically acceptable carrier or excipient.

29. A method of treating or preventing a disease or disorder in a subject in need thereof, the method comprising administering to the subject a therapeutically effective amount of a compound according to any one of claims 1 to 27, or a pharmaceutically acceptable salt thereof.

30. A method of modulating the complement alternative pathway activity in a subject, the method comprising administering to the subject a therapeutically effective amount of a compound according to any one of claims 1 to 27, or a pharmaceutically acceptable salt thereof.

31. A method of treating a disease or disorder mediated by complement activation, in particular mediated by activation of the complement alternative pathway, in a subject in need thereof, the method comprising administering to the subject a therapeutically effective amount of a compound according to any one of claims 1 to 27, or a pharmaceutically acceptable salt thereof.

32. A method of treating a disease or disorder that is affected by the modulation of complement alternative pathway comprising administering to the subject a therapeutically effective amount of a compound according to any one of claims 1 to 27, or a pharmaceutically acceptable salt thereof.

33. A method of treating a disease or disorder associated with dysregulation of the complement alternative pathway comprising administering to the subject a therapeutically effective amount of a compound according to any one of claims 1 to 27, or a pharmaceutically acceptable salt thereof.

34. A method of inhibiting the expression or activity of complement factor B, the method comprising administering to the subject a compound according to any one of claims 1 to 27, or a pharmaceutically acceptable salt thereof.

35. The method according to any one of claims 29 and 31 to 33, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

36. A method of treating age-related macular degeneration comprising administering to a subject in need thereof an effective amount of a composition comprising a compound according to any one of claims 1 to 27, or a pharmaceutically acceptable salt thereof.

37. A compound according to any one of claims 1 to 27, or a pharmaceutically acceptable salt thereof, for use as a medicament.

38. A compound according to any one of claims 1 to 27, or a pharmaceutically acceptable salt thereof, for use in inhibiting the expression or activity of complement factor B, in a subject in need thereof.

39. A compound according to any one of claims 1 to 27, or a pharmaceutically acceptable salt thereof, for use in treating a disease or disorder associated with dysregulation of the complement alternative pathway.

40. The compound for use according to claim 39, or a pharmaceutically acceptable salt thereof, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

41. Use of a compound according to any one of claims 1 to 27, or a pharmaceutically acceptable salt thereof, in the manufacture of a medicament for the treatment of a disease or disorder mediated by complement activation or activation of the complement alternative pathway.

42. Use of a compound according to any one of claims 1 to 27, or a pharmaceutically acceptable salt thereof, in the manufacture of a medicament for the treatment of a disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

43. Use of a compound according to any one of claims 1 to 27, or a pharmaceutically acceptable salt thereof, for the treatment of a disease or disorder mediated by complement activation or activation of the complement alternative pathway.

44. Use of a compound according to any one of claims 1 to 27, or a pharmaceutically acceptable salt thereof, for the treatment of a disease or disorder that is affected by the modulation of complement alternative pathway.

45. Use of a compound according to any one of claims 1 to 27, or a pharmaceutically acceptable salt thereof, for the treatment of a disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammatory diseases, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), IgA nephropathy, membranous nephropathy, including idiopathic membranous nephropathy, diabetic nephropathy, atypical hemolytic uremic syndrome, Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury, mesenteric artery reperfusion after aortic reconstruction, infectious disease or sepsis; COVID-19, immune complex disorders and autoimmune diseases, rheumatoid arthritis, osteoarthritis, Spondyloarthropathies including psoriatic arthritis, systemic lupus erythematosus (SLE), lupus nephritis, SLE nephritis, proliferative nephritis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis including anti-neutrophil cytoplasmic antibody (ANCA)-associated vasculitides, other vasculitides, including Henoch-Schönlein vasculitis, Buerger's vasculitis, cryoglobulinemia, Kawasaki disease, Takayasu arteritis, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenic purpura (TTP), abdominal aortic aneurisms, and Grave's disease.

46. Use of a compound according to any one of claims 1 to 25, or a pharmaceutically acceptable salt thereof, for the treatment of age-related macular degeneration.

47. A pharmaceutical combination comprising a compound according to any one of claims 1 to 25, or a pharmaceutically acceptable salt thereof, and one or more additional therapeutic agent(s).
